(19) United States
(12) Patent Application Publication
(10) Pub. No.: US 2026/0021095 A1
(43) Pub. Date: Jan. 22, 2026

(54) BIFUNCTIONAL COMPOUNDS CONTAINING PYRIDO[2,3-d]PYRIMIDIN-7(8H)-ONE DERIVATIVES FOR DEGRADING CYCLIN-DEPENDENT KINASE 2 VIA UBIQUITIN PROTEASOME PATHWAY

(71) Applicant: NIKANG THERAPEUTICS, INC., Wilmington, DE (US)

(72) Inventors: Yan LOU, Dallas, TX (US); Zhiyong YU, Wilmington, DE (US)

(21) Appl. No.: 18/875,529

(22) PCT Filed: Jun. 20, 2023
(86) PCT No.: PCT/US2023/025784

§ 371 Date: Dec. 16, 2024

Related U.S. Application Data
(60) Provisional application No. 63/354,669, filed on Jun. 22, 2022.

(51) Int. Cl. A61K 31/519; A61K 45/06; C07D 471/04

(52) U.S. Cl. CPC A61K 31/519; A61K 45/06; C07D 471/04

(57) ABSTRACT

The present disclosure provides certain bifunctional compounds that cause degradation of Cyclin-dependent kinase 2 (CDK2) via ubiquitin proteasome pathway and are therefore useful for the treatment of diseases mediated by CDK2. Also provided are pharmaceutical compositions containing such compounds and processes for preparing such compounds.

### CROSS-REFERENCE TO RELATED APPLICATIONS

This international application claims the benefit of U.S. Provisional Application No. 63/354,669 filed Jun. 22, 2022, the entire contents of which are incorporated herein for all purposes.

### FIELD OF THE DISCLOSURE

The present disclosure provides certain bifunctional compounds containing pyrido[2,3-d]-pyrimidin-7(8H)-one derivatives that cause degradation of Cyclin-dependent kinase 2 (CDK2) via ubiquitin proteasome pathway and are therefore useful for the treatment of diseases mediated by CDK2. Also provided are pharmaceutical compositions containing such compounds and processes for preparing such compounds.

### BACKGROUND

Cyclin-dependent kinases (CDKs) are cellular kinases that are critical for orchestrating signaling events such as DNA replication and protein synthesis to ensure faithful eukaryotic cell division and proliferation. To date, at least twenty-one mammalian CDKs have been identified (Malumbres M. Genome Biol. (2014) 15:122). Among these CDKs, at least CDK1/Cyclin B, CDK2/Cyclin E, CDK2/Cyclin A, CDK4/Cyclin D, and CDK6/Cyclin D complexes are known to be important regulators of cell cycle progression; while other CDKs are important in regulating gene transcription, DNA repair, differentiation and apoptosis (see Morgan, D. O. Annu. Rev. Cell. Dev. Biol. (1997) 13: 261-291).

Due to their roles in regulating cell cycle and other essential cellular processes, increased activity or temporally abnormal activation of CDKs has been shown to result in the development of various types of cancer. Human tumor development is commonly associated with alterations in either the CDK proteins themselves or their regulators (Cordon-Cardo C. Am. J. Pathol (1995) 147:545-560, Karp J E, Broder S. Nat. Med. (1995) 1:309-320; Hall M, Peters G. Adv. Cancer Res. (1996) 68:67-108). For example, amplifications of the regulatory subunits of CDKs and cyclins, and mutation, gene deletion, or transcriptional silencing of endogenous CDK inhibitory regulators have been reported (Smalley et al. Cancer Res. (2008) 68: 5743-52). A large body of research has established the role of these alterations in promoting tumorigenesis and progression. Thus, there has been great interest in the development of inhibitors of the Cyclin-dependent kinases (CDKs) for therapeutic purposes over the last two decades.

Selective CDK 4/6 inhibitors have changed the therapeutic management of hormone receptor-positive (HR+) metastatic breast cancer (MBC). Palbociclib, ribociclib, and abemaciclib, selective reversible inhibitors of CDK4 and CDK6, are approved for hormone receptor-positive (HR+) metastatic breast cancer in combination with endocrine therapies. Additional clinical trials with these CDK4/6 inhibitors are ongoing in both breast and other cancers, either as single agents or in combination with other therapeutics. (O'Leary et al. Nature Reviews (2016) 13:417-430). While CDK4/6 inhibitors have shown significant clinical efficacy in ER-positive metastatic breast cancer, the clinical benefit may be limited over time due to the development of primary or acquired resistance.

An important mechanism of resistance to CDK4/6 inhibitors is the abnormal activation of CDK2. It has been reported that high Cyclin E expression leads to overactivated CDK2/Cyclin E complex, which bypasses the requirement for CDK4/6 for cell cycle reentry (Asghar, U. et al. Clin. Cancer Res. (2017) 23:5561). In addition, it has been found that when CDK4/6 is inhibited, there is a noncanonical CDK2/cyclin D1 complex formation that promotes pRb phosphorylation recovery and drives cell cycle progression (Herrera-Abreu M T et al, Cancer Res. (2006) 15: 2301).

The CDK2/Cyclin E complex plays an important role in regulation of the G1/S transition, histone biosynthesis and centrosome duplication. Following the initial phosphorylation of Rb by Cdk4/6/cyclin D, Cdk2/Cyclin E further hyper-phosphorylates p-RB, releases E2F to transcribe genes required for S-phase entry. During S-phase, Cyclin E is degraded and CDK2 forms a complex with Cyclin A to promote phosphorylation of substrates that permit DNA replication and inactivation of E2F, for S-phase completion. (Asghar et al. Nat. Rev. Drug. Discov. (2015) 14: 130-146). In addition to cyclin bindings, the activity of CDK2 is also tightly regulated through its interaction with negative regulators, such as p21 and p27. In response to mitogenic stimulation, which signals optimal environment for cell cycle, p21 and p27 are phosphorylated and degraded, releasing the break on CDK2/Cyclin activation.

Cyclin E, the regulatory cyclin for CDK2, is frequently overexpressed in cancer, and its overexpression correlates with poor prognosis. For example, Cyclin E amplification or overexpression has been shown to associate with poor outcomes in breast cancer (Keyomarsi et al., N Engl J Med. (2002) 347:1566-75). Cyclin E2 (CCNE2) overexpression is associated with endocrine resistance in breast cancer cells and CDK2 inhibition has been reported to restore sensitivity to tamoxifen or CDK4/6 inhibitors in tamoxifen-resistant and CCNE2 overexpressing cells. (Caldon et al., Mol Cancer Ther. (2012) 11:1488-99; Herrera-Abreu et al, Cancer Res. (2016) 76:2301-2313). Cyclin E amplification also reportedly contributes to trastuzumab resistance in HER2+ breast cancer. (Scaltriti et al. Proc Natl Acad Sci. (2011) 108:3761-6). Cyclin E overexpression has also been reported to play a role in basal-like and triple negative breast cancer (TNBC), as well as inflammatory breast cancer (Elsawaf Z. et al. Breast Care (2011) 6:273-278, Alexander A. et al. Oncotarget (2017) 8:14897-14911.)

Amplification or overexpression of cyclin E1 (CCNE1) is also frequently found in ovarian, gastric, endometrial, uterus, bladder, esophagus, prostate, lung and other types of cancers (Nakayama et al. Cancer (2010) 116:2621-34; Etemadmoghadam et al. Clin Cancer Res (2013) 19: 5960-71; Au-Yeung et al. Chin. Cancer Res. (2017) 23:1862-1874; Ayhan et al. Modern Pathology (2017) 30: 297-303; Ooi et al. Hum Pathol. (2017) 61:58-67; Noske et al. Oncotarget (2017) 8: 14794-14805) and often correlates with poor clinical outcomes.

In some cancers loss-of-function mutations in FBXW7, a component of SCF^{Fbw7}ubiquitin E3 ligase responsible for cyclin E degradation, also leads to cyclin E overexpression and CDK2 activation. Alternatively, certain cancer cells express a hyperactive, truncated form of cyclin E. In addition, cyclin A amplification and overexpression have also been reported in various cancers such as hepatocellular carcinomas, colorectal and breast cancers.

In contrast to the frequent upregulation of Cyclin E, the inhibitory regulators of CDK2, p21 and p27 are often abnormally downregulated in cancers. It is postulated that the loss or decrease of these key endogenous inhibitors leads to high and/or abnormal temporal activation of CDK2, thereby promoting oncogenic growth.

In addition, CDC25A and CDC25B, protein phosphatases responsible for the dephosphorylations that activate the CDK2, are overexpressed in various tumors. These various mechanisms of CDK2 activation have been validated using mouse cancer models. Furthermore, CDK2/cyclin E phosphorylates oncogenic Myc to oppose ras-induced senescence, highlighting the importance of CDK2 in myc/ras-induced tumorigenesis. Inactivation of CDK2 has been shown to be synthetically lethal to Myc over-expressing cancer cells.

Recently, pharmacologic inhibition or genetic deletion of CDK2 was shown to preserve hearing function in animal models treated with cisplatin or noise (Teitz T et al. J Exp Med. 2018 Apr. 2; 215(4):1187-1203). Mechanistically, inhibition of CDK2 kinase activity reduces cisplatin-induced mitochondrial production of reactive oxygen species, thereby enhancing survival of inner ear cells. Therefore, in addition to anti-tumor therapies, CDK2 inhibition can also be used as a promising preventive treatment for noise-, cisplatin-, or antibiotic-induced or age-related hearing loss, for which no Food and Drug Administration-approved drugs are currently available.

Currently, there are a few CDK2 inhibitors in early phase of clinical trials. For example, Dinaciclib (MK-7%5) which inhibits CDK1, CDK2, CDK5 and CDK9 is in clinical development for solid tumors and hematological cancers in combination with other agents; CYC065, which potently inhibits CDK2, CDK3, CDK4, CDK9 and moderately inhibits CDK1, CDK5 and CDK7, is being investigated for the treatment of refractory CLL and other cancers; and PF-06873600, a CDK2 inhibitor with activities against other CDKs, is in clinical trial for the treatment of breast cancer either as single agent or in combination with endocrine therapies.

As an alternative to inhibition, removal of CDK2 protein would eliminate CDK2 activity as well as any protein interaction or scaffolding function of CDK2. Accordingly, there is a need for bifunctional molecules that could recruit CDK2 to a ubiquitin ligase and thereby causing ubiquitylation and proteasomal degradation of CDK2. The present disclosure fulfills this and related needs.

### SUMMARY

In a first aspect, provided is a compound of Formula (I):

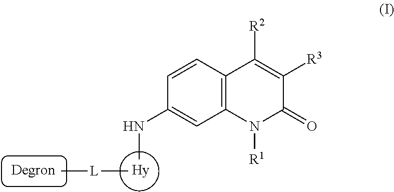

wherein:
Degron is an E3 ligase ligand selected from:
(a) a group of formula (i):

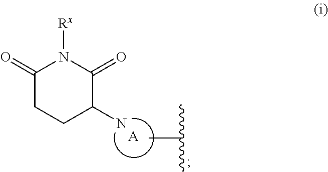

(b) a group of formula (ii):

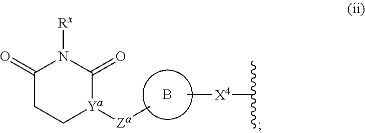

(c) a group of formula (iii):

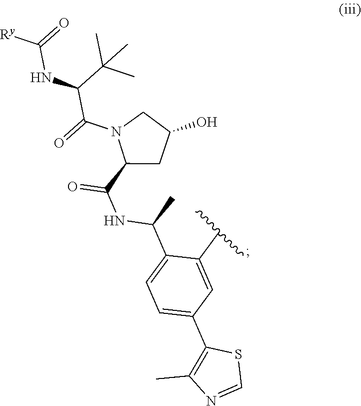

(d) a group of formula (iv):

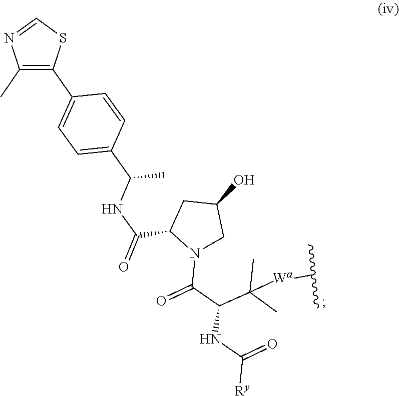

(e) a group of formula (v):

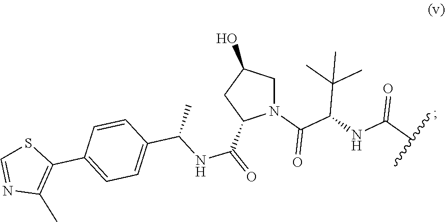 [SMILES: *C(=O)N[C@H](C(=O)N1C[C@H](O)C[C@H]1C(=O)N[C@@H](C)c1ccc(-c2scnc2C)cc1)C(C)(C)C]

and
(f) a group of formula (vi):

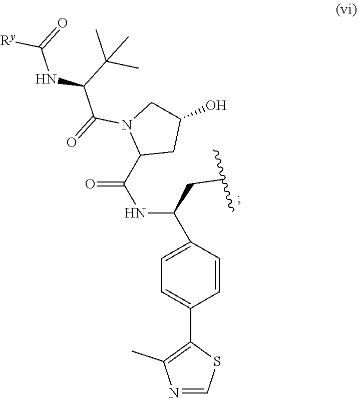

where:
   R^{x} is hydrogen, alkyl, cycloalkyl, or alkylcarbonyloxy;
   Y^{a} is CH or N;
   Z^{a} is a bond, —CH₂—, —NH—, O, or —NHC(O)— where NH of —NHC(O)— is attached to Y^{a};
   ring A of the E3 ligase ligand of formula (i) is a group of formula (a), (b), or (c):

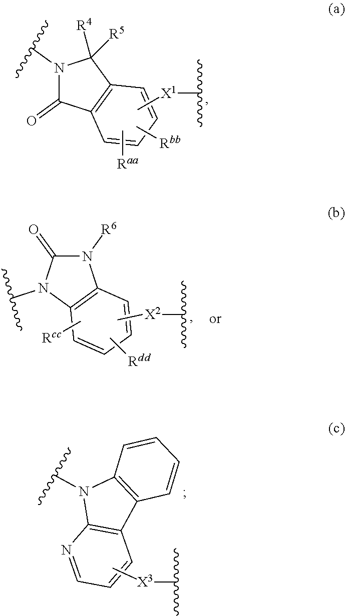

where:
   R^{aa}, R^{bb}, R^{cc}, and R^{dd} are independently selected from hydrogen, alkyl, alkoxy, halo, haloalkyl, haloalkoxy, and cyano;
   R⁴ and R⁵ are independently hydrogen or alkyl; or R⁴ and R⁵ together with the carbon to which they are attached form >C═O; and
   R⁶ is hydrogen or alkyl;
ring B of the E3 ligase ligand of formula (ii) is phenylene, cyclylaminylene, a 5- or 6-membered monocyclic heteroarylene, or a 9- or 10-membered fused bicyclic heteroarylene, wherein each heteroarylene ring contains one to three nitrogen ring atoms and further wherein the phenylene, cyclylaminylene, and each heteroarylene are independently substituted with R^{ee} and R^{ff} independently selected from hydrogen, alkyl, cycloalkyl, alkoxy, halo, haloalkyl, haloalkoxy, and cyano; and
X¹, X², X³, and X⁴ are independently a bon alkylene, —O—, —(O-alkylene)-, -(alkylene-O)—, —(NR^{gg}-alkylene)-, -(alkylene-NR^{hh})—,

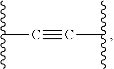 [SMILES: *C#C*]

—NH—, —N(alkyl)-, —C(═O)—, —NR^{jj}C(═O)—, or —C(═O)NR^{kk}— where R^{gg}, R^{hh}, R^{jj}, and R^{kk} independently hydrogen, alkyl, or cycloalkyl and each alkylene is optionally substituted with one or two fluoro;
R^{y} is alkyl, hydroxalkyl, cycloalkyl or heterocyclyl wherein cycloalkyl and heterocyclyl are substituted with R^{a} selected from hydrogen, halo, cyano, alkylcarbonyl, and alkylcarbonylamino; and
W^{a} is bond, O, S, or alkylene;
Hy is cycloalkylene, arylene, heterocyclylene, bicyclic heterocyclylene, spiro heterocyclylene, bridged heterocyclylene, or fused heterocyclylene, where each of the aforementioned rings is substituted with R^{b}, R^{c}, and R^{d} independently selected from hydrogen, deuterium, alkyl, halo, haloalkyl, alkoxy, hydroxy, and cyano;
R¹ is alkyl, haloalkyl, hydroxyalkyl, alkoxyalkyl, aminoalkyl, aryl, heteroaryl, aralkyl, heteroaralkyl, cycloalkylalkyl, or heterocyclylalkyl, wherein aryl, by itself or as part of aralkyl, heteroaryl, by itself or as part of heteroaralkyl, cycloalkyl of cycloalkylalkyl, and heterocyclyl of heterocyclylalkyl are substituted with R^{e}, R^{f}, and R^{g} independently selected from hydrogen, deuterium, alkyl, haloalkyl, alkoxy, haloalkoxy, cyano, halo, and hydroxy;
R² is hydrogen, alkyl, or haloalkyl;
R³ is hydrogen, cyano, halo, NH₂, alkyl, or haloalkyl where alkyl and haloalkyl are optionally substituted with hydroxy, cyano, alkoxy, haloalkoxy, C(O)NH₂, and —C(O)OH; and
L is —Z¹—Z²—Z³—Z⁴—Z⁵—Z⁶— where:
Z¹ is a bond, alkylene, —C(O)NR—, —NR′(CO)—, —S(O)₂NR—, —NR′S(O)₂—, —(O-alkylene)ₐ-, -(alkylene-O)ₐ—, phenylene, monocyclic heteroarylene, or heterocyclylene, where each ring is substituted with R^{h} and Rⁱ independently selected from hydrogen, deuterium, alkyl, alkoxy, halo, haloalkyl, and haloalkoxy;
Z² is a bond, alkylene, alkynylene, —C(O)—, —C(O)N(R)—, —NR′(CO)—, —(O-alkylene)_{b}-, -(alkylene-O)_{b}—, —O(CH₂)₇—, —O(CH₂)₈—, cycloalkylene, or heterocyclylene, where each ring is substituted with R^{j} and R^{k} independently selected from hydrogen, deuterium, alkyl, alkoxy, halo, haloalkyl, and haloalkoxy;
Z³ is a bond, alkylene, alkynylene, —C(O)NR—, —NR′(CO)—, —O—, —NR″—, —(O-alkylene)_{c}-, -(alkylene-O)_{c}—, cycloalkylene, spiro cyclolalkylene, phenylene, monocyclic heteroarylene, heterocyclylene, bicyclic heterocyclylene, bridged heterocyclylene, fused heterocyclylene, spiro heterocyclylene, or 11 to 13 membered spiro heterocyclylene, where each ring is substituted with R^{m} and Rⁿ independently selected from hydrogen, deuterium, alkyl, alkoxy, halo, haloalkyl, and haloalkoxy;
Z⁴ is a bond, alkylene, alkynylene, -(alkylene-NR″)—, —O—, —C(O)—, —NR″—, —(O-alkylene)_{d}-, -(alkylene-O)_{d}—, cycloalkylene, spiro cyclolalkylene, phenylene, heteroarylene, heterocyclylene, fused heterocyclylene, bridged heterocyclylene, or spiro heterocyclylene, where each ring is substituted with R^{o} and R^{p} independently selected from hydrogen, deuterium, alkyl, alkoxy, halo, haloalkyl, haloalkoxy, cyano, hydroxy, amino, alkylamino, and dialkylamino;
Z⁵ is a bond, alkylene, —NR″—, —O—, —C(O)—, —S(O)₂—, —NR′(CO)—, —C(O)NR—, phenylene, monocyclic heteroarylene, or heterocycylene, where each ring is substituted with R^{q} and R^{r} independently selected from hydrogen, deuterium, alkyl, alkoxy, halo, haloalkyl, and haloalkoxy, and
Z⁶ is a bond, alkylene, —NR″—, —O—, -(alkylene-O)—, —C(O)—, —S(O)₂—, —NR′(CO)—, or —C(O)NR—;
where each R, R′ and R″ is independently hydrogen or alkyl, each a, b, c, and d is independently an integer selected from 1 to 6, and each alkylene of —Z¹—, —Z²—, —Z³—, —Z⁴—, —Z⁵— and —Z⁶— is substituted with R^{s} and R^{t} where R^{s} is hydrogen or deuterium and R^{t} is hydrogen, deuterium, haloalkyl, hydroxy, alkoxy, cyano, cycloalkyl, heterocyclyl, aryl, or monocyclic heteroaryl, wherein cycloalkyl, heterocyclyl, aryl, and monocyclic heteroaryl are substituted with one or two substituents independently selected from hydrogen, alkyl, alkoxy, halo, haloalkyl, haloalkoxy, and cyano; provided that at least one of —Z¹—Z²—Z³—Z⁴—Z⁵—Z⁶— is not a bond; or
a pharmaceutically acceptable salt thereof.

In general, compounds of Formula (I) selectively inhibit CDK2 over CDK1. As such, in a second aspect, provided is a method of treating a disease mediated by CDK2 in a patient, preferably the patient is in need of such treatment, which method comprises administering to the patient, preferably a patient in need of such treatment, a therapeutically effective amount of a compound of Formula (I) (or any of the embodiments thereof described herein) or a pharmaceutically acceptable salt thereof. In a first embodiment of the second aspect, the disease is cancer. In a second subembodiment of the second aspect the disease is cancer selected from lung cancer (e.g. adenocarcinoma, small cell lung cancer and/or non-small cell lung carcinomas, parvicellular and non-parvicellular carcinoma, bronchial carcinoma, bronchial adenoma, pleuropulmonary blastoma), skin cancer (e.g., melanoma, squamous cell carcinoma, Kaposi sarcoma, and/or Merkel cell skin cancer), bladder cancer, breast cancer, cervical cancer, colorectal cancer, cancer of the small intestine, colon cancer, rectal cancer, cancer of the anus, endometrial cancer, gastric cancer, head and neck cancer (e.g., cancers of the larynx, hypopharynx, nasopharynx, oropharynx, lips, and/or mouth), liver cancer (e.g., hepatocellular carcinoma and/orcholangiocellular carcinoma), ovarian cancer, prostate cancer, testicular cancer, uterine cancer, esophageal cancer, gall bladder cancer, pancreatic cancer (e.g., exocrine pancreatic carcinoma), stomach cancer, thyroid cancer, and parathyroid cancer. In a third embodiment of the second aspect, the cancers are those that are resistant to CDK4/6 inhibitors through CDK2-mediated mechanisms. In a fourth embodiment of the second aspect, the therapeutically effective amount of a compound of Formula (I), or a pharmaceutically acceptable salt thereof, is administered in a pharmaceutical composition.

In a third aspect, provided is a method of treating noise-, cisplatin-, or antibiotic-induced-, or age-related hearing loss, which method comprises administering to the patient, preferably a patient in need of such treatment, a therapeutically effective amount of a compound of Formula (I) (or any of the embodiments thereof described herein) or a pharmaceutically acceptable salt thereof. In some embodiments, the amount of hearing loss is reduced when compared to an age-matched control. In some embodiments, the hearing loss is prevented when compared to an age-matched control.

In a fourth aspect, provided is a pharmaceutical composition comprising a compound of Formula (I) (or any of the embodiments thereof described herein) or a pharmaceutically acceptable salt thereof; and a pharmaceutically acceptable excipient.

In a fifth aspect, provided is a compound of Formula (I), (or any of the embodiments thereof described herein) or a pharmaceutically acceptable salt thereof for use as a medicament. In one embodiment, the compound of Formula (I) (and any of the embodiments thereof described herein) or a pharmaceutically acceptable salt thereof is useful for the treatment of one or more of diseases disclosed in the second aspect above.

In a sixth aspect, provided is the use of a compound of Formula (I) or a pharmaceutically acceptable salt thereof (and any of the embodiments thereof disclosed herein) in the manufacture of a medicament for treating a disease in a patient in which the activity of CDK2 contributes to the pathology and/or symptoms of the disease. In one embodiment the disease is one or more of diseases disclosed in the second aspect above.

In a seventh aspect, provided is a method of degrading CDK2 via ubiquitin proteasome pathway which method comprises contacting CDK2 with a compound of Formula (I) (or any of the embodiments thereof described herein) or a pharmaceutically acceptable salt thereof; or contacting CDK2 with a pharmaceutical composition comprising a compound of Formula (I) (or any of the embodiments thereof described herein) or a pharmaceutically acceptable salt thereof, and a pharmaceutically acceptable excipient. In some or any of the embodiments, the CDK2 is degraded in a cell or in a patient.

In the aforementioned aspect involving the treatment of cancer, further embodiments are provided comprising administering the compound of Formula (I) or a pharmaceutically acceptable salt thereof (or any of the embodiments thereof disclosed herein) in combination with at least one additional anticancer agent. When combination therapy is used, the agents can be administered simultaneously or sequentially.

### DETAILED DESCRIPTION

### Definitions

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like.

“Alkylcarbonyloxy” means an —OR^{z} group, where R^{z} is alkylcarbonyl, as defined herein.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkynylene” means a linear unsaturated divalent hydrocarbon radical of two to six carbon atoms or a branched saturated divalent hydrocarbon radical of three to six carbon atom containing a triple bond, e.g.,

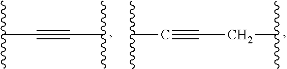

and the like.

“Alkylsulfonyl” means a —SO₂R^{z} radical where R^{z} is alkyl as defined above, e.g., methylsulfonyl, ethylsulfonyl, and the like.

“Alkylthio” means a —SR^{z} radical where R^{z} is alkyl as defined above, e.g., methylthio, ethylthio, and the like.

“Alkoxy” means a —OR^{z} radical where R^{z} is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with at least one alkoxy group, such as one or two alkoxy groups, as defined above, e.g., 2-methoxyethyl, 1-, 2-, or 3-methoxypropyl, 2-ethoxyethyl, and the like.

“Alkoxycarbonyl” means a —C(O)OR^{z} radical where R^{z} is alkyl as defined above, e.g., methoxycarbonyl, ethoxycarbonyl, and the like.

“Alkylcarbonylamino” means a —NR^{z′}C(O)R^{z} radical where R^{z} is alkyl and R^{z′} is H or alkyl, as defined above, e.g., methylcarbonylamino, ethylcarbonylamino, and the like.

“Acyl” means a —C(O)R^{z} radical where R^{z} is alkyl, haloalkyl, cycloalkyl, optionally substituted phenyl, optionally substituted heteroaryl, or optionally substituted heterocyclyl, as defined herein, e.g., methylcarbonyl, ethylcarbonyl, benzoyl, trifluoromethylcarbonyl, cyclopropylcarbonyl, and the like. When R^{z} is alkyl, acyl is also referred to herein as “alkylcarbonyl.”

“Amino” means a —NH₂.

“Alkylamino” means —NHR^{z} radical where R^{z} is alkyl as defined above e.g., methylamino, ethylamino, propylamino, and the like.

“Aminoalkyl” means a linear monovalent hydrocarbon radical of one to six carbon atoms or a branched monovalent hydrocarbon radical of three to six carbons substituted with —NR^{z′}R^{z″} where R^{z′} and R^{z″} are independently hydrogen, alkyl, deuteroalkyl, cycloalkyl, cycloalkylalkyl (wherein cycloalkyl and cycloalkyl ring in cycloalkylalkyl is optionally substituted with one, two, or three substituents independently selected from alkyl, hydroxyalkyl, haloalkyl, halo, hydroxy, alkoxy, —NH₂, alkylamino, dialkylamino, and cyano), hydroxyalkyl, alkoxyalkyl, haloalkoxyalkyl, alkylcarbonyl, optionally substituted aryl, optionally substituted heteroaryl, or optionally substituted heterocyclyl, each as defined herein, e.g., aminomethyl, aminoethyl, methylaminomethyl, and the like.

“Aryl” means a monovalent monocyclic or bicyclic aromatic hydrocarbon radical of 6 to 10 ring atoms e.g., phenyl or naphthyl.

“Arylene” means a divalent aryl (as defined above) radical e.g., phenylene or naphthylene.

“Aralkyl” means a -(alkylene)-R^{z} radical where R^{z} is aryl as defined above. Examples include, but are not limited to, benzyl, phenethyl, and the like.

“Bicyclic heterocyclylene” means a saturated or unsaturated divalent fused bicyclic group of 9 to 12 ring atoms in which one, two, or three ring atoms are heteroatoms independently selected from N, O, and S(O)ₙ, where n is an integer selected from 0 to 2 inclusive, the remaining ring atoms being carbon, unless stated otherwise. Additionally, one or two ring carbon atoms of the bicyclic heterocyclylene ring can optionally be replaced by a —CO— group. More specifically the term bicyclic heterocyclylene includes, but is not limited to, hexahydrofuro[3,2-b]furan-3,6-diyl, and the like. When the heterocyclylene ring is unsaturated it can contain one or two ring double bonds provided that the ring is not aromatic.

“Bridged heterocyclylene” means a saturated divalent bicyclic ring having 5 to 7 ring carbon ring atoms (exclusive of the atoms in the bridging group) in which two non-adjacent ring atoms are linked by a (CR^{z}R^{z′})ₙ group where n is an integer selected from 1 to 3 inclusive and R^{z} and R^{z′} are independently H or methyl (also may be referred to herein as “bridging” group) and further wherein one or two ring carbon atoms, including an atom in the bridging group, is replaced by a heteroatom selected from N, O, and S(O)ₙ, where n is an integer selected from 0 to 2 inclusive. Bridged heterocyclylene is optionally substituted with one or two substituents independently selected from alkyl, halo, alkoxy, hydroxy, and cyano unless stated otherwise. Examples include, but are not limited to, 3,8-diazabicyclo[3.2.1]octa-3,8-diyl, and the like.

“Cycloalkyl” means a monocyclic saturated monovalent hydrocarbon radical of three to ten carbon atoms. Examples include, but are not limited to, cyclopropyl, cyclobutyl, cyclopentyl, cyclohexyl, and the like.

“Cycloalkylalkyl” means an -(alkylene)-R^{z} radical where R^{z} is cycloalkyl as defined above. Examples include, but are not limited to, cyclopropylmethyl cyclobutylethyl, cyclopentylmethyl, cyclohexylmethyl, and the like.

“Cycloalkylene” means a divalent saturated hydrocarbon radical of three to six carbon atoms, otherwise e.g., 1,1-cyclopropylene, 1,1-cyclobutylene, 1,4-cyclohexylene, and the like.

“Cyanoalkyl” means a linear monovalent hydrocarbon radical of one to six carbon atoms or a branched monovalent hydrocarbon radical of three to six carbons substituted with cyano e.g., cyanomethyl, cyanoethyl, and the like.

“Carboxy” means —COOH.

“Cyclylaminylene” means a saturated divalent monocyclic ring of 4 to 8 ring atoms in which one ring atom is nitrogen, the remaining ring atoms being carbon. More specifically, the term cyclylaminyl includes, but is not limited to, pyrrolidinylene, piperidinylene, homopiperidinylene, and the like.

“Deuterium” mean refers to ²H or D.

“Deuteroalkyl” mean alkyl as defined above, which is substituted with one, two, or three deuterium.

“Dialkylamino” means —NR^{z′}R^{z′} radical where R^{z′} and R^{z″} is alkyl as defined above e.g., dimethylamino, diethylamino, methylpropylamino, and the like.

“Fused heterocyclylene” as used herein, refers to a divalent bicyclic ring in which two adjacent ring atoms of a saturated monocyclic ring of 4 to 7 ring atoms having one or two heteroatoms independently selected from N, O, and S(O)n (where n is 0, 1, or 2) and the remaining ring atoms being carbon, are fused to two adjacent ring members of phenyl or a five or six membered heteroaryl, each as defined herein, unless stated otherwise. The nitrogen atom is optionally oxidized or quaternized. The fused heterocyclylene can be attached at any two atoms of the ring. Representative examples include, but are not limited to, 1,2,3,4-tetrahydroquinolin-1,4-diyl, 3,4-dihydro-2H-benzo[b][1,4]oxazin-5,8-diyl, and the like.

“Halo” means fluoro, chloro, bromo, or iodo, preferably fluoro or chloro.

“Haloalkyl” means alkyl radical as defined above, which is substituted with one or more halogen atoms, e.g., one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkoxy” means a —OR^{z} radical where R^{z} is haloalkyl as defined above e.g., —OCF₃, —OCHF₂, and the like. When R^{z} is haloalkyl where the alkyl is substituted with only fluoro (in some examples, one or more fluoro), it is referred to in this Application as fluoroalkoxy.

“Haloalkoxyalkyl” means a -(alkylene)OR^{z} radical where R^{z} is haloalkyl as defined above, e.g., trifluoromethoxyalkyl, and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present, they are not both present on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Heteroaryl” means a monovalent monocyclic or fused bicyclic aromatic radical of 5 to 10 ring atoms, unless otherwise stated, where one or more, (in one embodiment, one, two, or three), ring atoms are heteroatom selected from N. O, and S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like. As defined herein, the terms “heteroaryl” and “aryl” are mutually exclusive. When the heteroaryl ring contains 5 or 6 ring atoms and is a monocyclic ring, it is also referred to herein as 5- or 6-membered monocyclic heteroaryl or monocyclic heteroaryl. When the heteroaryl ring contains 9- or 10 ring atoms and is a fused bicyclic ring, it is also referred to herein as 9- or 10-membered fused bicyclic heteroaryl.

“Heteroarylene” means a divalent heteroaryl radical as defined above, unless stated otherwise. Representative examples include, but are not limited to, benzimidazoldiyl e.g., benzimidazole-1,5-diyl, and the like. When the heteroarylene ring contains 5 or 6 ring atoms and is a monocyclic ring, it is also referred to herein as monocyclic heteroarylene or as 5- or 6-membered monocyclic heteroarylene e.g., pyrazolyl-1,4-diyl. When the heteroarylene ring contains 9- or 10 ring atoms and is a fused bicyclic ring, it is also referred to herein as 9- or 10-membered fused bicyclic heteroarylene.

“Heteroaralkyl” means a -(alkylene)-R^{z} radical where R^{z} is heteroaryl as defined above, e.g., pyridinylmethyl, and the like. When the heteroaryl ring in heteroaralkyl contains 5 or 6 ring atoms it is also referred to herein as 5- or 6-membered heteroaralkyl or monocyclic heteroaralkyl.

“Heterocyclyl” means a saturated or unsaturated monovalent monocyclic group of 4 to 8 ring atoms in which one or two ring atoms are heteroatom independently selected from N, O, and S(O)ₙ, where n is an integer selected from 0 to 2 inclusive, the remaining ring atoms being C, unless stated otherwise. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydro-pyranyl, thiomorpholino, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group.

“Heterocyclylalkyl” or “heterocycloalkyl” means a -(alkylene)-R^{z} radical where R^{z} is heterocyclyl ring as defined above e.g., tetraydrofuranylmethyl, piperazinylmethyl, morpholinylethyl, and the like.

“Heterocyclylene” means a saturated divalent monocyclic group of 4 to 6 ring atoms in which one or two ring atoms are heteroatom independently selected from N, O, and S(O)ₙ, where n is an integer selected from 0 to 2 inclusive, the remaining ring atoms being C, unless stated otherwise. Additionally, one or two ring carbon atoms in the heterocyclylene ring can optionally be replaced by a —CO— group. More specifically, the term heterocyclylene includes, but is not limited to,

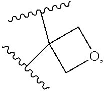 [SMILES: *C1(*)COC1]

piperidin-1,4-diyl, azetidin-1,3-diyl, and the like.

“Phenylene” refers to divalent phenyl.

The term “oxo,” as used herein, alone or in combination, refers to ═(O).

“Optionally substituted aryl” means aryl as defined above, that is optionally substituted with one, two, or three substituents independently selected from alkyl, hydroxyl, cycloalkyl, carboxy, alkoxycarbonyl, hydroxy, alkoxy, alkylthio, alkylsulfonyl, amino, alkylamino, dialkylamino, halo, haloalkyl, haloalkoxy, and cyano. In some embodiments, optionally substituted aryl is optionally substituted phenyl.

“Optionally substituted heteroaryl” means heteroaryl as defined above that is optionally substituted with one, two, or three substituents independently selected from alkyl, alkylthio, alkylsulfonyl, hydroxyl, cycloalkyl, carboxy, alkoxycarbonyl, hydroxy, alkoxy, halo, haloalkyl, haloalkoxy, amino, alkylamino, dialkylamino, and cyano.

“Optionally substituted heterocyclyl” means heterocyclyl as defined above that is optionally substituted with one, two, or three substituents independently selected from alkyl, alkylthio, alkylsulfonyl, alkylcarbonyl, hydroxyl, cycloalkyl, cycloalkylalkyl, carboxy, alkoxycarbonyl, hydroxy, hydroxyalkyl, alkoxy, alkoxyalkyl, aminoalkyl, cyanoalkyl, halo, haloalkyl, haloalkoxy, and cyano, unless stated otherwise.

The phrase “optionally” or “optional” as used herein means that the subsequently described event or circumstance may but need not occur, and that the description includes instances where the event or circumstance occurs and instances in which it does not. For example, the phrase “heteroaryl optionally substituted with alkyl” is intended to cover heteroaryl that is unsubstituted with alkyl and heteroaryl that is substituted with alkyl.

“Spiro cycloalkylene” means a saturated bicyclic divalent hydrocarbon ring having 6 to 12 ring atoms wherein the rings are connected through only one atom, the connecting atom is also called the spiroatom, most often a quaternary carbon (“spiro carbon”). Spiro cycloalkylene is optionally substituted with one or two substituents independently selected from alkyl, halo, alkoxy, hydroxy, and cyano, unless stated otherwise. Representative examples include, but are not limited to, spiro[3,5]nonandiyl e.g., spiro[3.5]nonane-2,7-diyl, and the like.

“Spiro heterocyclylene” means a saturated bicyclic divalent ring having 6 to 10 ring atoms in which one, two, or three ring atoms are heteroatom selected from N, O, and S(O)ₙ, where n is an integer selected from 0 to 2 inclusive, the remaining ring atoms being C and the rings are connected through only one atom, the connecting atom is also called the spiroatom, most often a quaternary carbon (“spiro carbon”). Spiro heterocyclylene is optionally substituted with one or two substituents independently selected from alkyl, halo, alkoxy, hydroxy, and cyano, unless stated otherwise.

“11 to 13 membered spiro heterocyclylene” means a saturated bicyclic divalent ring having 11 to 13 ring atoms in which one, two, or three ring atoms are heteroatom(s) selected from N, O, and S(O)ₙ, where n is an integer selected from 0 to 2 inclusive, the remaining ring atoms being C and the rings are connected through only one atom, the connecting atom is also called the spiroatom, most often a quaternary carbon (“spiro carbon”). The 11 to 13 membered spiro heterocyclylene is optionally substituted with one or two substituents independently selected from alkyl, halo, alkoxy, hydroxy, and cyano, unless stated otherwise. Representative examples include, but are not limited to, diazaspiro[5.5]undecan-diyl, 1-oxa-diazaspiro[5.5]undecan-diyl, and the like.

The present disclosure also includes protected derivatives of compounds of Formula (I). For example, when compounds of Formula (I) contain groups such as hydroxy, carboxy, or any group containing a nitrogen atom(s), these groups can be protected with suitable protecting groups. A comprehensive list of suitable protective groups can be found in T. W. Greene. Protective Groups in Organic Synthesis, 5^{th} Ed., John Wiley & Sons, Inc. (2014), the disclosure of which is incorporated herein by reference in its entirety. The protected derivatives of compounds of the present disclosure can be prepared by methods well known in the art.

The present disclosure also includes polymorphic forms and deuterated forms of the compound of Formula (I) or a pharmaceutically acceptable salt thereof.

The term “prodrug” refers to a compound that is made more active in vivo. Certain compounds of Formula (I) may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the active compound. Prodrugs are often useful because, in some situations, they may be easier to administer than the compound, or parent drug. They may, for instance, be bioavailable by oral administration whereas the parent drug is not. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

A “pharmaceutically acceptable salt” of a compound means a salt that is pharmaceutically acceptable and that possesses the desired pharmacological activity of the parent compound. Such salts include:
acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, PA, 1985, which is incorporated herein by reference in its entirety.

The compounds of Formula (I) may have asymmetric centers. Compounds of Formula (I) containing an asymmetrically substituted atom may be isolated in optically active or racemic forms. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. All chiral, diastereomeric, all mixtures of chiral or diastereomeric forms, and racemic forms are within the scope of this disclosure, unless the specific stereochemistry or isomeric form is specifically indicated. It will also be understood by a person of ordinary skill in the art that when a compound is denoted as (R) stereoisomer, it may contain the corresponding (S) stereoisomer as an impurity and vice versa.

Certain compounds of Formula (I) can exist as tautomers and/or geometric isomers. All possible tautomers and cis and trans isomers, as individual forms and mixtures thereof are within the scope of this disclosure. Additionally, as used herein the term alkyl includes all the possible isomeric forms of said alkyl group albeit only a few examples are set forth. Furthermore, when the cyclic groups such as aryl is substituted, it includes all the positional isomers albeit only a few examples are set forth. Furthermore, all hydrates of a compound of Formula (I) are within the scope of this disclosure.

The compounds of Formula (I) may also contain unnatural amounts of isotopes at one or more of the atoms that constitute such compounds. Unnatural amounts of an isotope may be defined as ranging from the amount found in nature to an amount 100% of the atom in question, that differ only in the presence of one or more isotopically enriched atoms. Exemplary isotopes that can be incorporated into compounds of the present disclosure, such as a compound of Formula (I) (and any embodiment thereof disclosed herein including specific compounds) include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³, and ¹²⁵I, respectively. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with (or isotopically enriched for) heavier isotopes such as deuterium (i.e., ²H) may afford certain therapeutic advantages resulting from greater metabolic stability (e.g., increased in vivo half-life or reduced dosage requirements). In some embodiments, in compounds of Formula (I), including in Table 1 or Table 1a below one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use.

“A pharmaceutically acceptable carrier/excipient” as used in the specification and claims includes both one and more than one such excipient.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

The phrase alkylene optionally substituted with one or two fluoro in the definition of X¹, X², X³, and X⁴ in Formula (I) (and similar phrases used to define other groups in Formula (I)) is intended to cover alkylene that is unsubstituted and alkylene that is substituted one or two fluoro.

Certain structures provided herein are drawn with one or more floating substituents. Unless provided otherwise or otherwise clear from the context, the substituent(s) may be present on any atom of the ring to which it is attached, where chemically feasible and valency rules permitting.

For example, in the structure:

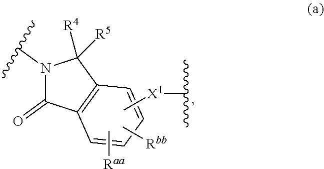

the R^{aa} substituent of R^{aa}, R^{bb} and X¹, and similarly the R^{bb} and X¹ substituents, can replace hydrogen of any CH that is part of the benzo portion of the bicyclic ring that is not already substituted with R^{bb} and X¹, and similarly R^{aa} and X¹, and R^{aa} and R^{bb} substituents with respect to R^{bb} and X¹, respectively.

Additionally, as used throughout the application, including in the embodiments, when a group is drawn out as divalent, the left bond of the divalent group is attached to the group which is to its left in the remainder of the molecule, and the right bond of the divalent group is attached to the group which is to its right in the remainder of the molecule. For example, in the following divalent groups

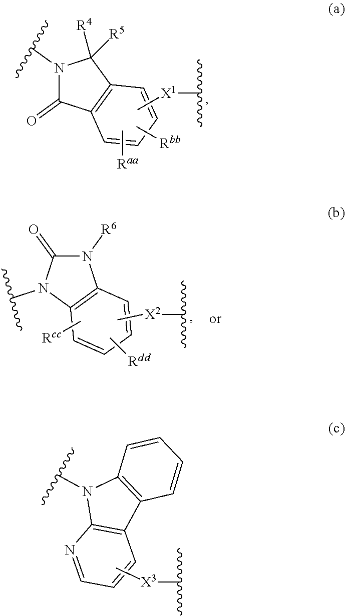

the

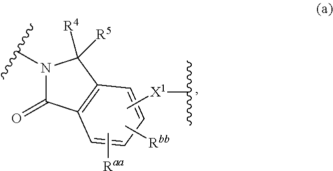

bond on the left of (a), (b) and (c) is attached to the following ring:

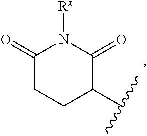

and the

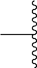

on the right side of (a), (b), and (c) (i.e., X¹, X², and X³) is attached to Z¹ of L of the following structure:

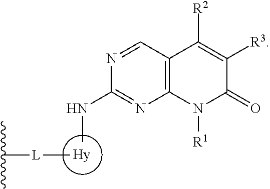

Similarly, for L i.e. —Z¹—Z²—Z³—Z⁴—Z⁵—Z⁶—, the left side in L (i.e., Z¹) is attached to X¹, X², X³, or X⁴ and Z⁶ is attached to an atom of Hy. For example, when L is a group of formula:

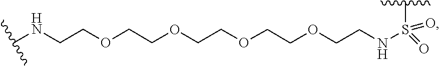 [SMILES: *NCCOCCOCCOCCOCCNS(*)(=O)=O]

and Degron is a group of formula (a), i.e.,

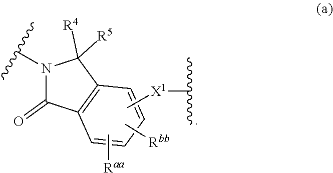

the left bond of L (i.e., the —NH— group) is attached to X¹ and the right hand bond of L (i.e., —SO₂—) is attached to an atom of the Hy as indicated in the following

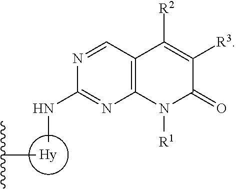

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a disease or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

“Treating” or “treatment” of a disease includes:
(1) preventing the disease, i.e., causing the clinical symptoms of the disease not to develop in a mammal that may be exposed to or predisposed to the disease but does not yet experience or display symptoms of the disease;
(2) inhibiting the disease, i.e., delaying, arresting or reducing the development or severity of the disease or its clinical symptoms; or
(3) relieving the disease, i.e., causing regression of the disease or its clinical symptoms.

In one embodiment, treating or treatment of a disease includes inhibiting the disease, i.e., delaying, arresting or reducing the development or severity of the disease or its clinical symptoms; or relieving the disease, i.e., causing regression of the disease or its clinical symptoms.

A “therapeutically effective amount” means the amount of a compound of the present disclosure and/or a pharmaceutically acceptable salt thereof that, when administered to a patient for treating a disease, is sufficient to affect such treatment for the disease. The “therapeutically effective amount” will vary depending on the compound, the disease and its severity and the age, weight, etc., of the mammal to be treated.

The terms “inhibiting” and “reducing,” or any variation of these terms in relation of CDK2 and/or CDK1, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of CDK2 and/or CDK1 activity respectively, compared to normal.

Representative compounds of the disclosure made are disclosed in Compound Table 1 below:

COMPOUND TABLE 1

| Cpd | | |
|---|---|---|
| # | Structure | Name |
| 1 | 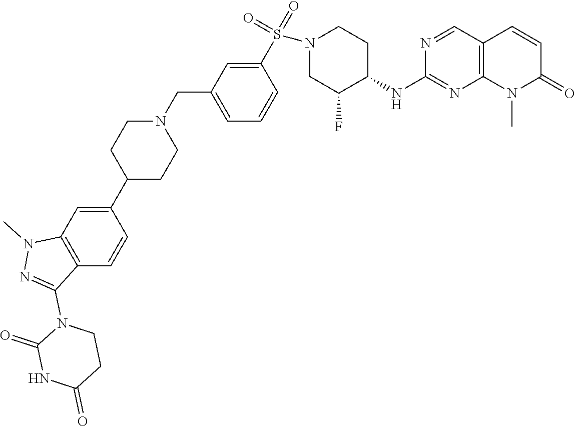 [SMILES: Cn1nc(N2CCC(=O)NC2=O)c2ccc(C3CCN(Cc4cccc(S(=O)(=O)N5CC[C@H](Nc6ncc7ccc(=O)n(C)c7n6)[C@H](F)C5)c4)CC3)cc21] | Rac-1-(6-(1-(3-(((3R,4S)-3-fluoro-4-((8-methyl- 7-oxo-7,8-dihydropyrido[2,3-d]pyrimidin-2- yl)amino)piperidin-1-yl)sulfonyl)benzyl)- piperidin-4-yl)-1-methyl-1H-indazol-3-yl)- dihydropyrimidine-2,4(1H,3H)-dione |
| 2 | 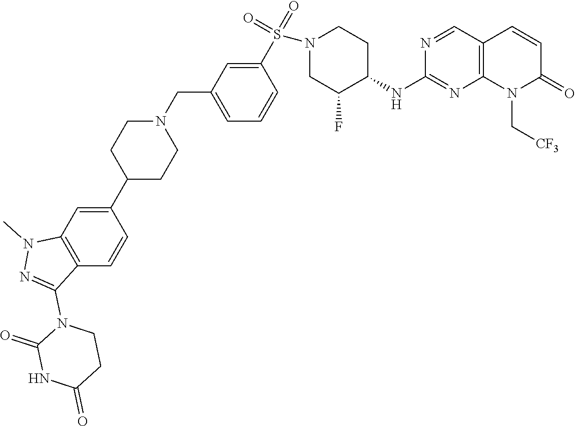 [SMILES: Cn1nc(N2CCC(=O)NC2=O)c2ccc(C3CCN(Cc4cccc(S(=O)(=O)N5CC[C@H](Nc6ncc7ccc(=O)n(CC(F)(F)F)c7n6)[C@H](F)C5)c4)CC3)cc21] | Rac-1-(6-(1-(3-(((3R,4S)-3-fluoro-4-((7-oxo-8- (2,2,2-trifluoroethyl)-7,8-dihydropyrido[2,3- d]pyrimidin-2-yl)amino)piperidin-1-yl)- sulfonyl)benzyl)piperidin-4-yl)-1-methyl-1H- indazol-3-yl)dihydropyrimidine-2,4(1H,3H)- dione |

Contemplated compounds of Formula (I) are disclosed in Table 1a below:

TABLE la

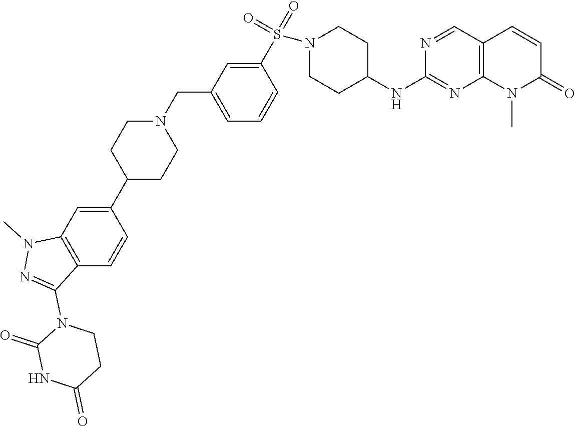 [SMILES: Cn1nc(N2CCC(=O)NC2=O)c2ccc(C3CCN(Cc4cccc(S(=O)(=O)N5CCC(Nc6ncc7ccc(=O)n(C)c7n6)CC5)c4)CC3)cc21]

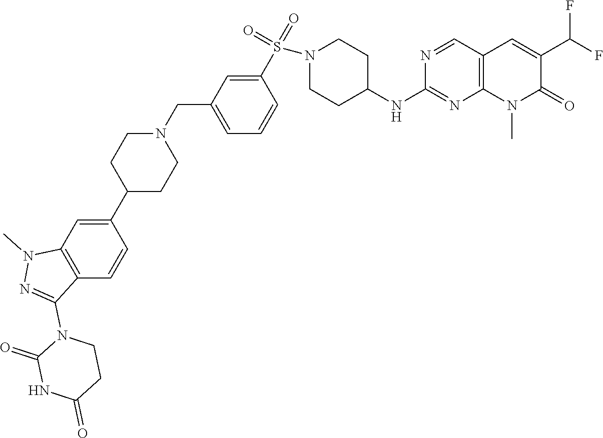 [SMILES: Cn1nc(N2CCC(=O)NC2=O)c2ccc(C3CCN(Cc4cccc(S(=O)(=O)N5CCC(Nc6ncc7cc(C(F)F)c(=O)n(C)c7n6)CC5)c4)CC3)cc21]

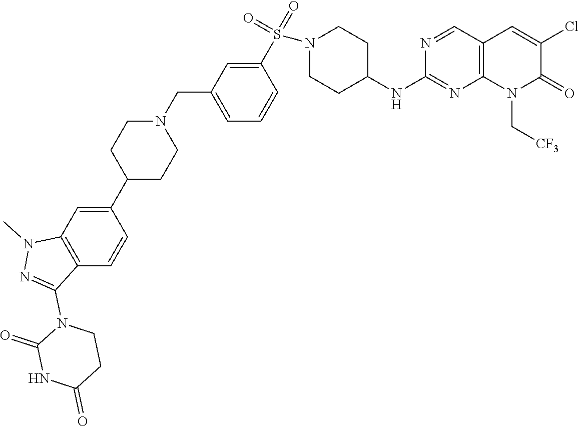 [SMILES: Cn1nc(N2CCC(=O)NC2=O)c2ccc(C3CCN(Cc4cccc(S(=O)(=O)N5CCC(Nc6ncc7cc(Cl)c(=O)n(CC(F)(F)F)c7n6)CC5)c4)CC3)cc21]

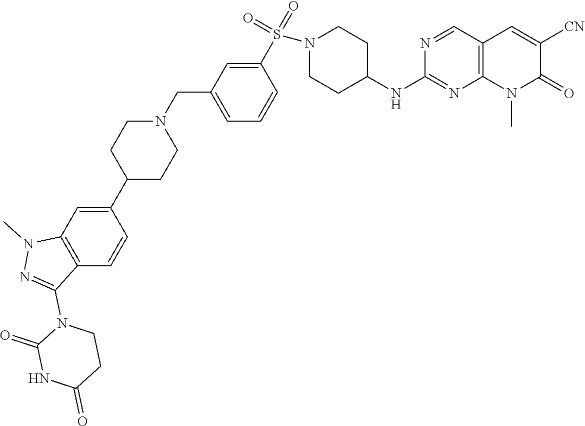 [SMILES: Cn1nc(N2CCC(=O)NC2=O)c2ccc(C3CCN(Cc4cccc(S(=O)(=O)N5CCC(Nc6ncc7cc(C#N)c(=O)n(C)c7n6)CC5)c4)CC3)cc21]

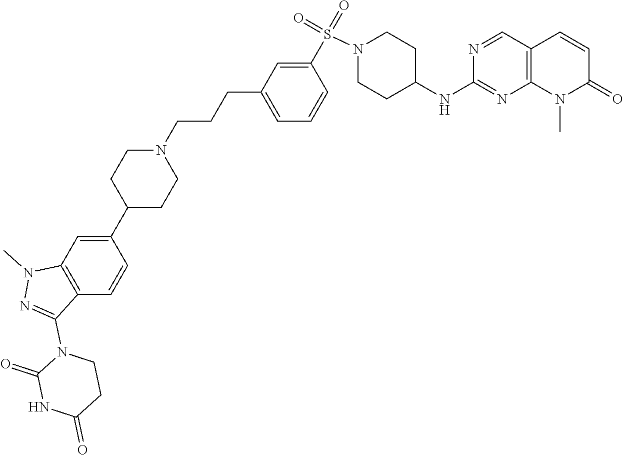 [SMILES: Cn1nc(N2CCC(=O)NC2=O)c2ccc(C3CCN(CCCc4cccc(S(=O)(=O)N5CCC(Nc6ncc7ccc(=O)n(C)c7n6)CC5)c4)CC3)cc21]

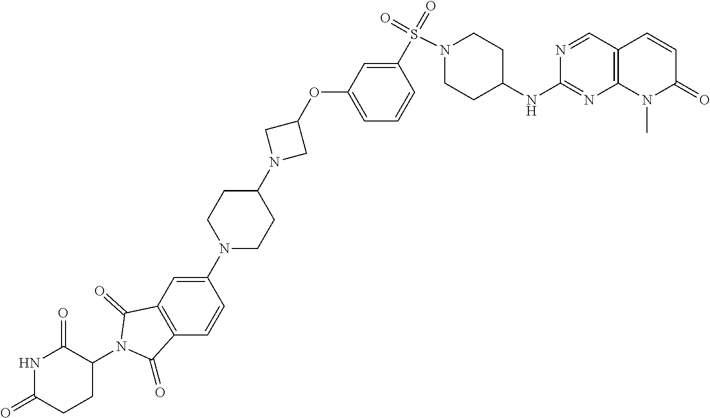 [SMILES: Cn1c(=O)ccc2cnc(NC3CCN(S(=O)(=O)c4cccc(OC5CN(C6CCN(c7ccc8c(c7)C(=O)N(C7CCC(=O)NC7=O)C8=O)CC6)C5)c4)CC3)nc21]

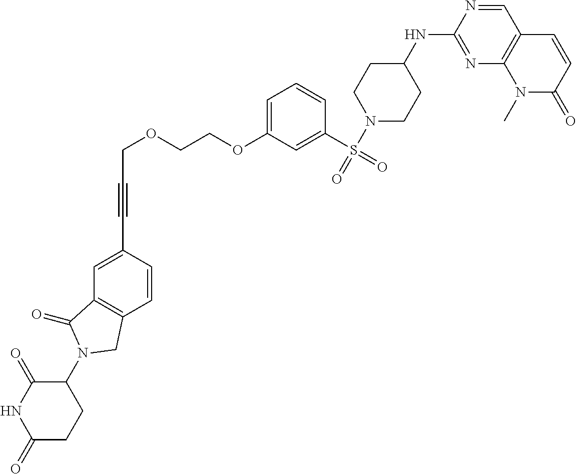 [SMILES: Cn1c(=O)ccc2cnc(NC3CCN(S(=O)(=O)c4cccc(OCCOCC#Cc5ccc6c(c5)C(=O)N(C5CCC(=O)NC5=O)C6)c4)CC3)nc21]

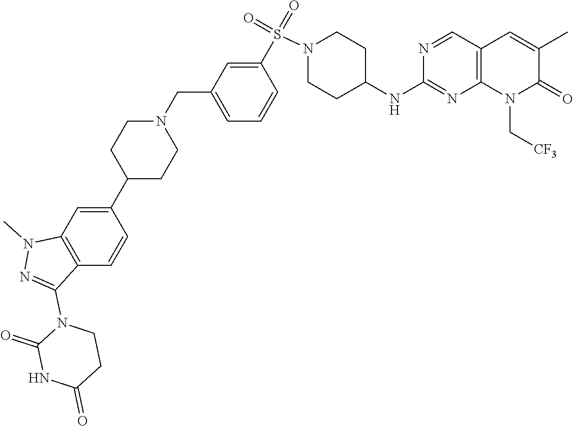

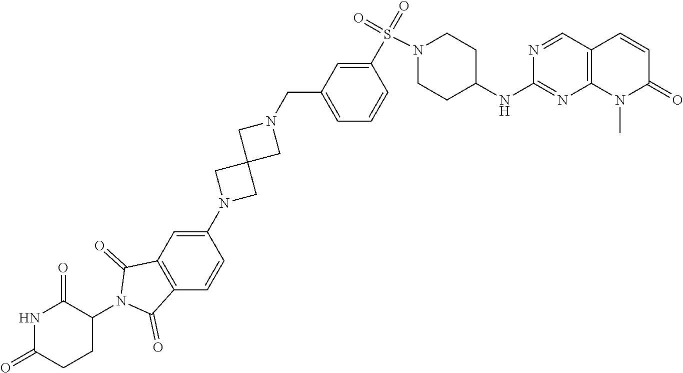 [SMILES: Cn1c(=O)ccc2cnc(NC3CCN(S(=O)(=O)c4cccc(CN5CC6(C5)CN(c5ccc7c(c5)C(=O)N(C5CCC(=O)NC5=O)C7=O)C6)c4)CC3)nc21]

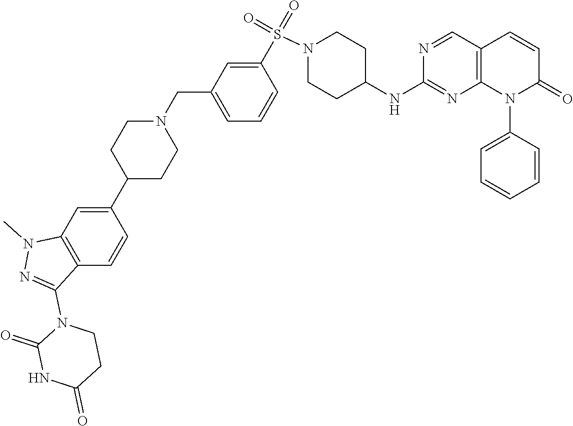 [SMILES: Cn1nc(N2CCC(=O)NC2=O)c2ccc(C3CCN(Cc4cccc(S(=O)(=O)N5CCC(Nc6ncc7ccc(=O)n(-c8ccccc8)c7n6)CC5)c4)CC3)cc21]

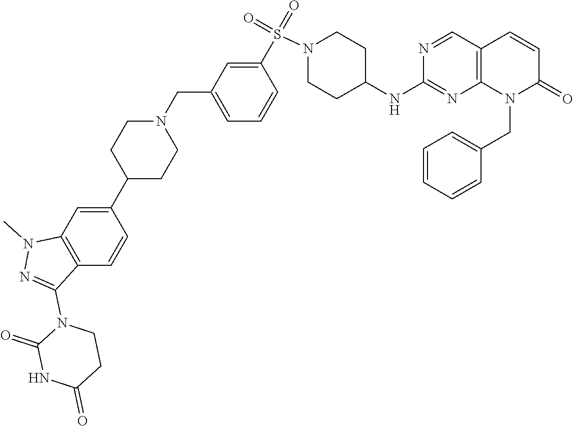 [SMILES: Cn1nc(N2CCC(=O)NC2=O)c2ccc(C3CCN(Cc4cccc(S(=O)(=O)N5CCC(Nc6ncc7ccc(=O)n(Cc8ccccc8)c7n6)CC5)c4)CC3)cc21]

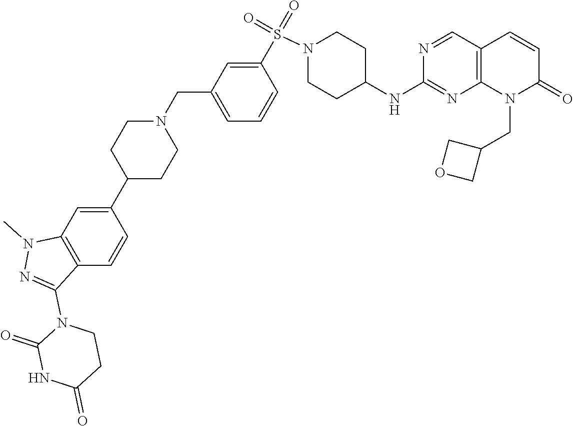 [SMILES: Cn1nc(N2CCC(=O)NC2=O)c2ccc(C3CCN(Cc4cccc(S(=O)(=O)N5CCC(Nc6ncc7ccc(=O)n(CC8COC8)c7n6)CC5)c4)CC3)cc21]

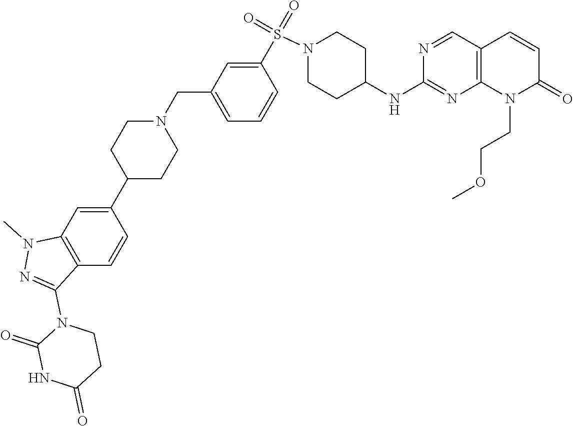 [SMILES: COCCn1c(=O)ccc2cnc(NC3CCN(S(=O)(=O)c4cccc(CN5CCC(c6ccc7c(N8CCC(=O)NC8=O)nn(C)c7c6)CC5)c4)CC3)nc21]

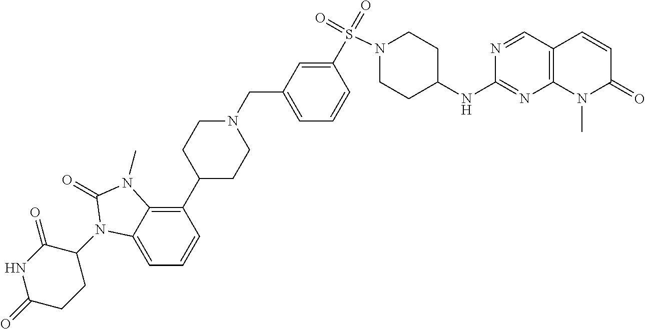 [SMILES: Cn1c(=O)ccc2cnc(NC3CCN(S(=O)(=O)c4cccc(CN5CCC(c6cccc7c6n(C)c(=O)n7C6CCC(=O)NC6=O)CC5)c4)CC3)nc21]

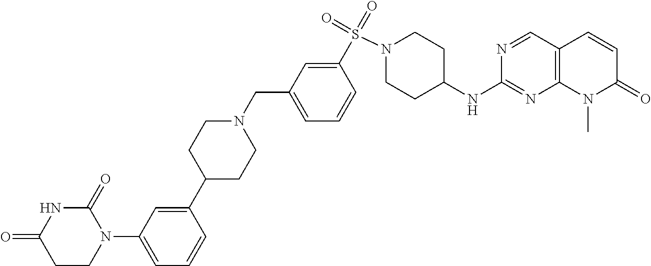 [SMILES: Cn1c(=O)ccc2cnc(NC3CCN(S(=O)(=O)c4cccc(CN5CCC(c6cccc(N7CCC(=O)NC7=O)c6)CC5)c4)CC3)nc21]

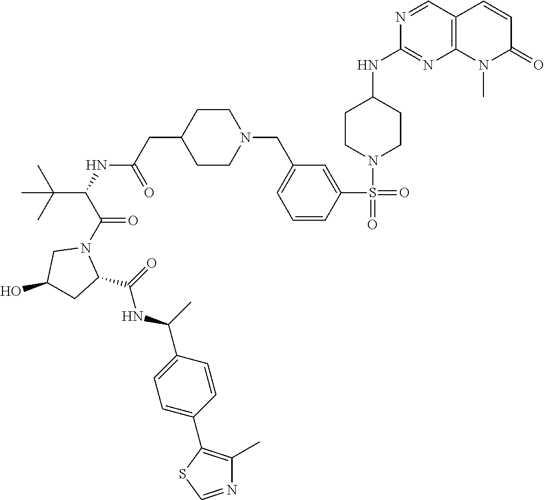 [SMILES: Cc1ncsc1-c1ccc([C@H](C)NC(=O)[C@@H]2C[C@@H](O)CN2C(=O)[C@@H](NC(=O)CC2CCN(Cc3cccc(S(=O)(=O)N4CCC(Nc5ncc6ccc(=O)n(C)c6n5)CC4)c3)CC2)C(C)(C)C)cc1]

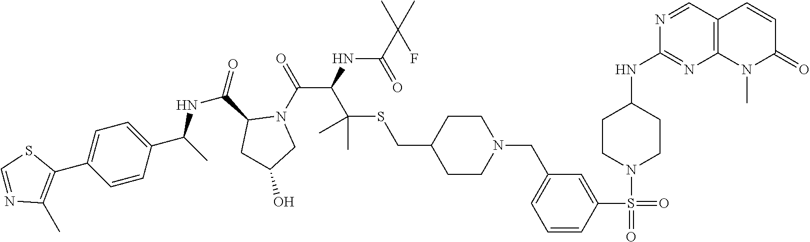 [SMILES: Cc1ncsc1-c1ccc([C@H](C)NC(=O)[C@@H]2C[C@@H](O)CN2C(=O)[C@@H](NC(=O)C(C)(C)F)C(C)(C)SCC2CCN(Cc3cccc(S(=O)(=O)N4CCC(Nc5ncc6ccc(=O)n(C)c6n5)CC4)c3)CC2)cc1]

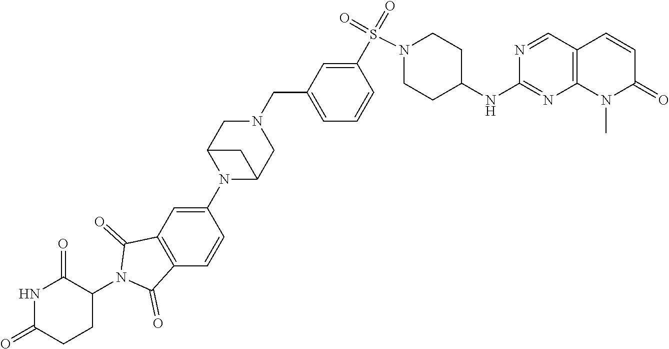 [SMILES: Cn1c(=O)ccc2cnc(NC3CCN(S(=O)(=O)c4cccc(CN5CC6CC(C5)N6c5ccc6c(c5)C(=O)N(C5CCC(=O)NC5=O)C6=O)c4)CC3)nc21]

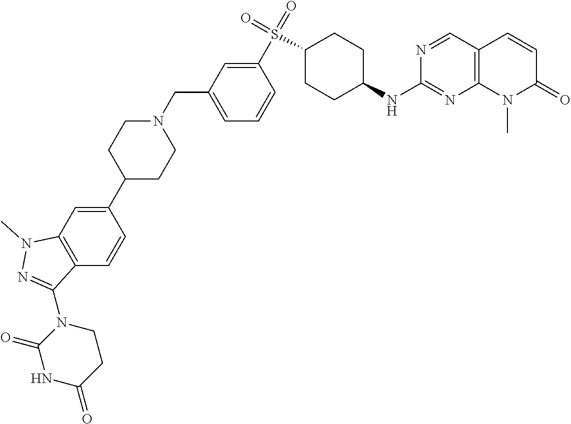 [SMILES: Cn1nc(N2CCC(=O)NC2=O)c2ccc(C3CCN(Cc4cccc(S(=O)(=O)[C@H]5CC[C@H](Nc6ncc7ccc(=O)n(C)c7n6)CC5)c4)CC3)cc21]

### EMBODIMENTS

In embodiments A1 to A152, the present disclosure includes:

A1. In embodiment A1, provided is a compound of Formula (I) or a pharmaceutically acceptable salt as defined in the first aspect of the Summary.

A2. In embodiment A2, the compound of embodiment A1, or a pharmaceutically acceptable salt thereof, is wherein R¹ is alkyl, haloalkyl, hydroxyalkyl, alkoxyalkyl, or aminoalkyl.

A3. In embodiment A3, the compound of embodiment A1, or a pharmaceutically acceptable salt thereof, is wherein R¹ is aryl, heteroaryl, aralkyl, heteroaralkyl, cycloalkylalkyl, or heterocyclylalkyl, wherein aryl, by itself or as part of aralkyl, heteroaryl, by itself or as part of heteroaralkyl, cycloalkyl of cycloalkylalkyl, and heterocyclyl of heterocyclylalkyl are substituted with R^{e}, R^{f}, and R^{g}.

A4a. In embodiment A4a, the compound of embodiment A1 or A2, or a pharmaceutically acceptable thereof, is wherein R¹ is alkyl.

A4. In embodiment A4, the compound of embodiment A1, A2, and A4a, or a pharmaceutically acceptable thereof, is wherein R¹ is methyl, ethyl, propyl (including isopropyl), or butyl.

A5a. In embodiment A5a, the compound of embodiment A1 or A2, or a pharmaceutically acceptable salt thereof, is wherein R¹ is haloalkyl.

A5. In embodiment A5, the compound of embodiment A1, A2, or A5a, or a pharmaceutically acceptable salt thereof, is wherein R¹ is 2,2-difluoroethyl or 2,2,2-trifluoroethyl.

A6a. In embodiment A6a, the compound of embodiment A1 or A2, or a pharmaceutically acceptable salt thereof, is wherein R¹ is hydroxyalkyl.

A6. In embodiment A6, the compound of embodiment A1 or A2, or a pharmaceutically acceptable salt thereof, is wherein R¹ is hydroxymethyl or hydroxyethyl.

A7a. In embodiment A7a, the compound of embodiment A1 or A2, or a pharmaceutically acceptable salt thereof, is wherein R¹ is alkoxyalkyl.

A7. In embodiment A7, the compound of embodiment A1 or A2, or a pharmaceutically acceptable salt thereof, is wherein R¹ is methoxymethyl or methoxyethyl.

A8. In embodiment A8, the compound of embodiment A1 or A2, or a pharmaceutically acceptable salt thereof, is wherein R¹ is aminoalkyl.

A9. In embodiment A9, the compound of embodiment A1 or A3, or a pharmaceutically acceptable salt thereof, is wherein R¹ is phenyl substituted with R^{e}, R^{f}, and R^{g}.

A10. In embodiment A10, the compound of embodiment A1 or A3, or a pharmaceutically acceptable salt thereof, is wherein R¹ is heteroaryl substituted with R^{e}, R^{f}, and R^{g}.

A11. In embodiment A11, the compound of embodiment A1 or A3, or a pharmaceutically acceptable salt thereof, is wherein R¹ is pyridyl substituted with R^{e}, R^{f}, and R^{g}.

A12a. In embodiment A12a, the compound of embodiment A1 or A3, or a pharmaceutically acceptable salt thereof, is wherein R¹ is aralkyl substituted with R^{e}, R^{f}, and R^{g}.

A12. In embodiment A12, the compound of embodiment A1 or A3, or a pharmaceutically acceptable salt thereof, is wherein R¹ is benzyl substituted with R^{e}, R^{f}, and R^{g}.

A13. In embodiment A13, the compound of any one of embodiments A1, A3, and A10 to A12, or a pharmaceutically acceptable salt thereof, is wherein R¹ is heteroaralkyl substituted with R^{e}, R^{f}, and R^{g}.

A14. In embodiment A14, the compound of any one of embodiments A1 or A3, or a pharmaceutically acceptable salt thereof, is wherein R¹ is cycloalkylalkyl substituted with R^{e}, R^{f}, and R^{g}.

A15a. In embodiment A15a, the compound of embodiment A1 or A3, or a pharmaceutically acceptable salt thereof, is wherein R¹ is heterocyclylalkyl substituted with R^{e}, R^{f}, and R^{g}.

A15. In embodiment A15, the compound of embodiment A1 or A3, or a pharmaceutically acceptable salt thereof, is wherein R¹ is oxetanylmethyl.

A16. In embodiment A16, the compound of embodiment A1 or A2, or a pharmaceutically acceptable salt thereof, is wherein R¹ is methyl, ethyl, isopropyl, or 2,2,2-trifluoroethyl.

A17. In embodiment A17, the compound of any one of embodiments A1 to A16, or a pharmaceutically acceptable salt thereof, is wherein R³ is hydrogen, cyano, halo, haloalkyl, or alkyl optionally substituted with hydroxy.

A18. In embodiment A18, the compound of any one of embodiments A1 to A17, or a pharmaceutically acceptable salt thereof, is wherein R³ is hydrogen, cyano, fluoro, chloro, difluoromethyl, trifluoromethyl, methyl, or hydroxymethyl.

A19. In embodiment A19, the compound of any one of embodiments A1 to A18, or a pharmaceutically acceptable salt thereof, is wherein R³ is hydrogen.

A20a. In embodiment A20a, the compound of any one of embodiments A1 to A17, or a pharmaceutically acceptable salt thereof, is wherein R³ is haloalkyl.

A20. In embodiment A20, the compound of embodiment A20a, or a pharmaceutically acceptable salt thereof, is wherein R³ is difluoromethyl.

A21. In embodiment A21, the compound of embodiment A17, or a pharmaceutically acceptable salt thereof, is wherein R³ is alkyl.

A22a. In embodiment A22a, the compound of any one of embodiments A1 to A17, or a pharmaceutically acceptable salt thereof, is wherein R³ is alkyl substituted with hydroxy.

A22. In embodiment A22, the compound of embodiment A22a, or a pharmaceutically acceptable salt thereof, is wherein R³ is hydroxymethyl.

A23a. In embodiment A23a, the compound of any one of embodiments A1 to A17, or a pharmaceutically acceptable salt thereof, is wherein R³ is halo.

A23. In embodiment A23, the compound of embodiment A23a, or a pharmaceutically acceptable salt thereof, is wherein R³ is fluoro or chloro.

A24. In embodiment A24, the compound of any one of embodiments A1 to A17, or a pharmaceutically acceptable salt thereof, or a pharmaceutically acceptable salt thereof, is wherein R³ is cyano.

A25. In embodiment A25, the compound of any one of embodiments A1 to A24, or a pharmaceutically acceptable salt thereof, or a pharmaceutically acceptable salt thereof, is wherein R² is hydrogen.

A26. In embodiment A26, the compound of any one of embodiments A1 to A25, or a pharmaceutically acceptable salt thereof, is wherein Hy is heterocyclylene, phenylene, or spiro heterocyclylene, or cycloalkylene, wherein each of the aforementioned ring is substituted with R^{b}, R^{c}, and R^{d} where R^{b} and R^{c} are independently selected from hydrogen, deuterium, alkyl, halo, haloalkyl, alkoxy, and hydroxy and R^{d} is hydrogen.

A27. In embodiment A27, the compound of any one of embodiments A1 to A26, or a pharmaceutically acceptable salt thereof, is wherein Hy is heterocyclylene substituted with R^{b}, R^{c}, and R^{d} where R^{b} and R^{c} are independently selected from hydrogen, deuterium, alkyl, halo, haloalkyl, alkoxy, and hydroxy and R^{d} is hydrogen.

A28. In embodiment A28, the compound of any one of embodiments A1 to A27, or a pharmaceutically acceptable salt thereof, is wherein Hy is pyrrolidin-1,3-diyl or piperidin-1,4-diyl substituted with R^{b}, R^{c}, and R^{d} where R^{b} and R^{c} are independently hydrogen, deuterium, methyl, fluoro, methoxy, or hydroxy, R^{d} is hydrogen, and L is attached to the nitrogen atom of the piperidin-1,4-diyl or pyrrolidin-1,3-diyl ring of Hy.

A29. In embodiment A29, the compound of any one of embodiments A1 to A28, or a pharmaceutically acceptable salt thereof, is wherein Hy is:

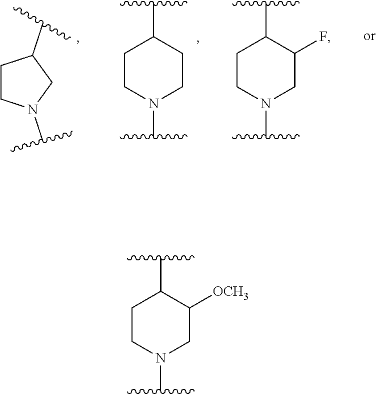 [SMILES: *C1CCN(*)C1 ; *C1CCN(*)CC1 ; *C1CCN(*)CC1F ; "or" ; *C1CCN(*)CC1OC]

where the N atom of the pyrrolidin-1,3-diyl or piperidin-1,4-diyl ring is attached to L.

A30. In embodiment A30, the compound of any one of embodiments A1 to A29, or a pharmaceutically acceptable salt thereof, is wherein Hy is:

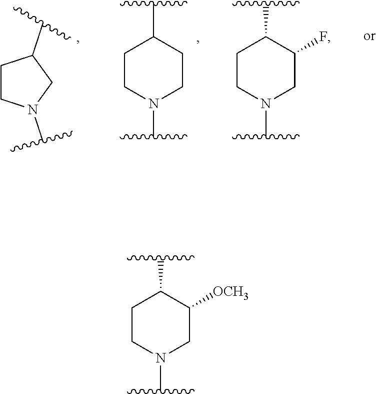 [SMILES: *C1CCN(*)C1 ; *C1CCN(*)CC1 ; *[C@H]1CCN(*)C[C@H]1F ; "or" ; *[C@H]1CCN(*)C[C@H]1OC]

where the N atom of the pyrrolidin-1,3-diyl or piperidin-1,4-diyl rings is attached to L.

A30a. In embodiment A30a, the compound of any one of embodiments A1 to A29, or a pharmaceutically acceptable salt thereof, is wherein Hy is:

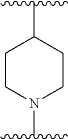 [SMILES: *C1CCN(*)CC1]

where the N atom of the piperidin-1,4-diyl ring is attached to L.

A31. In embodiment A31, the compound of any one of embodiments A1 to A25, or a pharmaceutically acceptable salt thereof, is wherein Hy is a bicyclic heterocyclylene substituted with R^{b}, R^{c}, and R^{d} independently selected from hydrogen, deuterium, alkyl, halo, haloalkyl, alkoxy, hydroxy, and cyano. In a subembodiment of embodiment A31, the compound is that wherein Hy is a ring of formula:

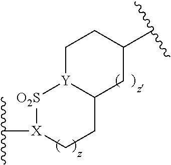

where X is CH or N and forms a bond with L; Y is CH, CMe, or N; provided at least one of X and Y is N; z is 0, 1, or 2; z′ is 0 or 1; provided at least one of z′ and z is 1; and Hy is substituted with R^{b}, R^{c}, and R^{d} independently selected from hydrogen, deuterium, alkyl, halo, haloalkyl, alkoxy, and hydroxy.

A32. In embodiment A32, the compound of embodiment A31, or a pharmaceutically acceptable salt thereof, is wherein X is N and Y is CH.

A33. In embodiment A33, the compound of embodiment A31, or a pharmaceutically acceptable salt thereof, is wherein Y is N and X is CH.

A34. In embodiment A34, the compound of any one of embodiments A1 to A26, or a pharmaceutically acceptable salt thereof, is wherein Hy is cycloalkylene substituted with R^{b}, R^{c}, and R^{d} where R^{b} is deuterium, methyl, fluoro, methoxy, or hydroxy and R^{c} and R^{d} are hydrogen.

A35. In embodiment A35, the compound of embodiment A34, or a pharmaceutically acceptable salt thereof, is wherein Hy is cyclohexylene.

A36. In embodiment A36, the compound of any one of embodiments A1 to A26, A34, and A35, or a pharmaceutically acceptable salt thereof, is wherein Hy is

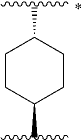 [SMILES: *[C@H]1CC[C@H](*)CC1]

where

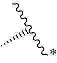

denotes bond to NH and

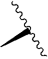

denotes bond of L.

A37. In embodiment A37, the compound of any one of embodiments A1 to A26, or a pharmaceutically acceptable salt thereof, is wherein Hy is phenylene substituted with R^{b}, R^{c} and R^{d} where R^{b} and R^{c} are independently selected from hydrogen, deuterium, alkyl, halo, haloalkyl, alkoxy, and hydroxy and R^{d} is hydrogen.

A38. In embodiment A38, the compound of any one of embodiments A1 to A26, or a pharmaceutically acceptable salt thereof, is wherein Hy is spiro heterocyclylene substituted with R^{b}, R^{c}, and R^{d} where R^{b} and R^{c} are independently selected from hydrogen, deuterium, alkyl, halo, haloalkyl, alkoxy, and hydroxy and R^{d} is hydrogen.

A39. In embodiment A39, the compound of any one of embodiments A1 to A25, or a pharmaceutically acceptable salt thereof, is wherein Hy is bridged heterocyclylene substituted with R^{b}, R^{c}, and R^{d} where R^{b} and R^{c} are independently selected from hydrogen, deuterium, alkyl, halo, haloalkyl, alkoxy, and hydroxy and R^{d} is hydrogen.

A39a1. In embodiment A39a1, the compound of any one of embodiments A1 to A25, or a pharmaceutically acceptable salt thereof, is wherein Hy is fused heterocyclylene substituted with R^{b}, R^{c}, and R^{d} where R^{b} and R^{c} are independently selected from hydrogen, deuterium, alkyl, halo, haloalkyl, alkoxy, and hydroxy and R^{d} is hydrogen.

A39a. In embodiment A39a, the compound of any one of embodiments A1 to A39a1, or a pharmaceutically acceptable salt thereof, is wherein the Degron is an E3 ligase ligand of formula (i) or (ii).

A40. In embodiment A40, the compound of any one of embodiments A1 to A39a, or a pharmaceutically acceptable salt thereof, is wherein the Degron is an E3 ligase ligand of formula (i):

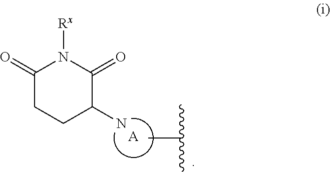

A41. In embodiment A41, the compound of any one of embodiments A1 to A40, or a pharmaceutically acceptable salt thereof, is wherein ring A of the E3 ligase ligand of formula (i) is a group of formula (a):

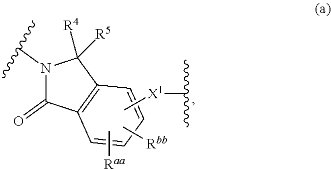

A42. In embodiment A42, the compound of any one of embodiments A1 to A41, or a pharmaceutically acceptable salt thereof, is wherein R⁴ and R⁵ are independently hydrogen or alkyl.

A43. In embodiment A43, the compound of any one of embodiments A1 to A42, or a pharmaceutically acceptable salt thereof, is wherein R⁴ and R⁵ are each hydrogen.

A44. In embodiment A44, the compound of any one of embodiments A1 to 42, or a pharmaceutically acceptable salt thereof, is wherein R⁴ is hydrogen and R⁵ is methyl.

A45. In embodiment A45, the compound of any one of embodiments A1 to A41, or a pharmaceutically acceptable salt thereof, is wherein R⁴ and R⁵ together with the carbon to which they are attached form >C═O.

A46. In embodiment A46, the compound of any one of embodiments A1 to A40, or a pharmaceutically acceptable salt thereof, is wherein the ring A of the E3 ligase ligand of formula (i) is a group of formula (b):

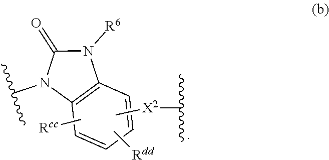

A47. In embodiment A47, the compound of any one of embodiments A1 to A40 and A46, or a pharmaceutically acceptable salt thereof, is wherein R⁶ is hydrogen.

A48. In embodiment A48, the compound of any one of embodiments A1 to A40 and A46, or a pharmaceutically acceptable salt thereof, is wherein R⁶ is alkyl.

A48a. In embodiment A48a, the compound of any one of embodiments A1 to A40, A46, and A48, or a pharmaceutically acceptable salt thereof, is wherein R⁶ is methyl.

A49. In embodiment A49, the compound of any one of embodiments A1 to A40, or a pharmaceutically acceptable salt thereof, is wherein ring A of the E3 ligase ligand of formula (i) is a group of formula (c):

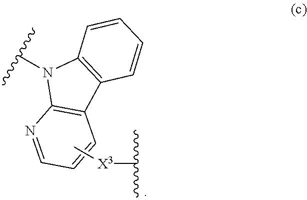

A50. In embodiment A50, the compound of any one of embodiments A1 to A48a, or a pharmaceutically acceptable salt thereof, is wherein ring A of the E3 ligase ligand of formula (i) is:

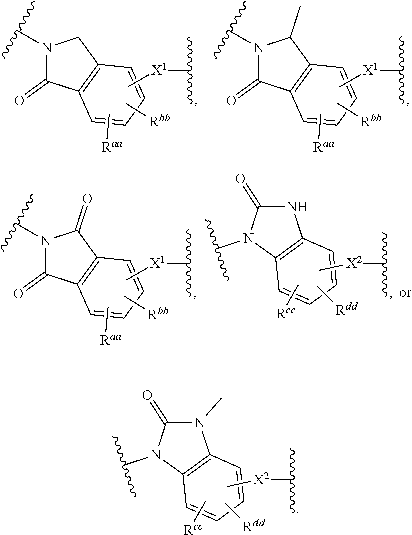

A51. In embodiment A51, the compound of any one of embodiments A1 to A48a and A50, or a pharmaceutically acceptable salt thereof, is wherein ring A of the E3 ligase ligand of formula (i) is:

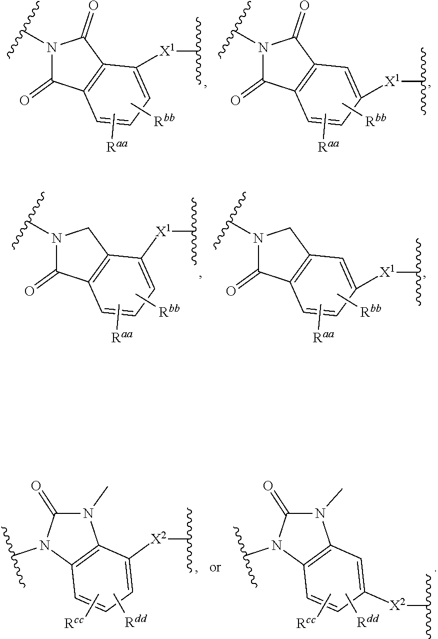

A52. In embodiment A52, the compound of any one of embodiments A1 to A48a, A50, and A51, or a pharmaceutically acceptable salt thereof, is wherein ring A of the E3 ligase ligand of formula (i) is:

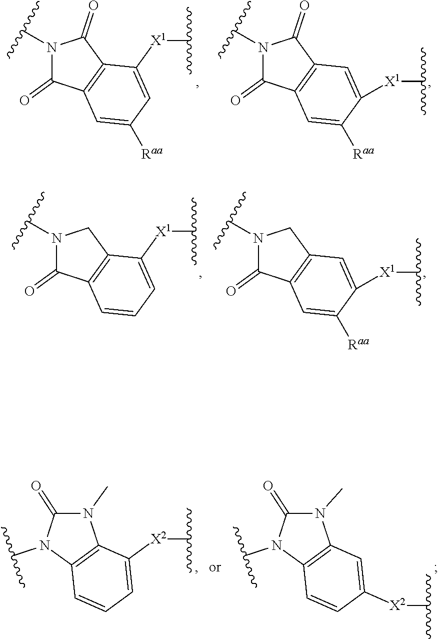

i.e., where R^{bb}, R^{cc}, and R^{dd} are hydrogen.

A52a. In embodiment A52a, the compound of any one of embodiments A1 to A41, A45, and A51 to A52, or a pharmaceutically acceptable salt thereof, is wherein ring A of the E3 ligase ligand of formula (i) is:

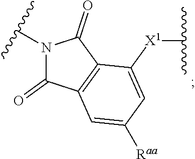

i.e., where R^{bb} is hydrogen.

A53. In embodiment A53, the compound of any one of embodiments A1 to A48a and A50 to A52, or a pharmaceutically acceptable salt thereof, is wherein ring A of the E3 ligase ligand of formula (i) is:

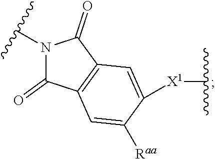

i.e., where R^{bb} is hydrogen.

A54. In embodiment A54, the compound of any one of embodiments A1 to A43 and A50 to A52, or a pharmaceutically acceptable salt thereof, is wherein ring A of the E3 ligase ligand of formula (i) is:

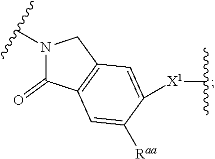

i.e., where R^{bb} is hydrogen.

A55. In embodiment A55, the compound of any one of embodiments A1 to A43 and A50 to A52, or a pharmaceutically acceptable salt thereof, is wherein ring A of the E3 ligase ligand of formula (i) is:

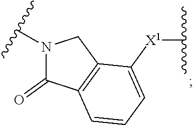

i.e., where R^{aa} and R^{bb} are hydrogen.

A56. In embodiment A56, the compound of any one of embodiments A1 to A40, A46, A48, A48a, A50 to A52, or a pharmaceutically acceptable salt thereof, is wherein ring A of the E3 ligase ligand of formula (i) is:

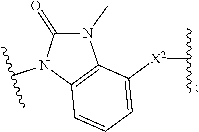

i.e., where R^{cc} and R^{dd} are hydrogen.

A57. In embodiment A57, the compound of any one of embodiments A1 to A40, or a pharmaceutically acceptable salt thereof, is wherein ring A of the E3 ligase ligand of formula (i) is:

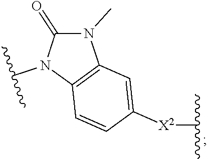

i.e., where R^{cc} and R^{dd} are hydrogen.

A58. In embodiment A58, the compound of any one of embodiments A1 to A54, or a pharmaceutically acceptable salt thereof, is wherein R^{aa}, R^{bb}, R^{cc}, and R^{dd}, when present, are independently selected from hydrogen, alkyl, alkoxy, halo, haloalkyl, and haloalkoxy.

A59. In embodiment A59, the compound of any one of embodiments A1 to A54, or a pharmaceutically acceptable salt thereof, is wherein R^{aa}, R^{bb}, R^{cc}, and R^{dd}, when present, are independently selected from hydrogen, alkyl, alkoxy, halo, haloalkyl, and cyano.

A60. In embodiment A60, the compound of any one of embodiments A1 to A54, or a pharmaceutically acceptable salt thereof, is wherein R^{aa}, R^{bb}, R^{cc}, and R^{dd}, when present, are independently selected from hydrogen, methyl, methoxy, ethoxy, fluoro, trifluoromethyl, difluoromethyl, and trifluoromethoxy.

A61. In embodiment A61, the compound of any one of embodiments A1 to A54, or a pharmaceutically acceptable salt thereof, is wherein R^{aa}, R^{bb}, R^{cc}, and R^{dd}, when present, are independently selected from hydrogen and methyl.

A62. In embodiment A62, the compound of any one of embodiments A1 to A54, or a pharmaceutically acceptable salt thereof, is wherein R^{aa}, R^{bb}, R^{cc}, and R^{dd}, when present, are independently selected from hydrogen and methoxy.

A63. In embodiment A63, the compound of any one of embodiments A1 to A54, or a pharmaceutically acceptable salt thereof, is wherein R^{aa}, R^{bb}, R^{cc}, and R^{dd}, when present, are independently selected from hydrogen and fluoro.

A64. In embodiment A64, the compound of any one of embodiments A1 to A54, or a pharmaceutically acceptable salt thereof, is wherein R^{aa}, R^{bb}, R^{cc}, and R^{dd}, when present, are independently selected from hydrogen, trifluoromethyl, and difluoromethyl.

A65. In embodiment A65, the compound of any one of embodiments A1 to A54, or a pharmaceutically acceptable salt thereof, is wherein R^{aa}, R^{bb}, R^{cc}, and R^{dd}, when present, are independently selected from hydrogen and trifluoromethoxy.

A66. In embodiment A66, the compound of any one of embodiments A1 to A54, or a pharmaceutically acceptable salt thereof, is wherein R^{aa}, R^{bb}, R^{cc}, and R^{dd}, when present, are independently selected from hydrogen, fluoro, and trifluoromethyl.

A67. In embodiment A67, the compound of any one of embodiments A1 to A39a, or a pharmaceutically acceptable salt thereof, is wherein the Degron is an E3 ligase ligand of formula (ii):

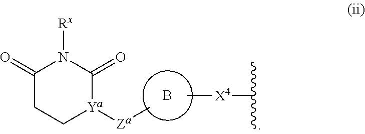

A68. In embodiment A68, the compound of any one of embodiments A1 to A39a and A67, or a pharmaceutically acceptable salt thereof, is wherein Y^{a} is CH.

A69. In embodiment A69, the compound of any one of embodiments A1 to A39a and A67, or a pharmaceutically acceptable salt thereof, is wherein Y^{a} is N.

A70. In embodiment A70, the compound of any one of embodiments A1 to A39a, and A67-A69, or a pharmaceutically acceptable salt thereof, is wherein Z^{a} is a bond, —NH—, O, or —NHC(O)—.

A71. In embodiment A71, the compound of any one of embodiments A1 to A39a, and A67-A70, or a pharmaceutically acceptable salt thereof, is wherein Z^{a} is a bond, —NH—, or —NHC(O)—.

A72. In embodiment A72, the compound of any one of embodiments A1 to A39a, and A67-A69, or a pharmaceutically acceptable salt thereof, is wherein Z^{a} is a bond.

A73. In embodiment A73, the compound of any one of embodiments A1 to A39a, and A67-A68 and A71, or a pharmaceutically acceptable salt thereof, is wherein Z^{a} is —NH—, or —NHC(O)—.

A74. In embodiment A74, the compound of any one of embodiments A1 to A39a, and A67-A68 and A71, or a pharmaceutically acceptable salt thereof, is wherein Z^{a} is —NH—.

A74a. In embodiment A74a, the compound of any one of embodiments A1 to A39a, and A67-A68 and A71, or a pharmaceutically acceptable salt thereof, is wherein Z^{a} is —NHC(O)—.

A75. In embodiment A75, the compound of any one of embodiments A1 to A39a, and A67-A74a, or a pharmaceutically acceptable salt thereof, is wherein ring B is phenylene substituted with R^{ee} and R^{ff} independently selected from hydrogen, alkyl, cycloalkyl, alkoxy, halo, haloalkyl, haloalkoxy, and cyano.

A76. In embodiment A76, the compound of any one of embodiments A1 to A39a, and A67-A74a, or a pharmaceutically acceptable salt thereof, is wherein ring B is cyclylaminylene substituted with R^{ee} and R^{ff} independently selected from hydrogen, alkyl, cycloalkyl, alkoxy, halo, haloalkyl, haloalkoxy, and cyano.

A77. In embodiment A77, the compound of any one of embodiments A1 to A39a, and A67-A74a, or a pharmaceutically acceptable salt thereof, is wherein ring B is 5 or 6-membered monocyclic heteroarylene or a 9- or 10-membered fused bicyclic heteroarylene, wherein each heteroarylene ring contains one to three nitrogen ring atoms and each ring is substituted with R^{ee} and R^{ff} independently selected from hydrogen, alkyl, cycloalkyl, alkoxy, halo, haloalkyl, haloalkoxy, and cyano.

A78. In embodiment A78, the compound of any one of embodiments A1 to A39a, and A67-A74a, or a pharmaceutically acceptable salt thereof, is wherein ring B is 5- or 6-membered monocyclic heteroarylene containing one or two nitrogen ring atoms substituted with R^{ee} and R^{ff} independently selected from hydrogen, alkyl, cycloalkyl, alkoxy, halo, haloalkyl, haloalkoxy, and cyano.

A79. In embodiment A79, the compound of any one of embodiments A1 to A39a, and A67-A74a, or a pharmaceutically acceptable salt thereof, is wherein ring B is a 9- or 10-membered fused bicyclic heteroarylene containing one to three nitrogen ring atoms and substituted with R^{ee} and R^{ff} independently selected from hydrogen, alkyl, cycloalkyl, alkoxy, halo, haloalkyl, haloalkoxy, and cyano.

A80. In embodiment A80, the compound of any one of embodiments A1 to A39a, and A67 to A74a and A79, or a pharmaceutically acceptable salt thereof, is wherein ring B is a 9- or 10-membered fused bicyclic heteroarylene containing two nitrogen ring atoms and substituted with R^{ee} and R^{ff} independently selected from hydrogen, alkyl, cycloalkyl, alkoxy, halo, haloalkyl, haloalkoxy, and cyano.

A81. In embodiment A81, the compound of any one of embodiments A1 to A39a, and A67 to A80, or a pharmaceutically acceptable salt thereof, is wherein the E3 ligase ligand of formula (ii) is:

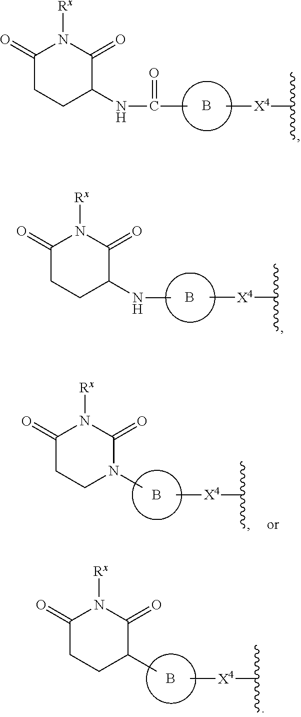

A82. In embodiment A82, the compound of any one of embodiments A1 to A39a and A67 to A81, or a pharmaceutically acceptable salt thereof, is wherein the E3 ligase ligand of formula (ii) is:

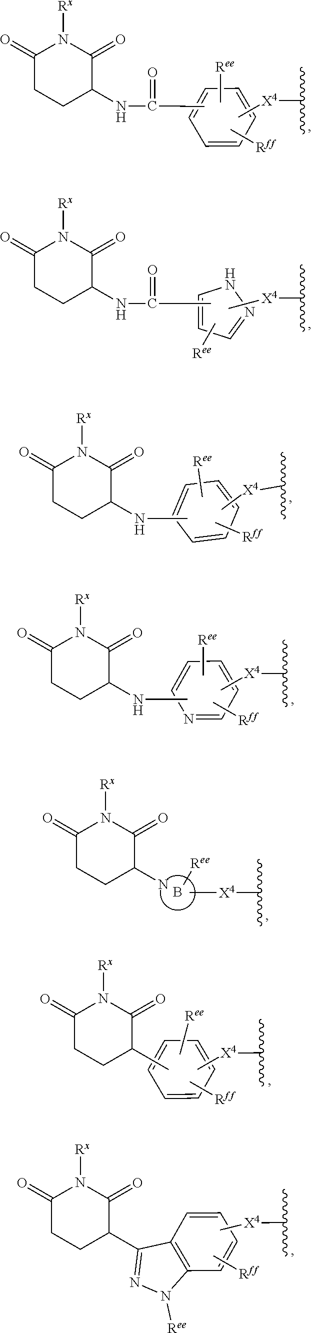

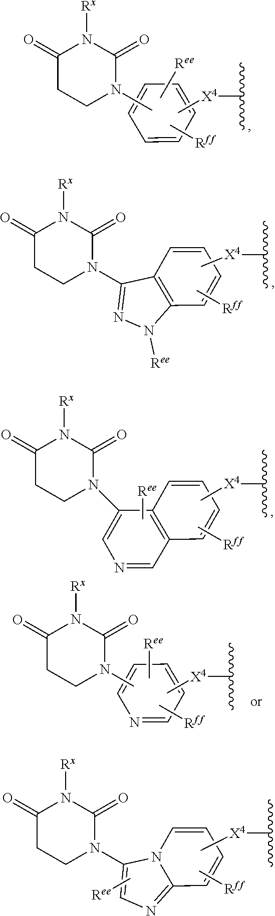

where ring B is cyclylaminylene.

A83. In embodiment A83, the compound of any one of embodiments A1 to A39a and A67 to A82, or a pharmaceutically acceptable salt thereof, is wherein the E3 ligase ligand of formula (ii) is:

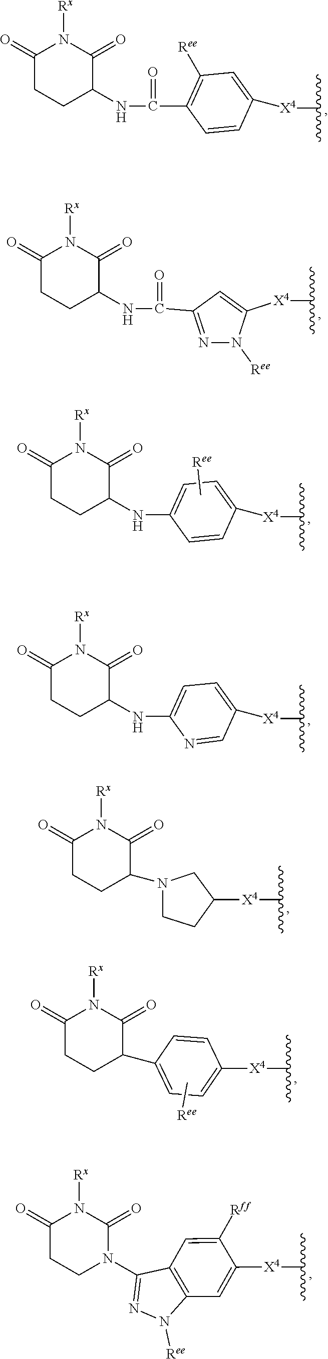

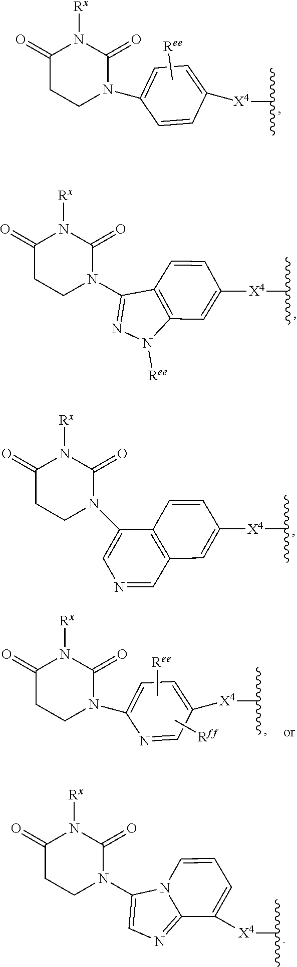

A84. In embodiment A84, the compound of any one of embodiments A1 to A39a, and A67 to A83, or a pharmaceutically acceptable salt thereof, is wherein each of R^{ee} and R^{ff} are independently selected from hydrogen, alkyl, cycloalkyl, alkoxy, halo, cyano, haloalkyl, and haloalkoxy.

A85. In embodiment A85, the compound of any one of embodiments A1 to A39a and A67 to A84, or a pharmaceutically acceptable salt thereof, is wherein each of R^{ee} and R^{ff} are independently selected from hydrogen, alkyl, cycloalkyl, alkoxy, halo, haloalkyl, and cyano.

A86. In embodiment A86, the compound of any one of embodiments A1 to A39a and A67 to A85, or a pharmaceutically acceptable salt thereof, is wherein each of R^{ee} and R^{ff} are independently selected from hydrogen, methyl, ethyl, isopropyl, cyclopropyl, methoxy, ethoxy, fluoro, chloro, trifluoromethyl, 2,2,2-trifluoroethyl, difluoromethyl, difluoromethoxy, trifluoromethoxy or cyano.

A87. In embodiment A87, the compound of any one of embodiments A1 to A39a and A67 to A86, or a pharmaceutically acceptable salt thereof, is wherein each of R^{ee} aid R^{ff} are independently selected from hydrogen and methyl, ethyl, or isopropyl.

A88. In embodiment A88, the compound of any one of embodiments A1 to A39a and A67 to A86, or a pharmaceutically acceptable salt thereof, is wherein each of R^{ee} and R^{ff} are independently selected from hydrogen and methoxy.

A89. In embodiment A89, the compound of any one of embodiments A1 to A39a and A67 to A86, or a pharmaceutically acceptable salt thereof, is wherein each of R^{ee} and R^{ff} are independently selected from hydrogen, methyl, ethyl, isopropyl, chloro, and fluoro.

A90 In embodiment A90, the compound of any one of embodiments A1 to A39a and A67 to A86, or a pharmaceutically acceptable salt thereof, is wherein one of R^{ee} and R^{ff} is hydrogen or fluoro and the other of R^{ee} and R^{ff} is selected from hydrogen, trifluoromethyl, 2,2,2-trifluoroethyl, and difluoromethyl.

A91. In embodiment A91, the compound of any one of embodiments A1 to A39a and A67 to A86, or a pharmaceutically acceptable salt thereof, is wherein each of R^{ee} and R^{ff} are independently selected from hydrogen, difluoromethoxy, and trifluoromethoxy.

A92. In embodiment A92, the compound of any one of embodiments A1 to A39a and A67 to A86, or a pharmaceutically acceptable salt thereof, is wherein each of R^{ee} and R^{ff} are independently selected from hydrogen, chloro, fluoro, and trifluoromethyl.

A93. In embodiment A93, the compound of any one of embodiments A1 to A39a and A67 to A86, or a pharmaceutically acceptable salt thereof, is wherein R^{ee} and R^{ff} are independently hydrogen.

A94. In embodiment A94, the compound of any one of embodiments A1 to A39a and A67 to A86, or a pharmaceutically acceptable salt thereof, is wherein R^{ee} and R^{ff} are independently chloro.

A95. In embodiment A95, the compound of any one of embodiments A1 to A39a and A67 to A86, or a pharmaceutically acceptable salt thereof, is wherein R^{ee} and R^{ff} are independently fluoro.

A96. In embodiment A96, the compound of any one of embodiments A1 to A39a and A67 to A86, or a pharmaceutically acceptable salt thereof, is wherein each of R^{ee} and R^{ff} are independently trifluoromethyl or 2,2,2-trifluoroethyl.

A96a. In embodiment A % a, the compound of any one of embodiments A1 to A39a, or a pharmaceutically acceptable salt thereof, is wherein the Degron is an E3 ligase ligand of formula (iii), (iv), (v), or (vi).

A96b. In embodiment A96b, the compound of any one of embodiments A1 to A39a, and A96a, or a pharmaceutically acceptable salt thereof, is wherein the Degron is an E3 ligase ligand of formula (iii), (iv), (v), or (vi) where R^{y} is 1-fluorocycloprop-1-yl and W^{a} is bond, S, or methylene.

A97. In embodiment A97, the compound of any one of embodiments A1 to A %, or a pharmaceutically acceptable salt thereof, is wherein X¹, X², X³, and X⁴ are independently a bond.

A98. In embodiment A98, the compound of any one of embodiments A1 to A96, or a pharmaceutically acceptable salt thereof, is wherein X¹, X², X³, and X⁴ are independently alkylene.

A98a. In embodiment A98a, the compound of any one of embodiments A1 to A96 and A98, or a pharmaceutically acceptable salt thereof, is wherein X¹, X², X³, and X⁴ are each methylene.

A99. In embodiment A99, the compound of any one of embodiments A1 to A %, or a pharmaceutically acceptable salt thereof, is wherein X¹, X², X³, and X⁴ are independently —O—.

A100a. In embodiment A100a, the compound of any one of embodiments A1 to A96, or a pharmaceutically acceptable salt thereof, is wherein X¹, X², X³, and X⁴ are independently —(O-alkylene)-.

A100. In embodiment A100, the compound of any one of embodiments A1 to A96 and A100a, or a pharmaceutically acceptable salt thereof, is wherein X¹, X², X³, and X⁴ are independently —(O—CH₂)—, —O—(CH₂)₂—, or —O—(CH₂)₃—.

A101a. In embodiment A101a, the compound of any one of embodiments A1 to A96, or a pharmaceutically acceptable salt thereof, is wherein X¹, X², X³, and X⁴ are independently -(alkylene-O)—.

A101. In embodiment A101, the compound of any one of embodiments A1 to A96 and A101, or a pharmaceutically acceptable salt thereof, is wherein X¹, X², X³, and X⁴ are independently —(CH₂—O)—, —(CH₂)₂—O—, or —(CH₂)₃—O—.

A102. In embodiment A102, the compound of any one of embodiments A1 to A96, or a pharmaceutically acceptable salt thereof, is wherein X¹, X², X³, and X⁴ are independently —(NR^{gg}-alkylene)-.

A103. In embodiment A103, the compound of any one of embodiments A1 to A96, or a pharmaceutically acceptable salt thereof, is wherein X¹, X², X³, and X⁴ are independently -(alkylene-NR^{hh})—.

A104. In embodiment A104, the compound of any one of embodiments A1 to A96, or a pharmaceutically acceptable salt thereof, is wherein X¹, X², X³, and X⁴ are

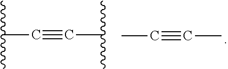

A105. In embodiment A105, the compound of any one of embodiments A1 to A96, or a pharmaceutically acceptable salt thereof, is wherein X¹, X², X³, and X⁴ are —NH—.

A106. In embodiment A106, the compound of any one of embodiments A1 to A96, or a pharmaceutically acceptable salt thereof, is wherein X¹, X², X³, and X⁴ are independently —N(alkyl)-.

A107. In embodiment A107, the compound of any one of embodiments A1 to A96, or a pharmaceutically acceptable salt thereof, is wherein X¹, X², X³, and X⁴ are —C(═O)—.

A108. In embodiment A108, the compound of any one of embodiments A1 to A96, or a pharmaceutically acceptable salt thereof, is wherein X¹, X², X³, and X⁴ are independently —NR^{jj}C(═O)—.

A109. In embodiment A109, the compound of any one of embodiments A1 to A96, or a pharmaceutically acceptable salt thereof, is wherein X¹, X², X³, and X⁴ are independently —C(═O)NR^{kk}—.

A110. In embodiment A110, the compound of any one of embodiments A1 to A96, A102, A103, A108, and A109, or a pharmaceutically acceptable salt thereof, is wherein R^{gg}, R^{hh}, R^{jj}, and R^{kk} are independently hydrogen or alkyl.

A111. In embodiment A111, the compound of any one of embodiments A1 to A110, or a pharmaceutically acceptable salt thereof, is wherein Z⁶ is —S(O)₂—.

A112. In embodiment A112, the compound of any one of embodiments A1 to A96, or a pharmaceutically acceptable salt thereof, is wherein L (when the Degron is a group of formula (iii) to (vi)), —X¹-L-, —X²-L-, —X³-L-, and —X⁴-L- are independently selected from:

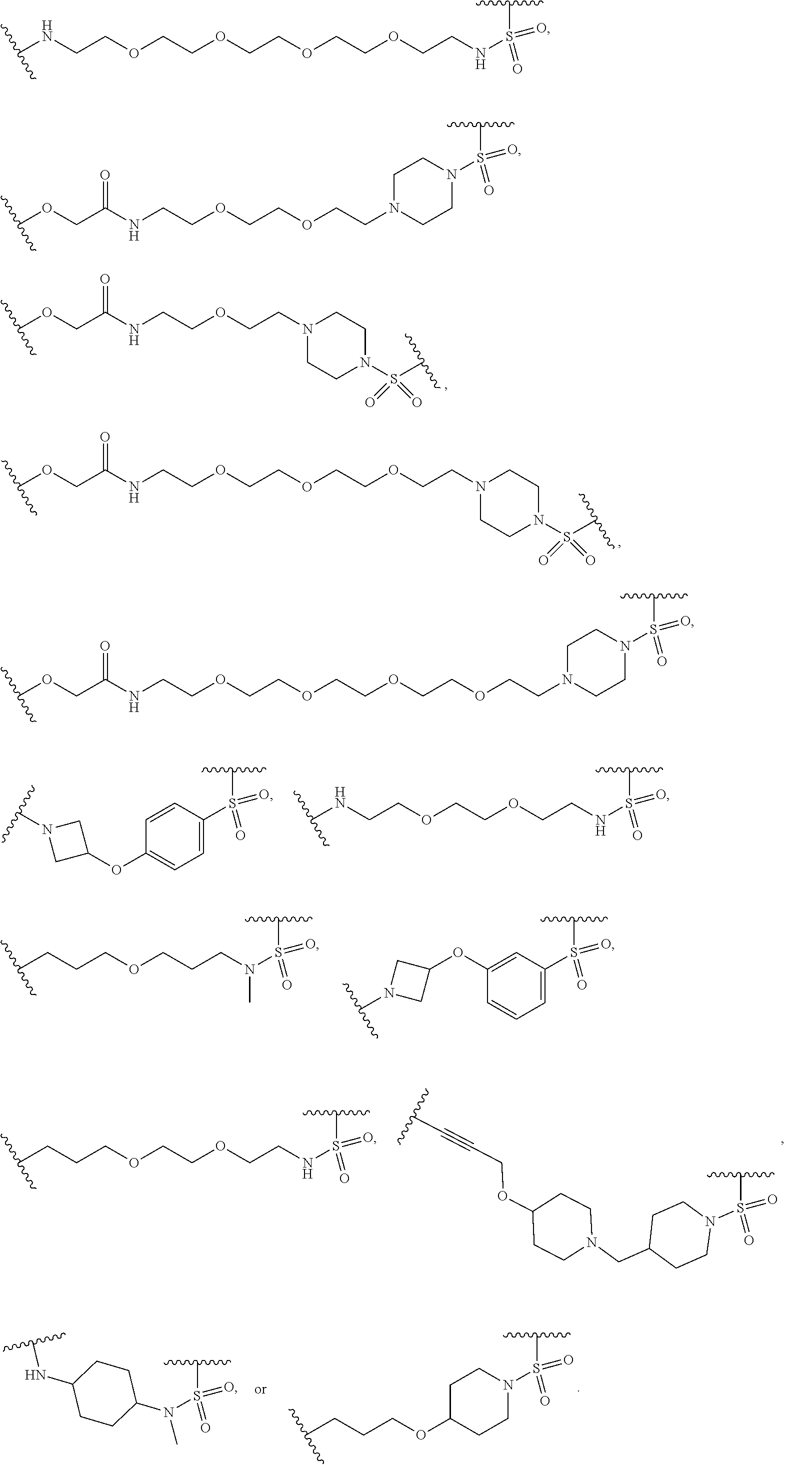

A113. In embodiment A113, the compound of any one of embodiments A1 to A111, or a pharmaceutically acceptable salt thereof, is wherein Z⁵ is a bond.

A114. In embodiment A114, the compound of any one of embodiments A1 to A96, A111, and A113, or a pharmaceutically acceptable salt thereof, is wherein Z⁵ is a bond and one of Z¹ and X¹ is a bond, one of Z¹ and X² is a bond, one of Z¹ and X³, and one of Z¹ and X⁴ is a bond.

A115. In embodiment A115, the compound of any one of embodiments A1 to A96b, or a pharmaceutically acceptable salt thereof, is wherein:

X¹, X², X³, and X⁴ are independently a bond, —(O-alkylene)-, —(NR^{gg}-alkylene)-,

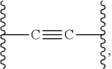 [SMILES: *C#C*]

—NH—, or —N(alkyl)-, where R^{gg} is hydrogen or alkyl and each alkylene is optionally substituted with one or two fluoro;
Z¹ is a bond, alkylene, —(CO)NR—, —(O-alkylene)ₐ-, -(alkylene-O)ₐ—, phenylene, or heterocyclylene, where each ring is substituted with R^{h} and Rⁱ independently selected from hydrogen, deuterium, alkyl, alkoxy, halo, haloalkyl, and haloalkoxy;
Z² is a bond, alkylene, —(O-alkylene)_{b}-, -(alkylene-O)_{b}—, cycloalkylene, or heterocyclylene, where each ring is substituted with R^{j} and R^{k} independently selected from hydrogen, deuterium, alkyl, alkoxy, halo, haloalkyl, and haloalkoxy;
Z³ is a bond, alkylene, —C(O)NR—, —NR′(CO)—, —O—, —NR″—, cycloalkylene, phenylene, monocyclic heteroarylene, heterocyclylene, fused heterocyclylene, or spiro heterocyclylene, where each ring is substituted with R^{m} and Rⁿ independently selected from hydrogen, deuterium, alkyl, alkoxy, halo, haloalkyl, and haloalkoxy;
Z⁴ is a bond, -(alkylene-NR″)—, —O—, —NR″—, cycloalkylene, phenylene, monocyclic heteroarylene, heterocyclylene, fused heterocyclylene, or spiro heterocyclylene, where each ring is substituted with R^{o} and R^{p} independently selected from hydrogen, deuterium, alkyl, alkoxy, halo, haloalkyl, and haloalkoxy;
Z⁵ is a bond; and
Z⁶ is —S(O)₂—; and
wherein each alkylene is substituted with R^{s} and R^{t}.

A116. In embodiment A116, the compound of any one of embodiments A1 to A96b, or a pharmaceutically acceptable salt thereof, is wherein:
X¹, X², X³, X⁴, and Z¹ are each a bond;
Z² is a bond, alkylene, cycloalkylene, or heterocyclylene, where each ring is substituted with R^{j} and R^{k} independently selected from hydrogen, deuterium, alkyl, alkoxy, halo, haloalkyl, and haloalkoxy;
Z³ is a bond, alkylene, —C(O)NR—, —NR′(CO)—, —O—, —NR″—, cycloalkylene, phenylene, monocyclic heteroarylene, heterocyclylene, bicyclic heterocyclylene, bridged heterocyclylene, fused heterocyclylene, or spiro heterocyclylene, where each ring is substituted with R^{m} and Rⁿ independently selected from hydrogen, deuterium, alkyl, alkoxy, halo, haloalkyl, and haloalkoxy;
Z⁴ is a bond, alkylene, —O—, cycloalkylene, phenylene, monocyclic heteroarylene, heterocyclylene, fused heterocyclylene, or spiro heterocyclylene, where each ring is substituted with Rₒ and R^{p} independently selected from hydrogen, deuterium, alkyl, alkoxy, halo, haloalkyl, and haloalkoxy;
Z⁵ is phenylene, monocyclic heteroarylene, or heterocycylene, where each ring is substituted with R^{q} and R^{r} independently selected from hydrogen, deuterium, alkyl, alkoxy, halo, haloalkyl, and haloalkoxy; and
Z⁶ is —S(O)₂—; and
wherein each alkylene is substituted with R^{s} and R^{t}.

A117. In embodiment A117, the compound of any one of embodiments A1 to A96b and A116, or a pharmaceutically acceptable salt thereof, is wherein:
X¹, X², X³, X⁴, Z¹, and Z² are each a bond;
Z³ is cycloalkylene, phenylene, monocyclic heteroarylene, heterocyclylene, bicyclic heterocyclylene, bridged heterocyclylene, fused heterocyclylene, or spiro heterocyclylene, where each ring is substituted with R^{m} and Rₙ independently selected from hydrogen, deuterium, alkyl, alkoxy, halo, haloalkyl, and haloalkoxy;
Z⁴ is a bond, alkylene, —O—, cycloalkylene, phenylene, monocyclic heteroarylene, heterocyclylene, fused heterocyclylene, or spiro heterocyclylene, where each ring is substituted with R^{o} and R^{p} independently selected from hydrogen, deuterium, alkyl, alkoxy, halo, haloalkyl, haloalkoxy, cyano, hydroxy, amino, alkylamino, and dialkylamino;
Z⁵ is phenylene, monocyclic heteroarylene, or heterocycylene, where each ring is substituted with R^{q} and R^{f} independently selected from hydrogen, deuterium, alkyl, alkoxy, halo, haloalkyl, and haloalkoxy; and
Z⁶ is —S(O)₂—; and
wherein alkylene is substituted with R^{s} and R^{t}.

A117a. In embodiment A117a, the compound of any one of embodiments A1 to A96b and A117, or a pharmaceutically acceptable salt thereof, is wherein R^{o} and R^{p} are independently selected from hydrogen, deuterium, alkyl, alkoxy, halo, haloalkyl, and haloalkoxy.

A118. In embodiment A118, the compound of any one of embodiments A1 to A96b, A116, and A117a, or a pharmaceutically acceptable salt thereof, is wherein:
X¹, X², X³, X⁴, Z¹, and Z² are each a bond;
Z³ is heterocyclylene, bicyclic heterocyclylene, bridged heterocyclylene, fused heterocyclylene, or spiro heterocyclylene, where each ring is substituted with R^{m} and Rₙ independently selected from hydrogen, deuterium, alkyl, alkoxy, halo, haloalkyl, and haloalkoxy;
Z⁴ is alkylene, —O—, cycloalkylene, monocyclic heteroarylene, heterocyclylene, fused heterocyclylene, or spiro heterocyclylene, where each ring is substituted with R^{o} and R^{p} independently selected from hydrogen, deuterium, alkyl, alkoxy, halo, haloalkyl, haloalkoxy, cyano, hydroxy, amino, alkylamino, and dialkylamino;
Z⁵ is phenylene, monocyclic heteroarylene, or heterocycylene, where each ring is substituted with R^{q} and R^{r} independently selected from hydrogen, deuterium, alkyl, alkoxy, halo, haloalkyl, and haloalkoxy; and
Z⁶ is —S(O)₂—; and
wherein alkylene substituted with substituted with R^{s} and R^{t}.

A118a. In embodiment A118a, the compound of any one of embodiments A1 to A96b, A116, and A118, or a pharmaceutically acceptable salt thereof, is wherein R^{o} and R^{p} are independently selected from hydrogen, deuterium, alkyl, alkoxy, halo, haloalkyl, and haloalkoxy.

A119. In embodiment A119, the compound of any one of embodiments A1 to A96b and A116 to A118a, or a pharmaceutically acceptable salt thereof, is wherein:
X¹, X², X³, and X⁴, Z¹, and Z² are each a bond;
Z³ is heterocyclylene, bridged heterocyclylene, or spiro heterocyclylene, where each ring is substituted with R^{m} and Rⁿ independently selected from hydrogen, deuterium, alkyl, alkoxy, halo, haloalkyl, and haloalkoxy;
Z⁴ is alkylene, —O—, cycloalkylene, or heterocyclylene, where each ring is substituted with R^{o} and Rₚ independently selected from hydrogen, deuterium, alkyl, alkoxy, halo, haloalkyl, haloalkoxy, cyano, hydroxy, amino, alkylamino, and dialkylamino;
Z⁵ is phenylene, monocyclic heteroarylene, or heterocycylene, where each ring is substituted with R^{q} and R^{r} independently selected from hydrogen, deuterium, alkyl, alkoxy, halo, haloalkyl, and haloalkoxy; and
Z⁶ is —S(O)₂—; and
wherein alkylene is substituted with R^{s} and R^{t}.

A119a. In embodiment A119a, the compound of any one of embodiments A1 to A96b and A116 to A119, or a pharmaceutically acceptable salt thereof, is wherein R^{o} and R^{p} are independently selected from hydrogen, deuterium, alkyl, alkoxy, halo, haloalkyl, and haloalkoxy.

A120. In embodiment A120, the compound of any one of embodiments A1 to A96b and A116 to A119a, or a pharmaceutically acceptable salt thereof, is wherein:
X¹, X², X³, X⁴, Z¹, and Z² are each a bond;
Z³ is heterocyclylene, bridged heterocyclylene, or spiro heterocyclylene, where each ring is substituted with R^{o} and R^{p} independently selected from hydrogen, deuterium, alkyl, alkoxy, halo, haloalkyl, and haloalkoxy;
Z⁴ is alkylene, —O—, cycloalkylene, or heterocyclylene, where each ring is substituted with R^{o} and R^{p} independently selected from hydrogen, deuterium, alkyl, alkoxy, halo, haloalkyl, haloalkoxy, cyano, hydroxy, amino, alkylamino, and dialkylamino;
Z⁵ is phenylene or monocyclic heteroarylene, each ring substituted with R^{q} and R^{r} independently selected from hydrogen, deuterium, alkyl, alkoxy, halo, haloalkyl, and haloalkoxy; and
Z⁶ is —S(O)₂—; and
wherein alkylene is substituted with R^{s} and R^{t}.
A120a. In embodiment A120a, the compound of any one of embodiments A1 to A96b and A116 to A120, or a pharmaceutically acceptable salt thereof, is wherein Z⁴ is alkylene or —O—.

A120b. In embodiment A120b, the compound of any one of embodiments A1 to A96b and A116 to A120, or a pharmaceutically acceptable salt thereof, is wherein R^{o} and R^{p} are independently selected from hydrogen, deuterium, alkyl, alkoxy, halo, haloalkyl, and haloalkoxy.

A121. In embodiment A121, the compound of any one of embodiments A1 to A96b and A116, or a pharmaceutically acceptable salt thereof, is wherein:
X¹, X², X³, X⁴, and Z¹ are each a bond;

Z² is cycloalkylene or heterocyclylene, where each ring is substituted with R^{j} and R^{k} independently selected from hydrogen, deuterium, alkyl, alkoxy, halo, haloalkyl, and haloalkoxy;
Z³ is cycloalkylene, phenylene, monocyclic heteroarylene, heterocyclylene, bicyclic heterocyclylene, bridged heterocyclylene, fused heterocyclylene, or spiro heterocyclylene, where each ring is substituted with R^{m} and Rⁿ independently selected from hydrogen, deuterium, alkyl, alkoxy, halo, haloalkyl, and haloalkoxy;
Z⁴ is a bond, alkylene, or —O—;
Z⁵ is phenylene, monocyclic heteroarylene (e.g., pyridindiyl), or heterocycylene, where each ring is substituted with R^{q} and R^{r} independently selected from hydrogen, deuterium, alkyl, alkoxy, halo, haloalkyl, and haloalkoxy; and
Z⁶ is —S(O)₂—; and
wherein alkylene is substituted with R^{s} and R^{t}.

A122. In embodiment A122, the compound of any one of embodiments A1 to A96b, A116, and A121, or a pharmaceutically acceptable salt thereof, is wherein:
X¹, X², X³, X⁴, and Z¹ are each a bond;
Z² is heterocyclylene substituted with R^{j} and R^{k} independently selected from hydrogen, deuterium, alkyl, alkoxy, halo, haloalkyl, and haloalkoxy;
Z³ is heterocyclylene substituted with R^{m} and Rⁿ independently selected from hydrogen, deuterium, alkyl, alkoxy, halo, haloalkyl, and haloalkoxy;
Z⁴ is a bond, alkylene, or —O—;
Z⁵ is phenylene or monocyclic heteroarylene, each ring substituted with R^{q} and R^{r} independently selected from hydrogen, deuterium, alkyl, alkoxy, halo, haloalkyl, and haloalkoxy; and
Z⁶ is —S(O)₂—; and
wherein alkylene is optionally substituted with R^{s} and R^{t}.

A123. In embodiment A123, the compound of any one of embodiments A1 to A96b and A116, or a pharmaceutically acceptable salt thereof, is wherein:
X¹, X², X³, X⁴, and Z¹ are each a bond;
Z² is heterocyclylene substituted with R^{j} and R^{k} independently selected from hydrogen, deuterium, alkyl, alkoxy, halo, haloalkyl, and haloalkoxy;
Z³ is a bond, alkylene, or —O—;
Z⁴ is heterocyclylene, bridged heterocyclylene, or spiro heterocyclylene, where each ring is substituted with R^{o} and R^{p} independently selected from hydrogen, deuterium, alkyl, alkoxy, halo, haloalkyl, and haloalkoxy;
Z⁵ is phenylene or monocylic heteroarylene, each ring substituted with R^{q} and R^{r} independently selected from hydrogen, deuterium, alkyl, alkoxy, halo, haloalkyl, and haloalkoxy; and
Z⁶ is —S(O)₂—.

A123a. In embodiment A123, the compound of any one of embodiments A1 to A96b, or a pharmaceutically acceptable salt thereof, is wherein Z⁴ is heterocyclylene, or spiro heterocyclylene, where each ring is substituted with R^{o} and R^{p} independently selected from hydrogen, deuterium, alkyl, alkoxy, halo, haloalkyl, and haloalkoxy.

A123b. In embodiment A123b, the compound of any one of embodiments A1 to A96b and A116 to A120, or a pharmaceutically acceptable salt thereof, is wherein:
X¹, X², X³, X⁴, Z¹ and Z² are each a bond;
Z³ is heterocyclylene, where each ring is substituted with R^{m} and Rⁿ independently selected from hydrogen, deuterium, alkyl, alkoxy, halo, haloalkyl, and haloalkoxy;
Z⁴ is cycloalkylene substituted with R^{o} and R^{p} independently selected from hydrogen, alkyl, alkoxy, halo, haloalkyl, and haloalkoxy;
Z⁵ is phenylene or monocyclic heteroarylene, each ring substituted with R^{q} and R^{r} independently selected from hydrogen, deuterium, alkyl, alkoxy, halo, haloalkyl, and haloalkoxy; and
Z⁶ is —S(O)₂—.

A124. In embodiment A124, the compound of any one of embodiments A1 to A111 and A116 to A123b, or a pharmaceutically acceptable salt thereof, is wherein —Z⁵— is

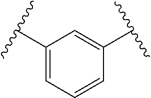 [SMILES: *c1cccc(*)c1]

(i.e., Z⁵ is phenylene where Z⁴ and Z⁶ are attached at meta position of the phenylene ring) substituted with R^{q} and R^{r} independently selected from hydrogen, deuterium, alkyl, alkoxy, halo, haloalkyl, and haloalkoxy.

A125. In embodiment A125, the compound of any one of embodiments A1 to A111 and A116 to A124, or a pharmaceutically acceptable salt thereof, is wherein —Z⁵— is

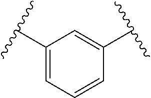 [SMILES: *c1cccc(*)c1]

substituted with R^{q} and R^{r} independently selected from hydrogen, deuterium, methyl, methoxy, fluoro, chloro, difluoromethyl, trifluoromethyl, difluoromethoxy, and trifluoromethoxy.

A126. In embodiment A126, the compound of any one of embodiments A1 to A111 and A116 to A125, or a pharmaceutically acceptable salt thereof, is wherein —Z⁵— is

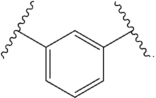 [SMILES: *c1cccc(*)c1]

substituted with R^{q} and R^{r} independently selected from hydrogen, deuterium, methyl, fluoro, trifluoromethyl, and trifluoromethoxy.

A127. In embodiment A127, the compound of any one of embodiments A1 to A111 and A116 to A123b, or a pharmaceutically acceptable salt thereof, is wherein —Z⁵— is imidazol-1,5-diyl, pyridin-2,4-diyl, pyridin-2,6-diyl, or pyridin-3,5-diyl, each ring substituted with R^{q} and R^{r} independently selected from hydrogen, alkyl, alkoxy, halo, haloalkyl, and haloalkoxy.

A128. In embodiment A128, the compound of any one of embodiments A1 to A111, A116 to A123b and A127, or a pharmaceutically acceptable salt thereof, is wherein —Z⁵— is imidazole-2,5-diyl, pyridin-2,4-diyl, pyridin-2,6-diyl, or pyridin-3,5-diyl, each ring substituted with R^{q} and R^{r} independently selected from hydrogen, methyl, methoxy, fluoro, chloro, difluoromethyl, trifluoromethyl, 2,2,2-trifluoroethyl, difluoromethoxy, and trifluoromethoxy.

A129. In embodiment A129, the compound of any one of embodiments A1 to A128, or a pharmaceutically acceptable salt thereof, is wherein each alkylene of —Z¹—Z²—Z³—Z⁴—Z^{S}—Z⁶—, by itself and when present, is methylene, ethylene, or propylene, each substituted with R^{s} and R^{t}.

A130. In embodiment A130, the compound of any one of embodiments A1 to A128, or a pharmaceutically acceptable salt thereof, is wherein each alkylene of —Z¹—Z²—Z³—Z⁴—Z⁵—Z⁶—, by itself and when present, is methylene substituted with R^{s} and R^{t}.

A131. In embodiment A131, the compound of any one of embodiments A1 to A130, or a pharmaceutically acceptable salt thereof, is wherein each alkylene of —Z¹—Z²—Z³—Z⁴—Z⁵—Z⁶—, as part of another group (e.g, —(O-alkylene)ₐ, -(alkylene-O)ₐ—, -(alkylene-NR″)—) and when present, is ethylene or propylene.

A132. In embodiment A132, the compound of any one of embodiments A1 to A131, or a pharmaceutically acceptable salt thereof, is wherein each alkylene of —Z¹—Z²—Z³—Z⁴—Z⁵—Z⁶—, as part of another group (e.g, —(O-alkylene)ₐ, -(alkylene-O)ₐ—, -(alkylene-NR″)—) and when present, is ethylene.

A133. In embodiment A133, the compound of any one of embodiments A1 to A132, or a pharmaceutically acceptable salt thereof, is wherein each R, R′ and R″ of —Z¹—Z²—Z³—Z⁴—Z^{S}—Z⁶—, when present, is independently hydrogen or methyl.

A134. In embodiment A134, the compound of any one of embodiments A1 to A133, or a pharmaceutically acceptable salt thereof, is wherein each R, R′ and R″ of —Z¹—Z²—Z³—Z⁴—Z⁵—Z⁶—, when present, is hydrogen.

A135. In embodiment A135, the compound of any one of embodiments A1 to A133, or a pharmaceutically acceptable salt thereof, is wherein each R, R′ and R″ of —Z¹—Z²—Z³—Z⁴—Z^{S}—Z⁶—, when present, is methyl.

A136. In embodiment A136, the compound of any one of embodiments A1 to A135, or a pharmaceutically acceptable salt thereof, is wherein each cycloalkylene of —Z²—Z³—Z⁴—, when present, is independently selected from cyclopropylene, cyclobutylene, cyclopentylene, and cyclohexylene.

A137. In embodiment A137, the compound of any one of embodiments A1 to A136, or a pharmaceutically acceptable salt thereof, is wherein each cycloalkylene of —Z²—Z³—Z⁴—, when present, is independently selected from 1,3-cyclopentylene, 1,3-cyclohexylene, and 1,4-cyclohexylene.

A138. In embodiment A138, the compound of any one of embodiments A1 to A137, or a pharmaceutically acceptable salt thereof, is wherein heteroarylene is monocyclic heteroarylene and each monocyclic heteroarylene of —Z¹—Z³—Z⁴—Z⁵—, when present, is independently selected from imidazoldiyl, pyridindiyl and pyrimidindiyl unless stated otherwise in any of the embodiments above.

A139. In embodiment A139, the compound of any one of embodiments A1 to A138, or a pharmaceutically acceptable salt thereof, is wherein heteroarylene is monocyclic heteroarylene and each monocyclic heteroarylene of —Z¹—Z³—Z⁴—Z⁵—, when present, is independently selected from imidazol-2,5-diyl, pyridin-2,4-diyl, pyridin-2,6-diyl, and pyridin-3,5-diyl, unless stated otherwise in any of the embodiments above.

A140. In embodiment A140, the compound of any one of embodiments A1 to A139, or a pharmaceutically acceptable salt thereof, is wherein each phenylene of —Z¹—Z³—Z⁴—Z⁵—, when present, is independently selected from 1,3-phenylene and 1,4-phenylene unless stated otherwise in any of the embodiments above.

A141. In embodiment A141, the compound of any one of embodiments A1 to A140, or a pharmaceutically acceptable salt thereof, is wherein each heterocyclylene, bridged heterocyclylene, and spiro heterocyclylene, of —Z¹—Z²—Z³—Z⁴—Z⁵—, when present, are independently selected from

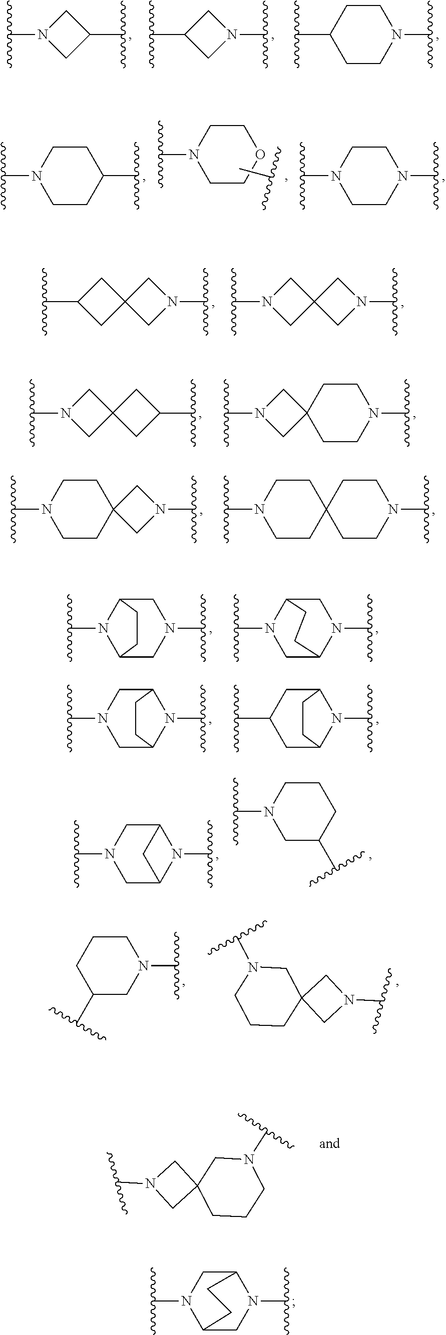

wherein each ring is optionally substituted with 1, 2, or 3 fluoro.

A142. In embodiment A142, the compound of any one of embodiments A1 to A96b, or a pharmaceutically acceptable salt thereof, is wherein L (when Degron is a group of any one of formula (iii) to (iv)), —X¹-L-, —X²-L-, —X³-L- and —X⁴-L- are independently:

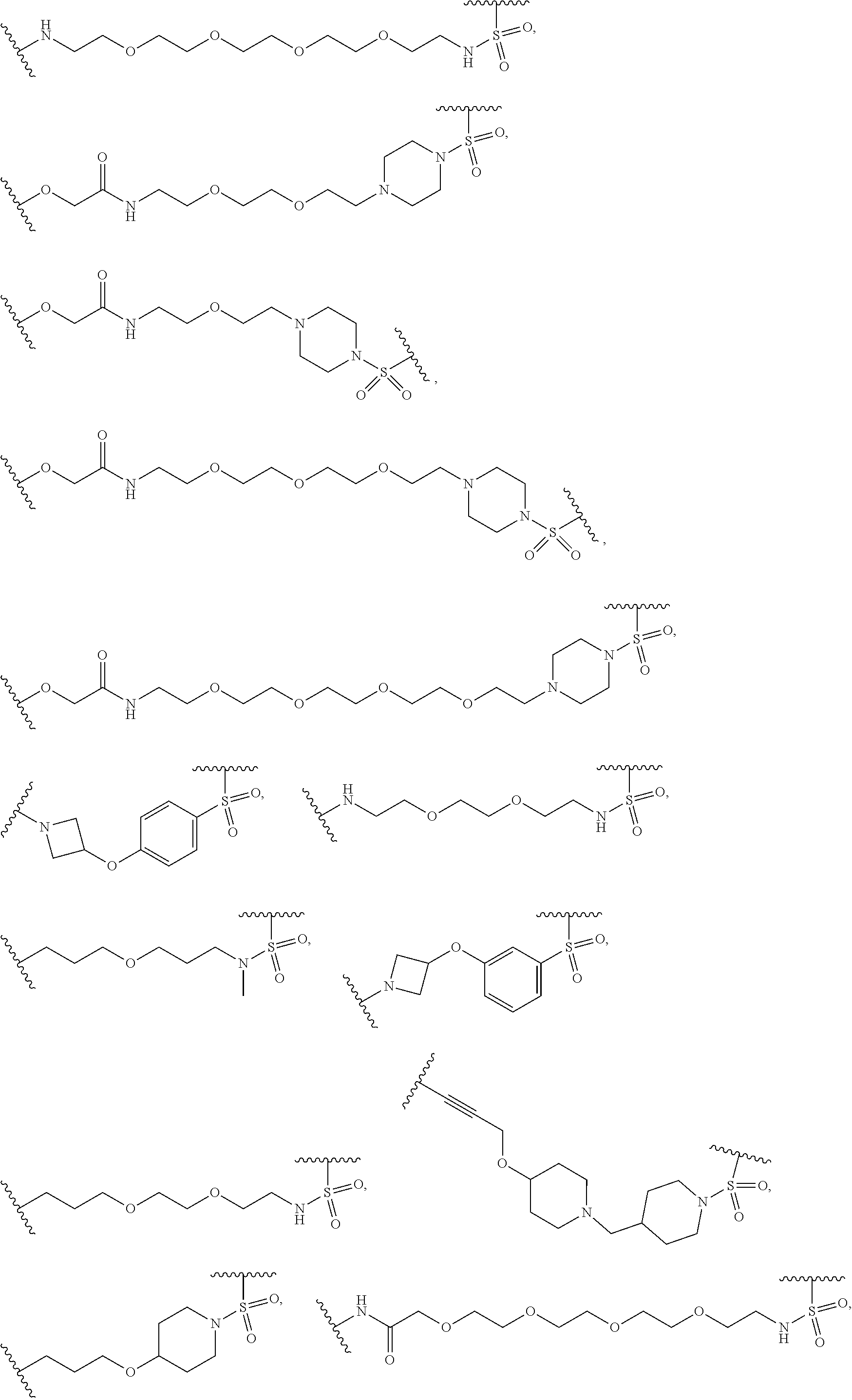

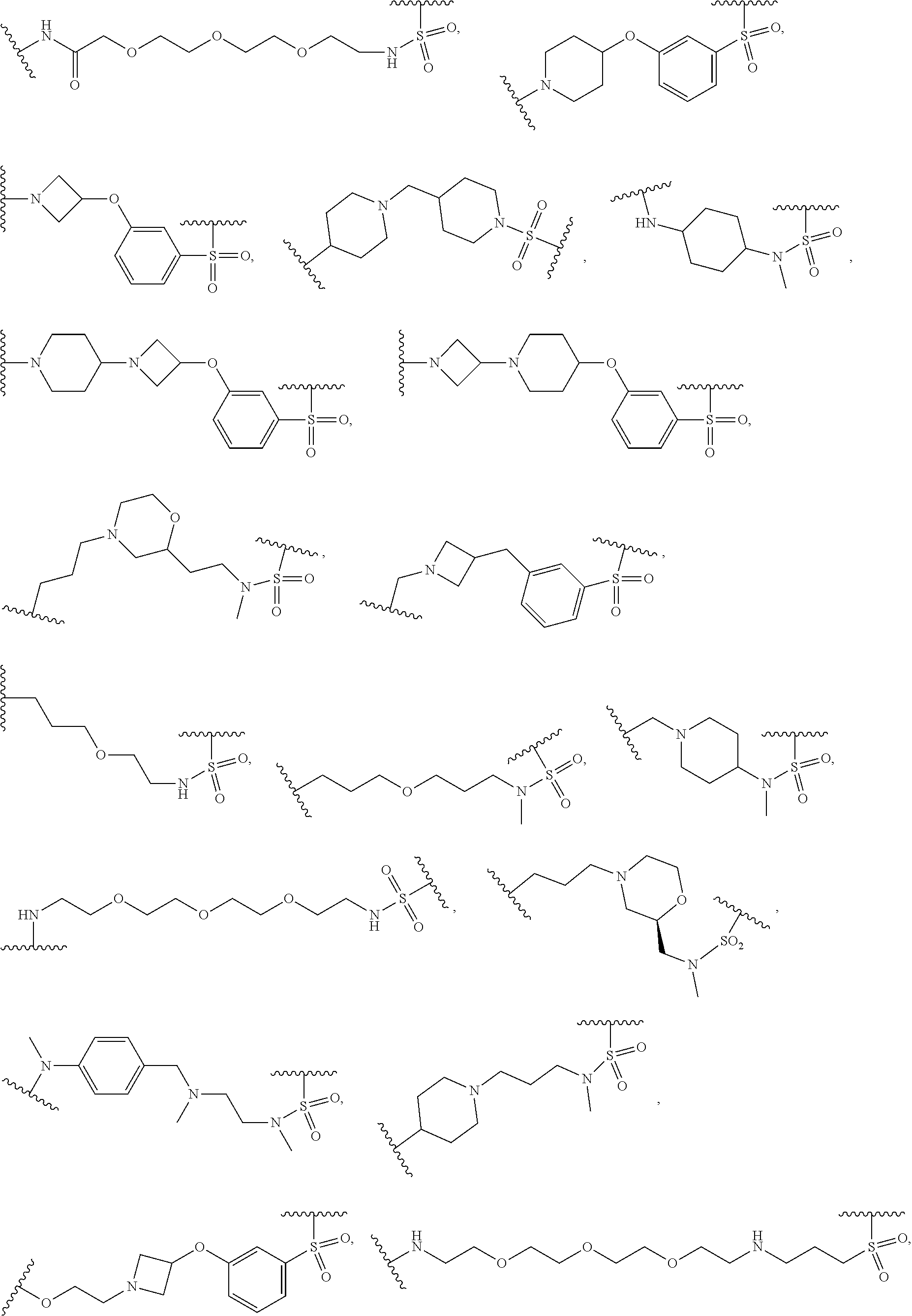

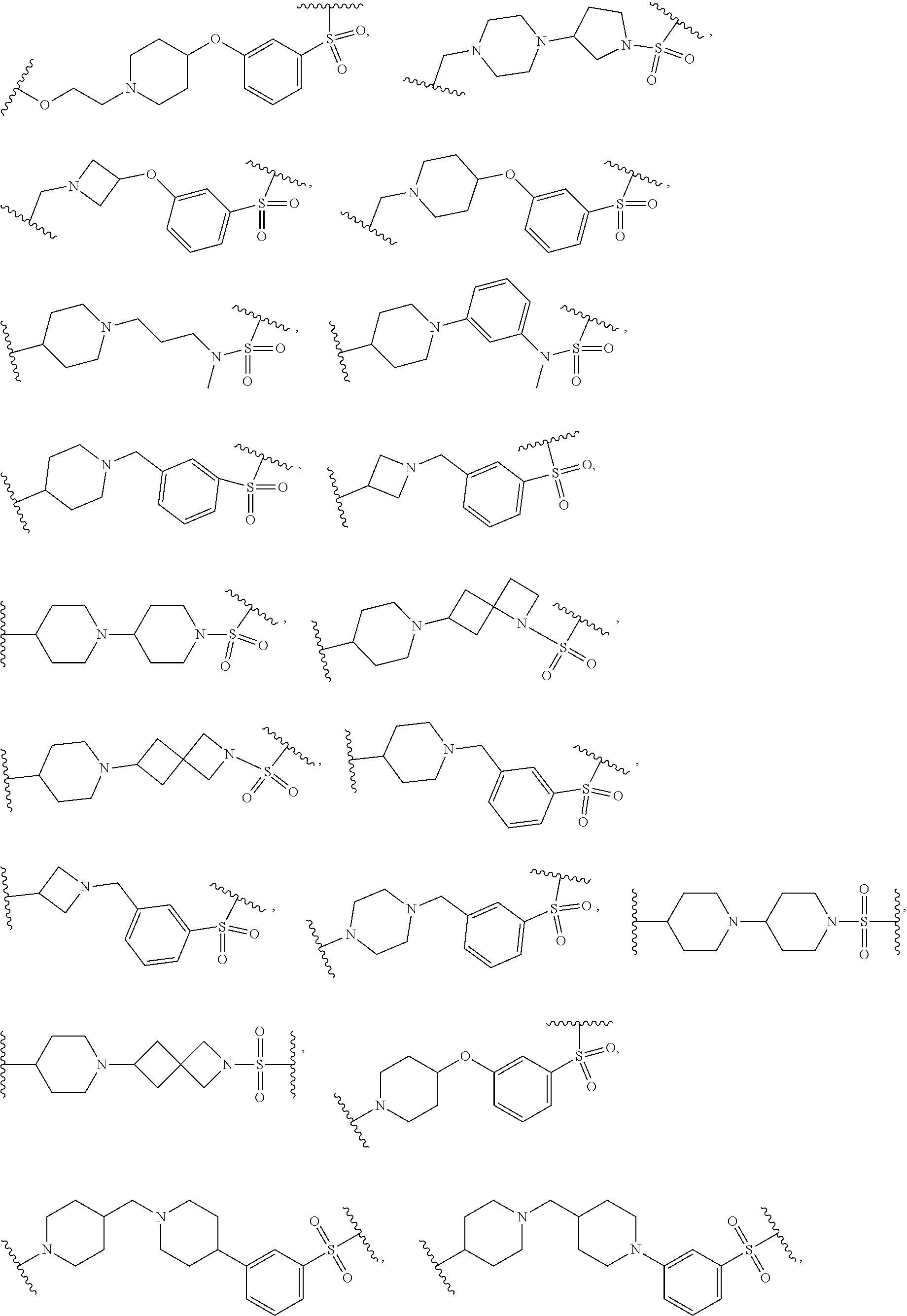

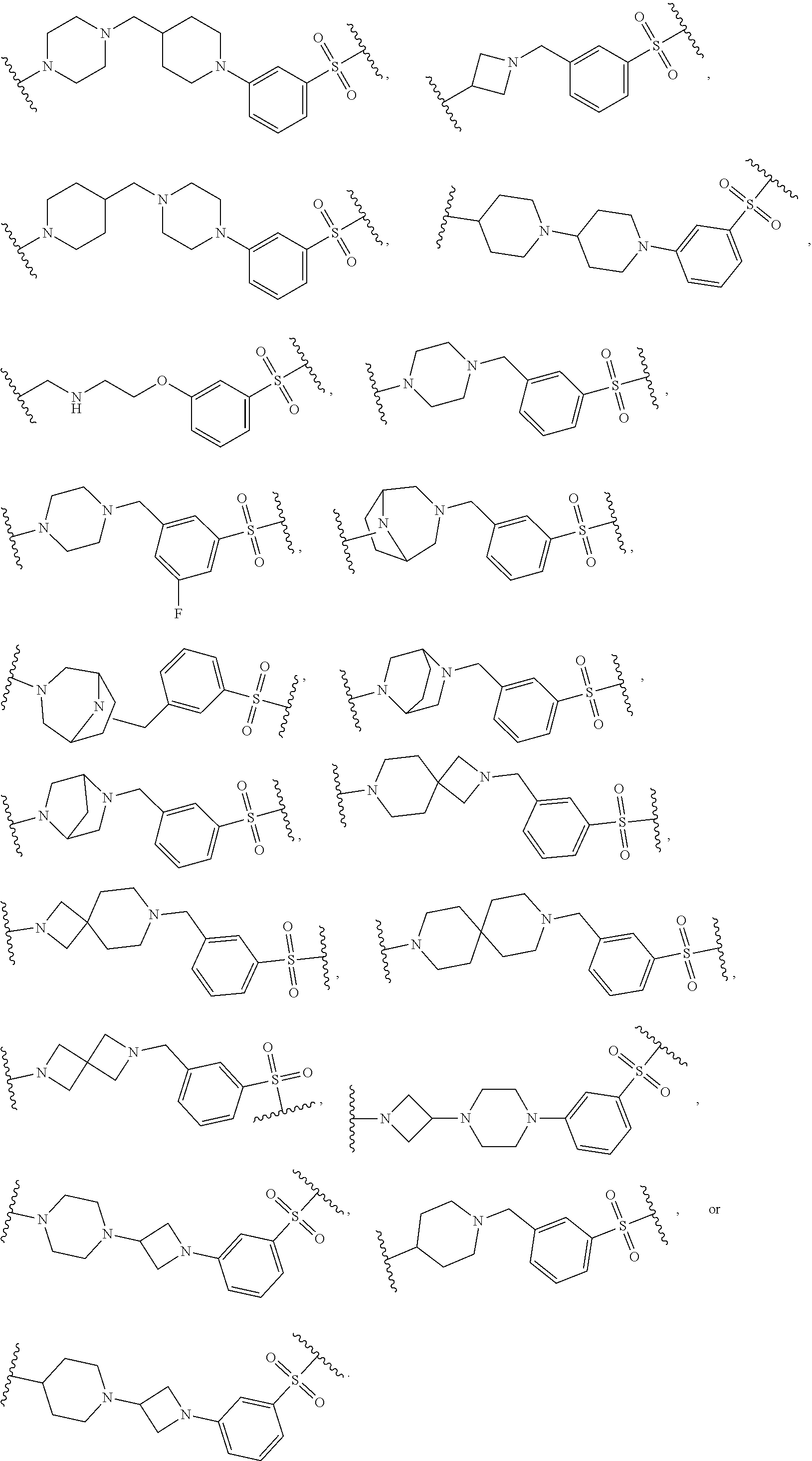

A143. In embodiment A143, the compound of any one of embodiments A1 to A96b and A142, or a pharmaceutically acceptable salt thereof, is wherein L (when Degron is a group of any one of formula (iii) to (iv)), —X¹-L-, —X²-L-, —X³-L-, and —X⁴-L- are independently:

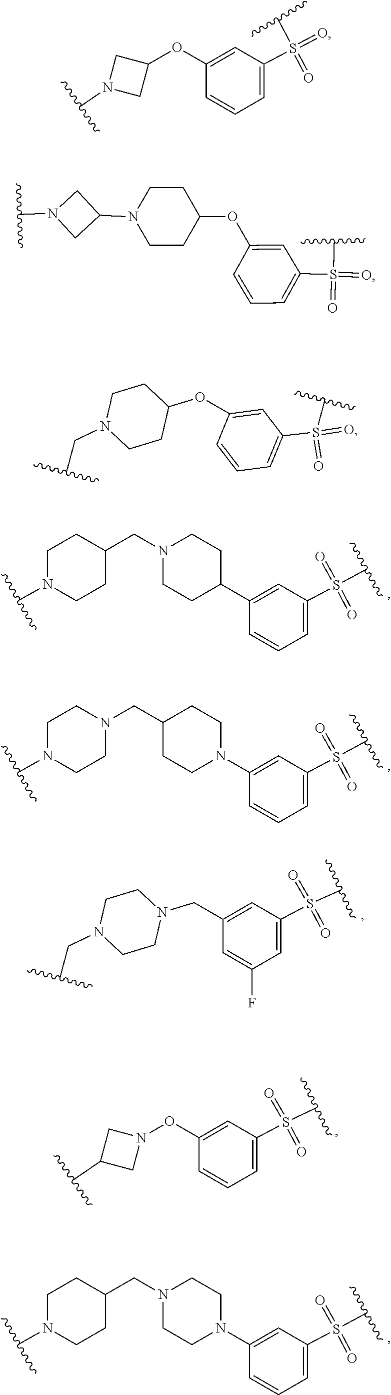

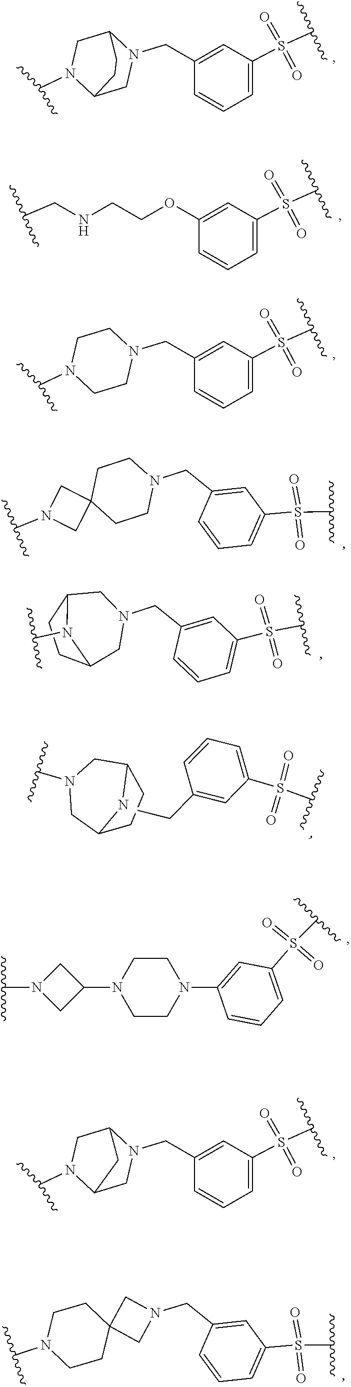

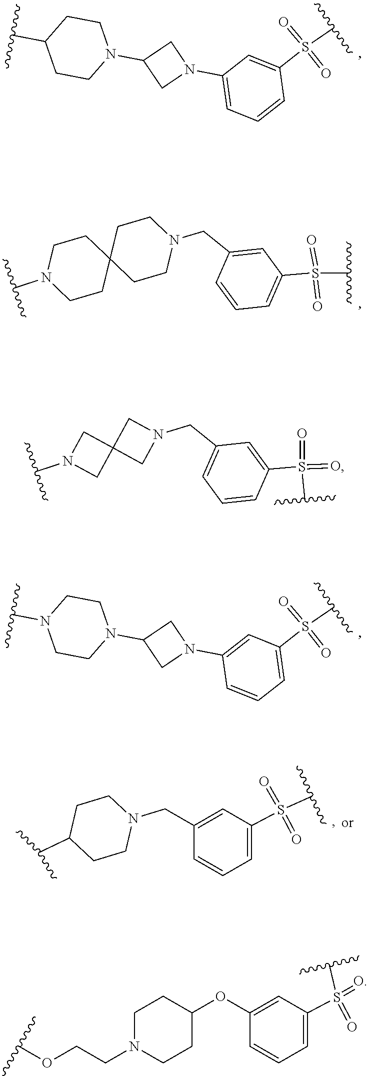 [SMILES: *C1CCN(C2CN(c3cccc(S(*)(=O)=O)c3)C2)CC1 ; *N1CCC2(CC1)CCN(Cc1cccc(S(*)(=O)=O)c1)CC2 ; *N1CC2(C1)CN(Cc1cccc(S(*)(=O)=O)c1)C2 ; *N1CCN(C2CN(c3cccc(S(*)(=O)=O)c3)C2)CC1 ; *C1CCN(Cc2cccc(S(*)(=O)=O)c2)CC1 ; *OCCN1CCC(Oc2cccc(S(*)(=O)=O)c2)CC1]

A144. In embodiment A144, the compound of any one of embodiments A1 to A110 and A120-A120b, or a pharmaceutically acceptable salt thereof, is wherein —Z³—Z⁴—Z⁵—Z⁶— is:

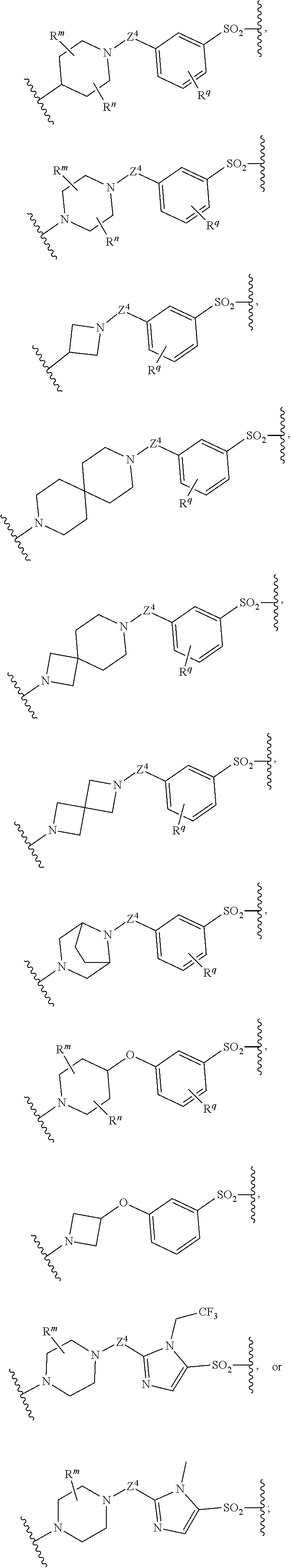

wherein R^{q}, R^{m}, and Rⁿ are independently selected from hydrogen, alkyl, cycloalkyl, halo, haloalkyl, haloalkoxy, alkoxy, and cyano.

A144a. In embodiment A144a, the compound of any one of embodiments A1 to A110, A120-A120b, and A144, or a pharmaceutically acceptable salt thereof, is wherein R^{q} and R^{m} are independently selected from hydrogen, methyl, fluoro, chloro, cyano, methoxy, difluoromethoxy, cyclopropyl, difluoromethyl, and trifluoromethyl.

A145. In embodiment A145, the compound of any one of embodiments A1 to A96b, A120-A120b, and A144, or a pharmaceutically acceptable salt thereof, is wherein —Z³—Z⁴—Z⁵—Z⁶— is:

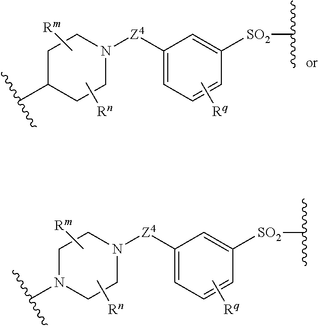

wherein R^{m}, Rⁿ, and R^{q} are independently selected from hydrogen, alkyl, cycloalkyl, halo, haloalkyl, haloalkoxy, alkoxy, and cyano.

A145a1. In embodiment A145a1, the compound of any one of embodiments A1 to A96b, A120-A120b, A144, and A145, or a pharmaceutically acceptable salt thereof, is wherein R^{q} and R^{m} are independently selected from hydrogen, methyl, fluoro, chloro, cyano, methoxy, difluoromethoxy, cyclopropyl, difluoromethyl, and trifluoromethyl.

A145a. In embodiment A145a, the compound of any one of embodiments A1 to A96b, A120-A120b, A144, to A145a1, or a pharmaceutically acceptable salt thereof, is wherein Rⁿ is hydrogen.

A146. In embodiment A146, the compound of any one of embodiments A1 to A110, A116 and A123, or a pharmaceutically acceptable salt thereof, is —Z²—Z³—Z⁴—Z⁵—Z⁶— is:

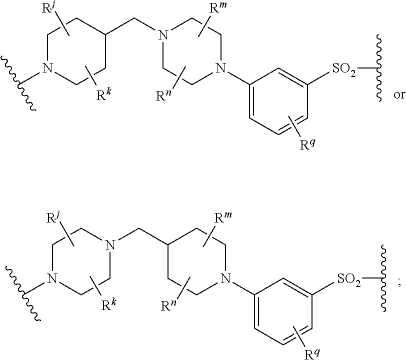

wherein R^{j}, R^{k}, R^{m}, Rⁿ, and R^{q} are independently selected from hydrogen, alkyl, cycloalkyl, halo, haloalkyl, haloalkoxy, alkoxy, and cyano.

A146a1. In embodiment A146a1, the compound of any one of embodiments A1 to A110, A116, A123, and A146, or a pharmaceutically acceptable salt thereof, is wherein R^{q} and R^{m} are independently selected from hydrogen, methyl, fluoro, chloro, cyano, methoxy, difluoromethoxy, cyclopropyl, difluoromethyl, and trifluoromethyl.

A146a. In embodiment A146a, the compound of any one of embodiments A1 to A110, A116, A123, and A146a1, or a pharmaceutically acceptable salt thereof, is wherein Rⁿ is hydrogen.

A147. In embodiment A147, the compound of any one of embodiments A1 to A111, A113, A114, A116-A122, A124 to A128, A133-A135, A144 to A145a, or a pharmaceutically acceptable salt thereof, is wherein Z⁴ is alkylene substituted with R^{s} and R^{t} where R^{s} and R^{t} are hydrogen.

A148. In embodiment A148, the compound of any one of embodiments A1 to A111, A113, A114, A116-A122, A124 to A128, A133-A135, A144 to A145a, and A147, or a pharmaceutically acceptable salt thereof, is wherein the alkylene of Z⁴ is —CH₂—, —(CH₂)₂—, —CH(CH₃)—, —CH₂CH(CH₃)—CH₂—, or —C(CH₃)₂—.

A148a. In embodiment A148a, the compound of embodiment A148, or a pharmaceutically acceptable salt thereof, is wherein Z⁴ is —CH₂—, —(CH₂)₂-, —CH(CH₃)—, or —C(CH₃)₂-.

A149. In embodiment A149, the compound of any one of embodiments A1 to A111, A113-A122, A124 to A128, A133-A135, A144 to A145a, or a pharmaceutically acceptable salt thereof, is wherein Z⁴ is —O—.

A150. In embodiment A150, the compound of any one of embodiments A1 to A111, A113, A114, A116-A122, A124 to A128, A133-A135, A144 to A145a, or a pharmaceutically acceptable salt thereof, is wherein Z⁴ is alkylene substituted with R^{s} and R^{t} where R^{s} is hydrogen and deuterium and R^{t} is hydrogen, deuterium, haloalkyl, hydroxy, alkoxy, or cyano.

A151. In embodiment A151, the compound of any one of embodiments A1 to A111, A113, A114, A116-A122, A124 to A128, A133-A135, A144 to A145a, and A150, or a pharmaceutically acceptable salt thereof, is wherein Z⁴ is —CR^{s}R^{t} where R^{s} is hydrogen or deuterium and R^{t} is hydrogen or deuterium.

A152. In embodiment A152, the compound of any one of embodiments A1 to A111, A113, A114, A116-A122, A124 to A128, A133-A135, A144 to A145a, and A150, or a pharmaceutically acceptable salt thereof, is wherein Z⁴ is alkylene substituted with R^{s} and R^{t} where R^{s} is hydrogen and R^{t} is haloalkyl.

A153. In embodiment A153, the compound of any one of embodiments A1 to A111, A113, A114, A116-A122, A124 to A128, A133-A135, A144 to -A145a, and A150, or a pharmaceutically acceptable salt thereof, is wherein Z⁴ is alkylene substituted with R^{s} and R^{t} where R^{s} is hydrogen and R^{t} is hydroxy.

A154. In embodiment A154, the compound of any one of embodiments A1 to A111, A113, A114, A116-A122, A124 to A128, A133-A135, A144 to A145a, and A150, or a pharmaceutically acceptable salt thereof, is wherein Z⁴ is alkylene substituted with substituted with R^{s} and R^{t} where R^{s} is hydrogen and R^{t} is alkoxy.

A155. In embodiment A155, the compound of any one of embodiments A1 to A111, A113, A114, A116-A122, A124 to A128, A133-A135, A144 to A145a, A150, and A152 to A154, or a pharmaceutically acceptable salt thereof, is wherein Z⁴ is —CH(CHF₂)—, —CH(CF₃)—, —C(CH₃)(CF₃)—, —CH(CH₂CF₃)—, —CH(CH₂CH₂CF₃)—, —CH(CH(CF₃)₂)—, —CH(CH₂OH)—, —CH(CH₂OCH₃)—, —CH(CH₂O-ethyl)-, or —CH(CH₂CN)—,

A156. In embodiment A156, the compound of any one of embodiments A1 to A111, A113, A114, A116-A122, A124 to A128, A133-A135, A144 to A145a, or a pharmaceutically acceptable salt thereof, is wherein Z is alkylene substituted with R^{s} and R^{t} where R^{s} is hydrogen and R^{t} is cycloalkyl, heterocyclyl, aryl, or monocyclic heteroaryl, wherein cycloalkyl, heterocyclyl, aryl, monocyclic heteroaryl are substituted with one or two substituents independently selected from hydrogen, alkyl, alkoxy, halo, haloalkyl, haloalkoxy, or cyano.

A157. In embodiment A157, the compound of any one of embodiments A1 to A111, A113, A114, A116-A122. A124 to A128, A133-A135, A144 to 145a, and A156, or a pharmaceutically acceptable salt thereof, is wherein Z⁴ is alkylene substituted with R^{s} and R^{t} where R^{s} is hydrogen and deuterium and R^{t} is cycloalkyl substituted with one or two substituents independently selected from hydrogen, alkyl, alkoxy, halo, haloalkyl, haloalkoxy, or cyano.

A158. In embodiment A158, the compound of any one of embodiments A1 to A111, A113, A114, A116-A122, A124 to A128, A133-A136, A144 to 145a, and A156, or a pharmaceutically acceptable salt thereof, is wherein Z⁴ is alkylene substituted with R^{s} and R^{t} where R^{s} is hydrogen and R^{t} is heterocyclyl substituted with one or two substituents independently selected from hydrogen, alkyl, alkoxy, halo, haloalkyl, haloalkoxy, or cyano.

A159. In embodiment A159, the compound of any one of embodiments A1 to A111, A113, A114, A116-A122, A124 to A128, A133-A135, A144 to 145a, and A156, or a pharmaceutically acceptable salt thereof, is wherein Z⁴ is alkylene substituted with R^{s} and R^{t} where R^{s} is hydrogen and R^{t} is aryl substituted with one or two substituents independently selected from hydrogen, alkyl, alkoxy, halo, haloalkyl, haloalkoxy, or cyano.

A160. In embodiment A160, the compound of any one of embodiments A1 to A111, A113, A114, A116-A122, A124 to A128, A133-A135, A144 to 145a, and A156, or a pharmaceutically acceptable salt thereof, is wherein Z⁴ is alkylene substituted with R^{s} and R^{t} where R^{s} is hydrogen and R^{t} is monocyclic heteroaryl, substituted with one or two substituents independently selected from hydrogen, alkyl, alkoxy, halo, haloalkyl, haloalkoxy, or cyano.

A161. In embodiment A161, the compound of any one of embodiments A1 to A111, A113, A114, A116-A122, A124 to A128, A133-A135, A144 to A145a, and A156 to 160, or a pharmaceutically acceptable salt thereof, is wherein Z⁴ is —CH(benzyl)-, —CH(phenyl)-, —CH(pyridin-4-yl)-, —CH(cyclopentyl)-, —CH(cyclohexyl)-, —CH(tetrahydropyran-4-yl)-, or —CH(piperidin-4-yl)-, wherein phenyl, either by itself or as part of benzyl, pyridine-4-yl, cyclopentyl, cyclohexyl, tetrahydropyran-4-yl, and piperidin-4-yl are substituted with one or two substituents independently selected from hydrogen, alkyl, alkoxy, halo, haloalkyl, haloalkoxy, or cyano.

A162. In embodiment A162, the compound of any one of embodiments A1 to A111, A113, A114, A116-A122, A124 to A128, A133-A135, A144 to 145a, and A156 to 160, or a pharmaceutically acceptable salt thereof, is wherein Z⁴ is —CH(benzyl)-, —CH(phenyl)-, —CH(pyrazol-4-yl)-, —CH(pyridin-4-yl)-, —CH(cyclopentyl)-, —CH(cyclohexyl)-, —CH(tetrahydropyran-4-yl)-, or —CH(piperidin-4-yl)-, wherein phenyl, either by itself or as part of benzyl, pyrazol-4-yl, pyridin-4-yl, cyclopentyl, cyclohexyl, tetrahydropyran-4-yl, and piperidin-4-yl are substituted with one or two substituents independently selected from hydrogen, fluoro, chloro, methyl, methoxy, difluoromethoxy, trifluoromethoxy, or cyano.

A163. In embodiment A163, the compound of any one of embodiments A1 to A111, A113-A120, A120b, A123b, A124 to A128, A133-A135, A144 to A145a, or a pharmaceutically acceptable salt thereof, is wherein Z⁴ is 1,1-cycloalkylene substituted with R^{o} and R^{p}.

A164. In embodiment A164, the compound of any one of embodiments A1 to A111, A113-A120, A120b, A123b, A124 to A128, A133, A144 to A145a, or a pharmaceutically acceptable salt thereof, is wherein Z⁴ is heterocyclylene substituted with R^{o} and R^{p}.

A165. In embodiment A165, the compound of any one of embodiments A1 to A120, A120b. A123b, A124 to A128, A133, A144 to A145a, and A163-A164, or a pharmaceutically acceptable salt thereof, is wherein Z⁴ is:

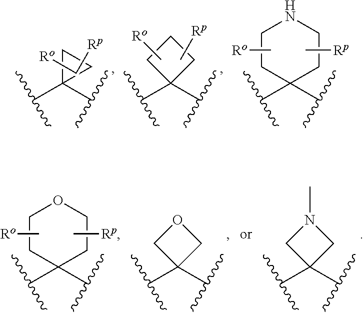

A166. In embodiment A166, the compound of any one of embodiments A1 to A165, or a pharmaceutically acceptable salt thereof, is wherein Degron is the E3 ligase ligand selected from:

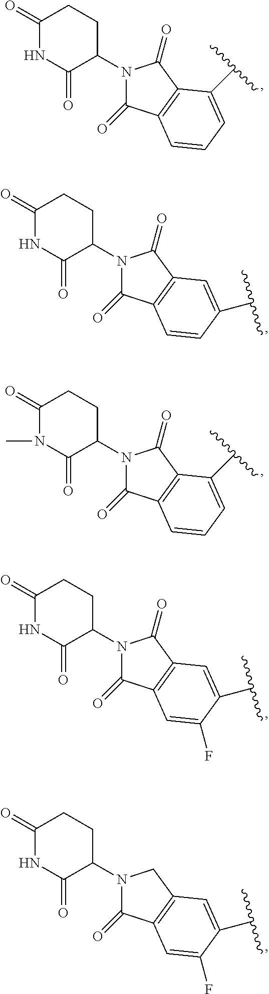 [SMILES: *c1cccc2c1C(=O)N(C1CCC(=O)NC1=O)C2=O ; *c1ccc2c(c1)C(=O)N(C1CCC(=O)NC1=O)C2=O ; *c1cccc2c1C(=O)N(C1CCC(=O)N(C)C1=O)C2=O ; *c1cc2c(cc1F)C(=O)N(C1CCC(=O)NC1=O)C2=O ; *c1cc2c(cc1F)C(=O)N(C1CCC(=O)NC1=O)C2]

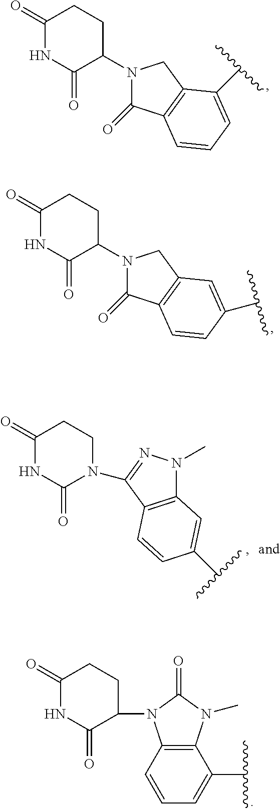 [SMILES: *c1cccc2c1CN(C1CCC(=O)NC1=O)C2=O ; *c1ccc2c(c1)CN(C1CCC(=O)NC1=O)C2=O ; *c1ccc2c(N3CCC(=O)NC3=O)nn(C)c2c1 ; *c1cccc2c1n(C)c(=O)n2C1CCC(=O)NC1=O]

A167. In embodiment A167, the compound of any one of embodiments A1 to A165, or a pharmaceutically acceptable salt thereof, is wherein Degron is the E3 ligase ligand selected from:

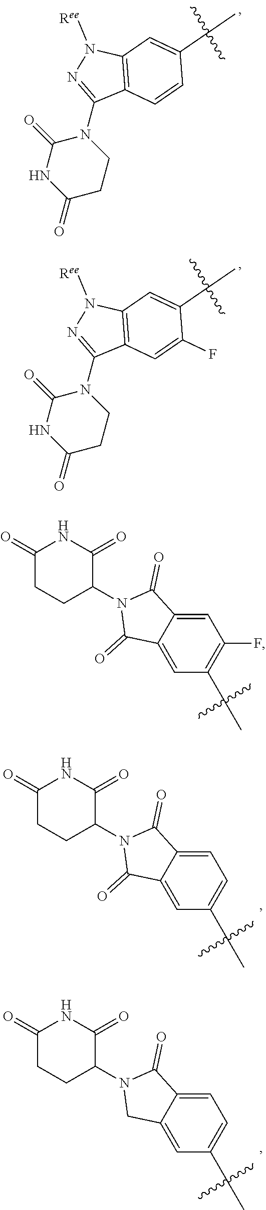

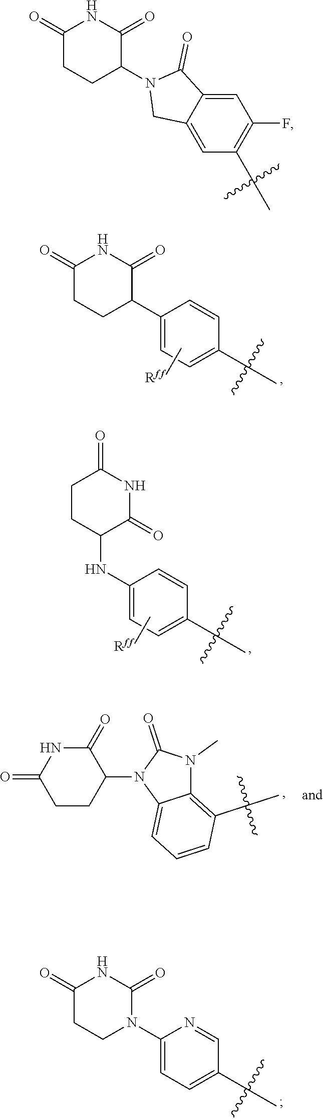

where R^{ee} is hydrogen, methyl, ethyl, cyclopropyl, or 2,2,2-trifluoroethyl and R^{ff} is hydrogen, methyl, cyclopropyl, fluoro, cyano, methoxy, difluoromethoxy, trifluoromethoxy, or trifluoromethyl.

A168. In embodiment A168, the compound of any one of embodiments A1 to A165, or a pharmaceutically acceptable salt thereof, is wherein Degron is the E3 ligase ligand selected from:

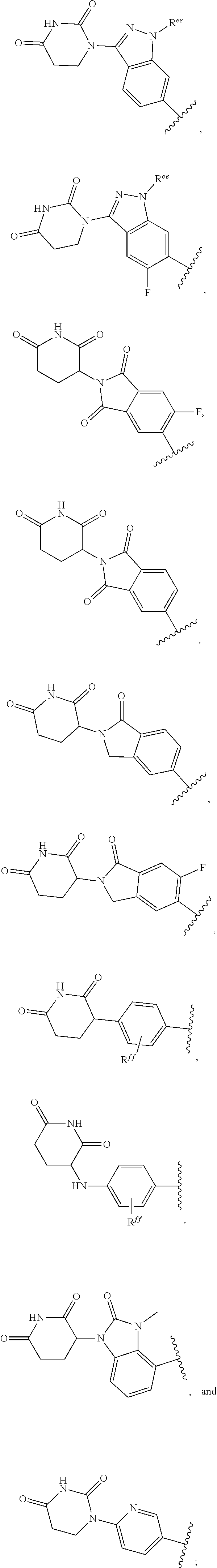

where R^{ee} is hydrogen, methyl, cyclopropyl, or 2,2,2-trifluoroethyl and R^{ff} is hydrogen, methyl, fluoro, or trifluoromethyl.

A169. In embodiment A169, the compound of any one of embodiments A1 to A165 is wherein R^{x} is hydrogen.

A170. In embodiment A170, provided is a pharmaceutical composition comprising a compound of any one of embodiments A1 to A169, or a pharmaceutically acceptable salt thereof, and a pharmaceutically acceptable excipient.

A171. In embodiment A171, provided is a method of degrading CDK2 in a cell which method comprises contacting the cell with a compound of any one of embodiments A1 to A169, or a pharmaceutically acceptable salt thereof, or a pharmaceutical composition of embodiment A170.

A172. In embodiment A172, provided is a method of treating a disease mediated by CDK2 in a patient which method comprises administering to the patient in recognized need thereof, a therapeutically effective amount of a pharmaceutical composition comprising a compound of any one of embodiments A1 to A169, or a pharmaceutically acceptable salt thereof, and a pharmaceutically acceptable excipient.

A173. In embodiment A173, provided is a method of treating cancer in a patient which method comprises administering to the patient in need thereof, a therapeutically effective amount a compound of any one of embodiments A1 to A169, or a pharmaceutically acceptable salt thereof, or a pharmaceutical composition of A170.

A174. In embodiment A174, the method of embodiment A173 is wherein the compound of any one of embodiments A1 to A169 or a pharmaceutically acceptable salt thereof, or a pharmaceutical composition of A170 is administered in combination with at least one other anticancer agent.

A175. In embodiment A175, the method of embodiments A173 or A174 is wherein the cancer is lung cancer, skin cancer, bladder cancer, breast cancer, cervical cancer, colorectal cancer, cancer of the small intestine, colon cancer, rectal cancer, cancer of the anus, endometrial cancer, gastric cancer, head and neck cancer, liver cancer, ovarian cancer, prostate cancer, testicular cancer, uterine cancer, esophageal cancer, gall bladder cancer, pancreatic cancer, stomach cancer, thyroid cancer, or parathyroid cancer.

It is understood that the embodiments and subembodiments set forth above include all combinations of embodiments and subembodiments listed therein.

### General Synthetic Scheme

Compounds Formula (I) can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds Formula (I) can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

Compounds of Formula (I) where Degron is an E3 ligase ligand of formula (i) where ring A is a group of formula (a), (b), or (c) where X¹, X², and X³ are —CONH—, L is attached to Hy via —NH— and other groups are as defined in the Summary can be prepared as described in Scheme 1 below

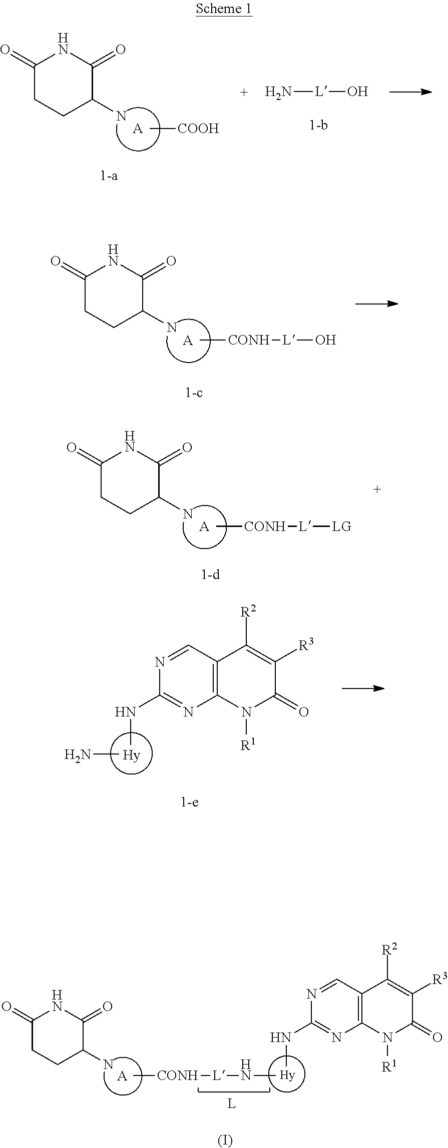

Coupling of carboxylic acid group in 1-a and a compound of formula 1-b where L′ is precursor group to L in the compound of Formula (I) as defined in the Summary, with a suitable coupling reagent such as HATU provides the corresponding amide of formula 1-c. The hydroxy group in 1-c is can be converted into a leaving group such as mesylate, triflate, and the like, by methods well known in the art to provide a compound of 1-d. Reaction of compound 1-d with a compound of formula 1-e in presence of a suitable base under conditions known in the art provides a compound of Formula (I) where X, X¹, X², and X³ are —CONH— and L is attached to Hy via —NH—.

Compounds of Formula (I) where Degron is an E3 ligase ligand of formula (i) where ring A is a group of formula (a), (b), or (c) where X¹, X², and X³ are —NH—, L is attached to Hy via —O— and other groups are as defined in the Summary can be prepared as described in Scheme 2 below.

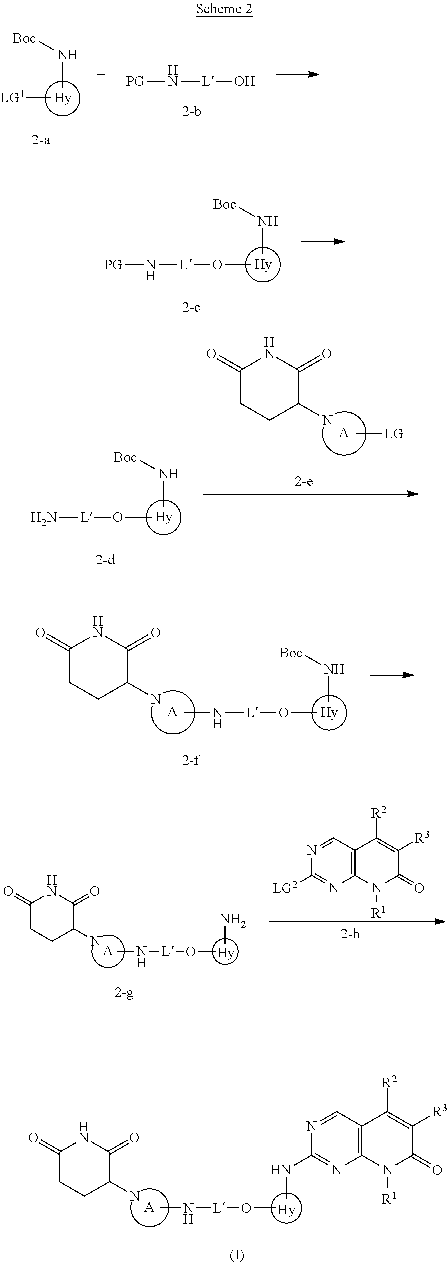

Displacement of a LG¹ (leaving group) such as halo, methylsulfonyl, and the like, in compound of formula 2-a by an alcohol compound of formula 2-b where PG is an a suitable amino protecting group such as benzyl or CBz and L′ is a precursor group to L as defined in the Summary, in presence of a suitable base such as NaH, and the like provides a compound of formula 2-c where -L′- is as precursor group of L in the compound of Formula (I) as defined in the Summary. Removal of the protecting group under suitable conditions provides an amine compound of formula 2-d. Displacement of a leaving group (LG) such as halide, sulfonate, and the like in a compound of formula 2-e where ring A is as defined in the Summary, by the amino group of formula 2-d provides a compound of formula 2-f. Removal of the Boc protecting group in compound 2-f using an acid like TFA provides a compound of formula 2-g. Reaction of compound 2-g with a compound of formula 2-h where LG² is a suitable leaving group such as Cl or SO₂Me and R¹, R² and R³ are as defined in the Summary under suitable conditions such as acidic, basic or transition metal catalyzed reaction conditions well known in the art, provides a compound of Formula (I).

Compounds of formula 2-h having the structure

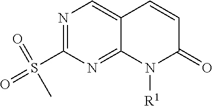

where R² and R³ are hydrogen, LG² is —SO₂Me, and R¹ are as defined in Summary can be synthesized by method illustrated in Method (a) or Method (b) below.

### Method (a)

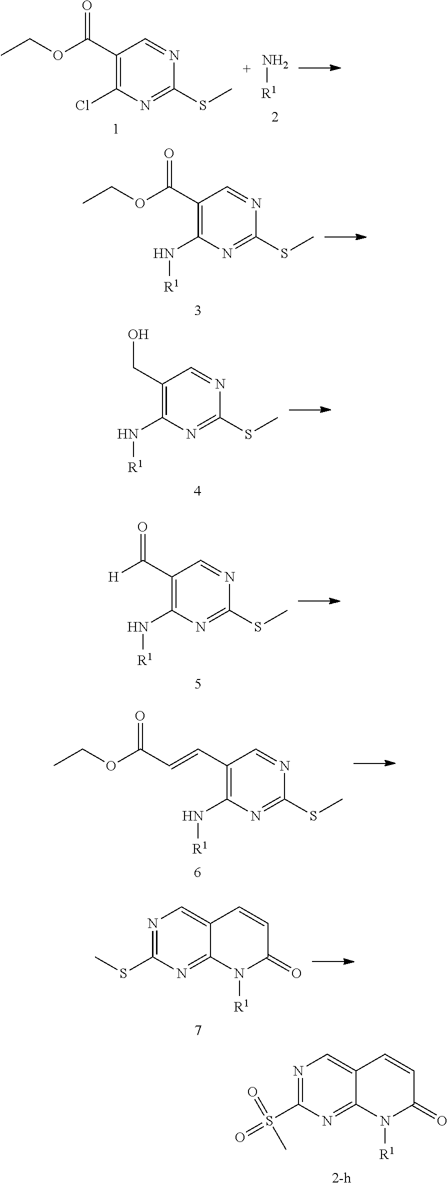

Displacement of the chloride in compound 1 with an amine of formula 2 under basic condition such as TEA provides a compound of formula 3. Compound 1 and amine 2 are commercially available or they can be prepared by methods well known the art. For example, methylamine, ethylamine, and benzylamine, are commercially available.

Reduction of the ester group in 3 with a suitable reducing reagent such LiAlH₄ provides a hydroxy compound of formula 4, which can be converted to corresponding aldehyde of formula 5 with an oxidizing agent such as MnO₂. Olefination of 5 with ethyl 2-(triphenyl-λ⁵-phosphanylidene)acetate provides a compound of formula 6, which can undergo cyclization with in the presence of abase such as DBU under heating condition to provide a compound of formula 7. Compound 7 can then be converted to a compound of formula 2-h where LG² is —SO₂Me by treating with an oxidant such as m-CPBA.

### Method (b)

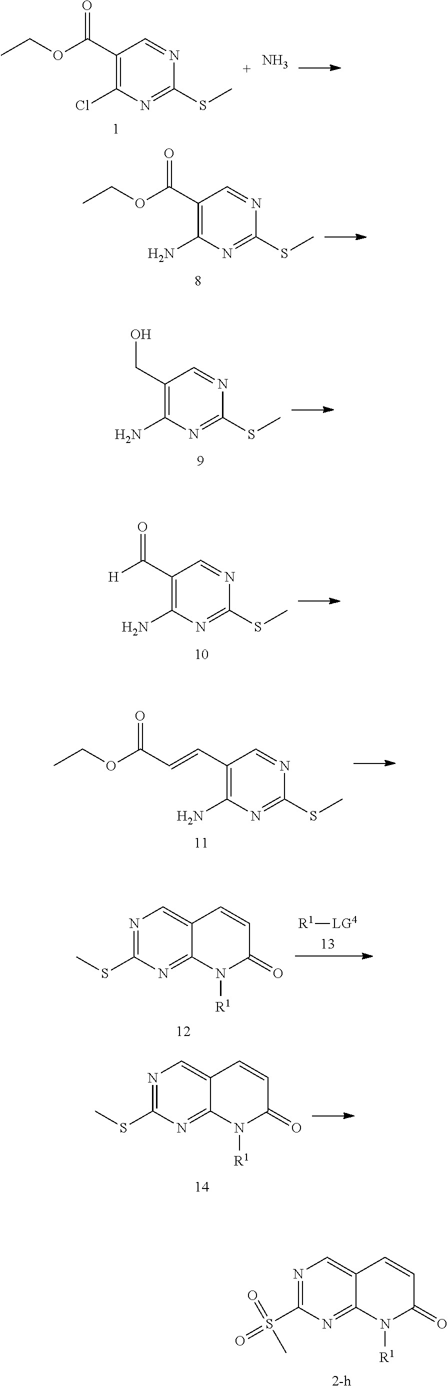

Compound of formula 12 can be synthesized by proceeding analogously to the synthesis of compound 7 in Method (a). Treatment of pyridone 12 with compound of formula 13 where R¹ is as defined in the Summary and LG⁴ is a proper leaving group, such as halogen, trifluoromethanesulfonate, under basic conditions, such as in the presence of NaH, Cs₂CO₃, provide compound of formula 14. Compound 14 can then be converted to a compound of formula 2-h where LG² is —SO₂Me by treating with an oxidant such as m-CPBA.

Compounds of Formula (I) where Degron is an E3 ligase ligand of formula (i) where ring A is a group of formula (a), (b), or (c), X¹, X², and X³ are ethylene, L is attached to Hy via —NH— and other groups are as defined in the Summary can be prepared as described in Scheme 3 below:

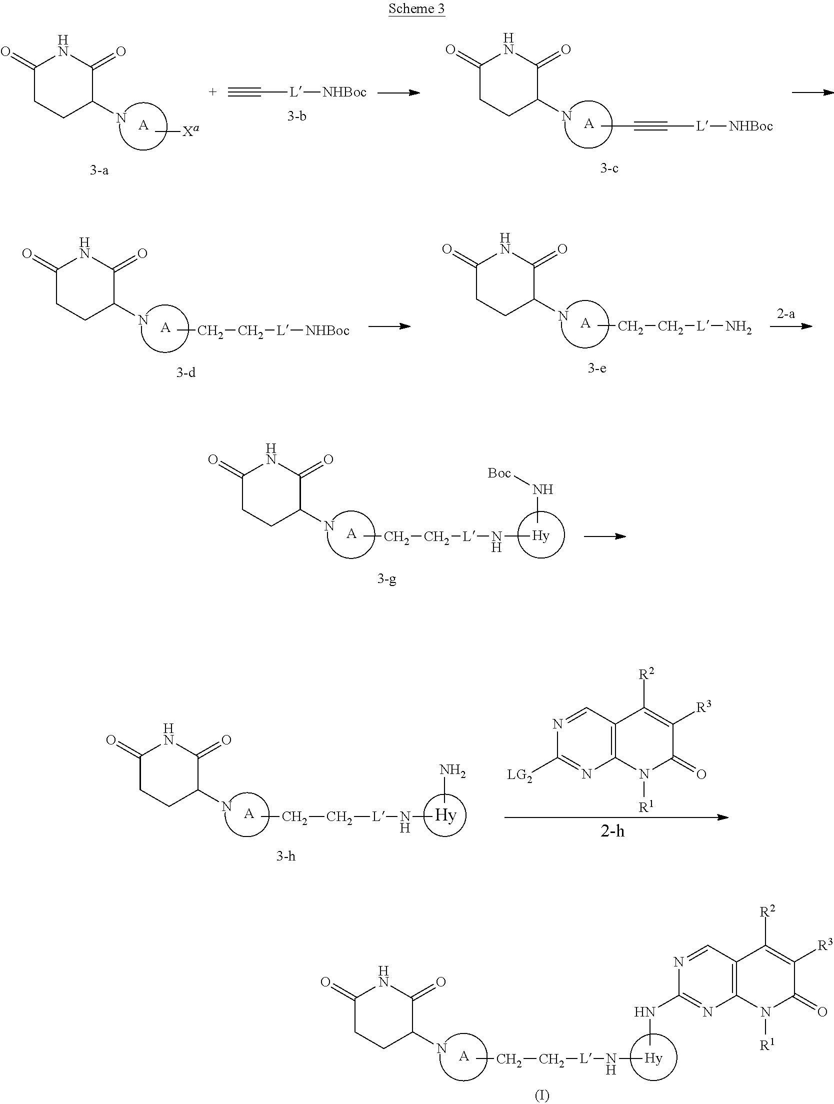

Sonogashira coupling of a compound of formula 3-a X^{a} is a halo with terminal alkyne group of a compound of formula 3-b where L′ is a precursor group of L in the compound of Formula (I) as defined in the Summary, in the presence of a palladium(0) catalyst, a copper(I) cocatalyst and a suitable base such as triethylamine, and the like provides a compound of formula 3-c. Hydrogenated of the triple bond provides a corresponding compound of formula 3-d. Removal of the Boc group in 3-d using an acid such as TFA provides an amine compound of formula 3-e, Reaction of 3-e with a compound of formula 2-a under suitable conditions provides a compound of formula 3-g. Removal of the Boc group in compound 3-g, followed by reaction of the resulting amine of formula 3-h with a compound of formula 2-h as described in above provides a compound of Formula (I).

Compounds of Formula (I) where Degron is an E3 ligase ligand of formula (ii) where X⁴ is a bond, L is attached to the E3 ligase ligand of formula (ii) via piperidinyl ring and to Hy via —SO₂— and other groups are as defined in the Summary can be prepared as described in Scheme 4 below:

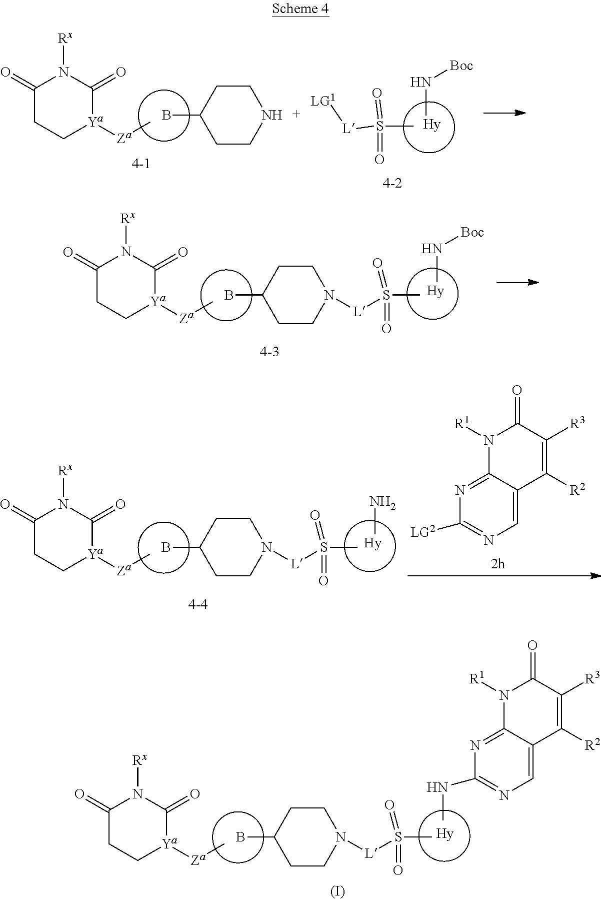

Displacement of a LG¹ (leaving group) such as halo, methylsulfonyl, and the like, in compound of formula 4-2 by an amine compound of formula 4-1 where ring B, ring Hy, R^{x}, Y^{a}, Z^{a} are as defined in the Summary and L′ is a precursor group to L as defined in the Summary, in the presence of a suitable base such as TEA, and the like, provides a compound of formula 4-3. Removal of the Boc protecting group in compound 4-3 using an acid like TFA provides an amine compound of formula 4-4. Reaction of compound 4-4 with a compound of formula 2-h where LG² is a suitable leaving group such as Cl or SO₂Me and R¹, R² and R³ are as defined in the Summary under suitable conditions such as acidic, basic or transition metal catalyzed reaction conditions well known in the art, provides a compound of Formula (I).

### Utility

The compound of Formula (I) could cause degradation of CDK2 protein via ubiquitin proteosome pathway and hence are useful in the treatment of diseases mediated by CDK2. Increasing evidence suggests that overactivated CDK2 leads to abnormal cell cycle regulation and proliferation in cancer cells. While CDK2 mutations are rarely found, the kinase activity of CDK2/Cyclin E or CDK2/Cyclin A complexes is elevated via several mechanisms in human cancers. Cyclin E has been found to be frequently amplified in human malignancies, for example, in ovarian cancer and breast cancer. In some cancer types loss-of-function mutations in FBXW7, a component of SCF^{Fbw7} ubiquitin E3 ligase responsible for cyclin E degradation, also leads to cyclin F overexpression and CDK2 activation. Alternatively, certain cancer cells express a hyperactive, truncated form of cyclin E. In addition, cyclin A amplification and overexpression have also been reported in various cancers such as hepatocellular carcinomas, colorectal and breast cancers. In some tumors, catalytic activity of CDK2 is increased following loss of the expression or alteration of the location of the endogenous CDK2 inhibitor p27 or p21 In addition, CDC25A and CDC25B, protein phosphatases responsible for the dephosphorylations that activate the CDK2, are overexpressed in various tumors. These various mechanisms of CDK2 activation have been validated using mouse cancer models. Furthermore, CDK2/cyclin E phosphorylates oncogenic Myc to oppose ras-induced senescence, highlighting the importance of CDK2 in myc/ras-induced tumorigenesis. Inactivation of CDK2 has been shown to be synthetically lethal to myc over-expressing cancer cells. Therefore, a compound of the invention may be particularly useful for treating tumors characterized by 1) overexpression of CDK2, 2) amplification of cyclin E or cyclin A, 3) loss-of-function of mutation in FBXW7, 4) expression of truncated cyclin E, 5) dysregulation of p21 or p27, and 6) hyperactive MYC/RAS.

CDK2 activation as a result of cyclin E amplification or overexpression has also been identified as a key primary or acquired resistance pathway to tumors treated by CDK4/6 inhibitors or trastuzumab.

In some embodiments, the cancer is ovarian cancer, endometrial cancer, breast cancer (e.g., triple negative breast cancer), lung cancer (e.g., adenocarcinoma, small cell lung cancer and non-small cell lung carcinomas, parvicellular and non-parvicellular carcinoma, bronchial carcinoma, bronchial adenoma, and/or pleuropulmonary blastoma), skin cancer (e.g. melanoma, squamous cell carcinoma, Kaposi sarcoma, and/or Merkel cell skin cancer), bladder cancer, cervical cancer, colorectal cancer, cancer of the small intestine, colon cancer, rectal cancer, cancer of the anus, gastric cancer, head and neck cancer (e.g., cancers of the larynx, hypopharynx, nasopharynx, oropharynx, lips, and/or mouth), liver cancer (e.g., hepatocellular carcinoma, and/or cholangiocellular carcinoma), prostate cancer, testicular cancer, uterine cancer, esophageal cancer, gall bladder cancer, pancreatic cancer (e.g. exocrine pancreatic carcinoma), stomach cancer, thyroid cancer, brain cancer, fallopian tube cancer, peritoneal cancer, AML, and parathyroid cancer. In some embodiments, the cancer is ovarian cancer. In some such embodiments, the ovarian cancer is characterized by amplification or overexpression of CCNE1 and/or CCNE2. In some embodiments, the cancer is hepatocellular carcinomas, colorectal and breast cancers. In some embodiments, the cancer is ovarian cancer. In some such embodiments, the ovarian cancer is characterized by amplification or overexpression of CCNE1 and/or CCNE2.

In other embodiments, the cancer is breast cancer, including, e.g., ER-positive/HR-positive breast cancer, HER2-negative breast cancer: ER-positive/HR-positive breast cancer, HER2-positive breast cancer; triple negative breast cancer (TNBC): or inflammatory breast cancer. In some embodiments, the breast cancer is endocrine resistant breast cancer, trastuzumab resistant breast cancer, or breast cancer demonstrating primary or acquired resistance to CDK4/CDK6 inhibition. In some embodiments, the breast cancer is advanced or metastatic breast cancer. In some embodiments of each of the foregoing, the breast cancer is characterized by amplification or overexpression of CCNE1 and/or CCNE2.

In other embodiments, compounds of Formula (I) can also be useful in autoimmune disease (e.g. rheumatoid arthritis (RA), systemic lupus erythematosus (SLE), primary Sjogren's syndrome (pSS), multiple sclerosis (MS), Crohn's disease (CD), gout, uveitis, pemphigus vulgaris) and sepsis.

### Testing

CDK2 potency and CDK2 degradation activities of the compounds of the present disclosure can be tested using the in vitro assays described in Biological Examples below.

### Pharmaceutical Compositions

In general, the compounds Formula (I) (unless stated otherwise, reference to compound/compounds of Formula (I) herein includes any of the embodiments thereof described herein or a pharmaceutically acceptable salt thereof) will be administered in a therapeutically effective amount by any of the accepted modes of administration for agents that serve similar utilities. Therapeutically effective amounts of compounds Formula (I) may range from about 0.01 to about 500 mg per kg patient body weight per day, which can be administered in single or multiple doses. A suitable dosage level may be from about 0.1 to about 250 mg/kg per day; about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of the compound Formula (I), i.e., the active ingredient, will depend upon numerous factors such as the severity of the disease to be treated, the age and relative health of the patient, the potency of the compound being utilized, the route and form of administration, and other factors.

In general, compounds Formula (I) will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance.

The compositions are comprised of in general, a compound of Formula (I) in combination with at least one pharmaceutically acceptable excipient. Acceptable excipients are generally non-toxic, aid administration, and do not adversely affect the therapeutic benefit of the compound of Formula (I). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

The compounds of Formula (I) may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds of Formula (I) may also be formulated as a depot preparation. Such long -acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds of Formula (I) may also be formulated in rectal compositions such as suppositories or retention enemas, e.g., containing conventional suppository bases such as cocoa butter, polyethylene glycol, or other glycerides.

Certain compounds of Formula (I) may be administered topically, that is by non-systemic administration. This includes the application of a compound of Formula (I) externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds of Formula (I) may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds of Formula (I) may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator. Other suitable pharmaceutical excipients and their formulations are described in Remington's Pharmaceutical Sciences, edited by E. W. Martin (Mack Publishing Company, 20th ed., 2000).

The level of the compound of Formula (I) in a formulation can vary within the full range employed by those skilled in the art. Typically, the formulation will contain, on a weight percent (wt. %) basis, from about 0.01-99.99 wt. % of a compound of Formula (I) based on the total formulation, with the balance being one or more suitable pharmaceutical excipients. For example, the compound is present at a level of about 1-80 wt. %.

### Combinations and Combination Therapies

The compounds of Formula (I) may be used in combination with one or more other drugs in the treatment of diseases or conditions for which compounds of Formula (I) or the other drugs may have utility. Such other drug(s) may be administered, by a route and in an amount commonly used therefore, contemporaneously or sequentially with a compound of Formula (I). When a compound of Formula (I) is used contemporaneously with one or more other drugs, a pharmaceutical composition in unit dosage form containing such other drugs and the compound of Formula (I) is preferred. However, the combination therapy may also include therapies in which the compound of Formula (I) and one or more other drugs are administered on different overlapping schedules. It is also contemplated that when used in combination with one or more other active ingredients, the compounds of Formula (I) and the other active ingredients may be used in lower doses than when each is used singly.

Accordingly, the pharmaceutical compositions of the present disclosure also include those that contain one or more other drugs, in addition to a compound of Formula (I).

The above combinations include combinations of a compound of Formula (I) not only with one other drug, but also with two or more other active drugs. Likewise, a compound of Formula (I) may be used in combination with other drugs that are used in the prevention, treatment, control, amelioration, or reduction of risk of the diseases or conditions for which a compound of Formula (I) is useful. Such other drugs may be administered, by a route and in an amount commonly used therefore, contemporaneously or sequentially with a compound of Formula (I). When a compound of Formula (I) is used contemporaneously with one or more other drugs, a pharmaceutical composition containing such other drugs in addition to the compound of Formula (I) can be used. Accordingly, the pharmaceutical compositions of the present disclosure also include those that also contain one or more other active ingredients, in addition to a compound of Formula (I). The weight ratio of the compound of this disclosure to the second active ingredient may be varied and will depend upon the effective dose of each ingredient. Generally, an effective dose of each will be used.

Where the subject in need is suffering from or at risk of suffering from cancer, the subject can be treated with a compound of Formula (I) in any combination with one or more other anti-cancer agents including but not limited to: MAP kinase pathway (RAS/RAF/MEK/ERK) inhibitors including but not limited to: Vemurafanib (PLX4032), Dabrafenib, Encorafenib (LGX818), TQ-B3233, XL-518 (Cas No. 1029872-29-4, available from ACC Corp); trametinib, selumetinib (AZD6244), TQ-B3234, PD184352, PD325901, TAK-733, pimasertinib, binimetinib, refametinib, cobimetinib (GDC-0973), AZD8330, BVD-523, LTT462, Ulixertinib, AMG510 (sotorasib), ARS853, adagrasib, opnurasib, divarasib, LY3537982 (2-amino-4-[(4aS)-8-chloro-10-fluoro-2,3,4,4a,5,6-hexahydro-12-oxo-3-(1-oxo-2-propen-1-yl)-1H,12H-pyrazino[2,1-d][1,5]benzoxazocin-9-yl]-7-fluorobenzo[b]thiophene-3-carbonitrile), MRTX1133 (4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol), RMC-6291, RMC-9805, RMC0708, RMC-8839 and any RAS inhibitors disclosed in PCT Applications WO2016049565, WO2016164675, WO2016168540, WO2017015562, WO2017058728, WO2017058768, WO2017058792, WO2017058805, WO2017058807, WO2017058902, WO2017058915, WO2017070256, WO2017087528, WO2017100546, WO2017172979, WO2017201161, WO2018064510, WO2018068017, WO2018119183, WO 2022/187528, and WO 2023/284730;
CSF1R inhibitors (PLX3397, LY3022855, etc.) and CSF1R antibodies (IMC-054, RG7155) TGF beta receptor kinase inhibitor such as LY2157299;
BTK inhibitor such as ibrutinib; BCR-ABL inhibitors: Imatinib (Gleevec®); Inilotinib hydrochloride; Nilotinib (Tasigna®); Dasatinib (BMS-345825); Bosutinib (SKI-606); Ponatinib (AP24534); Bafetinib (INNO406); Danusertib (PHA-739358), AT9283 (CAS 1133385-83-7); Saracatinib (AZD0530); and N-[2-[(1S,4R)-6-[[4-cyclobutylamino)-5-(trifluoromethyl)-2-pyrimidinyl]amino]-1,2,3,4-tetrahydronaphthalen-1,4-imin-9-yl]-2-oxoethyl]-acetamide (PF-03814735, CAS 942487-16-3);
ALK inhibitors: PF-2341066 (XALKOPJ®; crizotinib); 5-chloro-N4-(2- (isopropyl-sulfonyl)phenyl)-N2-(2-methoxy-4-(4-(4-methylpiperazin-4-yl)piperidin-1-yl)phenyl)pyrimidine-2,4-diamine; GSK1838705 A; CH5424802; Ceritinib (ZYKADIA); TQ-B3139, TQ-B3101 PI3K inhibitors: 4-[2-(1H-indazol-4-yl)-6-[[4-(methylsulfonyl)piperazin-1-yl]methyl]thieno[3,2-d]-pyrimidin-4-yl]morpholine (also known as GDC 0941 and described in PCT Publication Nos. WO 09/036082 and WO 09/055730), 2-methyl-2-[4-[3-methyl-2-oxo-8-(quinolin-3-yl)-2,3-dihydro-imidazo[4,5-c]quinolin-1-yl]phenyl]propionitrile (also known as BEZ 235 or NVP-BEZ 235, and described in PCT Publication No. WO 06/122806);
Vascular Endothelial Growth Factor (VEGF) receptor inhibitors: Bevacizumab (sold under the trademark Avastin® by Genentech/Roche), axitinib, (N-methyl-2-[[3-[(E)-2-pyridin-2-ylethenyl]-1H-indazol-6-yl]sulfanyl]benzamide, also known as AG013736, and described in PCT Publication No. WO 01/002369), Brivanib Alaninate ((S)-((R)-1-(4-(4-fluoro-2-methyl-1H-indol-5-yloxy)-5-methylpyrrolo[2,1-f][1,2,4]triazin-6-yloxy)propan-2-yl)-2-aminopropanoate, also known as BMS-582664), motesanib (N-(2,3-dihydro-3,3-dimethyl-1H-indol-6-yl)-2-[(4-pyridinyl-methyl)amino]-3-pyridinecarboxamide, and described in PCT Publication No. WO 02/066470), pasireotide (also known as SOM230, and described in PCT Publication No. WO 02/010192), sorafenib (sold under the tradename Nexavar®); AL-2846 MET inhibitor such as foretinib, carbozantinib, or crizotinib;
FLT3 inhibitors—sunitinib malate (sold under the tradename Sutent® by Pfizer); PKC412 (midostaurin); tanutinib, sorafenib, lestaurtinib, KW-2449, quizartinib (AC220) and crenolanib;
Epidermal growth factor receptor (EGFR) inhibitors: Gefitnib (sold under the tradename Iressat), N-[4-[(3-chloro-4-fluorophenyl)amino]-7-[[(3″S″)-tetrahydro-3-furanyl]oxy]-6-quinazolinyl]-4(dimethylamino)-2-butenamide, sold under the tradename Tovok® by Boehringer Ingelheim), cetuximab (sold under the tradename Erbitux® by Bristol-Myers Squibb), panitumumab (sold under the tradename Vectibix® by Amgen);
HER2 receptor inhibitors: Trastuzumab (sold under the trademark Herceptin® by Genentech/Roche), neratinib (also known as HKI-272, (2E)-N-[4-[[3-chloro-4-[(pyridin-2-yl)methoxy]phenyl]amino]-3-cyano-7-ethoxyquinolin-6-yl]-4-(dimethylamino)but-2-enamide, and described PCT Publication No. WO 05/028443), lapatinib or lapatinib ditosylate (sold under the trademark Tykerb® by GlaxoSmithKline); Trastuzumab emtansine (in the United States, ado-trastuzumab emtansine, trade name Kadcyla) - an antibody-drug conjugate consisting of the monoclonal antibody trastuzumab (Herceptin) linked to the cytotoxic agent mertansine (DM1);
HER dimerization inhibitors: Pertuzumab (sold under the trademark Omnitarg®, by Genentech);
CD20 antibodies: Rituximab (sold under the trademarks Riuxan® and MabThera® by Genentech/Roche), tositumomab (sold under the trademarks Bexxar® by GlaxoSmithKline), ofatumumab (sold under the trademark Arzerra® by GlaxoSmithKline);
Tyrosine kinase inhibitors: Erlotinib hydrochloride (sold under the trademark Tarceva® by Genentech/Roche), Linifanib (N-[4-(3-amino-1H-indazol-4-yl)phenyl]-N′-(2-fluoro-5-methylphenyl)urea, also known as ABT 869, available from Genentech), sunitinib malate (sold under the tradename Sutent® by Pfizer), bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996), dasatinib (sold under the tradename Sprycel® by Bristol-Myers Squibb), armala (also known as pazopanib, sold under the tradename Votrient(® by GlaxoSmithKline), imatinib and imatinib mesylate (sold under the tradenames Gilvec® and Gleevec® by Novartis);
DNA Synthesis inhibitors: Capecitabine (sold under the trademark Xeloda® by Roche), gemcitabine hydrochloride (sold under the trademark Gemzar® by Eli Lilly and Company), nelarabine ((2R3S,4R,5R)-2-(2-amino-6-methoxy-purin-9-yl)-5-(hydroxymethyl)oxolane-3,4-diol, sold under the tradenames Arranon® and Atriance® by GlaxoSmithKline);
Antineoplastic agents: oxaliplatin (sold under the tradename Eloxatin® ay Sanofi-Aventis and described in U.S. Pat. No. 4,169,846);
Human Granulocyte colony-stimulating factor (G-CSF) modulators: Filgrastim (sold under the tradename Neupogen® by Amgen);
Immunomodulators: Afutuzumab (available from Roche®), pegfilgrastim (sold under the tradename Neulasta® by Amgen), lenalidomide (also known as CC-5013, sold under the tradename Revlimid®), thalidomide (sold under the tradename Thalomid®);
CD40 inhibitors: Dacetuzumab (also known as SGN-40 or huS2C6, available from Seattle Genetics, Inc); Pro-apoptotic receptor agonists (PARAs): Dulanermin (also known as AMG-951, available from Amgen/Genentech);
Hedgehog antagonists: 2-chloro-N-[4-chloro-3-(2-pyridinyl)phenyl]-4-(methylsulfonyl)-benzamide (also known as GDC-0449, and described in PCT Publication No. WO 06/028958);
Phospholipase A2 inhibitors: Anagrelide (sold under the tradename Agrylin®);
BCL-2 inhibitors: 4-[4-[[2-(4-chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386);
MCI-1 inhibitors: MIK665, S64315, AMG 397, and AZD5991;
Aromatase inhibitors: Exemestane (sold under the trademark Aromasin® by Pfizer), letrozole (sold under the tradename Femara® by Novartis), anastrozole (sold under the tradename Arimidex®);
Topoisomerase I inhibitors: Irinotecan (sold under the trademark Camptosar® by Pfizer), topotecan hydrochloride (sold under the tradename Hycamtin(® by GlaxoSmithKline);
Topoisomerase II inhibitors: etoposide (also known as VP-16 and Etoposide phosphate, sold under the tradenames Toposar®, VePesid® and Etopophos®), teniposide (also known as VM-26, sold under the tradename Vumon®);
mTOR inhibitors: Temsirolimus (sold under the tradename Torisel® by Pfizer), ridaforolimus (formally known as deferolimus, (1R,2R,4S)-4-[(2R)-2[(1R,9S,12S,15R,16E,18R,19R,21R,23S,24E,26E,28Z,30S,32S,35R)-1,18-dihydroxy-19,30- dimethoxy-15,17,21,23,29,35-hexamethyl-2,3,10,14,20-pentaoxo-11,36-dioxa-4- azatricyclo[30.3.1.0 4′ 9]hexatria-conta-16,24,26,28-tetraen-12-yl]propyl]-2-methoxycyclohexyl dimethylphosphinate, also known as AP23573 and MK8669, and described in PCT Publication No. WO 03/064383), everolimus (sold under the tradename Afinitor® by Novartis);
Proteasome inhibitor such as carfilzomib, MLN9708, delanzomib, or bortezomib;
BET inhibitors such as INCB054329, OTX015, and CPI-0610;
LSD1 inhibitors such as GSK2979552, and INCB059872;
HIF-2a inhibitors such as PT2977 and PT2385;
Osteoclastic bone resorption inhibitors: 1-Hydroxy-2-imidazol-1-yl-phosphonoethyl) phosphonic acid monohydrate (sold under the tradename Zometa® by Novartis); CD33 Antibody Drug Conjugates: Gemtuzumab ozogamicin (sold under the tradename Mylotarg® by Pfizer/Wyeth);
CD22 Antibody Drug Conjugates: Inotuzumab ozogamicin (also referred to as CMC-544 and WAY-207294, available from Hangzhou Sage Chemical Co., Ltd.);
CD20 Antibody Drug Conjugates: Ibritumomab tiuxetan (sold under the tradename Zevalin®);
Somatostain analogs: octreotide (also known as octreotide acetate, sold under the tradenames Sandostatin® and Sandostatin LARV);
Synthetic Interleukin-11 (IL-11): oprelvekin (sold under the tradename Neumega® by Pfizer/Wyeth);
Synthetic erythropoietin: Darbepoetin alfa (sold under the tradename Aranesp® by Amgen);
Receptor Activator for Nuclear Factor κ B (RANK) inhibitors: Denosumab (sold under the tradename Prolia® by Amgen);
Thrombopoietin mimetic peptibodies: Romiplostim (sold under the tradename Nplate® by Amgen);
Cell growth stimulators: Palifermin (sold under the tradename Kepivance® by Amgen);
Anti-Insulin-like Growth Factor-1 receptor (IGF-1R) antibodies: Figitumumab (also known as CP-751,871, available from ACC Corp), robatumumab (CAS No. 934235-44-6);
Anti-CSl antibodies: Elotuzumab (HuLuc63, CAS No. 915296-00-3); CD52 antibodies: Alemtuzumab (sold under the tradename Campath®);
Histone deacetylase inhibitors (HDI): Voninostat (sold under the tradename Zolinza® by Merck);
Alkylating agents: Temozolomide (sold under the tradenames Temodar® and Temodal® by Schering-Plough/Merck), dactinomycin (also known as actinomycin-D and sold under the tradename Cosmegen®), melphalan (also known as L-PAM, L-sarcolysin, and phenylalanine mustard, sold under the tradename Alkeran®), altretamine (also known as hexamethylmelamine (HMM), sold under the tradename Hexalen®), carmustine (sold under the tradename BiCNU®), bendamustine (sold under the tradename Treanda®), busulfan (sold under the tradenames Busulfex® and Myleran®), carboplatin (sold under the tradename Paraplatin®), lomustine (also known as CCNU, sold under the tradename CeeNU®), cisplatin (also known as CDDP, sold under the tradenames Platinol® and Platinol®-AQ), chloranbucil (sold under the tradename Leukeran®), cyclophosphamide (sold under the tradenames Cytoxan® and Neosar®), dacarbazine (also known as DTIC, DIC and imidazole carboxamide, sold under the tradename DTIC-Dome®), altretamine (also known as hexamethylmelamine (HMM) sold under the tradename Hexalen®), ifosfamide (sold under the tradename Ifex®), procarbazine (sold under the tradename Matulane®), mechlorethamine (also known as nitrogen mustard, mustine and mechloroethamine hydrochloride, sold under the tradename Mustargen®), streptozocin (sold under the tradename Zanosar®), thiotepa (also known as thiophosphoamide, TESPA and TSPA, sold under the tradename Thioplex®; Biologic response modifiers: bacillus calmette-guerin (sold under the tradenames theraCys® and TICE® BCG), denileukin diftitox (sold under the tradename Ontak®);
Anti-tumor antibiotics: doxorubicin (sold under the tradenames Adriamycin® and Rubex®, bleomycin (sold under the tradename Lenoxane®), daunorubicin (also known as dauorubicin hydrochloride, daunomycin, and rubidomycin hydrochloride, sold under the tradename Cerubidine®), daunorubicin liposomal (daunorubicin citrate liposome, sold under the tradename DaunoXome®), mitoxantrone (also known as DHAD, sold under the tradename Novantrone®), epirubicin (sold under the tradename Ellence™), idarubicin (sold under the tradenames Idamycin®, Idamycin PFS®), mitomycin C (sold under the tradename Mutamycin®);
Anti-microtubule agents: Estramustine (sold under the tradename Emcyl®);
Cathepsin K inhibitors: Odanacatib (also known as MK-0822, N—(O-cyanocyclopropyl)-4-fluoro-N-2-{(1S)-2,2,2-trifluoro-1-[4′-(methylsulfonyl)biphenyl-4-yl]ethyl)-L-leucinamide, available from Lanzhou Chon Chemicals, ACC Corp., and ChemieTek, and described in PCT Publication no. WO 03/075836); Epothilone B analogs: Ixabepilone (sold under the tradename Lxempra® by Bristol-Myers Squibb);
Heat Shock Protein (HSP) inhibitors: Tanespimycin (17-allylamino-17- demethoxy-geldanamycin, also known as KOS-953 and 17-AAG, available from SIGMA, and described in U.S. Pat. No. 4,261,989), NVP—HSP990, AUY922, AT13387, STA-9090, Debio 0932, KW-2478, XL888, CNF2024, TAS-116
TpoR agonists: Eltrombopag (sold under the tradenames Promacta® and Revolade® by GlaxoSmithKline);
Anti-mitotic agents: Docetaxel (sold under the tradename Taxotere® by Sanofi-Aventis);
Adrenal steroid inhibitors: aminoglutethimide (sold under the tradename Cytadren®);
Anti-androgens: Nilutamide (sold under the tradenames Nilandron® and Anandron®), bicalutamide (sold under tradename Casodex®), flutamide (sold under the tradename Fulexin™);
Androgens: Fluoxymesterone (sold under the tradename Halotestin®);
CDK (CDK1, CDK2, CDK3, CDK5, CDK7, CDK8, CDK9, CDK11/12, or CDK16) inhibitors including but not limited to Alvocidib (pan-CDK inhibitor, also known as flovopirdol or HMR-1275, 2-(2-chlorophenyl)-5,7-dihydroxy-8-[(3S,4R)-3-hydroxy-1-methyl-4-piperidinyl]-4-chromenone, and described in U.S. Pat. No. 5,621,002);
CDK4/6 inhibitors pabociclib, ribociclib, abemaciclib, and Trilaciclib; CDK9 inhibitors AZD 4573, P276-00, AT7519M, TP-1287; CDK2/4/6 inhibitor such as PF-06873600;
SHP-2 inhibitor such as TNO155;
MDM2/MDMX, MDM2/p53 and/or MDMX/p53 modulators;
Gonadotropin-releasing hormone (GnRH) receptor agonists: Leuprolide or leuprolide acetate (sold under the tradenames Viadure® by Bayer AG, Eligard® by Sanofi-Aventis and Lupron® by Abbott Lab);
Taxane anti-neoplastic agents: Cabazitaxel (1-hydroxy-7,10-dimethoxy-9-oxo-5,20-epoxytax-1 l-ene-2a,4,13a-triyl-4-acetate-2-benzoate-13-[(2R,3S)-3-{[(tert-butoxy)carbonyl]amino)-2-hydroxy-3-phenylpropanoate), larotaxel ((2α,3ξ,4α,5β,7α,10β,13α)-4,10-bis(acetyloxy)-13-({(2R,3S)-3-[(tert-butoxycarbonyl) amino]-2-hydroxy-3-phenylpropanoyl}oxy)-1-hydroxy-9-oxo-5,20-epoxy-7,19-cyclotax-11-en-2-yl benzoate);
5HTla receptor agonists: Xaliproden (also known as SR57746, 1-[2-(2-naphthyl)ethyl]-4-[3-(trifluoromethyl)phenyl]-1,2,3,6-tetrahydropyridine, and described in U.S. Pat. No. 5,266,573); HPC vaccines: Cervarix® sold by GlaxoSmithKline, Gardasil® sold by Merck; Iron Chelating agents: Deferasinox (sold under the tradename Exjade® by Novartis);
Anti-metabolites: Claribine (2-chlorodeoxyadenosine, sold under the tradename Leustatin®), 5-fluorouracil (sold under the tradename Adrucil)), 6-thioguanine (sold under the tradename Purinethol®), pemetrexed (sold under the tradename Alimta®), cytarabine (also known as arabinosylcytosine (Ara-C), sold under the tradename Cytosar-U®), cytarabine liposomal (also known as Liposomal Ara-C, sold under the tradename DepoCyt™), decitabine (sold under the tradename Dacogen®), hydroxyurea (sold under the tradenames Hydrea®, Droxia™ and Mylocel™), fludarabine (sold under the tradename Fludara®), floxuridine (sold under the tradename FUDR®), cladribine (also known as 2-chlorodeoxyadenosine (2-CdA) sold under the tradename Leustatinτ), methotrexate (also known as amethopterin, methotrexate sodium (MTX), sold under the tradenames Rheumatrex® and Trexall™), pentostatin (sold under the tradename Nipent®);
Bisphosphonates: Pamidronate (sold under the tradename Aredia®), zoledronic acid (sold under the tradename Zometag); Demethylating agents: 5-azacitidine (sold under the tradename Vidazat), decitabine (sold under the tradename Dacogen®);
Plant Alkaloids: Paclitaxel protein-bound (sold under the tradename Abraxane®), vinblastine (also known as vinblastine sulfate, vincaleukoblastine and VLB, sold under the tradenames Alkaban-AQ® and Velban®), vincristine (also known as vincristine sulfate, LCR, and VCR, sold under the tradenames Oncovin® and Vincasar Pfs®), vinorelbine (sold under the tradename Navelbine®), paclitaxel (sold under the tradenames Taxol and Onxal™);
Retinoids: Ali tretinoin (sold under the tradename Panretin®), tretinoin (all-trans retinoic acid, also known as ATRA, sold under the tradename Vesanoid®), Isotretinoin (13-cis-retinoic acid, sold under the tradenames Accutane®, Amnesteem®, Claravis®, Clarus®, Decutan®, Isotane®, Izotech®, Oratane®, Isotrett, and Sotret®), bexarotene (sold under the tradename Targretin®);
Glucocorticosteroids: Hydrocortisone (also known as cortisone, hydrocortisone sodium succinate, hydrocortisone sodium phosphate, and sold under the tradenames Ala-Cort®, Hydrocortisone Phosphate, Solu-Cortef®, Hydrocort Acetate® and Lanacort®), dexamethazone ((8S,9R,10S,11S,13S,14S,16R,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one), prednisolone (sold under the tradenames Delta-Cortel®, Orapred), Pediapred® and Prelone®), prednisone (sold under the tradenames Deltasone®, Liquid Red®, Meticorten® and Orasone®), methylprednisolone (also known as 6-Methylprednisolone, Methylprednisolone Acetate, Methylprednisolone Sodium Succinate, sold under the tradenames Duralone®, Medralone®, MedrolS, M-Prednisol® and Solu-Medrol®);
Cytokines: interleukin-2 (also known as aldesleukin and IL-2, sold under the tradename Proleukin®), interleukin-11 (also known as oprevelkin, sold under the tradename Neumega®), alpha interferon alfa (also known as IFN-alpha, sold under the tradenames Intron® A, and Roferon-A®); [00209]Estrogen receptor downregulators: Fulvestrant (sold under the tradename Faslodex®);
Anti-estrogens: tamoxifen (sold under the tradename Novaldex®); Toremifene (sold under the tradename Fareston®);
Selective estrogen receptor modulators (SERMs): Raloxifene (sold under the tradename Evista®);
Leutinizing hormone releasing hormone (LHRH) agonists: Goserelin (sold under the tradename Zoladex®); Progesterones: megestrol (also known as megestrol acetate, sold under the tradename Megace®);
Miscellaneous cytotoxic agents: Arsenic trioxide (sold under the tradename Trisenox®), asparaginase (also known as L-asparaginase, Erwinia L-asparaginase, sold under the tradenames Elspar® and Kidrolase®);
One or more immune checkpoint inhibitors CD27, CD28, CD40, CD122, CD96, CD73, CD39, CD47, OX40, GITR, CSF1R, JAK, PI3K delta, PI3K gamma, TAM kinase, arginase, CD137 (also known as 4-1BB), ICOS, A2AR, A2BR, HIF-2α, B7-H3, B7-H4, BTLA, CTLA-4, LAG3, TIM3, VISTA, CD96, TIGIT, PD-1. PD-L 1 and PD-L2. In some embodiments, the immune checkpoint molecule is a stimulatory checkpoint molecule selected from CD27, CD28, CD40, ICOS, OX40, GITR, CD137 and STING. In some embodiments, the immune checkpoint molecule is an inhibitory checkpoint molecule selected from B7-H3, B7-H4, BTLA, CTLA-4, IDO, TDO, Arginase, KIR, LAG3, PD-1, TIM3, CD96, TIGIT and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab, or pembrolizumab or PDR001. In some embodiments, the anti-PD1 antibody is pembrolizumab.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A (atezolizumab) or MEDI4736 (durvalumab).

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab. In some embodiments, the inhibitor of an immune checkpoint molecule is an inhibitor of LAG3, e.g., an anti-LAG3 antibody. In some embodiments, the anti-LAG3 antibody is BMS-986016 or LAG525. In some embodiments, the inhibitor of an immune checkpoint molecule is an inhibitor of GITR, e.g., an anti-GITR antibody. In some embodiments, the anti-GITR antibody is TRX518 or, MK-4166, INCAGN01876 or MK-1248. In some embodiments, the inhibitor of an immune checkpoint molecule is an inhibitor of OX40, e.g., an anti-OX40 antibody or OX40L fusion protein. In some embodiments, the anti-OX40 antibody is MED10562 or, INCAGN01949, GSK2831781, GSK-3174998, MOXR-0916, PF-04518600 or LAG525. In some embodiments, the OX40L fusion protein is MEDI6383

Compounds of Formula (I) can also be used to increase or enhance an immune response, including increasing the immune response to an antigen; to improve immunization, including increasing vaccine efficacy; and to increase inflammation. In some embodiments, the compounds of the invention can be sued to enhance the immune response to vaccines including, but not limited, Listeria vaccines, oncolytic viral vaccines, and cancer vaccines such as GVAX® (granulocyte-macrophage colony-stimulating factor (GM-CF) gene-transfected tumor cell vaccine). Anti-cancer vaccines include dendritic cells, synthetic peptides, DNA vaccines and recombinant viruses. Other immune-modulatory agents also include those that block immune cell migration such as antagonists to chemokine receptors, including CCR2 and CCR4; Sting agonists and Toll receptor agonists.

Other anti-cancer agents also include those that augment the immune system such as adjuvants or adoptive T cell transfer. Compounds of this application may be effective in combination with CAR (Chimeric antigen receptor) T cell treatment as a booster for T cell activation.

A compound of Formula (I) can also be used in combination with the following adjunct therapies: anti-nausea drugs: NK-1 receptor antagonists: Casopitant (sold under the tradenames Rezonic® and Zunrisa®) by GlaxoSmithKline); and Cytoprotective agents: Amifostine (sold under the tradename Ethyol®), leucovorin (also known as calcium leucovorin, citrovorum factor and folinic acid).

### EXAMPLES

The following preparations of Intermediates (References) and compounds of Formula (I) (Examples) are given to enable those skilled in the art to more clearly understand and to practice the present disclosure. They should not be considered as limiting the scope of the disclosure, but merely as being illustrative and representative thereof.

### SYNTHETIC EXAMPLES

### Reference 1

### Synthesis of 4-((14-amino-3,6,9,12-tetraoxatetradecyl)amino)-2-(2,6-dioxopiperidin-3-yl)isoindoline-1,3-dione, 2,2,2-trifluoroacetate

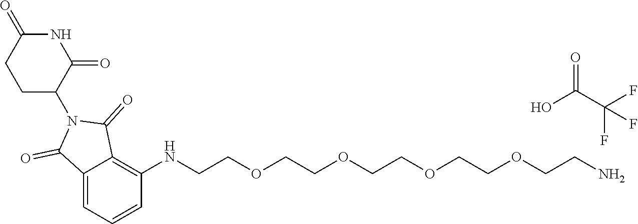

### Step 1: tert-Butyl (14-((2-(2,6-dioxopiperidin-3-yl)-1,3-dioxoisoindolin-4-yl)amino)-3,6,9,12-tetraoxatetradecyl)carbamate

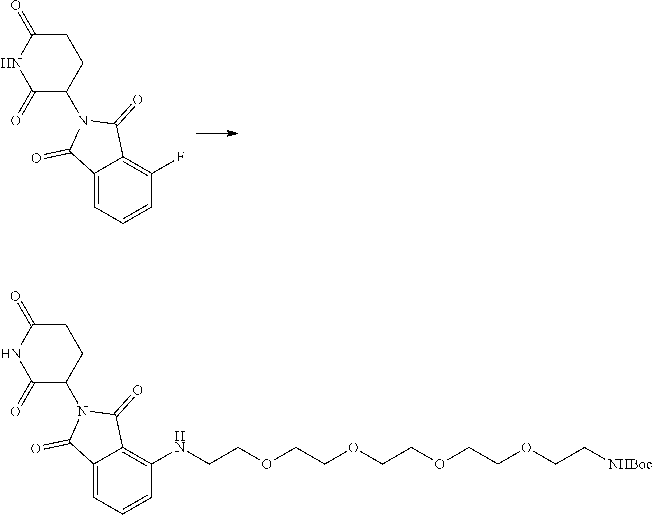 [SMILES: O=C1CCC(N2C(=O)c3cccc(F)c3C2=O)C(=O)N1 ; CC(C)(C)OC(=O)NCCOCCOCCOCCOCCNc1cccc2c1C(=O)N(C1CCC(=O)NC1=O)C2=O]

A mixture of 2-(2,6-dioxopiperidin-3-yl)-4-fluoroisoindoline-1,3-dione (500.0 mg, 1.81 mmol, 1.00 eq.), tert-butyl (14-amino-3,6,9,12-tetraoxatetradecyl)carbamate (609.0 mg, 1.81 mmol, 1.00 eq.), DIPEA (467.9 mg, 3.62 mmol, 2.00 eq.) in DMF (6.0 mL) was stirred for 16 h at 90° C. under nitrogen atmosphere. The mixture was cooled, diluted with water, and then extracted with EtOAc. The combined organic layer was washed with water, dried over anhydrous Na₂SO₄, filtered, and then concentrated. The residue was purified by silica gel column chromatography, eluted with PE/EtOAc (5:1), to afford the title compound as a yellow solid.

### Step 2: 4-((14-Amino-3,6,9,12-tetraoxatetradecyl)amino)-2-(2,6-dioxopiperidin-3-yl)isoindoline-1,3-dione, 2,2,2-trifluoroacetate

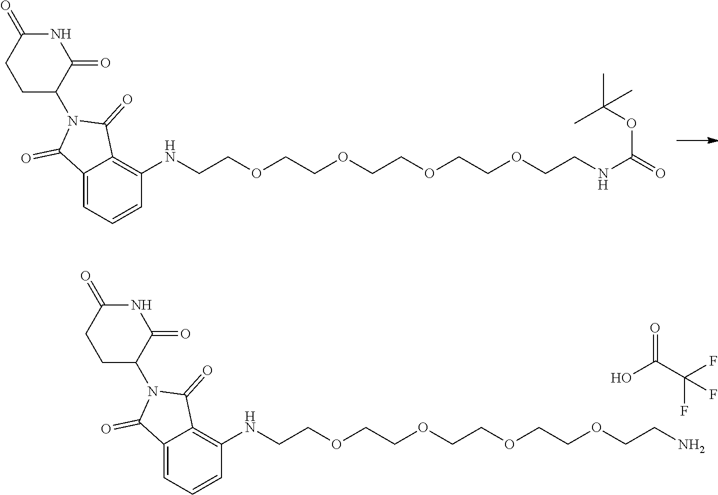

TFA (0.3 mL, 3.92 mmol, 46.67 eq.) was added to a stirred solution of tert-butyl (14-((2-(2,6-dioxopiperidin-3-yl)-1,3-dioxoisoindolin-4-yl)amino)-3,6,9,12-tetraoxatetradecyl)carbamate (50 mg, 0.084 mmol, 1.00 eq.) in DCM (1.0 mL) at 0° C. under nitrogen atmosphere. The mixture was stirred for 2 h, and then concentrated to give crude title compound as light yellow oil.

### Reference 2

### Synthesis of 2-(2-(2-(2-((2-(2,6-dioxopiperidin-3-yl)-1,3-dioxoisoindolin-4-yl)oxy)-acetamido)ethoxy) ethoxy)ethyl methanesulfonate

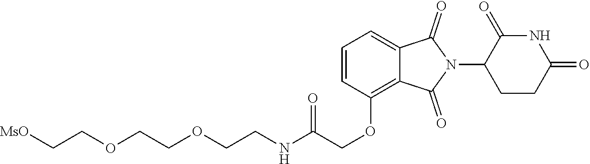 [SMILES: CS(=O)(=O)OCCOCCOCCNC(=O)COc1cccc2c1C(=O)N(C1CCC(=O)NC1=O)C2=O]

### Step 1: tert-Butyl 2-((2-(2,6-dioxopiperidin-3-yl)-1,3-dioxoisoindolin-4-yl)oxy)acetate

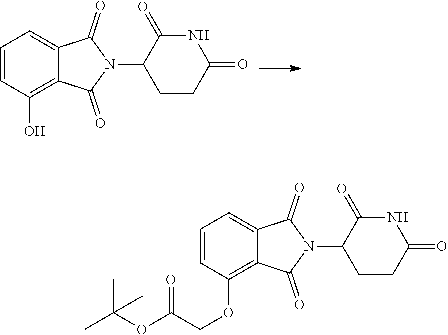 [SMILES: O=C1CCC(N2C(=O)c3cccc(O)c3C2=O)C(=O)N1 ; CC(C)(C)OC(=O)COc1cccc2c1C(=O)N(C1CCC(=O)NC1=O)C2=O]

A mixture of 2-(2,6-dioxopiperidin-3-yl)-4-hydroxyisoindoline-1,3-dione (1.5 g, 5.47 mmol, 1.00 eq.), tert-butyl 2-bromoacetate (1.3 g, 6.66 mmol, 1.22 eq.) and K₂CO₃ (1.1 g, 7.96 mmol, 1.46 eq.) in DMF (20.0 mL) was stirred at RT for 2 h. The mixture was diluted with H₂O and extracted with ethyl acetate. The combined organic phase was washed with brine, dried over Na₂SO₄, concentrated to get title compound as a white solid.

### Step 2: 2-((2-(2,6-Dioxopiperidin-3-yl)-1,3-dioxoisoindolin-4-yl)oxy)acetic acid

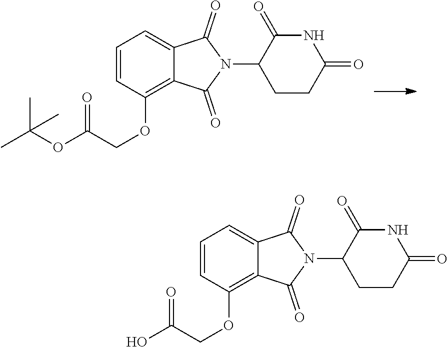 [SMILES: CC(C)(C)OC(=O)COc1cccc2c1C(=O)N(C1CCC(=O)NC1=O)C2=O ; O=C(O)COc1cccc2c1C(=O)N(C1CCC(=O)NC1=O)C2=O]

A solution of tert-butyl 2-((2-(2,6-dioxopiperidin-3-yl)-1,3-dioxoisoindolin-4-yl)oxy)-acetate (1.0 g, 2.57 mmol, 1.00 eq.) and TFA (5.0 mL) in DCM (10.0 mL) was stirred at rt for 2 h.

The mixture was concentrated and the residue was triturated with ether to get title compound as a white solid.

### Step 3: 2-((2-(2,6-Dioxopiperidin-3-yl)-1,3-dioxoisoindolin-4-yl)oxy)-N-(2-(2-(2-hydroxyethoxy) ethoxy)ethyl)acetamide

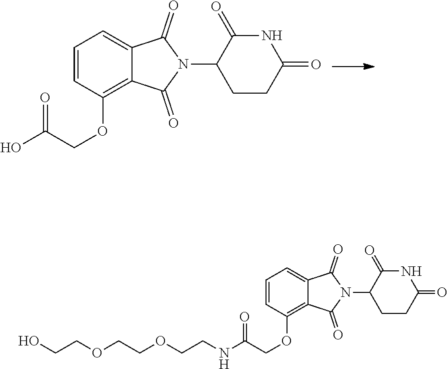 [SMILES: O=C(O)COc1cccc2c1C(=O)N(C1CCC(=O)NC1=O)C2=O ; O=C(COc1cccc2c1C(=O)N(C1CCC(=O)NC1=O)C2=O)NCCOCCOCCO]

HATU (513 mg, 1.35 mmol, 1.5 eq) was added to a stirred solution of 2-((2-(2,6-dioxopiperidin-3-yl)-1,3-dioxoisoindolin-4-yl)oxy)acetic acid (300 mg, 0.90 mmol, 1.00 eq.), 2-(2-(2-aminoethoxy)ethoxy)ethanol (201 mg, 1.35 mmol, 1.50 eq.) and DIPEA (348 mg, 2.69 mmol, 2.99 eq.) in DMF (6.0 mL) at 0° C. and the mixture was stirred at RT for 1 h. The mixture was diluted with H₂O and extracted with DCM. The combined organic phase was washed with brine, dried over Na₂SO₄, concentrated to get crude title compound as a yellow oil, which was used for next step without further purification.

### Step 4: 2-(2-(2-(2-((2-(2,6-Dioxopiperidin-3-yl)-1,3-dioxoisoindolin-4-yl)oxy)acetamido)ethoxy) ethoxy)ethyl methanesulfonate

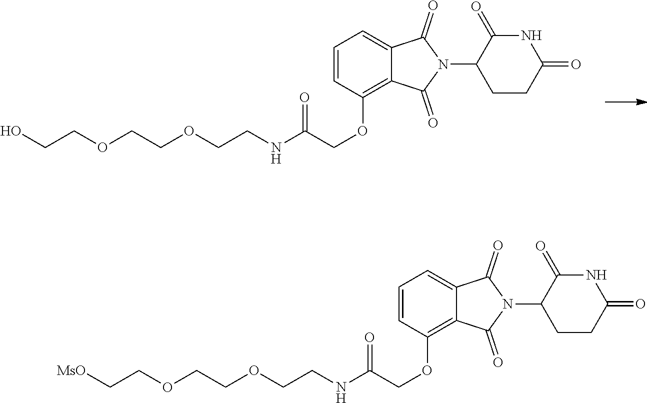 [SMILES: O=C(COc1cccc2c1C(=O)N(C1CCC(=O)NC1=O)C2=O)NCCOCCOCCO ; CS(=O)(=O)OCCOCCOCCNC(=O)COc1cccc2c1C(=O)N(C1CCC(=O)NC1=O)C2=O]

MsCl (298 mg, 2.60 mmol, 1.50 eq.) was added to a stirred solution of 2-((2-(2,6-dioxopiperidin-3-yl)-1,3-dioxoisoindolin-4-yl)oxy)-N-(2-(2-(2-hydroxyethoxy)ethoxy)ethyl)acetamide (800 mg, 1.73 mmol, 1.00 eq.) and TEA (524 mg, 5.18 mmol, 2.99 eq.) in DCM (8.0 mL) at 0° C. and the mixture was stirred at 0° C. for 1 h. The mixture was diluted with H₂O and extracted with DCM. The combined organic phase was washed with brine, dried over Na₂SO₄, concentrated and purified by flash silica gel chromatography (DCM:MeOH=50:1) to get title compound as a white solid.

### Reference 3

### Synthesis of 2-(2-(2-((2-(2,6-dioxopiperidin-3-yl)-1,3-dioxoisoindolin-4-yl)oxy)acetamido)ethoxy)-ethyl methanesulfonate

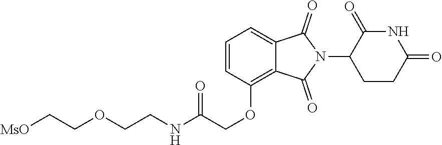 [SMILES: CS(=O)(=O)OCCOCCNC(=O)COc1cccc2c1C(=O)N(C1CCC(=O)NC1=O)C2=O]

### Step 1: 2-((2-(2,6-Dioxopiperidin-3-yl)-1,3-dioxoisoindolin-4-yl)oxy)-N-(2-(2-hydroxyethoxy)-ethyl) acetamide

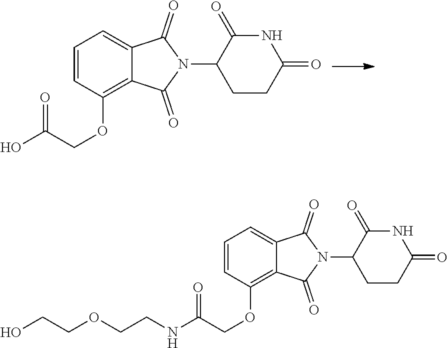 [SMILES: O=C(O)COc1cccc2c1C(=O)N(C1CCC(=O)NC1=O)C2=O ; O=C(COc1cccc2c1C(=O)N(C1CCC(=O)NC1=O)C2=O)NCCOCCO]

A mixture of 2-((2-(2,6-dioxopiperidin-3-yl)-1,3-dioxoisoindolin-4-yl)oxy)acetic acid (180 mg, 0.54 mmol, 1.00 eq.), 2-(2-aminoethoxy)ethan-1-ol (85 mg, 0.81 mmol, 1.50 eq.), HATU (308 mg, 0.81 mmol, 1.50 eq.) and DIPEA (209 mg, 1.62 mmol, 3.00 eq.) in DMF (5.0 mL) was stirred at 0° C. for 1 h. The reaction mixture was diluted with H₂O and extracted with DCM. The combined organic phase was washed with brine, dried over Na₂SO₄ and concentrated to give crude title compound as a yellow oil, which was used for next step without further purification.

### Step 2: 2-(2-(2-((2-(2,6-Dioxopiperidin-3-yl)-1,3-dioxoisoindolin-4-yl)oxy)acetamido)ethoxy)-ethyl methanesulfonate

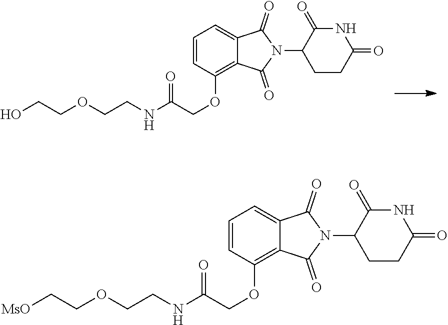 [SMILES: O=C(COc1cccc2c1C(=O)N(C1CCC(=O)NC1=O)C2=O)NCCOCCO ; CS(=O)(=O)OCCOCCNC(=O)COc1cccc2c1C(=O)N(C1CCC(=O)NC1=O)C2=O]

MsCl (162 mg, 1.41 mmol, 1.48 eq.) was added slowly to a stirred solution of 2-((2-(2,6-dioxopiperidin-3-yl)-1,3-dioxoisoindolin-4-yl)oxy)-N-(2-(2-hydroxyethoxy)ethyl) acetamide (400 mg, 0.95 mmol, 1.00 eq.) and TEA (288 mg, 2.85 mmol, 3.00 eq.) in DCM (8.0 mL) at 0° C. The resulting mixture was stirred at 0° C. for 1 h, diluted with H₂O and extracted with DCM. The combined organic phase was washed with brine, dried over Na₂SO₄, concentrated and the residue was purified by flash silica gel chromatography (DCM:MeOH=50:1) to give the title compound as a white solid.

### Reference 4

### Synthesis of 1-((2-(2,6-dioxopiperidin-3-yl)-1,3-dioxoisoindolin-4-yl)oxy)-2-oxo-6,9,12-trioxa-3-azatetradecan-14-yl methanesulfonate

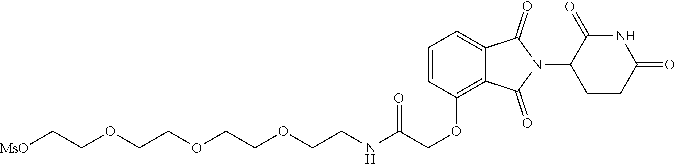 [SMILES: CS(=O)(=O)OCCOCCOCCOCCNC(=O)COc1cccc2c1C(=O)N(C1CCC(=O)NC1=O)C2=O]

### Step 1: 2-((2-(2,6-Dioxopiperidin-3-yl)-1,3-dioxoisoindolin-4-yl)oxy)-N-(2-(2-(2-(2-hydroxyethoxy) ethoxy)ethoxy)ethyl)acetamide

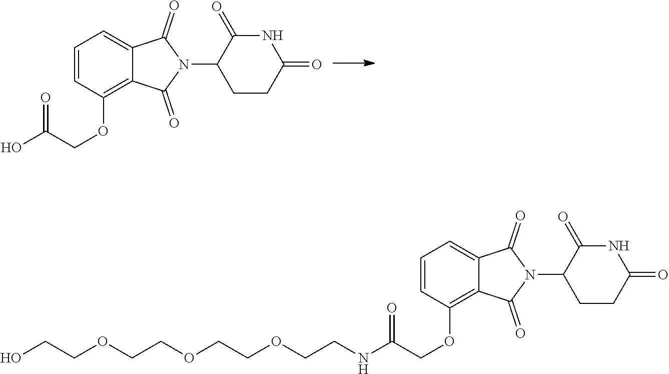 [SMILES: O=C(O)COc1cccc2c1C(=O)N(C1CCC(=O)NC1=O)C2=O ; O=C(COc1cccc2c1C(=O)N(C1CCC(=O)NC1=O)C2=O)NCCOCCOCCOCCO]

HATU (513 mg, 1.35 mmol, 1.50 eq.) and DIPEA (348 mg, 2.69 mmol, 3.00 eq.) were added to a mixture of 2-((2-(2,6-dioxopiperidin-3-yl)-1,3-dioxoisoindolin-4-yl)oxy)acetic acid (300 mg, 0.90 mmol, 1.00 eq.) and 2-(2-(2-(2-aminoethoxy)ethoxy)ethoxy)ethan-1-ol (259 mg, 1.34 mmol, 1.49 eq.) in DMF (5.0 mL) at 0° C. The mixture was stirred at 0° C. for 1 h, diluted with H₂O, and then extracted with DCM. The combined organic phase was washed with brine, dried over Na₂SO₄, filtered and concentrated to get crude title compound as a yellow oil, which was used for next step without further purification.

### Step 2: 1-((2-(2,6-Dioxopiperidin-3-yl)-1,3-dioxoisoindolin-4-yl)oxy)-2-oxo-6,9,12-trioxa-3-azatetradecan-14-yl methanesulfonate

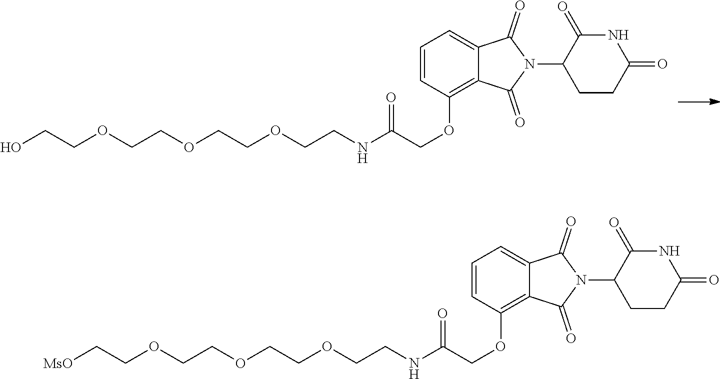 [SMILES: O=C(COc1cccc2c1C(=O)N(C1CCC(=O)NC1=O)C2=O)NCCOCCOCCOCCO ; CS(=O)(=O)OCCOCCOCCOCCNC(=O)COc1cccc2c1C(=O)N(C1CCC(=O)NC1=O)C2=O]

MsCl (271 mg, 2.37 mmol, 1.50 eq.) was added slowly to a stirred solution of 2-((2-(2,6-dioxopiperidin-3-yl)-1,3-dioxoisoindolin-4-yl)oxy)-N-(2-(2-(2-(2-hydroxyethoxy) ethoxy)ethoxy)ethyl)acetamide (800 mg, 1.58 mmol, 1.00 eq.) and TEA (479 mg, 4.73 mmol, 3.00 eq.) in DCM (8.0 mL) at 0° C. The mixture was stirred at 0° C. for 1 h, diluted with H₂O and extracted with DCM. The combined organic phase was washed with brine, dried over Na₂SO₄, concentrated and purified by flash silica gel chromatography (DCM:MeOH=50:1) to get the title compound as a white solid.

### Reference 5

### Synthesis of 2-(2-(2-((2-(1-methyl-2,6-dioxopiperidin-3-yl)-1,3-dioxoisoindolin-4-yl)oxy)-acetamido) ethoxy)ethyl methanesulfonate

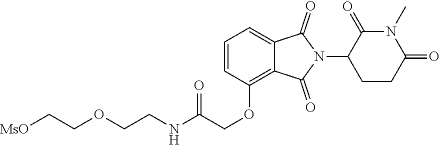 [SMILES: CN1C(=O)CCC(N2C(=O)c3cccc(OCC(=O)NCCOCCOS(C)(=O)=O)c3C2=O)C1=O]

### Step 1: tert-Butyl 2-((2-(1-methyl-2,6-dioxopiperidin-3-yl)-1,3-dioxoisoindolin-4-yl)oxy)acetate

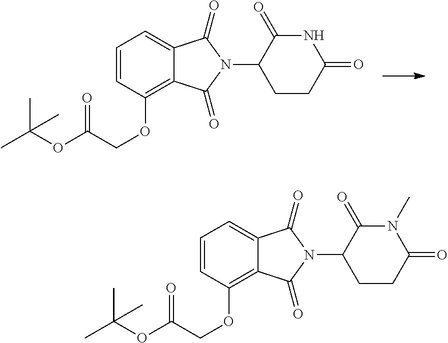 [SMILES: CC(C)(C)OC(=O)COc1cccc2c1C(=O)N(C1CCC(=O)NC1=O)C2=O ; CN1C(=O)CCC(N2C(=O)c3cccc(OCC(=O)OC(C)(C)C)c3C2=O)C1=O]

Di-tert-butyl azodicarboxylate (897 mg, 3.90 mmol, 3.02 eq.) was added slowly To a stirred solution of tert-butyl 2-((2-(2,6-dioxopiperidin-3-yl)-1,3-dioxoisoindolin-4-yl)oxy)acetate (500 mg, 1.29 mmol, 1.00 eq.), MeOH (125 mg, 3.90 mmol, 3.02 eq.) and PPh₃ (681 mg, 2.60 mmol, 2.02 eq.) in THF (80.0 mL) at 0° C. The resulting mixture was stirred at RT overnight, diluted with H₂O and extracted with ethyl acetate. The combined organic phase was washed with brine, dried over Na₂SO₄, concentrated and purified by flash silica gel chromatography (DCM:MeOH=100:1) to get the title compound as a yellow oil.

### Step 2: 2-((2-(1-Methyl-2,6-dioxopiperidin-3-yl)-1,3-dioxoisoindolin-4-yl)oxy)acetic acid

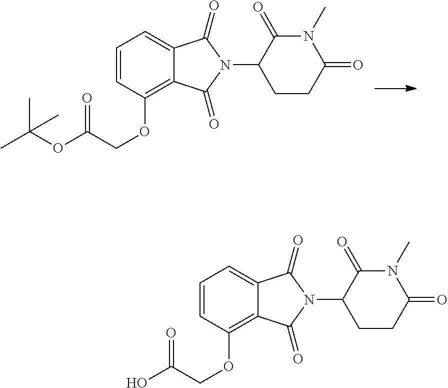 [SMILES: CN1C(=O)CCC(N2C(=O)c3cccc(OCC(=O)OC(C)(C)C)c3C2=O)C1=O ; CN1C(=O)CCC(N2C(=O)c3cccc(OCC(=O)O)c3C2=O)C1=O]

A solution of tert-butyl 2-((2-(1-methyl-2,6-dioxopiperidin-3-yl)-1,3-dioxoisoindolin-4-yl)oxy)acetate (400 mg, 0.99 mmol, 1.00 eq.) and TFA (2.0 mL) in DCM (4.0 mL) was stirred at RT for 1 h. The reaction mixture was concentrated and the residue was triturated with ether to get the title compound as a yellow solid.

### Step 3: N-(2-(2-Hydroxyethoxy)ethyl)-2-((2-(1-methyl-2,6-dioxopiperidin-3-yl)-1,3-dioxoisoindolin-4-yl)oxy)acetamide

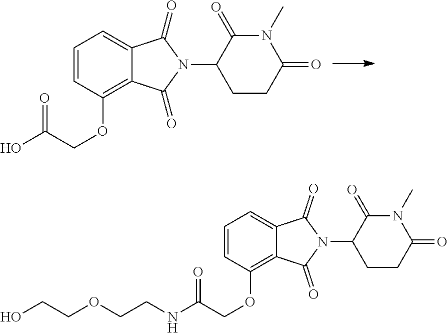 [SMILES: CN1C(=O)CCC(N2C(=O)c3cccc(OCC(=O)O)c3C2=O)C1=O ; CN1C(=O)CCC(N2C(=O)c3cccc(OCC(=O)NCCOCCO)c3C2=O)C1=O]

A solution of 2-((2-(1-methyl-2,6-dioxopiperidin-3-yl)-1,3-dioxoisoindolin-4-yl)oxy)acetic acid (350 mg, 1.01 mmol, 1.00 eq.), 2-(2-aminoethoxy)ethan-1-ol (158 mg, 1.50 mmol, 1.49 eq.), DIPEA (387 mg, 2.99 mmol, 2.96 eq.) and HATU (570 mg, 1.50 mmol, 1.49 eq.) in DMF (6.0 mL) was stirred at 0° C. for 1 h. The reaction mixture was diluted with H₂O and extracted with DCM. The combined organic phase was washed with brine, dried over Na₂SO₄, concentrated to get crude title compound as a brown oil.

### Step 4: 2-(2-(2-((2-(1-Methyl-2,6-dioxopiperidin-3-yl)-1,3-dioxoisoindolin-4-yl)oxy)acetamido) ethoxy)ethyl methanesulfonate

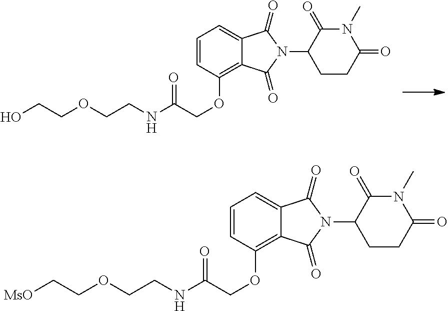 [SMILES: CN1C(=O)CCC(N2C(=O)c3cccc(OCC(=O)NCCOCCO)c3C2=O)C1=O ; CN1C(=O)CCC(N2C(=O)c3cccc(OCC(=O)NCCOCCOS(C)(=O)=O)c3C2=O)C1=O]

MsCl (275 mg, 2.40 mmol, 1.48 eq.) was added to a stirred solution of N-(2-(2-hydroxy-ethoxy)ethyl)-2-((2-(1-methyl-2,6-dioxopiperidin-3-yl)-1,3-dioxoisoindolin-4-yl)oxy)acetamide (700 mg, 1.62 mmol, 1.00 eq.) and TEA (485 mg, 4.79 mmol, 2.96 eq.) in DCM (8.0 mL) at 0° C. After stirring at 0° C. for 1 h, the reaction mixture was diluted with H₂O and extracted with DCM. The combined organic phase was washed with brine, dried over Na₂SO₄, concentrated and purified by flash silica gel chromatography (DCM:MeOH=30:1) to give the title compound as a white solid.

### Reference 6

### Synthesis of 1-((2-(2,6-dioxopiperidin-3-yl)-1,3-dioxoisoindolin-4-yl)oxy)-2-oxo-6,9,12,15-tetraoxa-3-azaheptadecan-17-yl methanesulfonate

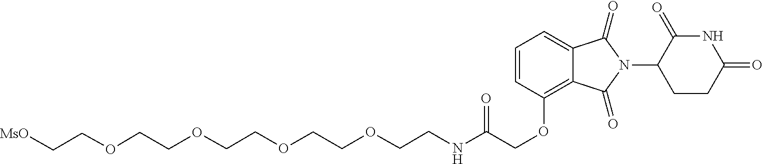 [SMILES: CS(=O)(=O)OCCOCCOCCOCCOCCNC(=O)COc1cccc2c1C(=O)N(C1CCC(=O)NC1=O)C2=O]

### Step 1: 2-((2-(2,6-Dioxopiperidin-3-yl)-1,3-dioxoisoindolin-4-yl)oxy)-N-(14-hydroxy-3,6,9,12-tetraoxatetradecyl)acetamide

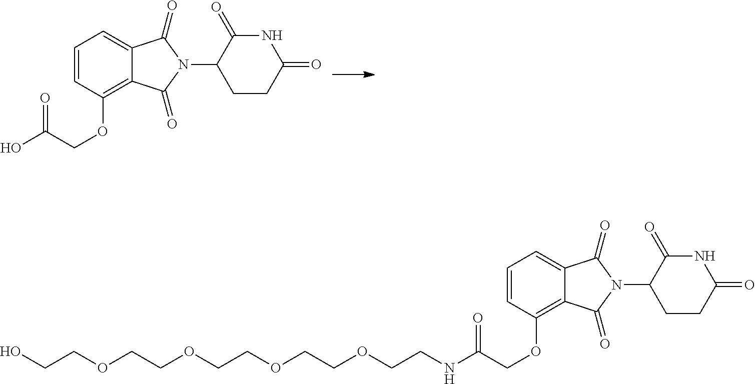 [SMILES: O=C(O)COc1cccc2c1C(=O)N(C1CCC(=O)NC1=O)C2=O ; O=C(COc1cccc2c1C(=O)N(C1CCC(=O)NC1=O)C2=O)NCCOCCOCCOCCOCCO]

A mixture of 2-((2-(2,6-dioxopiperidin-3-yl)-1,3-dioxoisoindolin-4-yl)oxy)acetic acid (300 mg, 0.90 mmol, 1.00 eq.), 14-amino-3,6,9,12-tetraoxatetradecan-1-ol (320 mg, 1.35 mmol, 1.50 eq.), HATU (513 mg, 1.35 mmol, 1.50 eq.) and DIPEA (348 mg, 2.69 mmol, 2.99 eq.) in DMF (6.0 mL) was stirred at 0° C. for 1 h. The reaction mixture was diluted with H₂O and extracted with DCM. The combined organic phase was washed with brine, dried over Na₂SO₄, concentrated to get crude title compound as a yellow oil, which was used for next step without further purification.

### Step 2: 1-((2-(2,6-Dioxopiperidin-3-yl)-1,3-dioxoisoindolin-4-yl)oxy)-2-oxo-6,9,12,15-tetraoxa-3-azaheptadecan-17-yl methanesulfonate

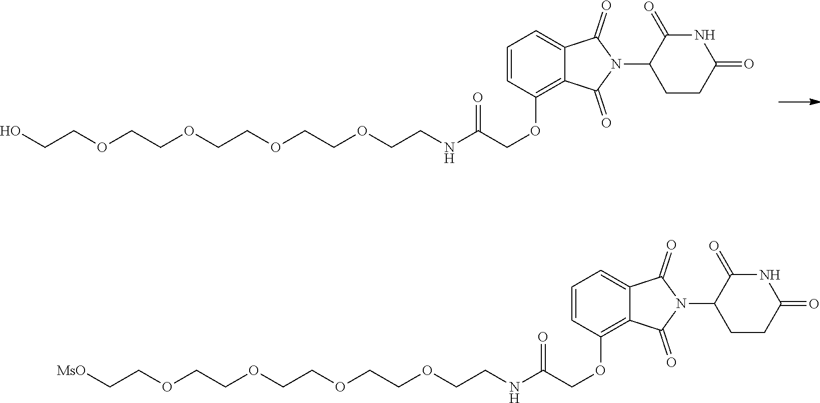 [SMILES: O=C(COc1cccc2c1C(=O)N(C1CCC(=O)NC1=O)C2=O)NCCOCCOCCOCCOCCO ; CS(=O)(=O)OCCOCCOCCOCCOCCNC(=O)COc1cccc2c1C(=O)N(C1CCC(=O)NC1=O)C2=O]

MsCl (271 mg, 2.37 mmol, 1.63 eq.) was added slowly to a stirred solution of 2-((2-(2,6-dioxopiperidin-3-yl)-1,3-dioxoisoindolin-4-yl)oxy)-N-(14-hydroxy-3,6,9,12-tetraoxatetradecyl)-acetamide (800 mg, 1.45 mmol, 1.00 eq.) and TEA (479 mg, 4.73 mmol, 326 eq.) in DCM (8.0 mL) at 0° C. The reaction mixture was stirred at 0° C. for 1 h, diluted with H₂O and extracted with DCM. The combined organic phase was washed with brine, dried over Na₂SO₄, concentrated and purified by flash silica gel chromatography (DCM:MeOH=50:1) to give the title compound as a white solid.

### Reference 7

### Synthesis of 5-(3-(4-((4-aminopiperidin-1-yl)sulfonyl)phenoxy)azetidin-1-yl)-2-(2,6-dioxo-piperidin-3-yl) isoindoline-1,3-dione

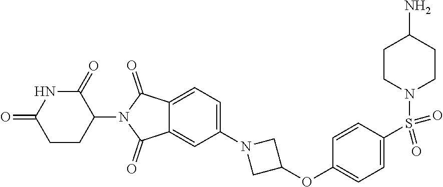 [SMILES: NC1CCN(S(=O)(=O)c2ccc(OC3CN(c4ccc5c(c4)C(=O)N(C4CCC(=O)NC4=O)C5=O)C3)cc2)CC1]

### Step 1: tert-Buty (1-((4-fluorophenyl)sulfonyl)piperidin-4-yl)carbamate

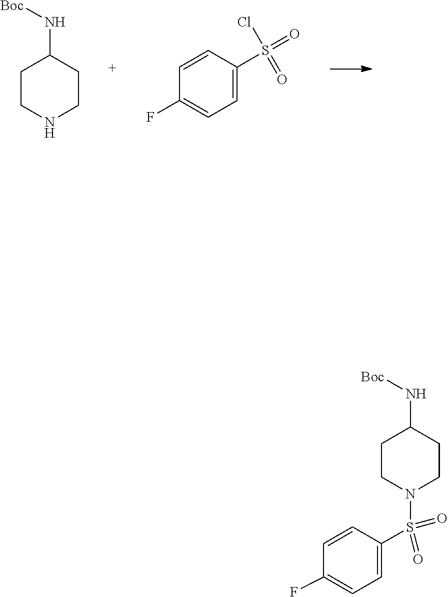 [SMILES: CC(C)(C)OC(=O)NC1CCNCC1 ; O=S(=O)(Cl)c1ccc(F)cc1 ; CC(C)(C)OC(=O)NC1CCN(S(=O)(=O)c2ccc(F)cc2)CC1]

A solution of 4-fluorobenzenesulfonyl chloride (2.6 g, 13.36 mmol, 1.07 eq.) in DCM (10.0 mL) was added dropwise to a stirred solution of tert-butyl piperidin-4-ylcarbamate (2.5 g, 12.48 mmol, 1.00 eq.) in DCM (10.0 mL) and TEA (5.2 mL) at 0° C. The resulting mixture was stirred at RT overnight, concentrated and diluted with DCM. The mixture was stirred at RT for 1 h and filtered to give the title compound as a white solid.

### Step 2: tert-Butyl (1-((4-((1-benzhydrylazetidin-3-yl)oxy)phenyl)sulfonyl)piperidin-4-yl)-carbamate

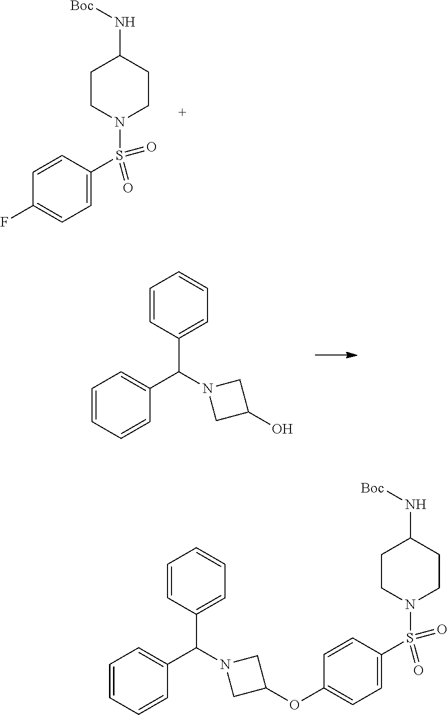 [SMILES: CC(C)(C)OC(=O)NC1CCN(S(=O)(=O)c2ccc(F)cc2)CC1 ; OC1CN(C(c2ccccc2)c2ccccc2)C1 ; CC(C)(C)OC(=O)NC1CCN(S(=O)(=O)c2ccc(OC3CN(C(c4ccccc4)c4ccccc4)C3)cc2)CC1]

To a stirred solution of 1-benzhydrylazetidin-3-ol (1.0 g, 4.18 mmol, 1.00 eq.) in THF (5.0 mL) was added NaH (60%, 251 mg, 6.28 mmol, 1.50 eq.) at 0° C. under N₂. The resulting mixture was stirred at RT for 15 min, then a solution of tert-butyl (1-((4-fluorophenyl)sulfonyl)piperidin-4-yl)carbamate (1.65 g, 4.60 mmol, 1.10 eq.) in THF (5.0 mL) was added slowly and the mixture was stirred at RT overnight. The mixture was diluted with H₂O, and then extracted with DCM. The combined organic layer was washed with aq. NaCl, dried over Na₂SO₄, filtered, and then concentrated. The residue was purified by silica gel flash column (PE:EA=3:1) to give the title compound as a white solid.

### Step 3: tert-Butyl (1-((4-(azetidin-3-yloxy)phenyl)sulfonyl)piperidin-4-yl)carbamate

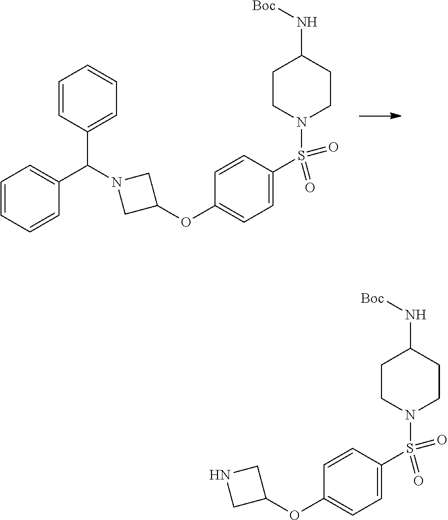 [SMILES: CC(C)(C)OC(=O)NC1CCN(S(=O)(=O)c2ccc(OC3CN(C(c4ccccc4)c4ccccc4)C3)cc2)CC1 ; CC(C)(C)OC(=O)NC1CCN(S(=O)(=O)c2ccc(OC3CNC3)cc2)CC1]

A mixture of tert-butyl (1-((4-((1-benzhydrylazetidin-3-yl)oxy)phenyl)sulfonyl) piperidin-4-yl)carbamate (500 mg, 0.87 mmol, 1.00 eq.), Pd(OH)₂ (300 mg, 20% on carbon) in THF (20.0 mL) was stirred at 50° C. under H₂ (50 psi) overnight. The mixture was cooled, filtrated, concentrated. The residue was purified by silica gel flash column (DCM:MeOH=10:1) to give the title compound as a white solid.

### Step 4: tert-Butyl (1-((4-((1-(2-(2,6-dioxopiperidin-3-yl)-1,3-dioxoisoindolin-5-yl)azetidin-3-yl)oxy)phenyl)sulfonyl)piperidin-4-yl)carbamate

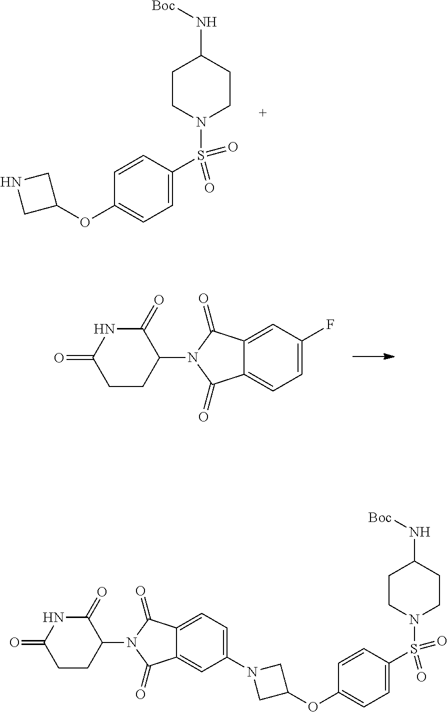 [SMILES: CC(C)(C)OC(=O)NC1CCN(S(=O)(=O)c2ccc(OC3CNC3)cc2)CC1 ; O=C1CCC(N2C(=O)c3ccc(F)cc3C2=O)C(=O)N1 ; CC(C)(C)OC(=O)NC1CCN(S(=O)(=O)c2ccc(OC3CN(c4ccc5c(c4)C(=O)N(C4CCC(=O)NC4=O)C5=O)C3)cc2)CC1]

A mixture of tert-butyl (1-((4-(-87-zetidine-3-yloxy)phenyl)sulfonyl)piperidin-4-yl) carbamate (100 mg, 0.24 mmol, 1.00 eq.), 2-(2,6-dioxopiperidin-3-yl)-5-fluoroisoindoline-1,3-dione (74 mg, 0.27 mmol, 1.13 eq.) and DIPEA (94 mg, 0.73 mmol, 3.04 eq.) in NMP (1.5 mL) was stirred at 140° C. 2 h under microwave irradiation. The mixture was cooled, diluted with water, extracted with DCM, and then concentrated. The residue was purified by silica gel flash column (PE:EA=1:1) to give the title product as a yellow solid.

### Step 5: 5-(3-(4-((4-Aminopiperidin-1-yl)sulfonyl)phenoxy)azetidin-1-yl)-2-(2,6-dioxopiperidin-3-yl) isoindoline-1,3-dione

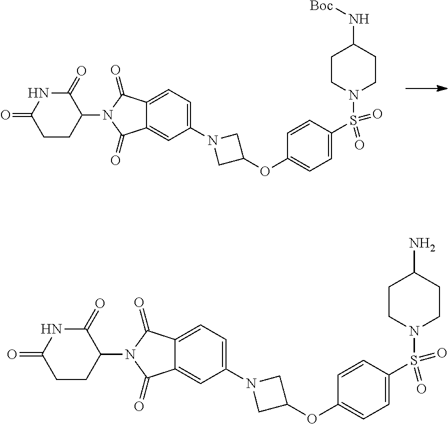 [SMILES: CC(C)(C)OC(=O)NC1CCN(S(=O)(=O)c2ccc(OC3CN(c4ccc5c(c4)C(=O)N(C4CCC(=O)NC4=O)C5=O)C3)cc2)CC1 ; NC1CCN(S(=O)(=O)c2ccc(OC3CN(c4ccc5c(c4)C(=O)N(C4CCC(=O)NC4=O)C5=O)C3)cc2)CC1]

A mixture of tert-butyl (1-((4-((1-(2-(2,6-dioxopiperidin-3-yl)-1,3-dioxo-isoindolin-5′-yl)-88-zetidine-3-yl)oxy)phenyl)sulfonyl)piperidin-4-yl)carbamate (144 mg, 0.21 mmol, 1.00 eq.), TFA (1.0 mL) in DCM (4.0 mL) was stirred at RT for 2 h. The mixture was concentrated to give the title compound as a yellow oil, which was used for next step without further purification.

### Reference 8

### Synthesis of 5-((3-(4-((4-aminopiperidin-1-yl)sulfonyl)phenoxy)azetidin-1-yl)methyl)-2-(2,6-dioxo-piperidin-3-yl)isoindoline-1,3-dione

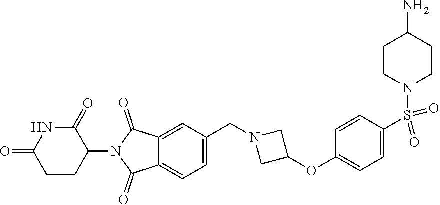 [SMILES: NC1CCN(S(=O)(=O)c2ccc(OC3CN(Cc4ccc5c(c4)C(=O)N(C4CCC(=O)NC4=O)C5=O)C3)cc2)CC1]

### Step 1: 5-(Bromomethyl)-2-(2,6-dioxopiperidin-3-yl)isoindoline-1,3-dione

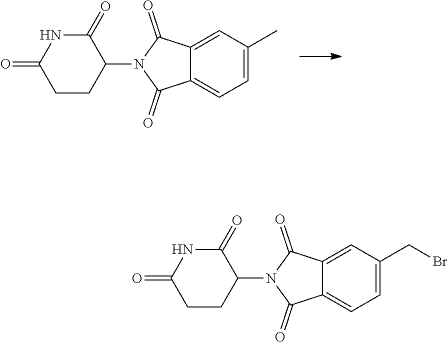 [SMILES: Cc1ccc2c(c1)C(=O)N(C1CCC(=O)NC1=O)C2=O ; O=C1CCC(N2C(=O)c3ccc(CBr)cc3C2=O)C(=O)N1]

NBS (1% mg, 1.10 mmol, 1.10 eq.) and AIBN (32.8 mg, 0.20 mmol, 0.20 eq.) were added to a stirred solution of 2-(2,6-dioxopiperidin-3-yl)-5-methylisoindoline-1,3-dione (272 mg, 1.00 mmol, 1.00 eq.) in MeCN (15.0 mL) and the mixture was stirred at 80° C. overnight under N₂. The mixture was cooled, and concentrated and the residue was purified by flash column chromatography (EA:PE=0-100%) gave the title compound as a white solid.

### Step 2: tert-Butyl (1-((4-((1-((2-(2,6-dioxopiperidin-3-yl)-1,3-dioxoisoindolin-5-yl)methyl) azetidin-3-yl)oxy)phenyl)sulfonyl)piperidin-4-yl)carbamate

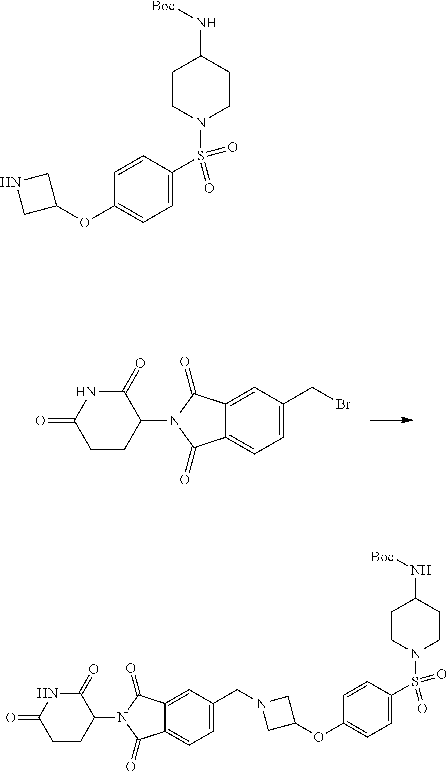 [SMILES: CC(C)(C)OC(=O)NC1CCN(S(=O)(=O)c2ccc(OC3CNC3)cc2)CC1 ; O=C1CCC(N2C(=O)c3ccc(CBr)cc3C2=O)C(=O)N1 ; CC(C)(C)OC(=O)NC1CCN(S(=O)(=O)c2ccc(OC3CN(Cc4ccc5c(c4)C(=O)N(C4CCC(=O)NC4=O)C5=O)C3)cc2)CC1]

A mixture of tert-butyl (1-((4-(azetidin-3-yloxy)phenyl)sulfonyl)piperidin-4-yl)carbamate (100 mg, 0.24 mmol, 1.00 eq., from Reference 7, Step 3), 5-(bromomethyl)-2-(2,6-dioxopiperidin-3-yl)isoindoline-1,3-dione (111 mg, 0.32 mmol, 1.33 eq.) and K₂CO₃ (67 mg, 0.48 mmol, 2.00 eq.) in MeCN (2.0 mL) was stirred at 80° C. overnight. The reaction mixture was cooled, concentrated and purified by silica gel flash column (DCM:MeOH=20:1) to give the title compound as a white solid.

### Step 3: 5-((3-(4-((4-Aminopiperidin-1-yl)sulfonyl)phenoxy)azetidin-1-yl)methyl)-2-(2,6-dioxo-piperidin-3-yl)isoindoline-1,3-dione

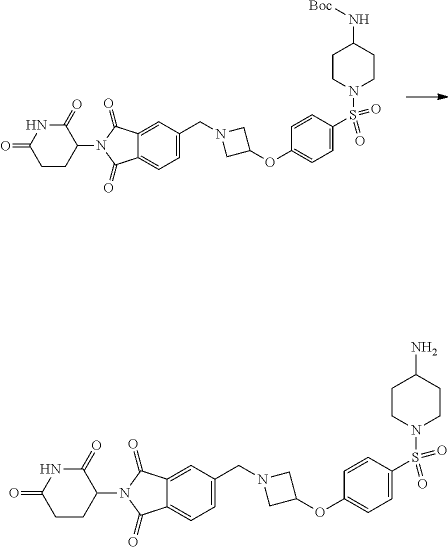 [SMILES: CC(C)(C)OC(=O)NC1CCN(S(=O)(=O)c2ccc(OC3CN(Cc4ccc5c(c4)C(=O)N(C4CCC(=O)NC4=O)C5=O)C3)cc2)CC1 ; NC1CCN(S(=O)(=O)c2ccc(OC3CN(Cc4ccc5c(c4)C(=O)N(C4CCC(=O)NC4=O)C5=O)C3)cc2)CC1]

A mixture of tert-butyl (1-((4-((1-((2-(2,6-dioxopiperidin-3-yl)-1,3-dioxoisoindolin-5-yl) methyl)azetidin-3-yl)oxy)phenyl)sulfonyl)piperidin-4-yl)carbamate (33 mg, 0.048 mmol, 1.00 eq.) and TFA (1.0 mL) in DCM (4.0 mL) was stirred at RT for 3 h. The mixture was concentrated to give the title compound as a yellow solid.

### Reference 9

### Synthesis of 4-amino-N-(2-(2-(2-((2-(2,6-dioxopiperidin-3-yl)-1,3-dioxoisoindolin-4-yl)amino)-ethoxy)ethoxy)ethyl)piperidine-1-sulfonamide

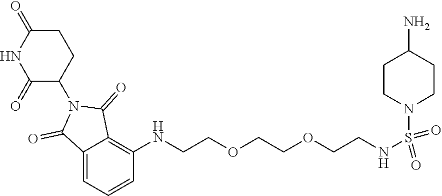 [SMILES: NC1CCN(S(=O)(=O)NCCOCCOCCNc2cccc3c2C(=O)N(C2CCC(=O)NC2=O)C3=O)CC1]

### Step 1: tert-Butyl (2-(2-(2-((2-(2,6-dioxopiperidin-3-yl)-1,3-dioxoisoindolin-4-yl)amino)ethoxy)-ethoxy)ethyl)carbamate

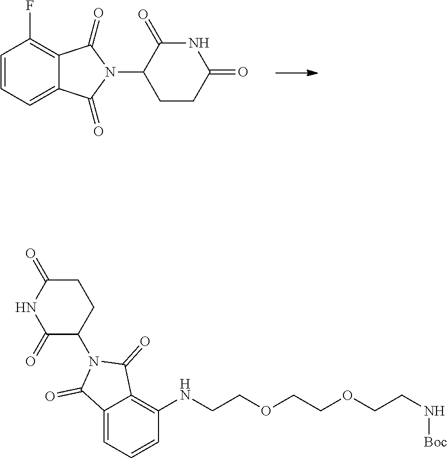 [SMILES: O=C1CCC(N2C(=O)c3cccc(F)c3C2=O)C(=O)N1 ; CC(C)(C)OC(=O)NCCOCCOCCNc1cccc2c1C(=O)N(C1CCC(=O)NC1=O)C2=O]

A mixture of 2-(2,6-dioxopiperidin-3-yl)-4-fluoroisoindoline-1,3-dione (100 mg, 0.36 mmol, 1.10 eq.), tert-butyl (2-(2-(2-aminoethoxy)ethoxy)ethyl)carbamate (81.7 mg, 0.33 mmol, 1.00 eq.), DIPEA (127 mg, 0.98 mmol, 2.97 eq.) in NMP (1.5 mL) was stirred at 140° C. under microwave for 2 h. The mixture was cooled and diluted with ethyl acetate, and then washed with water, brine, dried over Na₂SO₄, and concentrated. The residue was purified by silica gel chromatography (EA:PE=1:3) to give the title compound as a yellow oil.

### Step 2: 4-((2-(2-(2-Aminoethoxy)ethoxy)ethyl)amino)-2-(2,6-dioxopiperidin-3-yl)isoindoline-1,3-dione

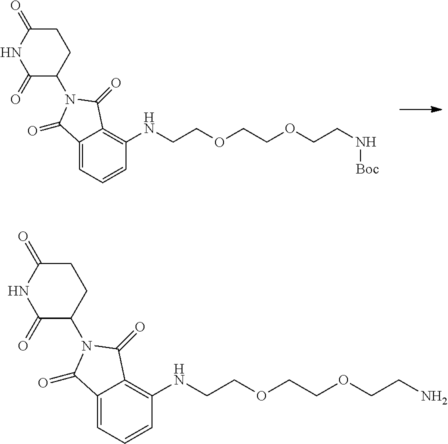 [SMILES: CC(C)(C)OC(=O)NCCOCCOCCNc1cccc2c1C(=O)N(C1CCC(=O)NC1=O)C2=O ; NCCOCCOCCNc1cccc2c1C(=O)N(C1CCC(=O)NC1=O)C2=O]

A mixture of tert-butyl (2-(2-(2-((2-(2,6-dioxopiperidin-3-yl)-1,3-dioxo-isoindolin-4-yl)amino)ethoxy)ethoxy)ethyl)carbamate (180 mg, 0.36 mmol, 1.00 eq.), TFA (0.5 mL) in DCM (2.0 mL) was stirred at RT for 2 h. The mixture was concentrated to give title compound as a yellow oil, which was used for next step without further purification.

### Step 3: tert-Butyl (1-(chlorosulfonyl)piperidin-4-yl)carbamate

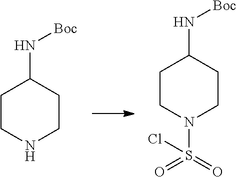 [SMILES: CC(C)(C)OC(=O)NC1CCNCC1 ; CC(C)(C)OC(=O)NC1CCN(S(=O)(=O)Cl)CC1]

Sulfuryl dichloride (81 mg, 0.60 mmol, 1.20 eq.) was added to a stirred solution of tert-butyl piperidin-4-ylcarbamate (100 mg, 0.50 mmol, 1.00 eq.) and TEA (76 mg, 0.75 mmol, 1.50 eq.) in DCM (2.0 mL) at 0° C. and the mixture was stirred at 0° C. for 3 h. The mixture was diluted with water, and then extracted with DCM. The organic layer was washed with brine, dried over Na₂SO₄, and then concentrated to give the title compound as a white solid, which was used for next step directly.

### Step 4: tert-Butyl (1-(N-(2-(2-(2-((2-(2,6-dioxopiperidin-3-yl)-1,3-dioxoisoindolin-4-yl)amino)-ethoxy)ethoxy)ethyl)sulfamoyl)piperidin-4-yl)carbamate

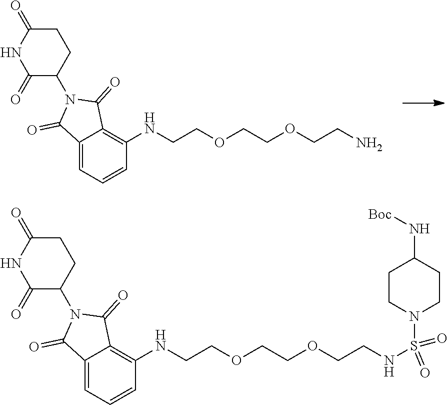 [SMILES: NCCOCCOCCNc1cccc2c1C(=O)N(C1CCC(=O)NC1=O)C2=O ; CC(C)(C)OC(=O)NC1CCN(S(=O)(=O)NCCOCCOCCNc2cccc3c2C(=O)N(C2CCC(=O)NC2=O)C3=O)CC1]

To a stirred solution of 4-((2-(2-(2-aminoethoxy)ethoxy)ethyl)amino)-2-(2,6-dioxo-piperidin-3-yl)isoindoline-1,3-dione (70 mg, 0.17 mmol, 1.00 eq.) and tert-butyl (1-(chloro-sulfonyl)piperidin-4-yl)carbamate (51.9 mg, 0.17 mmol, 1.00 eq.) in DCM (2.0 mL) was added TEA (52.4 mg, 0.52 mmol, 3.00 eq.). The mixture was stirred at 35° C. overnight, and then concentrated. The residue was purified by silica gel chromatography (DCM:MeOH=30:1) to give the title compound as a yellow oil.

### Step 5: 4-Amino-N-(2-(2-(2-((2-(2,6-dioxopiperidin-3-yl)-1,3-dioxoisoindolin-4-yl)amino)-ethoxy)ethoxy)ethyl)piperidine-1-sulfonamide

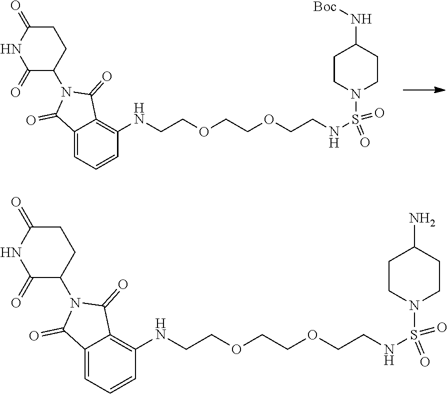 [SMILES: CC(C)(C)OC(=O)NC1CCN(S(=O)(=O)NCCOCCOCCNc2cccc3c2C(=O)N(C2CCC(=O)NC2=O)C3=O)CC1 ; NC1CCN(S(=O)(=O)NCCOCCOCCNc2cccc3c2C(=O)N(C2CCC(=O)NC2=O)C3=O)CC1]

A mixture of tert-butyl (1-(N-(2-(2-(2-((2-(2,6-dioxopiperidin-3-yl)-1,3-dioxoisoindolin-4-yl)amino)ethoxy)ethoxy)ethyl)sulfamoyl)piperidin-4-yl)carbamate (60 mg, 0.090 mmol, 1.00 eq.) in DCM (2.0 mL) and TFA (0.5 mL) was stirred at RT for 2 h. The mixture was concentrated to give the title compound as a yellow oil.

### Reference 10

### Synthesis of 4-amino-N-(3-(3-(2-(2,6-dioxopiperidin-3-yl)-1,3-dioxoisoindolin-4-yl)propoxy)-propyl)-N-methylpiperidine-1-sulfonamide

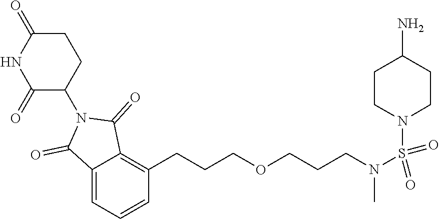 [SMILES: CN(CCCOCCCc1cccc2c1C(=O)N(C1CCC(=O)NC1=O)C2=O)S(=O)(=O)N1CCC(N)CC1]

### Step 1: 4-Bromo-2-(2,6-dioxopiperidin-3-yl)isoindoline-1,3-dione

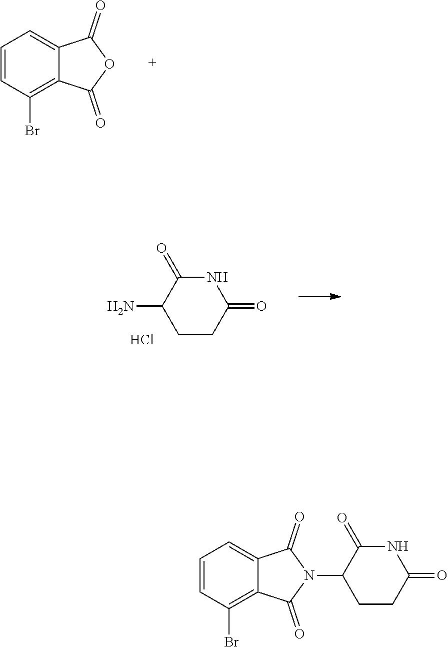 [SMILES: O=C1OC(=O)c2c(Br)cccc21 ; NC1CCC(=O)NC1=O ; Cl ; O=C1CCC(N2C(=O)c3cccc(Br)c3C2=O)C(=O)N1]

A mixture of 4-bromoisobenzofuran-1,3-dione (22.8 g, 100.44 mmol, 1.00 eq.), 3-aminopiperidine-2,6-dione (18.0 g, 109.36 mmol, HCl, 1.09 eq.) and KOAc (29.4 g, 299.54 mmol, 2.98 eq.) in HOAc (200.0 mL) was stirred at 90° C. for 16 h. The reaction mixture was cooled, diluted with ice water and then stirred at 0° C. for 1 h. The mixture was filtered and the filter cake was dried in vacuo to give the title compound as gray solid.

### Step 2: tert-Butyl methyl(3-(prop-2-yn-1-yloxy)propyl)carbamate

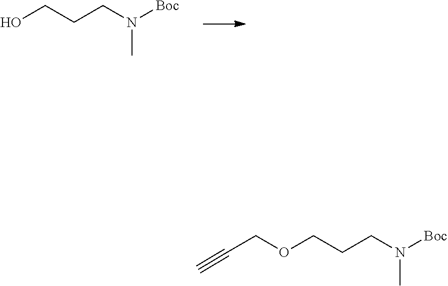 [SMILES: CN(CCCO)C(=O)OC(C)(C)C ; C#CCOCCCN(C)C(=O)OC(C)(C)C]

A mixture of tert-butyl (3-hydroxypropyl)(methyl)carbamate (3.0 g, 15.85 mmol, 1.00 eq.) in DCM (50.0 mL), 3-bromoprop-1-yne (3.0 g, 25.22 mmol, 1.59 eq.), 40% aqueous NaOH (30.0 mL) and tetrabutylammonium hydrogen sulfate (270 mg, 0.80 mmol, 0.050 eq.) was stirred at RT overnight under N₂. The mixture was diluted with water, and then extracted with DCM. The organic layer was washed with water, brine, dried over Na₂SO₄, concentrated. The residue was purified by flash column chromatography (EA:PE=0 to 100%) to give the title compound as a yellow oil.

### Step 3: tert-Butyl (3-((3-(2-(2,6-dioxopiperidin-3-yl)-1,3-dioxoisoindolin-4-yl)prop-2-yn-1-yl)oxy)propyl)(methyl)carbamate

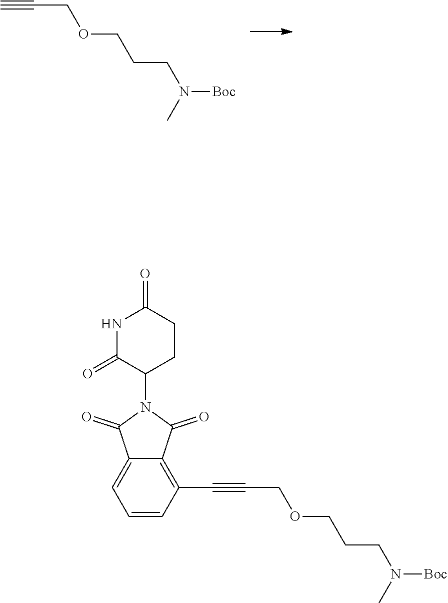 [SMILES: C#CCOCCCN(C)C(=O)OC(C)(C)C ; CN(CCCOCC#Cc1cccc2c1C(=O)N(C1CCC(=O)NC1=O)C2=O)C(=O)OC(C)(C)C]

A mixture of 4-bromo-2-(2,6-dioxopiperidin-3-yl)isoindoline-1,3-dione (1.38 g, 4.09 mmol, 1.00 eq.), tert-butyl methyl(3-(prop-2-yn-1-yloxy)-propyl)carbamate (1.4 g, 6.16 mmol, 1.51 eq.), CuI (78 mg, 0.41 mmol, 0.10 eq.), TEA (7.5 g, 74.12 mmol, 18.12 eq.) and Pd(PPh₃)₂Cl₂ (288 mg, 0.41 mmol, 0.10 eq.) in DMF (15.0 mL) was stirred at 80° C. for 2 h under N₂. The mixture was cooled, diluted with water and then extracted with ethyl acetate. The organic layer was washed with water, brine, dried over Na₂SO₄, and concentrated. Purification by flash column chromatography (EA:PE=0 to 100%) to give the title compound as a yellow oil.

### Step 4: tert-Butyl (3-(3-(2-(2,6-dioxopiperidin-3-yl)-1,3-dioxoisoindolin-4-yl)propoxy)propyl)-(methyl)carbamate

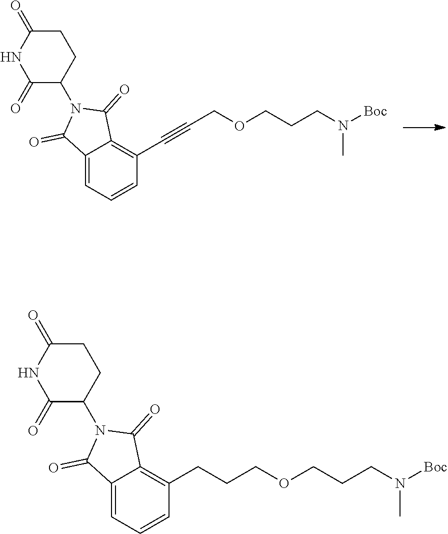 [SMILES: CN(CCCOCC#Cc1cccc2c1C(=O)N(C1CCC(=O)NC1=O)C2=O)C(=O)OC(C)(C)C ; CN(CCCOCCCc1cccc2c1C(=O)N(C1CCC(=O)NC1=O)C2=O)C(=O)OC(C)(C)C]

A mixture of tert-butyl (3-((3-(2-(2,6-dioxopiperidin-3-yl)-1,3-dioxoisoindolin-4-yl)prop-2-yn-1-yl)oxy)propyl)(methyl)carbamate (1.86 g, 3.85 mmol, 1.00 eq.), Pd(OH)-2/C (0.93 g, 50% w/w) in THF (50.0 mL) was stirred at RT overnight under H₂ atmosphere. The reaction mixture was filtered, concentrated and the residue was purified by flash chromatography (EA:PE=0 to 100%) to give the title compound as a yellow oil.

### Step 5: 2-(2,6-Dioxopiperidin-3-yl)-4-(3-(3-(methylamino)propoxy)propyl)isoindoline-1,3-dione

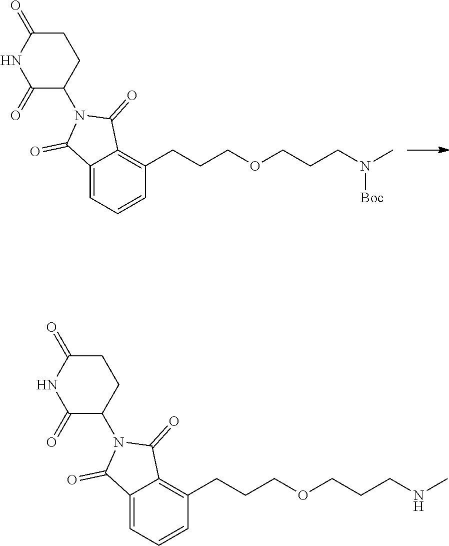 [SMILES: CN(CCCOCCCc1cccc2c1C(=O)N(C1CCC(=O)NC1=O)C2=O)C(=O)OC(C)(C)C ; CNCCCOCCCc1cccc2c1C(=O)N(C1CCC(=O)NC1=O)C2=O]

A mixture of tert-butyl (3-(3-(2-(2,6-dioxopiperidin-3-yl)-1,3-dioxoisoindolin-4-yl)propoxy)propyl)(methyl)carbamate (1.45 g, 2.97 mmol, 1.00 eq.), TFA (1.0 mL) in DCM (10.0 mL) was stirred at RT for 2 h under N₂. The mixture was concentrated and adjusted pH to 9 using aqueous Na₂CO₃, and then the mixture was extracted with DCM. The organic layer was washed with water, brine, dried over Na₂SO₄, and then concentrated to give the title compound as a yellow oil.

### Step 6: tert-Butyl (1-(N-(3-(3-(2-(2,6-dioxopiperidin-3-yl)-1,3-dioxoisoindolin-4-yl)propoxy)-propyl)-N-methylsulfamoyl)piperidin-4-yl)carbamate

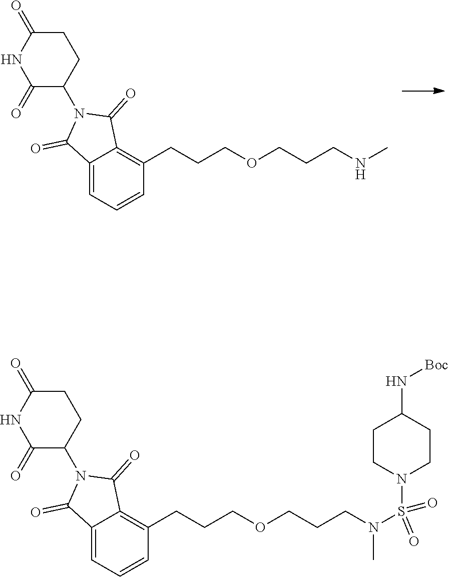 [SMILES: CNCCCOCCCc1cccc2c1C(=O)N(C1CCC(=O)NC1=O)C2=O ; CN(CCCOCCCc1cccc2c1C(=O)N(C1CCC(=O)NC1=O)C2=O)S(=O)(=O)N1CCC(NC(=O)OC(C)(C)C)CC1]

A mixture of 2-(2,6-dioxopiperidin-3-yl)-4-(3-(3-(methylamino)propoxy)-propyl)isoindoline-1,3-dione (150 mg, 0.39 mmol, 1.00 eq.), tert-butyl (1-(chlorosulfonyl)-piperidin-4-yl)carbamate (173 mg, 0.58 mmol, 1.49 eq.) and TEA (118 mg, 1.17 mmol, 3.00 eq.) in DCM (2.0 mL) was stirred at 40° C. overnight under N₂. The mixture was cooled, diluted with water and then extracted with ethyl acetate. The organic layer was washed with water, brine, dried over Na₂SO₄, concentrated to give the title compound as a yellow solid.

### Step 7: 4-Amino-N-(3-(3-(2-(2,6-dioxopiperidin-3-yl)-1,3-dioxoisoindolin-4-yl)propoxy)propyl)-N-methylpiperidine-1-sulfonamide

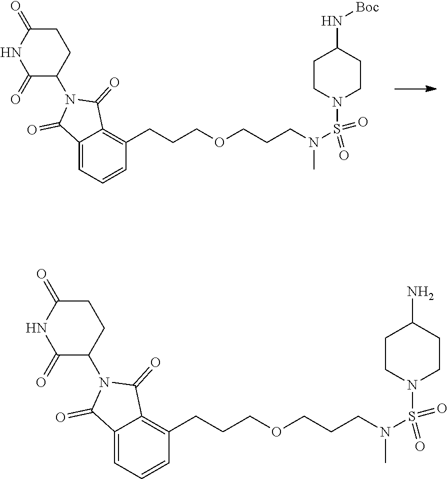 [SMILES: CN(CCCOCCCc1cccc2c1C(=O)N(C1CCC(=O)NC1=O)C2=O)S(=O)(=O)N1CCC(NC(=O)OC(C)(C)C)CC1 ; CN(CCCOCCCc1cccc2c1C(=O)N(C1CCC(=O)NC1=O)C2=O)S(=O)(=O)N1CCC(N)CC1]

To a stirred solution of tert-butyl (1-(N-(3-(3-(2-(2,6-dioxopiperidin-3-yl)-1,3-dioxoisoindolin-4-yl)propoxy)propyl)-N-methylsulfamoyl)piperidin-4-yl)carbamate (200 mg, 0.31 mmol, 1.00 eq.) in DCM (2.0 mL) was added TFA (0.5 mL). The resulting mixture was stirred at RT for 3 h under N₂, concentrated and adjusted pH to 9 using aqueous Na₂CO₃, and then extracted with DCM. The organic layer was washed with water, brine, dried over Na₂SO₄, concentrated to give the title compound as a yellow oil, which was used for next step without further purification.

### Reference 11

### Synthesis of 4-(3-(2-(2-aminoethoxy)ethoxy)propyl)-2-(2,6-dioxopiperidin-3-yl)-isoindoline-1,3-dione

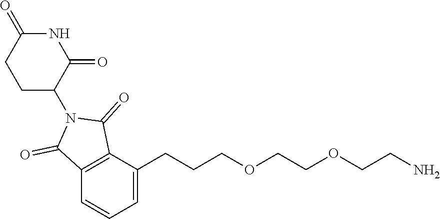 [SMILES: NCCOCCOCCCc1cccc2c1C(=O)N(C1CCC(=O)NC1=O)C2=O]

### Step 1: tert-Butyl (2-(2-(prop-2-yn-1-yloxy)ethoxy)ethyl)carbamate

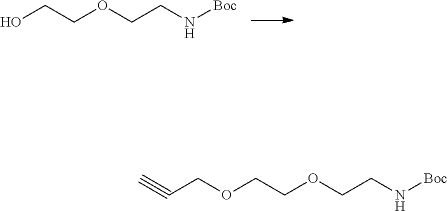 [SMILES: CC(C)(C)OC(=O)NCCOCCO ; C#CCOCCOCCNC(=O)OC(C)(C)C]

NaH (1.2 g, 60%, 30.00 mmol, 1.50 eq.) was added to a stirred solution of tert-butyl (2-(2-hydroxyethoxy)ethyl)carbamate (4.1 g, 19.98 mmol, 1.00 eq.) in THF (50.0 mL) in portions at 0° C. and the mixture was stirred for 1 h. Then 3-bromoprop-1-yne (2.83 g, 23.79 mmol, 1.19 eq.) was added at 0° C. The reaction mixture was warmed to RT and stirred for 16 h, poured into water and extracted with DCM. The organic layer was washed with water, brine, dried over Na₂SO₄, concentrated and purified by flash column chromatography (EA:PE=0 to 100%) to give the title compound as a yellow oil.

### Step 2: 4-Amino-N-(2-(2-(3-(2-(2,6-dioxopiperidin-3-yl)-1,3-dioxoisoindolin-4-yl)propoxy)-ethyl)ethyl)piperidine-1-sulfonamide

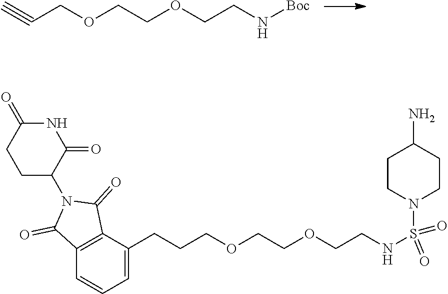 [SMILES: C#CCOCCOCCNC(=O)OC(C)(C)C ; NC1CCN(S(=O)(=O)NCCOCCOCCCc2cccc3c2C(=O)N(C2CCC(=O)NC2=O)C3=O)CC1]

Proceeding analogously as described in Reference 10, Step 3-7 above, but using tert-Butyl (2-(2-(prop-2-yn-1-yloxy)ethoxy)ethyl)carbamate provided the title compound.

### Reference 12

### Synthesis of 5-(3-(3-((4-aminopiperidin-1-yl)sulfonyl)phenoxy)azetidin-1-yl)-2-(2,6-dioxopiperidin-3-yl)isoindoline-1,3-dione

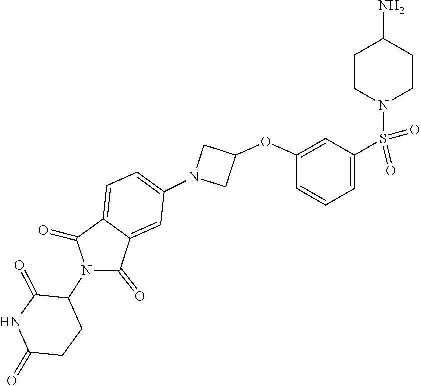 [SMILES: NC1CCN(S(=O)(=O)c2cccc(OC3CN(c4ccc5c(c4)C(=O)N(C4CCC(=O)NC4=O)C5=O)C3)c2)CC1]

### Step 1: Benzyl (1-((3-methoxyphenyl)sulfonyl)piperidin-4-yl)carbamate

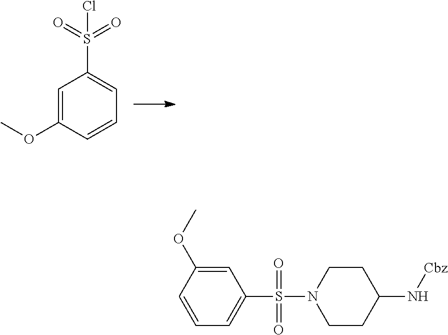 [SMILES: COc1cccc(S(=O)(=O)Cl)c1 ; COc1cccc(S(=O)(=O)N2CCC(NC(=O)OCc3ccccc3)CC2)c1]

A solution of 3-methoxybenzene-1-sulfonyl chloride (3.24 g, 15.68 mmol, 1.05 eq.) in DCM (20.0 mL) was added dropwise to a stirred solution of benzyl piperidin-4-ylcarbamate (3.5 g, 14.94 mmol, 1.00 eq.) and TEA (4.52 g, 44.82 mmol, 3.00 eq.) in DCM (50.0 mL) at 0° C. and the mixture was stirred at RT for 3 h. The mixture was diluted with DCM and the organic layer was washed with water and brine, dried over Na₂SO₄, and concentrated. The residue was purified by silica gel chromatography (EA:PE=1:3) gave the title compound as a white solid.

### Step 2: 3-((4-Aminopiperidin-1-yl)sulfonyl)phenol

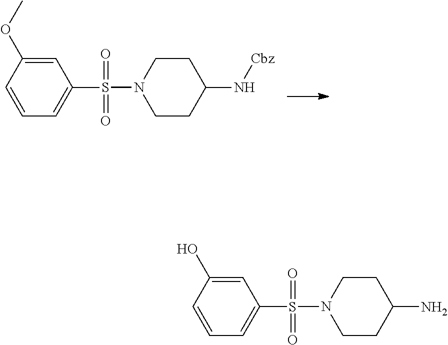 [SMILES: COc1cccc(S(=O)(=O)N2CCC(NC(=O)OCc3ccccc3)CC2)c1 ; NC1CCN(S(=O)(=O)c2cccc(O)c2)CC1]

The solution of benzyl (1-((3-methoxyphenyl)sulfonyl)piperidin-4-yl)carbamate (3.5 g, 8.66 mmol, 1.00 eq.) in CF₃SO₃H (20.0 mL) was stirred under N₂ at 100° C. for 3 h. The reaction mixture was cooled and concentrated to give the title compound as a brown oil, which was used for next step without further purification.

### Step 3: tert-Butyl (1-((3-hydroxyphenyl)sulfonyl)piperidin-4-yl)carbamate

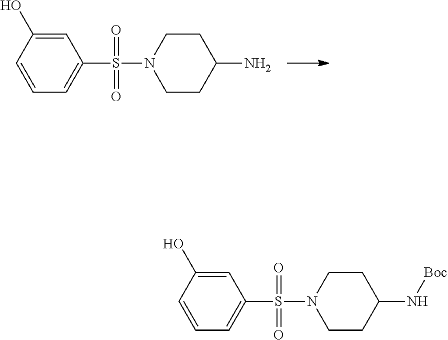 [SMILES: NC1CCN(S(=O)(=O)c2cccc(O)c2)CC1 ; CC(C)(C)OC(=O)NC1CCN(S(=O)(=O)c2cccc(O)c2)CC1]

A solution of (Boc)₂O (852 mg, 3.91 mmol, 1.00 eq.) in DCM (5.0 mL) was added to a stirred solution of 3-((4-aminopiperidin-1-yl)sulfonyl)phenol (1.0 g, 3.91 mmol, 1.00 eq.) in DCM (20.0 mL) and TEA (1.18 g, 11.73 mmol, 3.00 eq.) at 0° C. The mixture was stirred at RT for 2 h, diluted with DCM and the organic layer was washed with water, brine, dried over Na₂SO₄, and concentrated. The residue was purified by flash silica gel chromatography (ACN/water=(35%-75%)) to give the title compound as a white solid.

### Step 4: 1-Benzhydrylazetidin-3-yl methanesulfonate

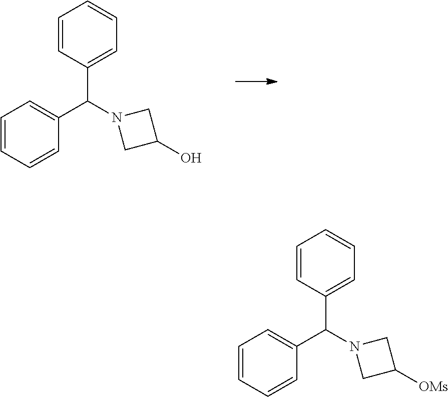 [SMILES: OC1CN(C(c2ccccc2)c2ccccc2)C1 ; CS(=O)(=O)OC1CN(C(c2ccccc2)c2ccccc2)C1]

To a stirred solution of 1-benzhydrylazetidin-3-ol (500 mg, 2.09 mmol, 1.00 eq.) in DCM (10.0 mL) was added TEA (633 mg, 6.27 mmol, 3.00 eq.) and MsCl (479 mg, 4.18 mmol, 2.00 eq.) at 0° C. The mixture was stirred at RT overnight. diluted with DCM and then washed with water. The organic layer was washed with brine, dried over Na₂SO₄, concentrated and then purified by silica gel chromatography (EA:PE=1:3) to give the title compound as a white solid.

### Step 5: tert-Butyl (1-((3-((1-benzhydrylazetidin-3-yl)oxy)phenyl)sulfonyl)piperidin-4-yl)-carbamate

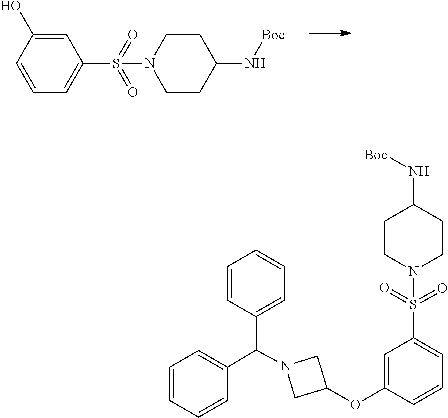 [SMILES: CC(C)(C)OC(=O)NC1CCN(S(=O)(=O)c2cccc(O)c2)CC1 ; CC(C)(C)OC(=O)NC1CCN(S(=O)(=O)c2cccc(OC3CN(C(c4ccccc4)c4ccccc4)C3)c2)CC1]

A mixture of tert-butyl (1-((3-hydroxyphenyl)sulfonyl)piperidin-4-yl)carbamate (533 mg, 1.49 mmol, 1.00 eq.), 1-benzhydrylazetidin-3-yl methanesulfonate (570 mg, 1.79 mmol, 1.20 eq.), Cs₂CO₃ (1.46 g, 4.49 mmol, 3.00 eq.) in DMSO (10.0 mL) was stirred at 90° C. under N₂ for 3 h. The mixture was cooled, diluted with EtOAc and the organic layer was washed with brine, dried over Na₂SO₄, concentrated. The residue was purified by silica gel chromatography (EA:PE=1:3) to give the title compound as a pale yellow solid.

### Step 6: tert-Butyl (1-((3-(azetidin-3-yloxy)phenyl)sulfonyl)piperidin-4-yl)carbamate

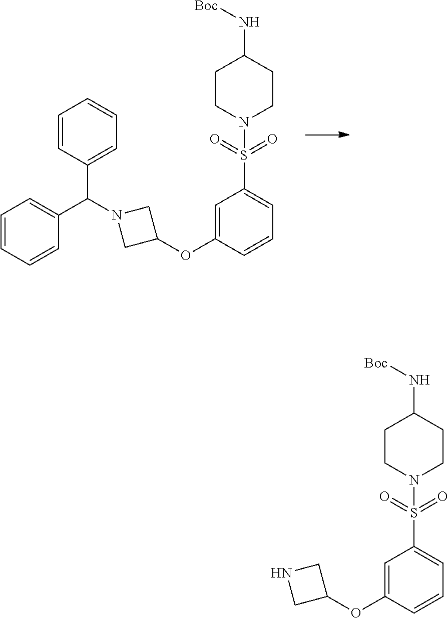 [SMILES: CC(C)(C)OC(=O)NC1CCN(S(=O)(=O)c2cccc(OC3CN(C(c4ccccc4)c4ccccc4)C3)c2)CC1 ; CC(C)(C)OC(=O)NC1CCN(S(=O)(=O)c2cccc(OC3CNC3)c2)CC1]

To a stirred solution of tert-butyl (1-((3-((1-benzhydrylazetidin-3-yl)oxy)phenyl)sulfonyl)-piperidin-4-yl)carbamate (400 mg, 0.69 mmol, 1.00 eq.) in MeOH (15.0 mL) were added Pd(OH)₂/C (20 wt. %, 250 mg) and AcOH (0.5 mL) at RT. The resulting mixture was stirred at 50° C. under H₂ (50 psi) overnight. The reaction mixture was cooled and filtered, and the filtrate was concentrated. The residue was purified by silica gel chromatography (MeOH:DCM=1:15) to give the title compound as a white solid.

### Step 7: tert-Butyl (1-((3-((1-(2-(2,6-dioxopiperidin-3-yl)-1,3-dioxoisoindolin-5-yl)azetidin-3-yl)-oxy)phenyl)sulfonyl)piperidin-4-yl)carbamate

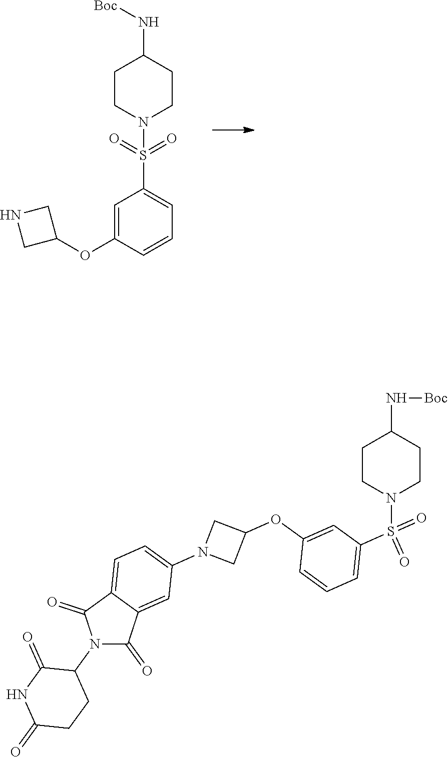 [SMILES: CC(C)(C)OC(=O)NC1CCN(S(=O)(=O)c2cccc(OC3CNC3)c2)CC1 ; CC(C)(C)OC(=O)NC1CCN(S(=O)(=O)c2cccc(OC3CN(c4ccc5c(c4)C(=O)N(C4CCC(=O)NC4=O)C5=O)C3)c2)CC1]

Proceeding analogously as described in Reference 7, Step 4 above, but using tert-butyl (1-((3-(azetidin-3-yloxy)phenyl)sulfonyl)piperidin-4-yl)carbamate and 2-(2,6-dioxopiperidin-3-yl)-5-fluoroisoindoline-1,3-dione provided the title compound.

### Step 8: 5-(3-(3-((4-Aminopiperidin-1-yl)sulfonyl)phenoxy)azetidin-1-yl)-2-(2,6-dioxopiperidin-3-yl)isoindoline-1,3-dione

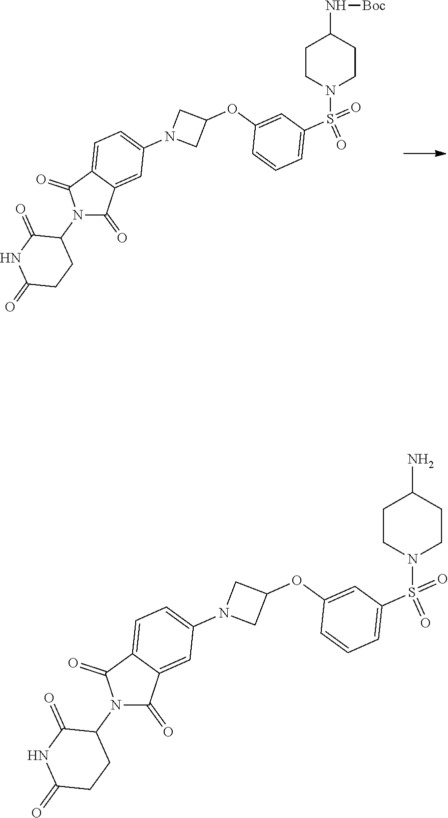 [SMILES: CC(C)(C)OC(=O)NC1CCN(S(=O)(=O)c2cccc(OC3CN(c4ccc5c(c4)C(=O)N(C4CCC(=O)NC4=O)C5=O)C3)c2)CC1 ; NC1CCN(S(=O)(=O)c2cccc(OC3CN(c4ccc5c(c4)C(=O)N(C4CCC(=O)NC4=O)C5=O)C3)c2)CC1]

Proceeding analogously as described in Reference 7, Step 5 above, but using tert-butyl (1-((3-((1-(2-(2,6-dioxopiperidin-3-yl)-1,3-dioxoisoindolin-5-yl)azetidin-3-yl)oxy)phenyl)-sulfonyl)piperidin-4-yl)carbamate provided the title compound.

### Reference 13

### Synthesis of 3-(4-(3-((1-((1-((4-aminopiperidin-1-yl)sulfonyl)piperidin-4-yl)methyl)piperidin-4-yl)-oxy)prop-1-yn-1-yl)-3-methyl-2-oxo-2,3-dihydro-1H-benzo[d]imidazol-1-yl)piperidine-2,6-dione

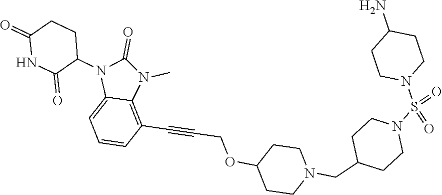 [SMILES: Cn1c(=O)n(C2CCC(=O)NC2=O)c2cccc(C#CCOC3CCN(CC4CCN(S(=O)(=O)N5CCC(N)CC5)CC4)CC3)c21]

### Step 1: tert-Butyl 4-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)methyl)piperidine-1-carboxylate

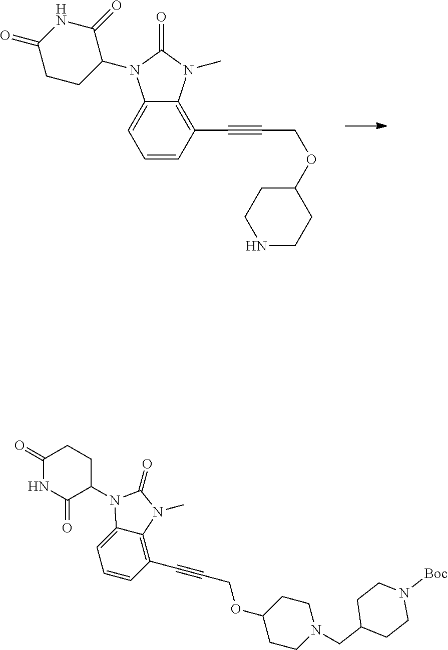 [SMILES: Cn1c(=O)n(C2CCC(=O)NC2=O)c2cccc(C#CCOC3CCNCC3)c21 ; Cn1c(=O)n(C2CCC(=O)NC2=O)c2cccc(C#CCOC3CCN(CC4CCN(C(=O)OC(C)(C)C)CC4)CC3)c21]

A mixture of 3-(3-methyl-2-oxo-4-(3-(piperidin-4-yloxy)prop-1-yn-1-yl)-2,3-dihydro-1H-benzo[d]imidazol-1-yl)piperidine-2,6-dione (300 mg, 0.76 mmol, 1.00 eq., prepared by proceeding as described in Reference 11, Steps 1 and 2 above), tert-butyl 4-(bromomethyl)-piperidine-1-carboxylate (421 mg, 1.51 mmol, 2.00 eq.), NaI (114 mg, 0.76 mmol, 1.00 eq.), K₂CO₃ (634.8 mg, 4.59 mmol, 6.00 eq.) in ACN (5.0 mL) was stirred at 70° C. overnight. The reaction mixture was cooled, concentrated and then purified with chromatograph on silica gel (DCM/MeOH=20/1) to give the title compound as a yellow solid.

### Step 2: 3-(3-Methyl-2-oxo-4-(3-((1-(piperidin-4-ylmethyl)piperidin-4-yl)oxy)prop-1-yn-1-yl)-2,3-dihydro-1H-benzo[d]imidazol-1-yl)piperidine-2,6-dione

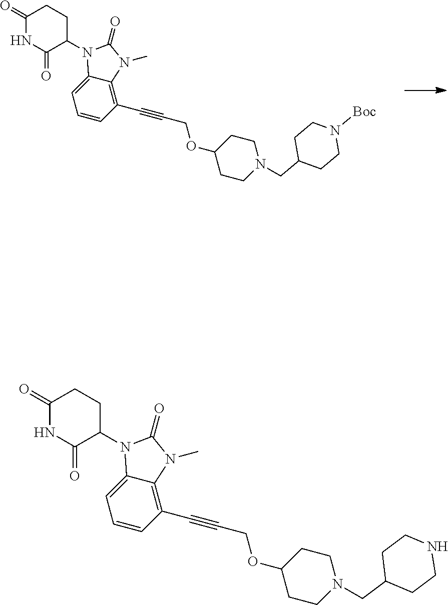 [SMILES: Cn1c(=O)n(C2CCC(=O)NC2=O)c2cccc(C#CCOC3CCN(CC4CCN(C(=O)OC(C)(C)C)CC4)CC3)c21 ; Cn1c(=O)n(C2CCC(=O)NC2=O)c2cccc(C#CCOC3CCN(CC4CCNCC4)CC3)c21]

Proceeding analogously as described in Reference 10, Step 5 above, but using tert-butyl 4-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)methyl)piperidine-1-carboxylate provided the title compound.

### Step 3: tert-Butyl (1-((4-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)methyl)piperidin-1-yl)sulfonyl)piperidin-4-yl)carbamate

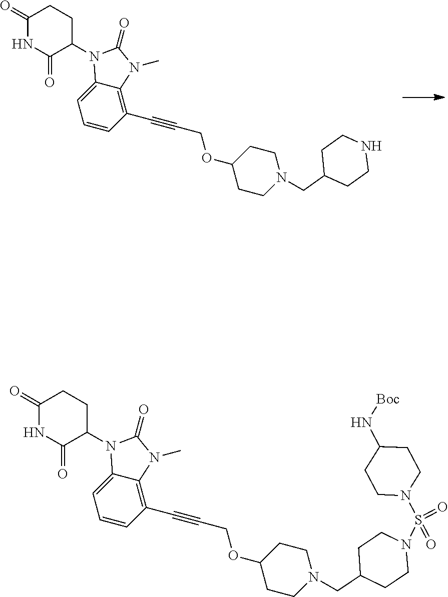 [SMILES: Cn1c(=O)n(C2CCC(=O)NC2=O)c2cccc(C#CCOC3CCN(CC4CCNCC4)CC3)c21 ; Cn1c(=O)n(C2CCC(=O)NC2=O)c2cccc(C#CCOC3CCN(CC4CCN(S(=O)(=O)N5CCC(NC(=O)OC(C)(C)C)CC5)CC4)CC3)c21]

Proceeding analogously as described in Reference 10, Step 6 above, but using 3-(3-methyl-2-oxo-4-(3-((1-(piperidin-4-ylmethyl)piperidin-4-yl)oxy)prop-1-yn-1-yl)-2,3-dihydro-1H-benzo[d]imidazol-1-yl)piperidine-2,6-dione and tert-butyl (1-(chlorosulfonyl)piperidin-4-yl)carbamate provided the title compound.

### Step 4: 3-(4-(3-((1-((1-((4-Aminopiperidin-1-yl)sulfonyl)piperidin-4-yl)methyl)piperdine-4-yl)-oxy)prop-1-yn-1-yl)-3-methyl-2-oxo-2,3-dihydro-1H-benzo[d]imidazol-1-yl)piperidine-2,6-dione

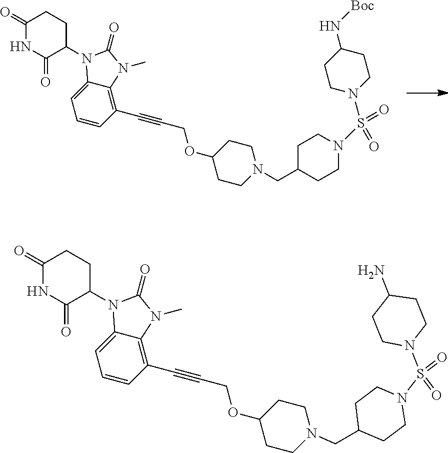 [SMILES: Cn1c(=O)n(C2CCC(=O)NC2=O)c2cccc(C#CCOC3CCN(CC4CCN(S(=O)(=O)N5CCC(NC(=O)OC(C)(C)C)CC5)CC4)CC3)c21 ; Cn1c(=O)n(C2CCC(=O)NC2=O)c2cccc(C#CCOC3CCN(CC4CCN(S(=O)(=O)N5CCC(N)CC5)CC4)CC3)c21]

Proceeding analogously as described in Reference 10, Step 7 above, but using tert-butyl (1-((4-((4-((3-(1-(2,6-dioxopiperidin-3-yl)-3-methyl-2-oxo-2,3-dihydro-1H-benzo[d]imidazol-4-yl)-prop-2-yn-1-yl)oxy)piperidin-1-yl)methyl)piperidin-1-yl)sulfonyl)piperidin-4-yl)carbamate provided the title compound.

### Reference 14

### Synthesis of 3-(4-(3-((1-((4-aminopiperidin-1-yl)sulfonyl)piperidin-4-yl)oxy)prop-1-yn-1-yl)-3-methyl-2-oxo-2,3-dihydro-1H-benzo[d]imidazol-1-yl)piperidine-2,6-dione

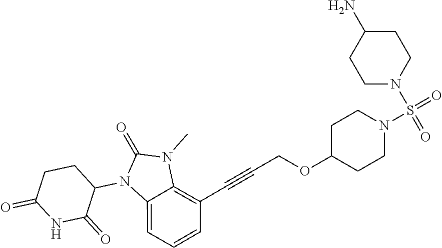 [SMILES: Cn1c(=O)n(C2CCC(=O)NC2=O)c2cccc(C#CCOC3CCN(S(=O)(=O)N4CCC(N)CC4)CC3)c21]

### Step 1: 3-Hydroxy-1-(4-methoxybenzyl)piperidine-2,6-dione

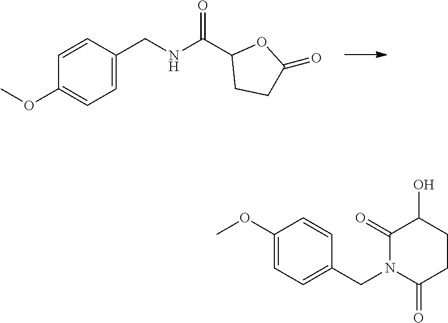 [SMILES: COc1ccc(CNC(=O)C2CCC(=O)O2)cc1 ; COc1ccc(CN2C(=O)CCC(O)C2=O)cc1]

t-BuOK (2.3 g, 20.50 mmol, 1.02 eq.) was added to a stirred mixture of N-(4-methoxybenzyl)-5-oxotetrahydrofuran-2-carboxamide (5.0 g, 20.06 mmol, 1.00 eq.) in THF (50.0 mL) at −78° C. After stirring at −78° C. for 1 h, the mixture was quenched with saturated aqueous NH₄Cl and then extracted with EtOAc. The organic layer was washed with brine, dried over Na₂SO₄, concentrated and then purified with silica gel chromatograph (PE/EA=3/1) to give the title compound as a white solid.

### Step 2: 1-(4-Methoxybenzyl)-2,6-dioxopiperidin-3-yl trifluoromethanesulfonate

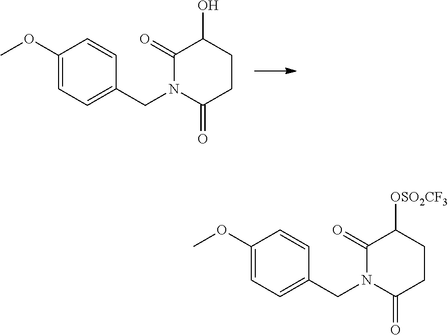

Trifluoromethanesulfonic anhydride (3.2 g, 11.34 mmol, 1.49 eq.) was added slowly to a stirred solution of 3-hydroxy-1-(4-methoxybenzyl)piperidine-2,6-dione (1.9 g, 7.62 mmol, 1.00 eq.) and pyridine (1.2 g, 15.17 mmol, 1.99 eq.) in DCM (40.0 mL) at 0° C. After stirring at 0° C. for 2 h, the reaction mixture was quenched with water and then extracted with DCM. The organic layer was washed with brine, dried over Na₂SO₄, concentrated and then purified by silica gel chromatograph (PE/EA=5/1) to give the title compound as a yellow oil.

### Step 3: 3-(4-Bromo-3-methyl-2-oxo-2,3-dihydro-1H-benzo[d]imidazol-1-yl)-1-(4-methoxy-benzyl)piperidine-2,6-dione

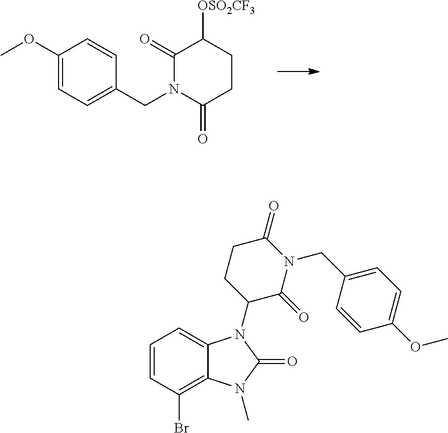

To a stirred solution of 7-bromo-1-methyl-1H-benzo[d]imidazol-2(3H)-one (1.1 g, 4.84 mmol, 1.23 eq.) in THF (30.0 mL) was added t-BuOK (632 mg, 5.63 mmol, 1.43 eq.) at 0° C. After stirring at 0° C. for 0.5 h, a solution of 1-(4-methoxybenzyl)-2,6-dioxopiperidin-3-yl trifluoromethanesulfonate (1.5 g, 3.93 mmol, 1.00 eq.) in THF (10.0 mL) was added at 0° C. The reaction mixture was stirred at 0° C. for 1 h, diluted with water and then extracted with EtOAc. The organic layer was washed with brine, dried over Na₂SO₄, concentrated and the residue was purified by silica gel chromatograph (PE/EA=2/1) to give the title compound as a white solid.

### Step 4: 3-(4-Bromo-3-methyl-2-oxo-2,3-dihydro-1H-benzo[d]imidazol-1-yl)piperidine-2,6-dione

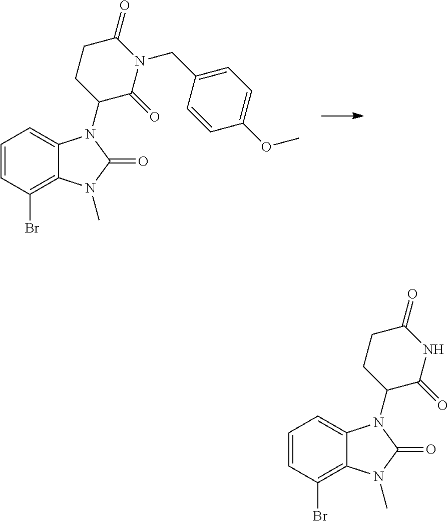 [SMILES: COc1ccc(CN2C(=O)CCC(n3c(=O)n(C)c4c(Br)cccc43)C2=O)cc1 ; Cn1c(=O)n(C2CCC(=O)NC2=O)c2cccc(Br)c21]

A mixture of 3-(4-bromo-3-methyl-2-oxo-2,3-dihydro-1H-benzo[d]imidazol-1-yl)-1-(4-methoxybenzyl)piperidine-2,6-dione (900 mg, 1.96 mmol, 1.00 eq.) in toluene/methanesulfonic acid=2/1 (3.0 mL) was stirred at 120° C. for 3 h. The reaction mixture was cooled, concentrated and poured into ice water. The resulting mixture was filtered, and the cake was dried to give the title compound as a white solid.

### Step 5: tert-Butyl 4-(prop-2-yn-1-yloxy)piperidine-1-carboxylate

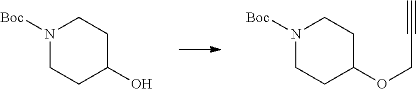 [SMILES: CC(C)(C)OC(=O)N1CCC(O)CC1 ; C#CCOC1CCN(C(=O)OC(C)(C)C)CC1]

NaH (60%, 240 mg, 6.00 mmol, 1.21 eq.) was added to a stirred solution of tert-butyl 4-hydroxypiperidine-1-carboxylate (1.0 g, 4.97 mmol, 1.00 eq.) in THF (20.0 mL) at 0° C., followed by 3-bromoprop-1-yne (704 mg, 5.92 mmol 1.19 eq.). The resulting mixture was stirred at RT for 2 h, quenched with water and then extracted with EtOAc. The organic layer was washed with brine, dried over Na₂SO₄, concentrated and then purified by silica gel chromatograph (PE/EA=10/1) to give the title compound as a white solid.

### Step 6: tert-Butyl 4-((3-(1-(2,6-dioxopiperidin-3-yl)-3-methyl-2-oxo-2,3-dihydro-1H-benzo[d]imidazol-4-yl)prop-2-yn-1-yl)oxy)piperidine-1-carboxylate

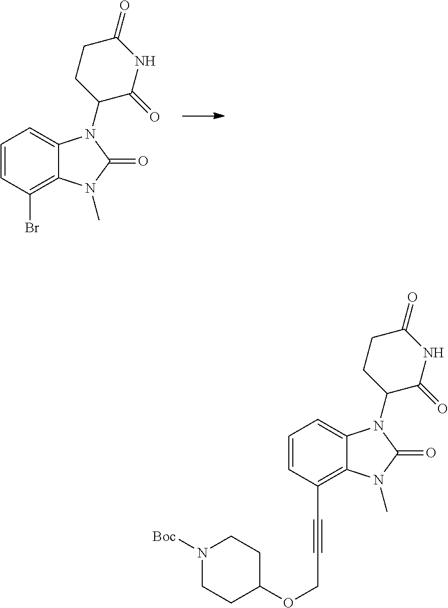 [SMILES: Cn1c(=O)n(C2CCC(=O)NC2=O)c2cccc(Br)c21 ; Cn1c(=O)n(C2CCC(=O)NC2=O)c2cccc(C#CCOC3CCN(C(=O)OC(C)(C)C)CC3)c21]

Proceeding analogously as described in Reference 10, Step 3 above, but using 3-(4-bromo-3-methyl-2-oxo-2,3-dihydro-1H-benzo[d]imidazol-1-yl)piperidine-2,6-dione and tert-butyl 4-(prop-2-yn-1-yloxy)piperidine-1-carboxylate in DMF provided the title compound.

### Step 7: 3-(3-Methyl-2-oxo-4-(3-(piperidin-4-yloxy)prop-1-yn-1-yl)-2,3-dihydro-1H-benzo[d]imidazol-1-yl)piperidine-2,6-dione

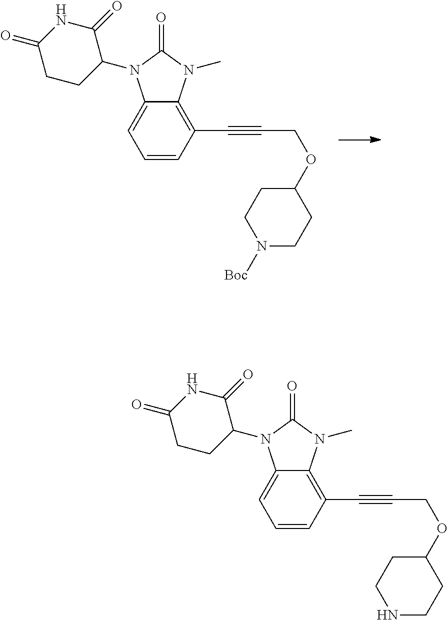 [SMILES: Cn1c(=O)n(C2CCC(=O)NC2=O)c2cccc(C#CCOC3CCN(C(=O)OC(C)(C)C)CC3)c21 ; Cn1c(=O)n(C2CCC(=O)NC2=O)c2cccc(C#CCOC3CCNCC3)c21]

Proceeding analogously as described in Reference 10, Step 5 above, but using tert-butyl 4-((3-(1-(2,6-dioxopiperidin-3-yl)-3-methyl-2-oxo-2,3-dihydro-1H-benzo[d]imidazol-4-yl)prop-2-yn-1-yl)oxy)piperidine-1-carboxylate provided the title compound.

### Step 8: tert-Butyl (1-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)sulfonyl)piperidin-4-yl)carbamate

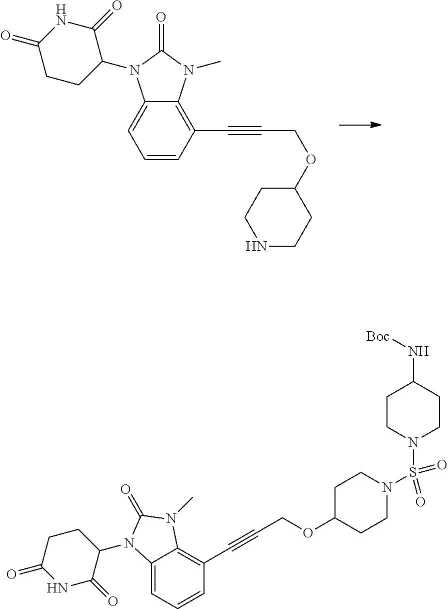 [SMILES: Cn1c(=O)n(C2CCC(=O)NC2=O)c2cccc(C#CCOC3CCNCC3)c21 ; Cn1c(=O)n(C2CCC(=O)NC2=O)c2cccc(C#CCOC3CCN(S(=O)(=O)N4CCC(NC(=O)OC(C)(C)C)CC4)CC3)c21]

Proceeding analogously as described in Reference 10, Step 6 above, but using 3-(3-methyl-2-oxo-4-(3-(piperidin-4-yloxy)prop-1-yn-1-yl)-2,3-dihydro-1H-benzo[d]imidazol-1-yl)piperidine-2,6-dione and tert-butyl (1-(chlorosulfonyl)piperidin-4-yl)carbamate in DMF provided the title compound.

### Step 9: 3-(4-(3-((1-((4-aminopiperidin-1-yl)sulfonyl)piperidin-4-yl)oxy)prop-1-yn-1-yl)-3-methyl-2-oxo-2,3-dihydro-1H-benzo[d]imidazol-1-yl)piperidine-2,6-dione

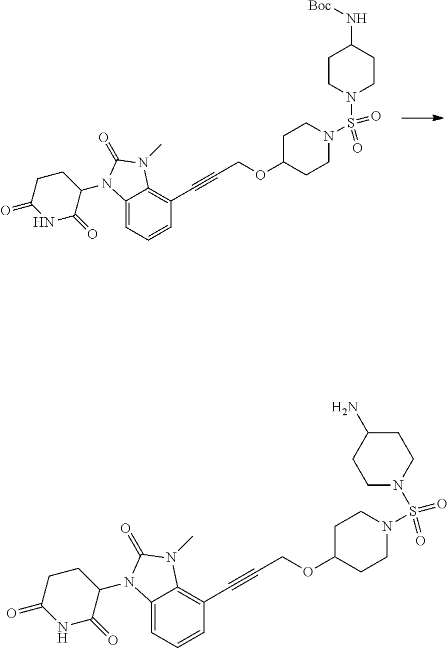 [SMILES: Cn1c(=O)n(C2CCC(=O)NC2=O)c2cccc(C#CCOC3CCN(S(=O)(=O)N4CCC(NC(=O)OC(C)(C)C)CC4)CC3)c21 ; Cn1c(=O)n(C2CCC(=O)NC2=O)c2cccc(C#CCOC3CCN(S(=O)(=O)N4CCC(N)CC4)CC3)c21]

Proceeding analogously as described in Reference 10, Step 7 above, but using tert-butyl (1-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)sulfonyl)piperidin-4-yl)carbamate provided the title compound.

### Reference 15

### Synthesis of 4-amino-N-(4-((2-(2,6-dioxopiperidin-3-yl)-1,3-dioxoisoindolin-4-yl)amino)-cyclohexyl)-N-methylpiperidine-1-sulfonamide 2,2,2-trifluoroacetate

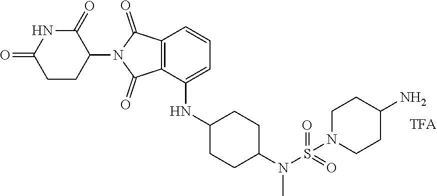 [SMILES: CN(C1CCC(Nc2cccc3c2C(=O)N(C2CCC(=O)NC2=O)C3=O)CC1)S(=O)(=O)N1CCC(N)CC1]

### Step 1: 4-Aminocyclohexanone hydrochloride

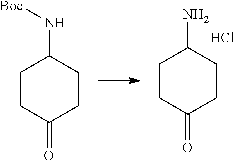 [SMILES: CC(C)(C)OC(=O)NC1CCC(=O)CC1 ; NC1CCC(=O)CC1 ; Cl]

A mixture of tert-butyl (4-oxocyclohexyl)carbamate (500 mg, 2.34 mmol, 1.00 eq.) in a solution of HCl in ethyl acetate (1.0 M, 10.0 mL) was stirred at RT for 1 h. The reaction mixture was concentrated to give the title compound, which was used for next step without further purification.

### Step 2: 2-(2,6-Dioxopiperidin-3-yl)-4-((4-oxocyclohexyl)amino)isoindoline-1,3-dione

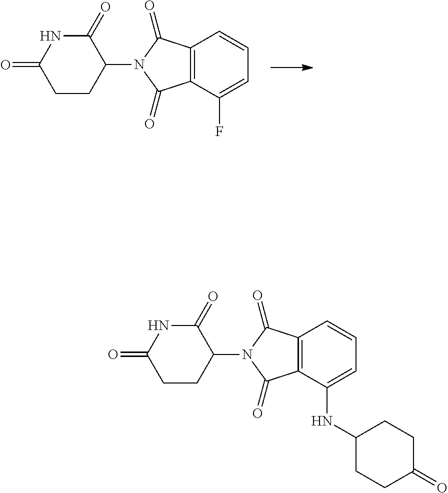 [SMILES: O=C1CCC(N2C(=O)c3cccc(F)c3C2=O)C(=O)N1 ; O=C1CCC(Nc2cccc3c2C(=O)N(C2CCC(=O)NC2=O)C3=O)CC1]

A mixture of 2-(2,6-dioxopiperidin-3-yl)-4-fluoroisoindoline-1,3-dione (276 ng, 1.00 mmol, 1.00 eq.) and 4-aminocyclohexanone hydrochloride (300 mg, 2.00 mmol, 2.00 eq.) in NMP (2.5 mL) was stirred at 140° C. under microwave for 3 h. The reaction mixture was cooled, diluted with DCM and then washed with brine. The organic layer was concentrated, and then the residue was triturated with DCM, filtered to give the title compound as a yellow solid.

### Step 3: 2-(2,6-Dioxopiperidin-3-yl)-4-((4-(methylamino)cyclohexyl)amino)isoindoline-1,3-dione

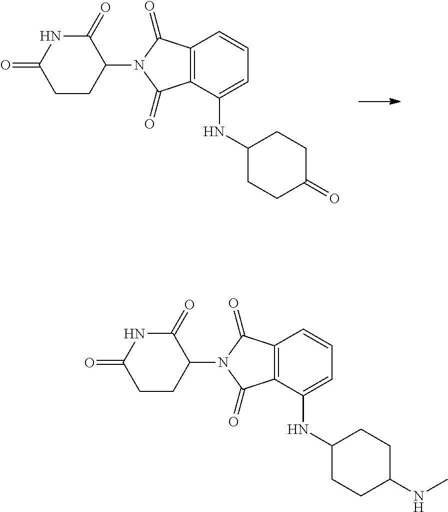 [SMILES: O=C1CCC(Nc2cccc3c2C(=O)N(C2CCC(=O)NC2=O)C3=O)CC1 ; CNC1CCC(Nc2cccc3c2C(=O)N(C2CCC(=O)NC2=O)C3=O)CC1]

To a stirred mixture of 2-(2,6-dioxopiperidin-3-yl)-4-((4-oxocyclohexyl)amino)-isoindoline-1,3-dione (200 mg, 0.54 mmol, 1.00 eq.) and methylamine (40% in MeOH, 210 mg, 2.71 mmol, 5.02 eq.) in MeOH/DCE (2.0 mL/2.0 mL) was added one drop of AcOH. The resulting mixture was stirred at RT for 1 h, and then NaBH(OAc)₃ (345 mg, 1.63 mmol, 3.02 eq.) was added. The reaction mixture was stirred at RT overnight, diluted with DCM, washed with saturated aqueous NaHCO₃ and then brine. The organic layer was dried over Na₂SO₄ and then concentrated to give the title compound as a yellow solid.

### Step 4: tert-Butyl (1-(N-(4-((2-(2,6-dioxopiperidin-3-yl)-1,3-dioxoisoindolin-4-yl)amino)-cyclohexyl)-N-methylsulfamoyl)piperidin-4-yl)carbamate

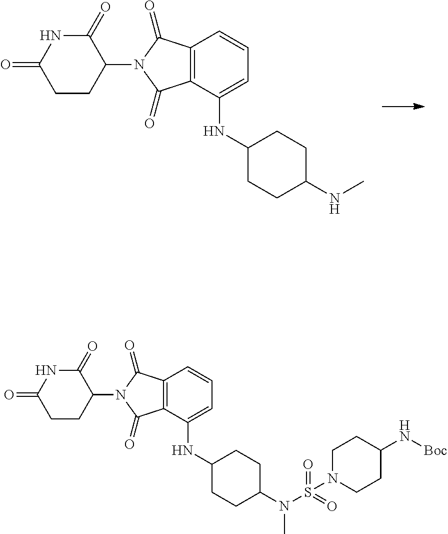 [SMILES: CNC1CCC(Nc2cccc3c2C(=O)N(C2CCC(=O)NC2=O)C3=O)CC1 ; CN(C1CCC(Nc2cccc3c2C(=O)N(C2CCC(=O)NC2=O)C3=O)CC1)S(=O)(=O)N1CCC(NC(=O)OC(C)(C)C)CC1]

Proceeding analogously as described in Reference 10, Step 6 above, but using 2-(2,6-dioxopiperidin-3-yl)-4-((4-(methylamino)cyclohexyl)amino)isoindoline-1,3-dione and tert-butyl (1-(chlorosulfonyl)piperidin-4-yl)carbamate provided the title compound.

### Step 5: 4-Amino-N-(4-((2-(2,6-dioxopiperidin-3-yl)-1,3-dioxoisoindolin-4-yl)amino)cyclohexyl)-N-methylpiperidine-1-sulfonamide 2,2,2-trifluoroacetate

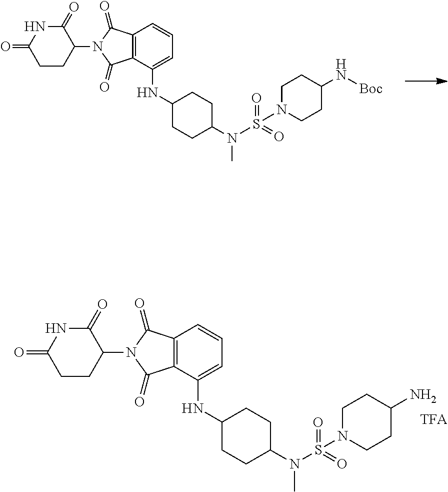 [SMILES: CN(C1CCC(Nc2cccc3c2C(=O)N(C2CCC(=O)NC2=O)C3=O)CC1)S(=O)(=O)N1CCC(NC(=O)OC(C)(C)C)CC1 ; CN(C1CCC(Nc2cccc3c2C(=O)N(C2CCC(=O)NC2=O)C3=O)CC1)S(=O)(=O)N1CCC(N)CC1]

Proceeding analogously as described in Reference 10, Step 7 above, but using tert-butyl (1-(N-(4-((2-(2,6-dioxopiperidin-3-yl)-1,3-dioxoisoindolin-4-yl)amino)cyclohexyl)-N-methylsulfamoyl)piperidin-4-yl)carbamate provided the title compound.

### Reference 16

### Synthesis of tert-butyl 4-((3-(1-(2,6-dioxopiperidin-3-yl)-3-methyl-2-oxo-2,3-dihydro-1H-benzo[d]-imidazol-4-yl)prop-2-yn-1-yl)oxy)piperidine-1-carboxylate

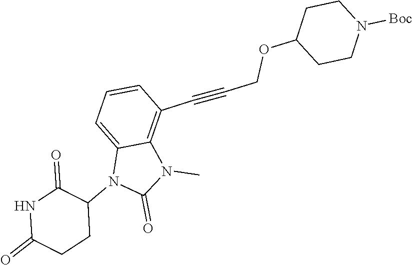 [SMILES: Cn1c(=O)n(C2CCC(=O)NC2=O)c2cccc(C#CCOC3CCN(C(=O)OC(C)(C)C)CC3)c21]

### Step 1: tert-Butyl 4-((3-(1-(2,6-dioxopiperidin-3-yl)-3-methyl-2-oxo-2,3-dihydro-1H-benzo[d]-imidazol-4-yl)prop-2-yn-1-yl)oxy)piperidine-1-carboxylate

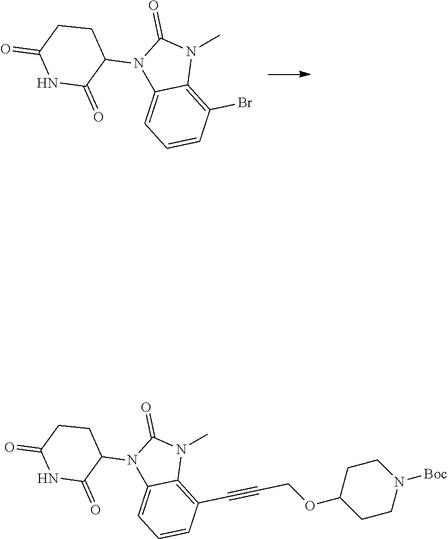 [SMILES: Cn1c(=O)n(C2CCC(=O)NC2=O)c2cccc(Br)c21 ; Cn1c(=O)n(C2CCC(=O)NC2=O)c2cccc(C#CCOC3CCN(C(=O)OC(C)(C)C)CC3)c21]

Proceeding analogously as described in Reference 10, Step 3 above, but using 3-(4-bromo-3-methyl-2-oxo-2,3-dihydro-1H-benzo[d]imidazol-1-yl)piperidine-2,6-dione and tert-butyl 4-(prop-2-yn-1-yloxy)piperidine-I-carboxylate provided the title compound.

### Reference 17

### Synthesis of 5-((3-(3-((4-aminopiperidin-1-yl)sulfonyl)phenoxy)azetidin-1-yl)methyl)-2-(2,6-dioxopiperidin-3-yl)isoindoline-1,3-dione

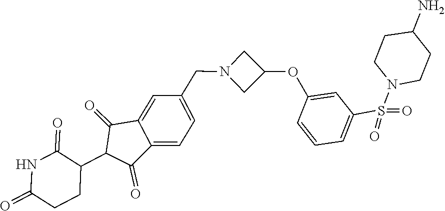 [SMILES: NC1CCN(S(=O)(=O)c2cccc(OC3CN(Cc4ccc5c(c4)C(=O)C(C4CCC(=O)NC4=O)C5=O)C3)c2)CC1]

### Step 1: tert-Butyl (1-((3-((1-((2-(2,6-dioxopiperidin-3-yl)-1,3-dioxoisoindolin-5-yl)methyl)-azetidin-3-yl)oxy)phenyl)sulfonyl)piperidin-4-yl)carbamate

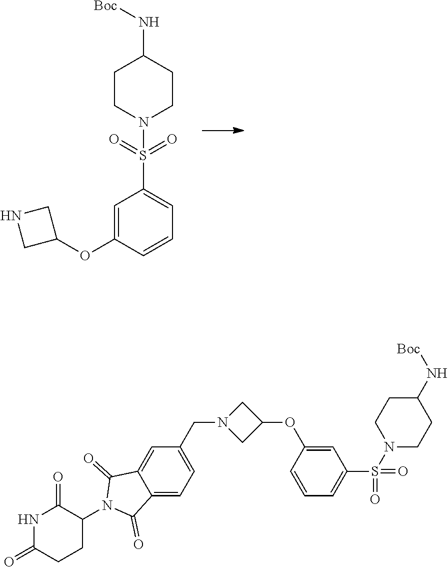 [SMILES: CC(C)(C)OC(=O)NC1CCN(S(=O)(=O)c2cccc(OC3CNC3)c2)CC1 ; CC(C)(C)OC(=O)NC1CCN(S(=O)(=O)c2cccc(OC3CN(Cc4ccc5c(c4)C(=O)N(C4CCC(=O)NC4=O)C5=O)C3)c2)CC1]

Proceeding analogously as described in Reference 8, Step 5 above, but using tert-butyl (1-((3-(azetidin-3-yloxy)phenyl)sulfonyl)piperidin-4-yl)carbamate and 5-(bromomethyl)-2-(2,6-dioxopiperidin-3-yl)isoindoline-1,3-dione provided the title compound.

### Step 2: 5-((3-(3-((4-Aminopiperidin-1-yl)sulfonyl)phenoxy)azetidin-1-yl)methyl)-2-(2,6-dioxopteridin-3-yl)isoindoline-1,3-dione

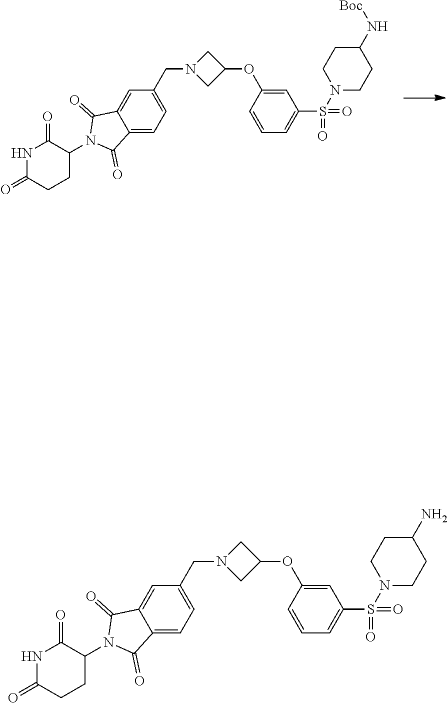 [SMILES: CC(C)(C)OC(=O)NC1CCN(S(=O)(=O)c2cccc(OC3CN(Cc4ccc5c(c4)C(=O)N(C4CCC(=O)NC4=O)C5=O)C3)c2)CC1 ; NC1CCN(S(=O)(=O)c2cccc(OC3CN(Cc4ccc5c(c4)C(=O)N(C4CCC(=O)NC4=O)C5=O)C3)c2)CC1]

Proceeding analogously as described in Reference 8, Step 6 above, but using (1-((3-((1-((2-(2,6-dioxopiperidin-3-yl)-1,3-dioxoisoindolin-5-yl)methyl)azetidin-3-yl)oxy)phenyl)-sulfonyl)piperidin-4-yl)carbamate and TFA provided the title compound.

### Reference 18

### Synthesis of 4-amino-N-(2-(3-(1-(2,6-dioxopiperidin-3-yl)-3-methyl-2-oxo-2,3-dihydro-1H-benzo[d]imidazol-4-yl)propoxy)ethyl)piperidine-1-sulfonamide

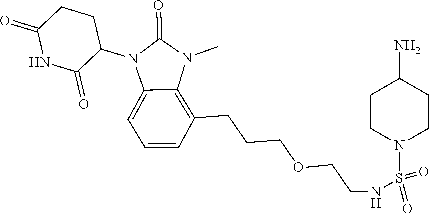 [SMILES: Cn1c(=O)n(C2CCC(=O)NC2=O)c2cccc(CCCOCCNS(=O)(=O)N3CCC(N)CC3)c21]

### Step 1: tert-Butyl (2-(prop-2-yn-1-yloxy)ethyl)carbaminate

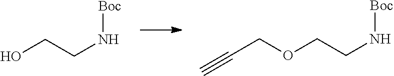

Proceeding analogously as described in Reference 10, Step 2 above, but using tert-butyl (2-hydroxyethyl)carbamate and 3-bromoprop-1-yne provided the title compound.

### Step 2: 4-Amino-N-(2-(3-(1-(2,6-dioxopiperidin-3-yl)-3-methyl-2-oxo-2,3-dihydro-1H-benzo[d]imidazol-4-yl)propoxy)ethyl)piperidine-1-sulfonamide

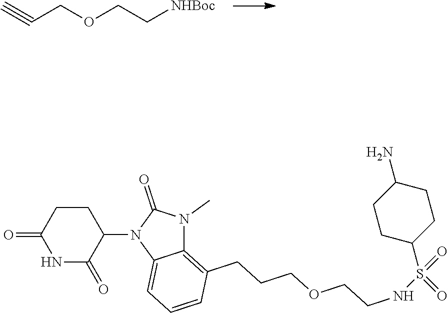 [SMILES: C#CCOCCNC(=O)OC(C)(C)C ; Cn1c(=O)n(C2CCC(=O)NC2=O)c2cccc(CCCOCCNS(=O)(=O)C3CCC(N)CC3)c21]

Proceeding analogously as described in Reference 10, Step 3-7 above, but using tert-butyl (2-(prop-2-yn-1-yloxy)ethyl)carbamate provided the title compound.

### Reference 19

### Synthesis of tert-butyl (1-((1-(2,6-dioxopiperidin-3-yl)-3-methyl-2-oxo-2,3-dihydro-1H-benzo[d]imidazol-4-yl)methyl)piperidin-4-yl)(methyl)carbamate

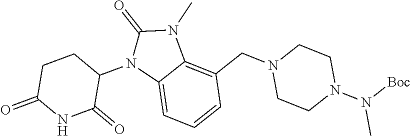 [SMILES: CN(C(=O)OC(C)(C)C)N1CCN(Cc2cccc3c2n(C)c(=O)n3C2CCC(=O)NC2=O)CC1]

### Step 1: 1-(2,6-Dioxopiperidin-3-yl)-3-methyl-2-oxo-2,3-dihydro-1H-benzo[d]imidazole-4-carbaldehyde

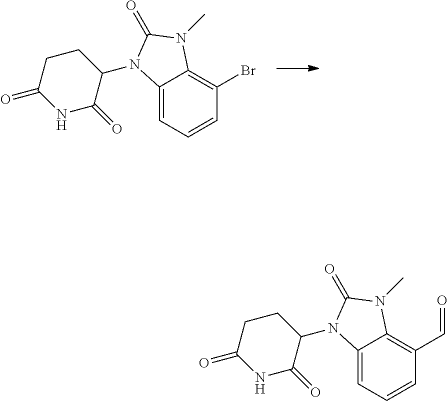 [SMILES: Cn1c(=O)n(C2CCC(=O)NC2=O)c2cccc(Br)c21 ; Cn1c(=O)n(C2CCC(=O)NC2=O)c2cccc(C=O)c21]

A mixture of 3-(4-bromo-3-methyl-2-oxo-2,3-dihydro-1H-benzo[d]imidazol-1-yl)-piperidine-2,6-dione (700 mg, 2.07 mmol, 1.00 eq.), TEA (630 mg, 6.23 mmol, 3.01 eq.), Pd(dppf)Cl₂ (230.6 mg, 0.32 mmol, 0.15 eq.), Et₃SiH (733 mg, 6.30 mmol, 3.04 eq.) in DMF (10 mL) was stirred at 80° C. under 15 psi carbon monoxide atmosphere overnight. The reaction mixture was diluted with water and then extracted with EtOAc. The organic layer was washed with brine, dried over Na₂SO₄, filtered and then concentrated. The residue was purified by chromatograph on silica gel (DCM/MeOH=20/1) to give the title compound as a yellow oil.

### Step 2: tert-Butyl (1-((1-(2,6-dioxopiperidin-3-yl)-3-methyl-2-oxo-2,3-dihydro-1H-benzo[d]imidazol-4-yl)methyl)piperidin-4-yl)(methyl)carbamate

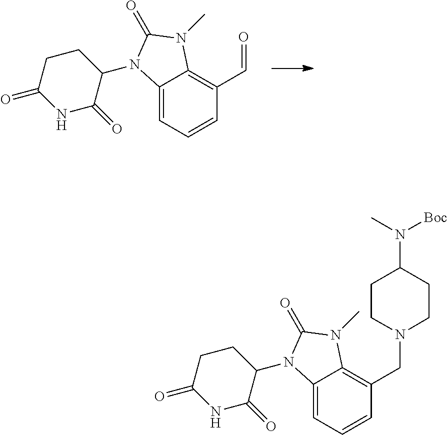 [SMILES: Cn1c(=O)n(C2CCC(=O)NC2=O)c2cccc(C=O)c21 ; CN(C(=O)OC(C)(C)C)C1CCN(Cc2cccc3c2n(C)c(=O)n3C2CCC(=O)NC2=O)CC1]

A mixture of 1-(2,6-dioxopiperidin-3-yl)-3-methyl-2-oxo-2,3-dihydro-1H-benzo[d]-imidazole-4-carbaldehyde (360 mg, 1.25 mmol, 1.00 eq.), tert-butyl N-methyl (piperidin-4-yl)carbamate (403 mg, 1.88 mmol, 1.50 eq.) in THF/DMF=2/1 (5 mL) was stirred at RT for 2 h. NaBH(OAC)₃ (413 mg, 1.95 mmol, 1.60 eq.) was added at RT. After the reaction was complete, the mixture was diluted with water and then extracted with EtOAc. The organic layer was washed with brine, dried over Na₂SO₄ and then concentrated. The residue was purified by Prep-HPLC to give the title compound as a yellow solid.

### Reference 20

### Synthesis of tert-butyl (2-((4-((2-(2,6-dioxopiperidin-3-yl)-1,3-dioxoisoindolin-4-yl)(methyl)amino)benzyl)(methyl)amino)ethyl)(methyl)carbamate

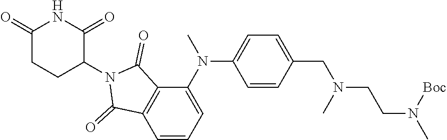 [SMILES: CN(CCN(C)C(=O)OC(C)(C)C)Cc1ccc(N(C)c2cccc3c2C(=O)N(C2CCC(=O)NC2=O)C3=O)cc1]

### Step 1: Dimethyl 3-iodophthalate

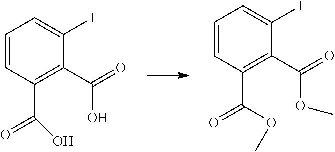

To a stirred mixture of 3-iodophthalic acid (5.00 g, 17.12 mmol, 1.00 eq.), Na₂CO₃ (5.40 g, 50.95 mmol, 2.98 eq.) in DMF (30 mL) was added iodomethane (7.30 g, 51.43 mmol, 3.00 eq.) at RT. The reaction mixture was stirred at 70° C. overnight, cooled, diluted with water, and extracted with EtOAc. The organic layer was washed with brine, dried over Na₂SO₄, filtered, and concentrated. The residue was purified by chromatograph on silica gel (PE/EA=10/1) to give the title compound as a white solid.

### Step 2: 4-(((tert-Butyldimethylsilyl)oxy)methyl)aniline

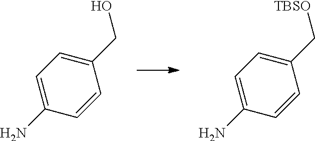 [SMILES: Nc1ccc(CO)cc1 ; CC(C)(C)[Si](C)(C)OCc1ccc(N)cc1]

A mixture of (4-aminophenyl)methanol (2.00 g, 16.24 mmol, 1.00 eq.), DMAP (595 mg, 4.87 mmol, 0.30 eq.), TEA (2.00 g, 19.76 mmol, 1.22 eq.) and TBSCl (2.70 g, 17.91 mmol, 1.10 eq.) in DMF (40 mL) was stirred at RT overnight. The reaction mixture was diluted with water and then extracted with EtOAc. The organic layer was washed with brine, dried over Na₂SO₄, filtered. and then concentrated. The residue was purified by chromatograph on silica gel (PE/EA=10/1) to give the title compound as a colorless oil.

### Step 3: Dimethyl 3-((4-(((tert-butyldimethylsilyl)oxy)methyl)phenyl)amino)phthalate

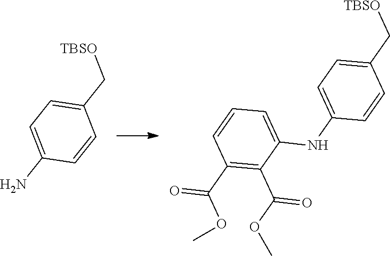 [SMILES: CC(C)(C)[Si](C)(C)OCc1ccc(N)cc1 ; COC(=O)c1cccc(Nc2ccc(CO[Si](C)(C)C(C)(C)C)cc2)c1C(=O)OC]

A mixture of 3-iodo-phthalic acid dimethyl ester (3.00 g, 9.37 mmol, 1.00 eq.), 4-(tert-butyl-dimethyl-silanyloxymethyl)-phenylamine (2.67 g, 11.25 mmol, 1.20 eq.), Pd₂(dba)₃ (436 mg, 0.48 mmol, 0.051 eq.), Cs₂CO₃ (6.11 g, 18.75 mmol, 2.00 eq.), BINAP (143 mg, 0.23 mmol, 0.025 eq.) in toluene (30.0 mL) was stirred at 120° C. overnight under nitrogen atmosphere. The reaction mixture was cooled, concentrated and the residue was purified by chromatograph on silica gel (PE/EA=10/1) to give the title compound as a yellow oil.

### Step 4: Dimethyl 3-((4-(((tert-butyldimethylsilyl)oxy)methyl)phenyl)(methyl)amino)phthalate

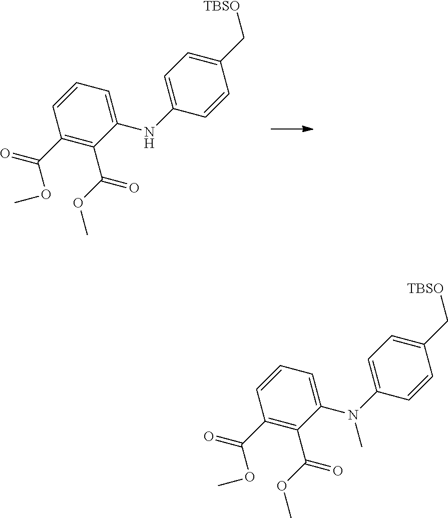 [SMILES: COC(=O)c1cccc(Nc2ccc(CO[Si](C)(C)C(C)(C)C)cc2)c1C(=O)OC ; COC(=O)c1cccc(N(C)c2ccc(CO[Si](C)(C)C(C)(C)C)cc2)c1C(=O)OC]

A mixture of dimethyl 3-((4-(((tert-butyl dimethylsilyl)oxy)methyl)phenyl)-amino)phthalate (1.50 g, 3.49 mmol, 1.00 eq.), iodomethane (991 mg, 6.98 mmol, 2.00 eq.), Cs₂CO₃ (3.41 g, 10.47 mmol, 3.00 eq.) in DMF (30.0 mL) was stirred at 20° C. for 8 h under nitrogen atmosphere. The reaction mixture was diluted with water and extracted with EtOAc. The organic layer was washed with brine, dried over Na₂SO₄, filtered, and then concentrated. The residue was purified by chromatograph on silica gel (PE/EA=5/1) to give the title compound as a yellow oil.

### Step 5: 3-[(4-Hydroxymethyl-phenyl)-methyl-amino]-phthalic acid dimethyl ester

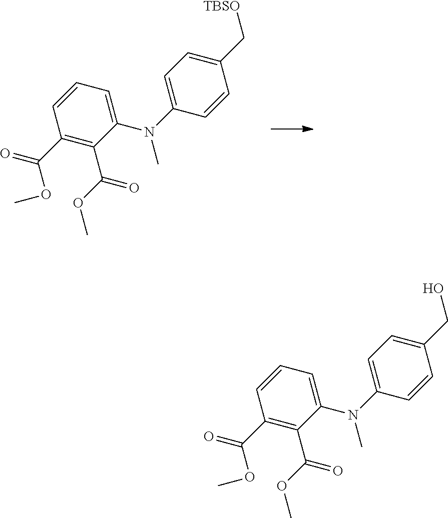 [SMILES: COC(=O)c1cccc(N(C)c2ccc(CO[Si](C)(C)C(C)(C)C)cc2)c1C(=O)OC ; COC(=O)c1cccc(N(C)c2ccc(CO)cc2)c1C(=O)OC]

A solution of TBAF in THF (3.0 M, 2.0 mL) was added To a stirred solution of dimethyl 3-((4-(((tert-butyl dimethylsilyl)oxy)methyl)phenyl)-(methyl)amino)phthalate (500 mg, 1.13 mmol, 1.00 eq.) in THF (5.0 mL) at rt. After 2 h, the mixture was diluted with water and then extracted with EtOAc. The organic layer was washed with brine, dried over Na₂SO₄, filtered and then concentrated. The residue was purified by chromatograph on silica gel (PE/EA=2/1) to give the title compound as a yellow oil.

### Step 6: Dimethyl 3-((4-formylphenyl)(methyl)amino)phthalate

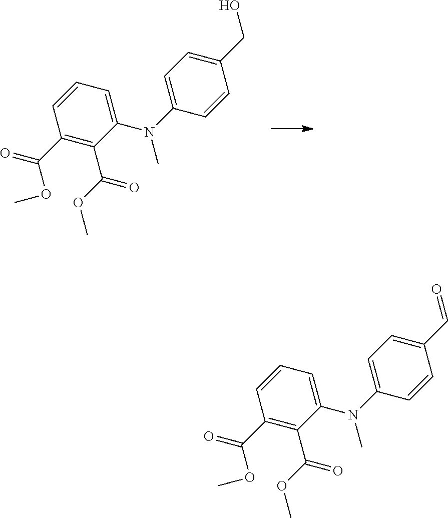 [SMILES: COC(=O)c1cccc(N(C)c2ccc(CO)cc2)c1C(=O)OC ; COC(=O)c1cccc(N(C)c2ccc(C=O)cc2)c1C(=O)OC]

A mixture of 3-[(4-hydroxymethyl-phenyl)methylamino]phthalic acid dimethyl ester (300 mg, 0.91 mmol, 1.00 eq.) and MnO₂ (800 mg, 9.20 mmol, 10.11 eq.) in DCM (10.0 mL) was stirred at rt overnight. The reaction mixture was filtered and concentrated to give the title compound as a yellow oil, which was used for next step without further purification.

### Step 7: Dimethyl 3-((4-(((2-((tert-butoxycarbonyl)(methyl)amino)ethyl)(methyl)amino)methyl) phenyl)(methyl)amino)phthalate

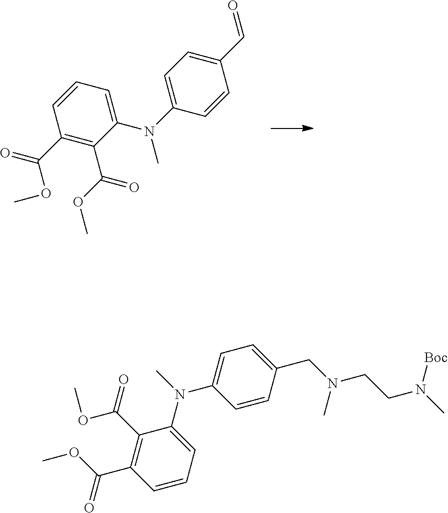

A mixture of dimethyl 3-((4-formylphenyl)(methyl)amino)phthalate (300 mg, 0.92 mmol, 1.00 eq.), methyl-(2-methylamino-ethyl)-carbamic acid tert-butyl ester (205 mg, 1.09 mmol, 1.18 eq.) and a drop of AcOH in DCE (5.0 mL) was stirred at RT for 2 h. NaBH(OAc)₃ (290 mg, 1.37 mmol, 1.49 eq.) was then added and stirred for 4 h. The reaction mixture was concentrated and purified by prep-HPLC to give the title compound as a white solid.

### Step 8: 3-((4-(((2-((tert-Butoxycarbonylxmethyl)amino)ethylXmethyl)amino)methyl)phenyl)(methyl)amino )phthalic acid

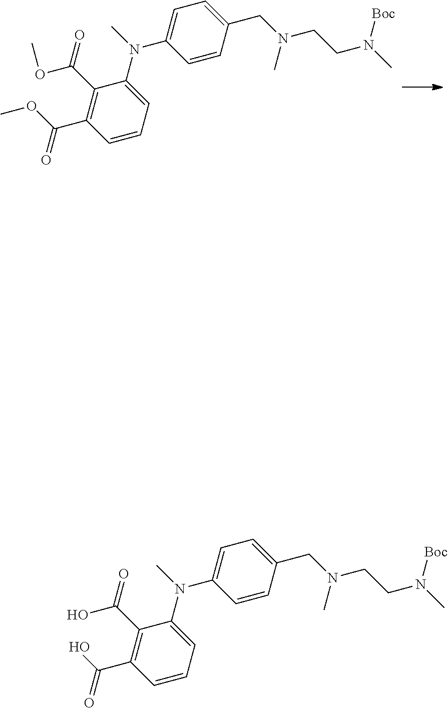

A mixture of dimethyl 3-((4-(((2-((tert-butoxycarbonyl)(methyl)amino)ethyl)(methyl) amino)methyl)phenyl)(methyl)amino)phthalate (250 mg, 0.50 mmol, 1.00 eq.) and NaOH (40 mg, 1.00 mmol, 2.00 eq.) in EtOH/H₂O=2/1 (5.0 mL) was stirred at 80° C. for 5 h. The reaction mixture was concentrated and purified by prep-HPLC to give the title compound as a white solid.

### Step 9: tert-Butyl (2-((4-((2-(2,6-dioxopiperidin-3-yl)-1,3-dioxoisoindolin-4-yl)(methyl)amino) benzyl)(methyl)amino)ethyl)(methyl)carbamate

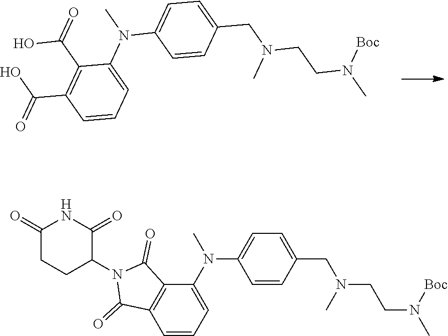 [SMILES: CN(CCN(C)C(=O)OC(C)(C)C)Cc1ccc(N(C)c2cccc(C(=O)O)c2C(=O)O)cc1 ; CN(CCN(C)C(=O)OC(C)(C)C)Cc1ccc(N(C)c2cccc3c2C(=O)N(C2CCC(=O)NC2=O)C3=O)cc1]

A mixture of 3-((4-(((2-((tert-butoxycarbonyl)(methyl)amino)ethyl)(methyl)amino)methyl)phenyl)(methyl)amino)phthalic acid (120 mg, 0.25 mmol, 1.00 eq.) and 3-aminopiperidine-2,6-dione hydrochloride (41 mg, 0.25 mmol, 1.00 eq.) in pyridine (3.0 mL) was stirred at 100° C. overnight. The reaction mixture was cooled and concentrated. The residue was purified by chromatograph on silica gel (DCM/MeOH=30/1) to give the title compound as a yellow solid.

### Reference 21

### Synthesis of tert-butyl (3-(4-(1-(2,6-dioxopiperidin-3-yl)-3-methyl-2-oxo-2,3-dihydro-1H-benzo[d]-imidazol-4-yl)piperidin-1-yl)propyl)(methyl)carbamate

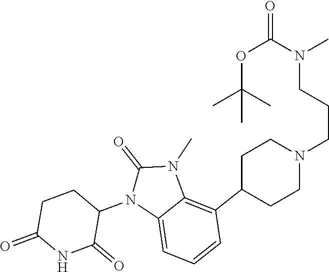 [SMILES: CN(CCCN1CCC(c2cccc3c2n(C)c(=O)n3C2CCC(=O)NC2=O)CC1)C(=O)OC(C)(C)C]

### Step 1: tert-Butyl 4-(1-(2,6-dioxopiperidin-3-yl)-3-methyl-2-oxo-2,3-dihydro-1H-benzo[d]-imidazol-4-yl)-3,6-dihydrouridine-1(2H)-carboxylate

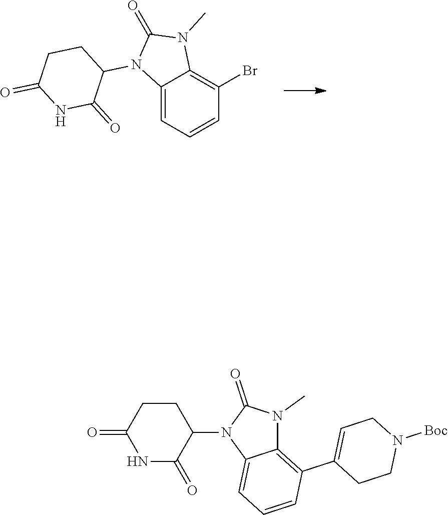 [SMILES: Cn1c(=O)n(C2CCC(=O)NC2=O)c2cccc(Br)c21 ; Cn1c(=O)n(C2CCC(=O)NC2=O)c2cccc(C3=CCN(C(=O)OC(C)(C)C)CC3)c21]

A mixture of 3-(4-bromo-3-methyl-2-oxo-2,3-dihydro-1H-benzo[d]imidazol-1-yl)-piperidine-2,6-dione (100 mg, 0.30 mmol, 1.00 eq.), tert-butyl 4-(4,4,5,5-tetramethyl-1,3,2-dioxaborolan-2-yl)-5,6-dihydropyridine-1(2H)-carboxylate (119 mg, 0.38 mmol, 1.27 eq.), X-Phos-Pd-G3 (38 mg, 0.045 mmol, 0.15 eq.), and K₃PO₄ (191 mg, 0.90 mmol, 3.0 eq.) in 1,4-dioxane/H₂O=10/1 (2.2 mL) was stirred at 60° C. for 3 h. The reaction mixture was diluted with water and then extracted with EtOAc. The organic layer was washed with brine, dried over Na₂SO₄, filtered, and then concentrated. The residue was purified by chromatograph on silica gel (DCM/MeOH=20/1) to give the title compound as a brown solid.

### Step 2: tert-Butyl 4-(1-(2,6-dioxopiperidin-3-yl)-3-methyl-2-oxo-2,3-dihydro-1H-benzo[d]imidazol-4-yl)piperidine-1-carboxylate

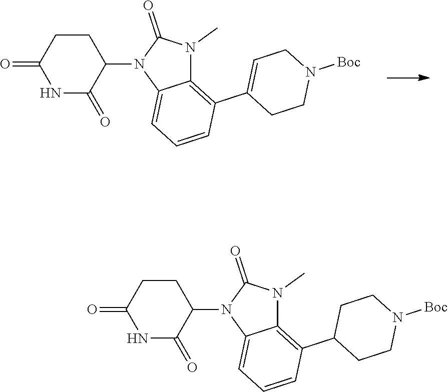 [SMILES: Cn1c(=O)n(C2CCC(=O)NC2=O)c2cccc(C3=CCN(C(=O)OC(C)(C)C)CC3)c21 ; Cn1c(=O)n(C2CCC(=O)NC2=O)c2cccc(C3CCN(C(=O)OC(C)(C)C)CC3)c21]

A mixture of tert-butyl 4-(1-(2,6-dioxopiperidin-3-yl)-3-methyl-2-oxo-2,3-dihydro-1H-benzo[d]imidazol-4-yl)-5,6-dihydropyridine-1(2H)-carboxylate (70 mg, 0.16 mmol, 1.00 eq.), 10% Pd/C (30 mg) and Pd(OH)₂ (30 mg) in TIF (10 mL) was stirred at 50° C. under 50 psi H₂ pressure. The reaction mixture was filtered and then concentrated to give the title as a white solid.

### Step 3: 3-(3-Methyl-2-oxo-4-(piperidin-4-yl)-2,3-dihydro-1H-benzo[d]imidazol-1-yl)piperidine-2,6-dione TFA salt

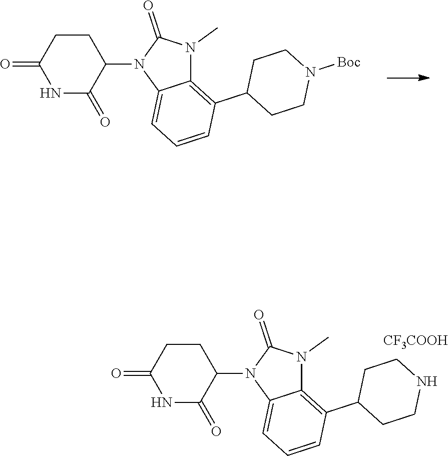 [SMILES: Cn1c(=O)n(C2CCC(=O)NC2=O)c2cccc(C3CCN(C(=O)OC(C)(C)C)CC3)c21 ; Cn1c(=O)n(C2CCC(=O)NC2=O)c2cccc(C3CCNCC3)c21 ; "CF3COOH"]

A mixture of tert-butyl 4-(1-(2,6-dioxopiperidin-3-yl)-3-methyl-2-oxo-2,3-dihydro-1H-benzo[d]imidazol-4-yl)piperidine-1-carboxylate (60 mg, 0.14 mmol, 1.00 eq.) and TFA (0.5 mL) in DCM (2 mL) was stirred at RT for 2 h. The reaction mixture was concentrated to give the title compound as a yellow oil.

### Step 4: tert-Butyl (3-(4-(1-(2,6-dioxopiperidin-3-yl)-3-methyl-2-oxo-2,3-dihydro-1H-benzo[d]-imidazol-4-yl)piperidin-1-yl)propyl)(methyl)carbamate

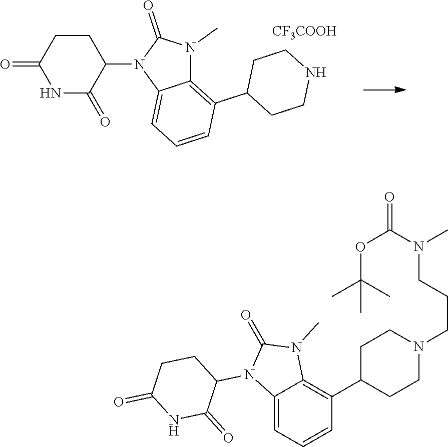 [SMILES: Cn1c(=O)n(C2CCC(=O)NC2=O)c2cccc(C3CCNCC3)c21 ; "CF3COOH" ; CN(CCCN1CCC(c2cccc3c2n(C)c(=O)n3C2CCC(=O)NC2=O)CC1)C(=O)OC(C)(C)C]

To a stirred mixture of 3-(3-methyl-2-oxo-4-(piperidin-4-yl)-2,3-dihydro-1H-benzo[d]imidazol-1-yl)piperidine-2,6-dione TFA salt (60 mg, 0.13 mmol, 1.00 eq.) in THF (5.0 mL) and DMF (1.0 mL) was added one drop of AcOH. After stirring at RT for 0.5 h, tert-butyl methyl(3-oxopropyl)carbamate (63.6 mg, 0.34 mmol, 2.0 eq) was added at RT. The mixture was stirred at 20° C. for 2 h followed by addition of NaBH(OAC)₃ (72 mg, 0.34 mmol, 2.62 eq.). After stirring at RT overnight, the mixture was diluted with water and then extracted with EtOAc. The organic layer was washed with brine, dried over Na₂SO₄, filtered, and then concentrated. The residue was purified by chromatograph on silica gel (DCM/MeOH=50/1) to give the title compound as a yellow solid.

### Reference 22

### Synthesis of tert-butyl (14-((2-(2,6-dioxopiperidin-3-yl)-1,3-dioxoisoindolin-4-yl)amino)-14-oxo-3,6,9,12-tetraoxatetradecyl)carbamate

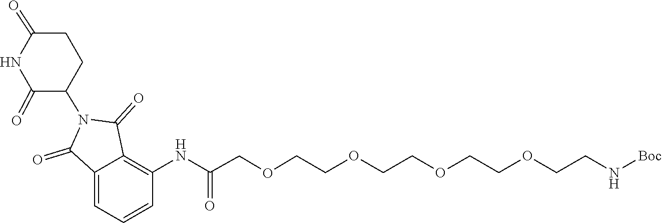 [SMILES: CC(C)(C)OC(=O)NCCOCCOCCOCCOCC(=O)Nc1cccc2c1C(=O)N(C1CCC(=O)NC1=O)C2=O]

### Step 1: 2,2-Dimethyl-4-oxo-3,8,11,14,17-pentaoxa-5-azanonadecan-19-oic acid

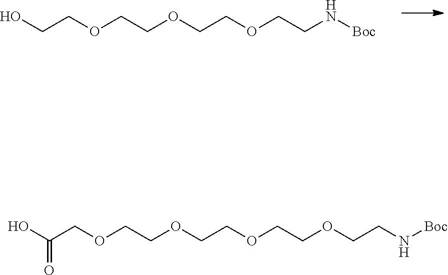 [SMILES: CC(C)(C)OC(=O)NCCOCCOCCOCCO ; CC(C)(C)OC(=O)NCCOCCOCCOCCOCC(=O)O]

NaH (60% in mineral oil, 204 mg, 5.10 mmol, 3.00 eq.) was added to a stirred solution of tert-butyl (2-(2-(2-(2-hydroxyethoxy)ethoxy)ethoxy)ethyl)carbamate (500 mg, 1.70 mmol, 1.00 eq.) in DMF (2 mL) at 0° C. under nitrogen. After stirring at 0° C. for 1 h, 2-iodoacetic acid (793 mg, 4.26 mmol, 2.51 eq.) was added at 0° C. The resulting mixture was slowly warmed to RT and then stirred overnight. This reaction mixture was quenched with H₂O at 0° C., the pH was adjusted to 2-3 with 1 N aqueous HCl and then extracted with EtOAc. The organic layer was washed with brine, dried over Na₂SO₄, filtered, and then concentrated to give the title compound as a yellow oil.

### Step 2: tert-Butyl (14-((2-(2,6-dioxopiperidin-3-yl)-1,3-dioxoisoindolin-4-yl)amino)-14-oxo-3,6,9,12-tetraoxatetradecyl)carbamate

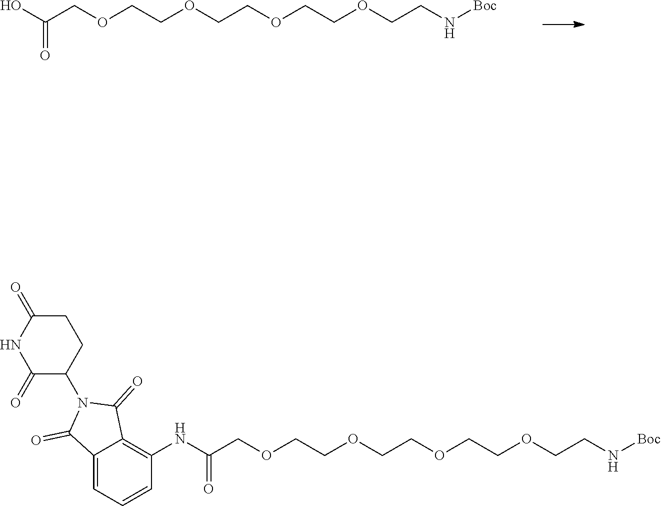 [SMILES: CC(C)(C)OC(=O)NCCOCCOCCOCCOCC(=O)O ; CC(C)(C)OC(=O)NCCOCCOCCOCCOCC(=O)Nc1cccc2c1C(=O)N(C1CCC(=O)NC1=O)C2=O]

To a stirred solution of 2,2-dimethyl-4-oxo-3,8,11,14,17-pentaoxa-5-azanonadecan-19-oic acid (372 mg, 1.06 mmol, 2.00 eq.) in THF (6 mL) was added isobutyl chloroformate (109 mg, 0.80 mmol, 1.51 eq.) and N-methylmorpholine (161 mg, 1.59 mmol, 3.00 eq.), followed by a solution of 4-amino-2-(2,6-dioxopiperidin-3-yl)isoindoline-1,3-dione (145 mg, 0.53 mmol, 1.00 eq.) in DMF (2 mL) dropwise at 0° C. The resulting mixture was stirred at 30° C. overnight, quenched with saturated NaHCO₃, extracted with EtOAc. The organic layer was washed with brine, dried over Na₂SO₄, filtered and then concentrated. The residue was purified by column chromatography on silica gel (PE:EA=1:1) to give the title compound as a yellow solid.

### Reference 23

### Synthesis of tert-butyl (1-((3-((1-(1-(2-(2,6-dioxopiperidin-3-yl)-1-oxoisoindolin-5-yl)piperidin-4-yl)azetidin-3-yl)oxy)phenyl)sulfonyl)piperidin-4-yl)carbamate

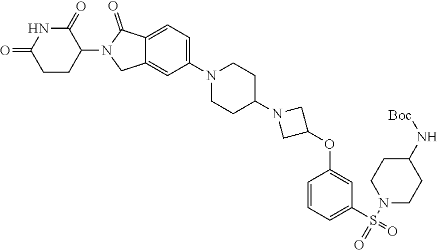 [SMILES: CC(C)(C)OC(=O)NC1CCN(S(=O)(=O)c2cccc(OC3CN(C4CCN(c5ccc6c(c5)CN(C5CCC(=O)NC5=O)C6=O)CC4)C3)c2)CC1]

### Step 1: Benzyl 4-(3-(3-((4-((tert-butoxycarbonyl)amino)piperidin-1-yl)sulfonyl)phenoxy)-azetidin-1-yl)piperidine-1-carboxylate

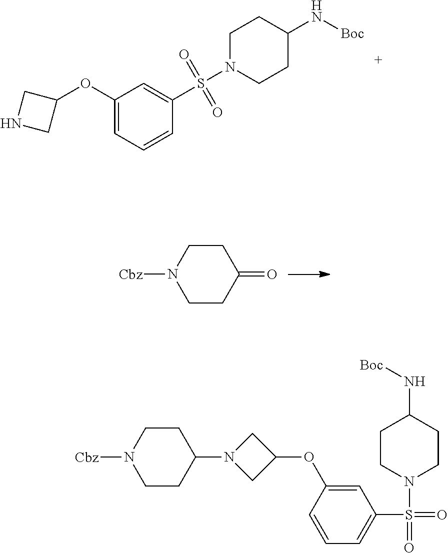 [SMILES: CC(C)(C)OC(=O)NC1CCN(S(=O)(=O)c2cccc(OC3CNC3)c2)CC1 ; O=C1CCN(C(=O)OCc2ccccc2)CC1 ; CC(C)(C)OC(=O)NC1CCN(S(=O)(=O)c2cccc(OC3CN(C4CCN(C(=O)OCc5ccccc5)CC4)C3)c2)CC1]

A solution of tert-butyl (1-((3-(azetidin-3-yloxy)phenyl)sulfonyl)piperidin-4-yl)carbamate (100 mg, 0.24 mmol, 1.00 eq.), benzyl 4-oxopiperidine-1-carboxylate (113 mg, 0.48 mmol, 2.00 eq.) and 1 drop of AcOH in THF (3.0 mL) was stirred at RT for 1 h, followed by addition of NaBH(OAc)₃ (102 mg, 0.48 mmol, 2.00 eq.). The reaction mixture was stirred at RT overnight, diluted with water and then extracted with DCM. The organic layer was concentrated and then purified by silica gel flash column (DCM/MeOH=20/1) to give the title compound as a white solid.

### Step 2: tert-Butyl (1-((3-((1-(piperidin-4-yl)azetidin-3-yl)oxy)phenyl)sulfonyl)piperidin-4-yl)-carbamate

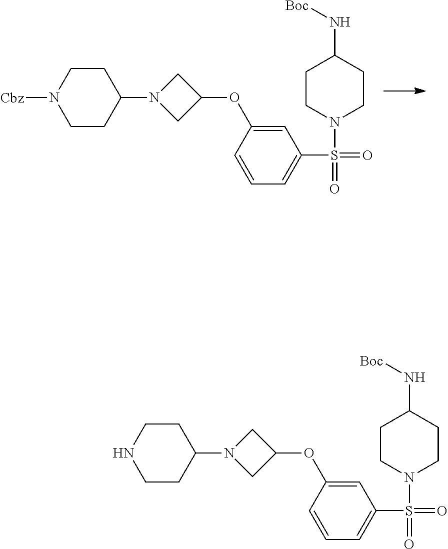 [SMILES: CC(C)(C)OC(=O)NC1CCN(S(=O)(=O)c2cccc(OC3CN(C4CCN(C(=O)OCc5ccccc5)CC4)C3)c2)CC1 ; CC(C)(C)OC(=O)NC1CCN(S(=O)(=O)c2cccc(OC3CN(C4CCNCC4)C3)c2)CC1]

To a stirred solution of benzyl 4-(3-(3-((4-((tert-butoxycarbonyl)amino)piperidin-1-yl)sulfonyl)phenoxy)azetidin-1-yl)piperidine-1-carboxylate (60 mg, 0.095 mmol, 1.00 eq.) in MeOH (10.0 mL) was added 10% Pd/C (20 mg). The resulting mixture was stirred at 45° C. under H₂ atmosphere overnight. The reaction mixture was filtered and concentrated to give the title compound as a white solid.

### Step 3: tert-Butyl (1-((3-((1-(1-(2-(2,6-dioxopiperidin-3-yl)-1-oxoisoindolin-5-yl)piperidin-4-yl)azetidin-3-yl)oxy)phenyl)sulfonyl)piperidin-4-yl)carbamate

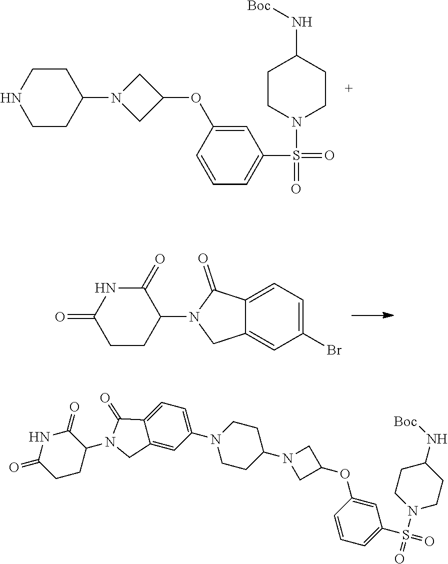 [SMILES: CC(C)(C)OC(=O)NC1CCN(S(=O)(=O)c2cccc(OC3CN(C4CCNCC4)C3)c2)CC1 ; O=C1CCC(N2Cc3cc(Br)ccc3C2=O)C(=O)N1 ; CC(C)(C)OC(=O)NC1CCN(S(=O)(=O)c2cccc(OC3CN(C4CCN(c5ccc6c(c5)CN(C5CCC(=O)NC5=O)C6=O)CC4)C3)c2)CC1]

A mixture of tert-butyl (1-((3-((1-(piperidin-4-yl)azetidin-3-yl)oxy)phenyl)-sulfonyl)piperidin-4-yl)carbamate (39.6 mg, 0.080 mmol, 1.00 eq.), 3-(5-bromo-1-oxoisoindolin-2-yl)piperidine-2,6-dione (38 mg, 0.12 mmol, 1.50 eq.), Cs₂CO₃ (78 mg, 0.24 mmol, 3.00 eq.), Xantphos (15 mg, 0.027 mmol, 0.34 eq.) and Pd(OAc)₂ (15 mg, 0.067 mmol, 0.84 eq.) in 1,4-dioxane (2.0 mL) was stirred at 100° C. overnight under N₂ atmosphere. The mixture was cooled and then filtered. The filtrate was diluted with water and then extracted with DCM. The organic layer was concentrated and then purified by prep-TLC (DCM/MeOH=10/1) to give the title compound as a yellow solid.

### Reference 24

### Synthesis of tert-butyl (1-((3-((1-(azetidin-3-yl)piperidin-4-yl)oxy)phenyl)sulfonyl)piperidin-4-yl)-carbamate

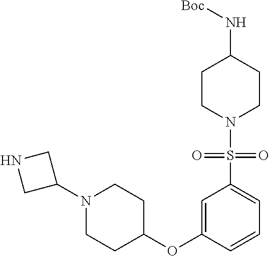 [SMILES: CC(C)(C)OC(=O)NC1CCN(S(=O)(=O)c2cccc(OC3CCN(C4CNC4)CC3)c2)CC1]

### Step 1: tert-Butyl (1-((3-(piperidin-4-yloxy)phenyl)sulfonyl)piperidin-4-yl)carbamate

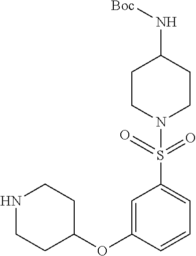 [SMILES: CC(C)(C)OC(=O)NC1CCN(S(=O)(=O)c2cccc(OC3CCNCC3)c2)CC1]

The title compound was prepared by proceeding as described in Reference 12, Steps 1 to 6 using 1-benzhydrylpiperidin-4-yl methanesulfonate.

### Step 2: tert-Butyl (1-((3-((1-(azetidin-3-yl)piperidin-4-yl)oxy)phenyl)sulfonyl)piperidin-4-yl)-carbamate

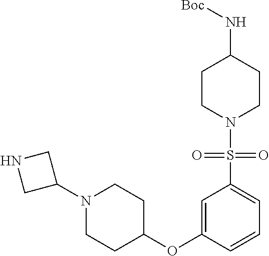 [SMILES: CC(C)(C)OC(=O)NC1CCN(S(=O)(=O)c2cccc(OC3CCN(C4CNC4)CC3)c2)CC1]

tert-Butyl (1-((3-(piperidin-4-yloxy)phenyl)sulfonyl)piperidin-4-yl)carbamate was converted to the title compound by proceeding analogously as described in Reference 23, Steps 1 and 2 above using benzyl 3-oxoazetidine-1-carboxylate.

### Reference 25

### Synthesis of tert-butyl (1-((3-((1-(2-((2-(2,6-dioxopiperidin-3-yl)-1-oxoisoindolin-4-yl)-oxy)ethyl)piperidin-4-yl)oxy)phenyl)sulfonyl)piperidin-4-yl)carbamate

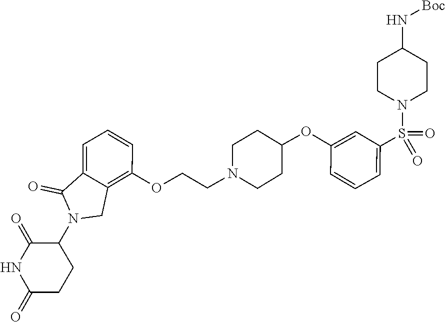 [SMILES: CC(C)(C)OC(=O)NC1CCN(S(=O)(=O)c2cccc(OC3CCN(CCOc4cccc5c4CN(C4CCC(=O)NC4=O)C5=O)CC3)c2)CC1]

### Step 1: Methyl 3-(2-hydroxyethoxy)-2-methylbenzoate

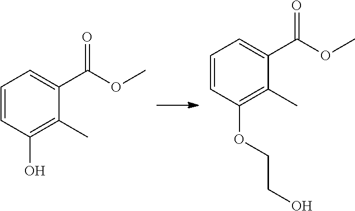

A mixture of methyl 3-hydroxy-2-methylbenzoate (2.50 g, 15.04 mmol, 1.00 eq.) and 1,3-dioxolan-2-one (1.98 g, 22.48 mmol, 1.50 eq.), K₂CO₃ (2.07 g, 14.98 mmol, 1.00 eq.) in DMF (30.0 mL) was stirred at 120° C. under N₂ for 2 h. The reaction mixture was cooled, diluted with water and then extracted with EtOAc. The organic layer was washed water, brine, dried over Na₂SO₄, filtered, and then concentrated. The residue was purified by silica gel chromatography (EA:PE=1:4) to give the title compound as a white solid.

### Step 2: Methyl 2-(bromomethyl)-3-(2-hydroxyethoxy)benzoate

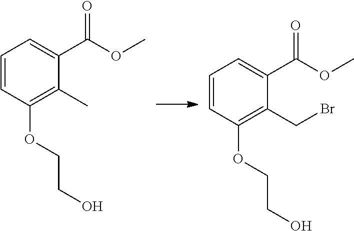 [SMILES: COC(=O)c1cccc(OCCO)c1C ; COC(=O)c1cccc(OCCO)c1CBr]

A mixture of methyl 3-(2-hydroxyethoxy)-2-methy benzoate (1.50 g, 7.14 mmol, 1.00 eq.) in CCl₄ (45.0 mL), NBS (1.46 g, 8.20 mmol, 1.15 eq.) and AIBN (117 mg, 0.71 mmol, 0.10 eq was stirred under N₂ at 75° C. for 3 h. The mixture was cooled and then concentrated. The residue was purified by silica gel chromatography (EA PE=1:3) to give the title compound as a white solid.

### Step 3: 3-(4-(2-Hydroxyethoxy)-1-oxoisoindolin-2-yl)piperidine-2,6-dione

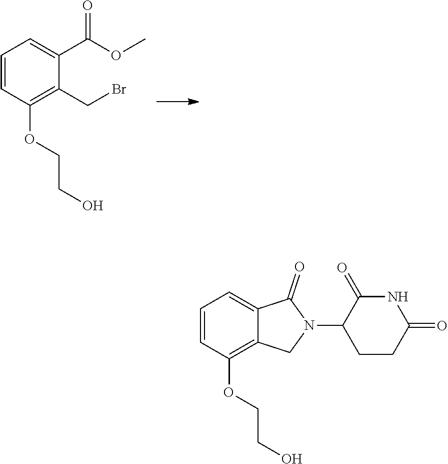 [SMILES: COC(=O)c1cccc(OCCO)c1CBr ; O=C1CCC(N2Cc3c(OCCO)cccc3C2=O)C(=O)N1]

To a stirred solution of methyl 2-(bromomethyl)-3-(2-hydroxyethoxy)benzoate (2.00 g, 6.92 mmol, 1.00 eq.) in ACN (70.0 mL) was added 3-aminopiperidine-2,6-dione hydrochloride (1.48 g, 8.99 mmol, 1.30 eq.) and TEA (1.04 g, 10.28 mmol, 1.49 eq.). The resulting mixture was stirred under N₂ at 80° C. overnight, cooled and then concentrated. The residue was purified by silica gel chromatography (DCM:MeOH=20:1) to give the title compound as a blue solid.

### Step 4: 2-((2-(2,6-Dioxopiperidin-3-yl)-1-oxoisoindolin-4-yl)oxy)ethyl 4-methylbenzenesulfonate

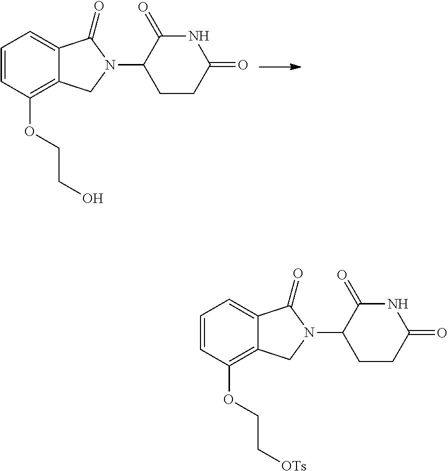 [SMILES: O=C1CCC(N2Cc3c(OCCO)cccc3C2=O)C(=O)N1 ; Cc1ccc(S(=O)(=O)OCCOc2cccc3c2CN(C2CCC(=O)NC2=O)C3=O)cc1]

To a stirred solution of 3-(4-(2-hydroxyethoxy)-1-oxoisoindolin-2-yl)piperidine-2,6-dione (500 mg, 1.64 mmol, 1.00 eq.) in DCM (10.0 mL) was added TEA (333 mg, 3.29 mmol, 2.00 eq.), TsCl (377 mg, 1.98 mmol, 1.21 eq.) and DMAP (20 mg, 0.16 mmol, 0.10 eq.) at 0° C. The resulting mixture was stirred at RT overnight, diluted with DCM, washed with water, brine, dried over Na₂SO₄, filtered, and then concentrated. The residue was purified by silica gel chromatography (DCM:MeOH=30:1) to give the title compound as a green solid.

### Step 5: Benzyl 4-((methylsulfonyl)oxy)piperidine-1-carboxylate

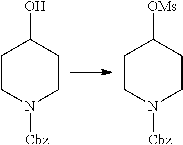 [SMILES: O=C(OCc1ccccc1)N1CCC(O)CC1 ; CS(=O)(=O)OC1CCN(C(=O)OCc2ccccc2)CC1]

To a stirred solution of benzyl 4-hydroxypiperidine-1-carboxylate (2.00 g, 8.50 mmol, 1.00 eq.) in DCM (20.0 mL) was added TEA (2.57 g, 25.40 mmol, 3.00 eq.) and MsCl (1.16 g, 10.13 mmol, 1.20 eq.) at 0° C. The resulting mixture was stirred at RT overnight, diluted with water and then extracted with DCM. The organic layer was washed with water, brine, dried over Na₂SO₄, filtered, and concentrated to give the crude title compound as a yellow oil, which was used for next step without further purification.

### Step 6: tert-Butyl (1-((3-(piperidin-4-yloxy)phenyl)sulfonyl)piperidin-4-yl)carbamate

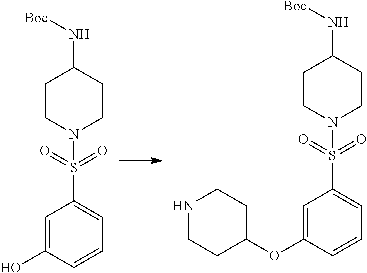 [SMILES: CC(C)(C)OC(=O)NC1CCN(S(=O)(=O)c2cccc(O)c2)CC1 ; CC(C)(C)OC(=O)NC1CCN(S(=O)(=O)c2cccc(OC3CCNCC3)c2)CC1]

Benzyl 4-((methylsulfonyl)oxy)piperidine-1-carboxylate was converted to the title compound by proceeding analogously as described in Reference 12, Steps 5-6 above.

### Step 7: tert-Butyl (1-((3-((1-(2-((2-(2,6-dioxopiperidin-3-yl)-1-oxoisoindolin-4-yl)oxy)ethyl)piperidin-4-yl)oxy)phenyl)sulfonyl)piperidin-4-yl)carbamate

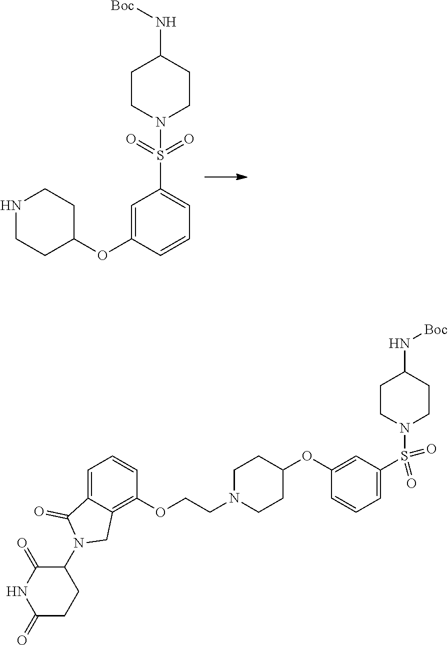 [SMILES: CC(C)(C)OC(=O)NC1CCN(S(=O)(=O)c2cccc(OC3CCNCC3)c2)CC1 ; CC(C)(C)OC(=O)NC1CCN(S(=O)(=O)c2cccc(OC3CCN(CCOc4cccc5c4CN(C4CCC(=O)NC4=O)C5=O)CC3)c2)CC1]

A mixture of 2-((2-(2,6-dioxopiperidin-3-yl)-1-oxoisoindolin-4-yl)oxy)ethyl 4-methylbenzenesulfonate (50 mg, 0.11 mmol, 1.10 eq.), tert-butyl (1-((3-(piperidin-4-yloxy)phenyl)-sulfonyl)piperidin-4-yl)carbamate (44 mg, 0.10 mmol, 1.00 eq.), KI (15 mg, 0.090 mmol, 0.90 eq.) and DIPEA (35 mg, 0.27 mmol, 2.70 eq.) in ACN (2.0 mL) was stirred at 100° C. under microwave for 3 h. The reaction mixture was cooled and concentrated, and the residue was purified by silica gel chromatography (DCM:MeOH=20:1) to give the title as a yellow oil.

### Reference 26

### Synthesis of tert-butyl (1-((3-((1-(2-((2-(2,6-dioxopiperidin-3-yl)-1-oxoisoindolin-4-yl)oxy)-ethyl)azetidin-3-yl)oxy)phenyl)sulfonyl)piperidin-4-yl)carbamate

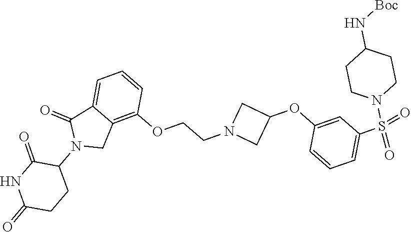 [SMILES: CC(C)(C)OC(=O)NC1CCN(S(=O)(=O)c2cccc(OC3CN(CCOc4cccc5c4CN(C4CCC(=O)NC4=O)C5=O)C3)c2)CC1]

tert-Butyl (1-((3-(azetidin-3-yloxy)phenyl)sulfonyl)piperidin-4-yl)carbamate was converted to the title compound by proceeding analogously as described in Reference 25, Step 7.

### Reference 27

### Synthesis of tert-butyl (1-((3-((1-(2-(2,6-dioxopiperidin-3-yl)-1-oxoisoindolin-5-yl)piperidin-4-yl)oxy)phenyl)sulfonyl)piperidin-4-yl)carbamate

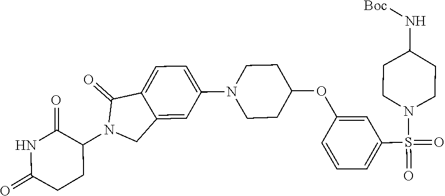 [SMILES: CC(C)(C)OC(=O)NC1CCN(S(=O)(=O)c2cccc(OC3CCN(c4ccc5c(c4)CN(C4CCC(=O)NC4=O)C5=O)CC3)c2)CC1]

A mixture of tert-butyl (1-((3-(piperidin-4-yloxy)phenyl)sulfonyl)piperidin-4-yl) carbamate (300 mg, 0.93 mmol, 1.00 eq.), 3-(5-bromo-1-oxoisoindolin-2-yl)piperidine-2,6-dione (448 mg, 1.02 mmol, 1.10 eq.), Cs₂CO₃ (603 mg, 1.86 mmol, 2.00 eq.), Pd(OAc)₂ (41 mg, 0.19 mmol, 0.20 eq.) and X-Phos (176 mg, 0.37 mmol, 0.40 eq.) in 1,4-dioxane (10.0 mL) was stirred at 105° C. under N₂ for 2 days. The reaction mixture was diluted with water and extracted with DCM. The combined organic layer was washed with brine and dried over Na₂SO₄ and concentrated. The residue was purified by flash chromatography gave title compound as a yellow solid.

### Reference 28

### Synthesis of tert-butyl (1-((3-(1-((1-(2-(2,6-dioxopiperidin-3-yl)-1-oxoisoindolin-5-yl)piperidin-4-yl)methyl)piperidin-4-yl)phenyl)sulfonyl)piperidin-4-yl)carbamate

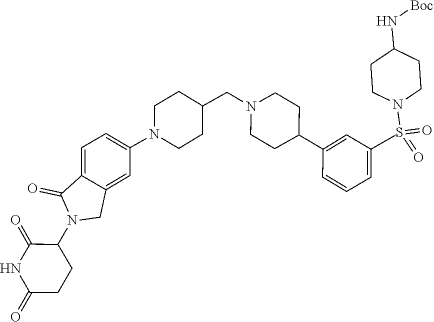

### Step 1: Benzyl 4-(3-((4-((tert-butoxycarbonyl)amino)piperidin-1-yl)sulfonyl)phenyl)-5,6-dihydropyridine-1(2H)-carboxylate

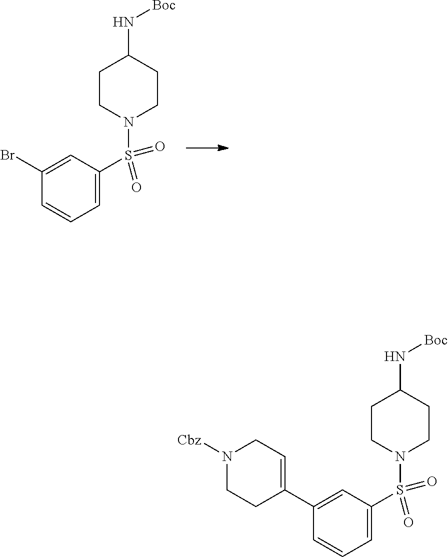 [SMILES: CC(C)(C)OC(=O)NC1CCN(S(=O)(=O)c2cccc(Br)c2)CC1 ; CC(C)(C)OC(=O)NC1CCN(S(=O)(=O)c2cccc(C3=CCN(C(=O)OCc4ccccc4)CC3)c2)CC1]

A mixture of tert-butyl (1-((3-bromophenyl)sulfonyl)piperidin-4-yl)carbamate (3.00 g, 7.18 mmol, 1.00 eq.), benzyl 4-(4,4,5,5-tetramethyl-1,3,2-dioxaborolan-2-yl)-5,6-dihydropyridine-1(2H)-carboxylate (3.20 g, 9.33 mmol, 1.30 eq.), X-Phos-Pd-G3 (608.0 mg, 0.72 mmol, 0.10 eq.) and K₃PO₄ (4.57 g, 21.54 mmol, 3.00 eq.) in 1,4-dioxane (70.0 mL) and H₂O (7.0 mL) was stirred at 60° C. under N₂ for 6 h. The resulting mixture was concentrated and the residue was purified by silica gel column chromatography, eluted with PE/EtOAc (4:1), to afford the title compound as a yellow solid.

### Step 2: tert-Butyl (1-((3-(piperidin-4-yl)phenyl)sulfonyl)piperidin-4-yl)carbamate

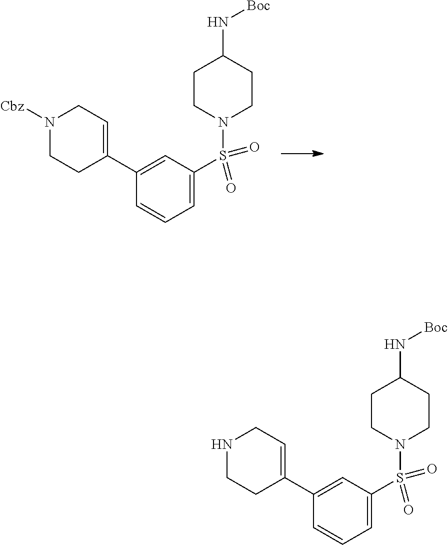 [SMILES: CC(C)(C)OC(=O)NC1CCN(S(=O)(=O)c2cccc(C3=CCN(C(=O)OCc4ccccc4)CC3)c2)CC1 ; CC(C)(C)OC(=O)NC1CCN(S(=O)(=O)c2cccc(C3=CCNCC3)c2)CC1]

A mixture of benzyl 4-(3-((4-((tert-butoxycarbonyl)amino)piperidin-1-yl)sulfonyl)phenyl)-5,6-dihydropyridine-1(2H)-carboxylate (4.00 g, 7.20 mmol, 1.00 eq.) and Pd/C (800 mg) in MeOH (40.0 mL) was stirred at 50° C. under H₂ (50 psi) for 16 h. The mixture was filtered and concentrated to afford the title compound as a white solid.

### Step 3: Benzyl 4-((4-(3-((4-((tert-butoxycarbonyl)amino)piperidin-1-yl)sulfonyl)phenyl)piperidin-1-yl)methyl)piperidine-1-carboxylate

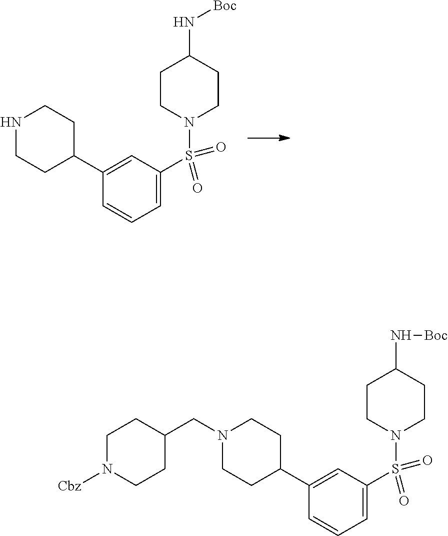 [SMILES: CC(C)(C)OC(=O)NC1CCN(S(=O)(=O)c2cccc(C3CCNCC3)c2)CC1 ; CC(C)(C)OC(=O)NC1CCN(S(=O)(=O)c2cccc(C3CCN(CC4CCN(C(=O)OCc5ccccc5)CC4)CC3)c2)CC1]

To a solution of tert-butyl (1-((3-(piperidin-4-yl)phenyl)sulfonyl)piperidin-4-yl)carbamate (3.00 g, 7.10 mmol, 1.00 eq.) in DCE (20.0 mL) and MeOH (20.0 mL) was added benzyl 4-formylpiperidine-1-carboxylate (2.63 g, 10.65 mmol, 1.50 eq.) and AcOH (426.0 mg, 7.10 mmol, 1.00 eq.) and the solution was stirred at RT for 1 h. NaBH₃CN (1.34 g, 21.30 mmol, 3.00 eq.) was added and the mixture was stirred at RT for 3 h. The resulting mixture was concentrated and the residue was purified by silica gel column chromatography, eluted with DCM/MeOH (60:1), to afford the title compound as a white solid.

### Step 4: tert-Butyl (1-((3-(1-(piperidin-4-ylmethyl)piperidin-4-yl)phenyl)sulfonyl)piperidin-4-yl)-carbamate

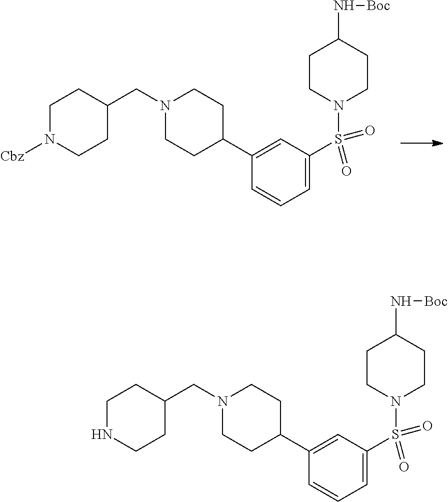 [SMILES: CC(C)(C)OC(=O)NC1CCN(S(=O)(=O)c2cccc(C3CCN(CC4CCN(C(=O)OCc5ccccc5)CC4)CC3)c2)CC1 ; CC(C)(C)OC(=O)NC1CCN(S(=O)(=O)c2cccc(C3CCN(CC4CCNCC4)CC3)c2)CC1]

A mixture of benzyl 4-((4-(3-((4-((tert-butoxycarbonyl)amino)piperidin-1-yl)-sulfonyl)phenyl)piperidin-1-yl)methyl)piperidine-1-carboxylate (3.80 g, 5.81 mmol, 1.00 eq.) and Pd/C (800 mg) in MeOH (40.0 mL) was stirred at 50° C. under H₂ (50 psi) for 16 h. The mixture was filtered and concentrated to afford the title compound as a white solid.

### Step 5: Methyl 4-(4-((4-(3-((4-((tert-butoxycarbonyl)amino)piperidin-1-yl)sulfonyl)-phenyl)piperidin-1-yl)methyl)piperidin-1-yl)-2-cyanobenzoate

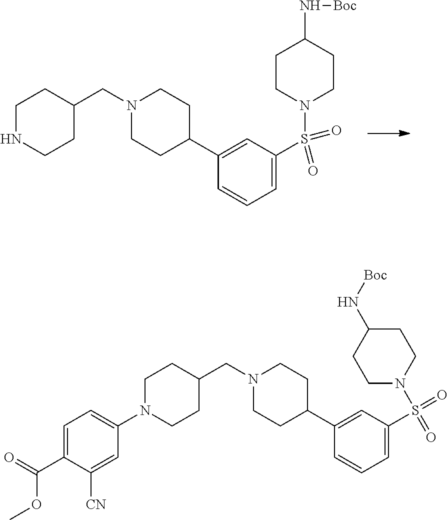 [SMILES: CC(C)(C)OC(=O)NC1CCN(S(=O)(=O)c2cccc(C3CCN(CC4CCNCC4)CC3)c2)CC1 ; COC(=O)c1ccc(N2CCC(CN3CCC(c4cccc(S(=O)(=O)N5CCC(NC(=O)OC(C)(C)C)CC5)c4)CC3)CC2)cc1C#N]

A solution of tert-butyl (1-((3-(1-(piperidin-4-ylmethyl)piperidin-4-yl)phenyl)-sulfonyl)piperidin-4-yl)carbamate (2.80 g, 5.40 mmol, 1.00 eq.), methyl 2-cyano-4-fluorobenzoate (1.06 g, 5.94 mmol, 1.10 eq.) and DIEA (2.09 g, 16.20 mmol, 3.00 eq.) in DMSO (30.0 mL) was stirred at 120° C. under N₂ for 16 h. The mixture was cooled to RT, diluted with water, and then extracted with EtOAc. The combined organic layer was washed with water, dried over anhydrous Na₂SO₄, filtered, and then concentrated. The residue was purified by silica gel column chromatography, eluted with DCM/MeOH (100:1), to afford the title compound as a brown solid.

### Step 6: Methyl 4-(4-((4-(3-((4-((tert-butoxycarbonyl)amino)piperidin-1-yl)sulfonyl)-phenyl)piperidin-1-yl)methyl)piperidin-1-yl)-2-formylbenzoate

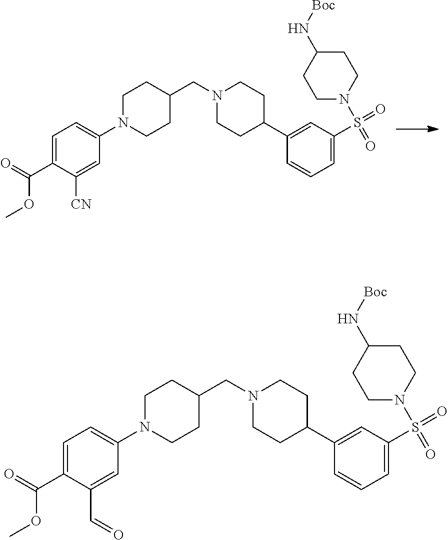 [SMILES: COC(=O)c1ccc(N2CCC(CN3CCC(c4cccc(S(=O)(=O)N5CCC(NC(=O)OC(C)(C)C)CC5)c4)CC3)CC2)cc1C#N ; COC(=O)c1ccc(N2CCC(CN3CCC(c4cccc(S(=O)(=O)N5CCC(NC(=O)OC(C)(C)C)CC5)c4)CC3)CC2)cc1C=O]

A mixture of methyl 4-(4-((4-(3-((4-((tert-butoxycarbonyl)amino)piperidin-1-yl)-sulfonyl)phenyl)piperidin-1-yl)methyl)piperidin-1-yl)-2-cyanobenzoate (1.01 g, 1.50 mmol, 1.00 eq.), NaH₂PO₂·H₂O (1.59 g, 15.00 mmol, 10.00 eq.) and Raney Ni (1.60 g) in pyridine (10.0 mL), H₂O (5.0 mL) and AcOH (5.0 mL) was stirred for 16 h at 70° C. under nitrogen atmosphere. The resulting mixture was diluted with EtOAc and washed with water, brine, dried over anhydrous Na₂SO₄ and concentrated. The residue was purified by silica gel column chromatography, eluted with DCM/MeOH (80:1), to afford the title compound as a light-yellow solid.

### Step 7: tert-Butyl (1-((3-(1-((1-(2-(2,6-dioxopiperidin-3-yl)-1-oxoisoindolin-5-yl)piperidin-4-yl)methyl)piperidin-4-yl)phenyl)sulfonyl)piperidin-4-yl)carbamate

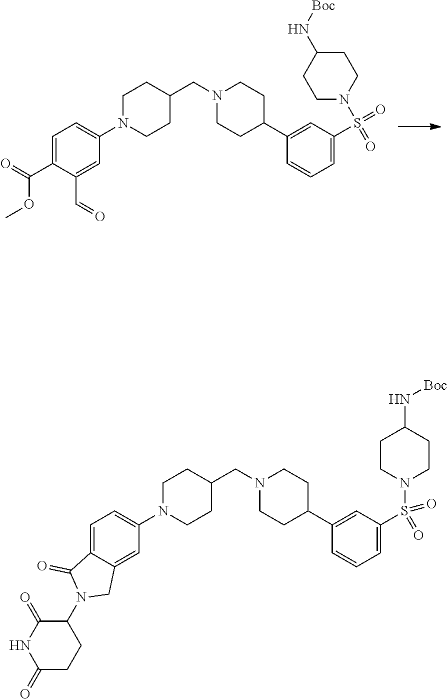 [SMILES: COC(=O)c1ccc(N2CCC(CN3CCC(c4cccc(S(=O)(=O)N5CCC(NC(=O)OC(C)(C)C)CC5)c4)CC3)CC2)cc1C=O ; CC(C)(C)OC(=O)NC1CCN(S(=O)(=O)c2cccc(C3CCN(CC4CCN(c5ccc6c(c5)CN(C5CCC(=O)NC5=O)C6=O)CC4)CC3)c2)CC1]

A mixture of 3-aminopiperidine-2,6-dione hydrochloride (126 mg, 0.77 mmol, 1.30 eq.) and DIEA (184 mg, 1.43 mmol, 2.40 eq.) in dry DCM (5.0 mL) was stirred at RT for 10 min and then a solution of methyl 4-(4-((4-(3-((4-((tert-butoxycarbonyl)amino)piperidin-1-yl)sulfonyl)-phenyl)piperidin-1-yl)methyl)piperidin-1-yl)-2-formylbenzoate (400 mg, 0.59 mmol, 1.00 eq.) in dry DCM (5.0 mL) and AcOH (134 mg, 2.23 mmol, 3.80 eq.) was added. The mixture was stirred at 45° C. under N₂ for 3 h. The mixture was cooled to 0° C. and NaBH(OAc)₃ (375 mg, 1.77 mmol, 3.00 eq.) was added. The mixture was stirred at rt for 1 h and then at 45° C. under N₂ for 16 h. The mixture was cooled, diluted with water, and then extracted with DCM. The combined organic layer was washed with water, dried over anhydrous Na₂SO₄, filtered, and concentrated. The residue was purified by silica gel column chromatography, eluted with DCM/MeOH (40:1), to afford the title compound as a yellow solid.

### Reference 29

### Synthesis of tert-butyl (1-((3-(4-((4-(2-(2,6-dioxopiperidin-3-yl)-1-oxoisoindolin-5-yl)piperidin-1-yl)methyl)piperidin-1-yl)phenyl)sulfonyl)piperidin-4-yl)carbamate

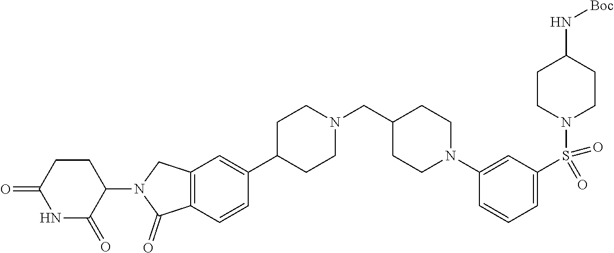 [SMILES: CC(C)(C)OC(=O)NC1CCN(S(=O)(=O)c2cccc(N3CCC(CN4CCC(c5ccc6c(c5)CN(C5CCC(=O)NC5=O)C6=O)CC4)CC3)c2)CC1]

### Step 1: 3-5-Bromo-1-oxoisoindolin-2-ylpiperidine-2,6-dione

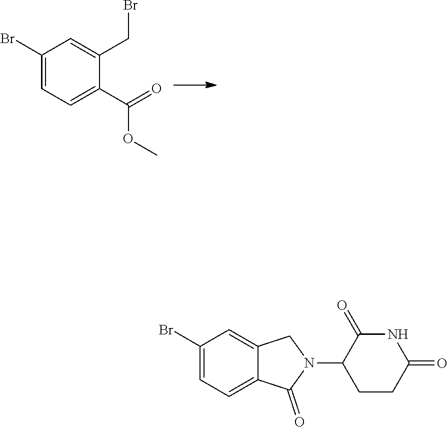 [SMILES: COC(=O)c1ccc(Br)cc1CBr ; O=C1CCC(N2Cc3cc(Br)ccc3C2=O)C(=O)N1]

A mixture of methyl 4-bromo-2-(bromomethyl)benzoate (20.00 g, 64.91 mmol, 1.00 eq.) and 3-aminopiperidine-2,6-dione (11.71 g, 71.41 mmol, 1.10 eq.), K₂CO₃ (26.87 g, 194.71 mmol, 3.00 eq.) in DMF was stirred at 70° C. overnight under N₂ atmosphere. The mixture was poured into water after the reaction was complete and extracted with DCM. The combined organic layer was washed with water, dried over anhydrous Na₂SO₄, filtered, and concentrated. The residue was purified by flash column (PE:EA=2:1) to give the title compound as a white solid.

### Step 2: tert-Butyl 4-(2-(2,6-dioxopiperidin-3-yl)-1-oxoisoindolin-5-yl)-5,6-dihydropyridine-1(2H)-carboxylate

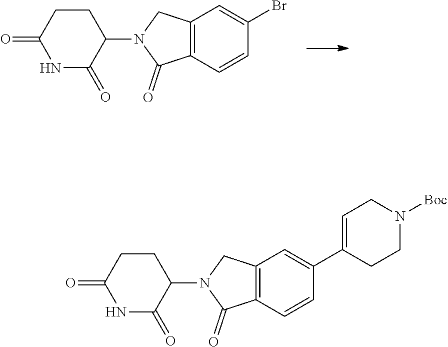 [SMILES: O=C1CCC(N2Cc3cc(Br)ccc3C2=O)C(=O)N1 ; CC(C)(C)OC(=O)N1CC=C(c2ccc3c(c2)CN(C2CCC(=O)NC2=O)C3=O)CC1]

A mixture of 3-(5-bromo-1-oxoisoindolin-2-yl)piperidine-2,6-dione (1.00 g, 3.11 mmol, 1.00 eq.), tert-butyl 4-(4,4,5,5-tetramethyl-1,3,2-dioxaborolan-2-yl)-5,6-dihydropyridine-1(2H)-carboxylate (1.25 g, 4.04 mmol, 1.30 eq.), K₃PO₄ (800 mg, 3.73 mmol, 1.20 eq) and Pd(dppf)Cl₂ (114 mg, 0.16 mmol, 0.05 eq) in DMF (10.0 mL) was stirred at 90° C. for 12 h. The mixture was concentrated and purified by silica gel column chromatography eluting with PE/EA (1:2) to give title compound as yellow solid.

### Step 3: tert-Butyl 4-(2-(2,6-dioxopiperidin-3-yl)-1-oxoisoindolin-5-yl)piperidine-1-carboxylate

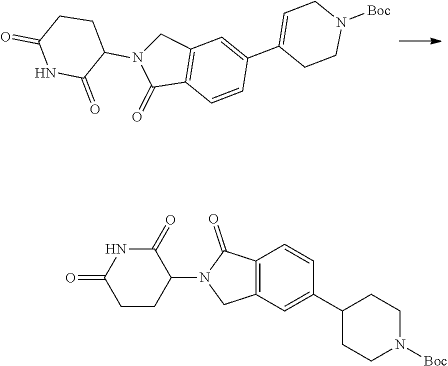 [SMILES: CC(C)(C)OC(=O)N1CC=C(c2ccc3c(c2)CN(C2CCC(=O)NC2=O)C3=O)CC1 ; CC(C)(C)OC(=O)N1CCC(c2ccc3c(c2)CN(C2CCC(=O)NC2=O)C3=O)CC1]

To a stirred solution of tert-butyl 4-(2(2,6-dioxopiperidin-3-yl)-1-oxoisoindolin-5-yl)-5,6-dihydropyridine-1(2H)-carboxylate (200 mg, 0.47 mmol, 1.00 eq.) in THF (2.0 mL) was added Pd/C (40 mg, 20% w/w). The resulting mixture was stirred at 40° C. for 12 h under H₂, filtered and concentrated to give the title compound as white solid.

### Step 4: 3-(1-Oxo-5-(piperidin-4-yl)isoindolin-2-yl)piperidine-2,6-dione

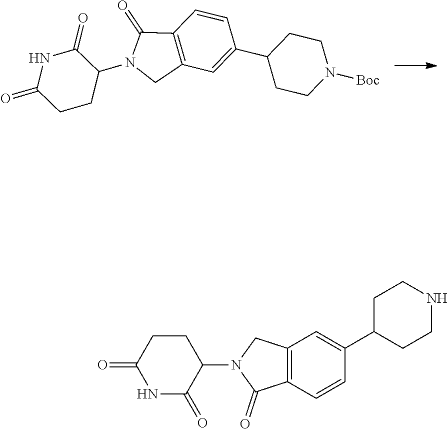 [SMILES: CC(C)(C)OC(=O)N1CCC(c2ccc3c(c2)CN(C2CCC(=O)NC2=O)C3=O)CC1 ; O=C1CCC(N2Cc3cc(C4CCNCC4)ccc3C2=O)C(=O)N1]

DCM/TFA=4:1 (2.5 mL) was added to a stirred solution of tert-butyl 4-(2-(2,6-dioxopiperidin-3-yl)-1-oxoisoindolin-5-yl)piperidine-1-carboxylate (100 mg, 0.234 mmol, 1.00 eq.) and the mixture was stirred at RT for 2 h. The mixture was concentrated to give the title compound as brown solid.

### Step 5: tert-Butyl (1-((3-(4-((4-(2-(2,6-dioxopiperidin-3-yl)-1-oxoisoindolin-5-yl)piperidin-1-yl)methyl)piperidin-1-yl)phenyl)sulfonyl)piperidin-4-yl)carbamate

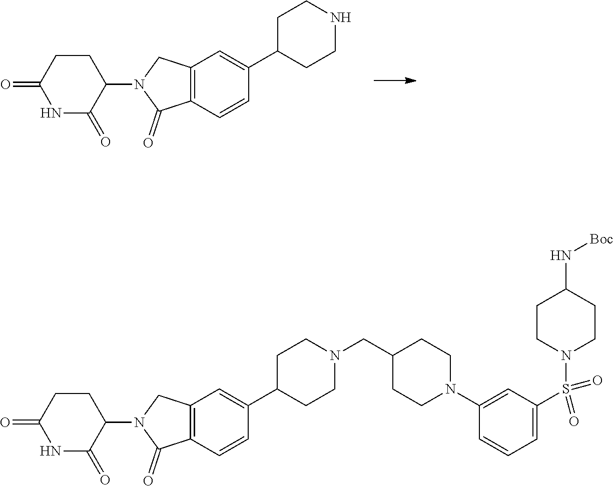 [SMILES: O=C1CCC(N2Cc3cc(C4CCNCC4)ccc3C2=O)C(=O)N1 ; CC(C)(C)OC(=O)NC1CCN(S(=O)(=O)c2cccc(N3CCC(CN4CCC(c5ccc6c(c5)CN(C5CCC(=O)NC5=O)C6=O)CC4)CC3)c2)CC1]

To a stirred solution of 3-(1-oxo-5-(piperidin-4-yl)isoindolin-2-yl)piperidine-2,6-dione (76.60 mg, 0.23 mmol, 1.00 eq) in THF (1.0 mL) was added DMF (1.0 mL), HCOOH (1 drop) and tert-butyl (1-((3-(4-formylpiperidin-1-yl)phenyl)sulfonyl)piperidin-4-yl)carbamate (105.60 mg, 0.23 mmol 1.00 eq). The resulting mixture was stirred at 45° C. for 0.5 h. NaBH₃CN (29.40 mg, 0.47 mmol, 2.00 eq) was added at RT and the reaction mixture was stirred at RT for 12 h. The mixture was diluted with water and extracted with EtOAc. The combined organic layer was washed with water and brine and concentrated. The residue was purified by silica gel column chromatography, eluted with DCM:MeOH (0-100%), to give the title compound as a white solid.

### Reference 30

### Synthesis of tert-butyl (1-((3-(4-((4-(2-(2,6-dioxopiperidin-3-yl)-1-oxoisoindolin-5-yl)piperazin-1-yl)-methyl)piperidin-1-yl)phenyl)sulfonyl)piperidin-4-yl)carbamate

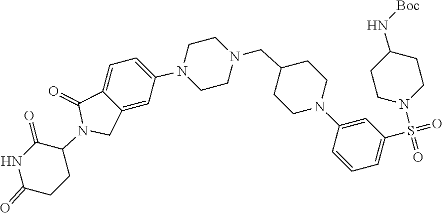 [SMILES: CC(C)(C)OC(=O)NC1CCN(S(=O)(=O)c2cccc(N3CCC(CN4CCN(c5ccc6c(c5)CN(C5CCC(=O)NC5=O)C6=O)CC4)CC3)c2)CC1]

### Step 1: Benzyl 4-(dimethoxymethyl)piperidine-1-carboxylate

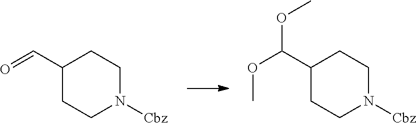 [SMILES: O=CC1CCN(C(=O)OCc2ccccc2)CC1 ; COC(OC)C1CCN(C(=O)OCc2ccccc2)CC1]

To a mixture of benzyl 4-formylpiperidine-1-carboxylate (1.00 g, 2.40 mmol, 1.00 eq.) in MeOH (9.0 mL) was added p-TsOH (38 mg, 0.20 mmol, 0.05 eq.) and trimethoxymethane (2.14 g, 20.22 mmol, 5.00 eq.). The mixture was stirred at RT for 12 h and then extracted with EtOAc. Purification of the crude product by silica gel column chromatography eluting with PE/EtOAc (10:1) gave the title compound as a colorless oil.

### Step 2: 4-(Dimethoxymethyl)piperidine

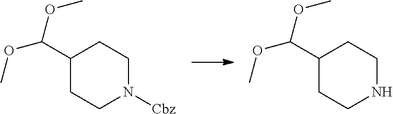 [SMILES: COC(OC)C1CCN(C(=O)OCc2ccccc2)CC1 ; COC(OC)C1CCNCC1]

To a mixture of benzyl 4-(dimethoxymethyl) piperidine-1-carboxylate (948 mg, 3.23 mmol, 1.00 eq.) in MeOH (10.0 mL) was added Pd/C (400 mg) and the reaction mixture was stirred at RT under H₂ for overnight. The resulting mixture was filtered through Celite and the filtrate was concentrated to give the title compound as a colorless oil.

### Step 3: tert-Butyl (1-((3-(4-(dimethoxymethyl) piperidin-1-yl)phenyl)sulfonyl)piperidin-4-yl)-carbamate

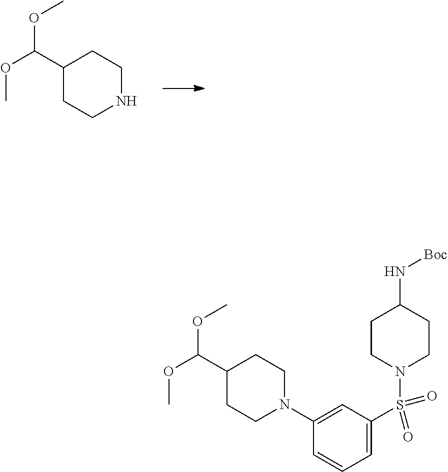 [SMILES: COC(OC)C1CCNCC1 ; COC(OC)C1CCN(c2cccc(S(=O)(=O)N3CCC(NC(=O)OC(C)(C)C)CC3)c2)CC1]

A mixture of 4-(dimethoxymethyl) piperidine (100 mg, 0.63 mmol, 1.20 eq.), K₂CO₃ (215 mg, 1.56 mmol, 3.00 eq.), CuI (20 mg, 0.104 mmol, 0.20 eq.), L-proline (18 mg, 0.16 mmol, 0.30 eq.) and tert-butyl (1-((3-bromophenyl)sulfonyl)piperidin-4-yl)carbamate (219 mg, 0.52 mmol, 1.00 eq.) in DMSO (4.0 mL) was stirred at 90° C. overnight. The reaction mixture was diluted with water and extracted with EtOAc. The combined organic layer was washed with water and brine and concentrated. The residue was purified by silica gel column chromatography eluting with PE/EtOAc (1:1) to give the title compound as white solid.

### Step 4: tert-Butyl (1-((3-(4-formylpiperidin-1-yl)phenyl)sulfonyl)piperidin-4-yl)carbamate

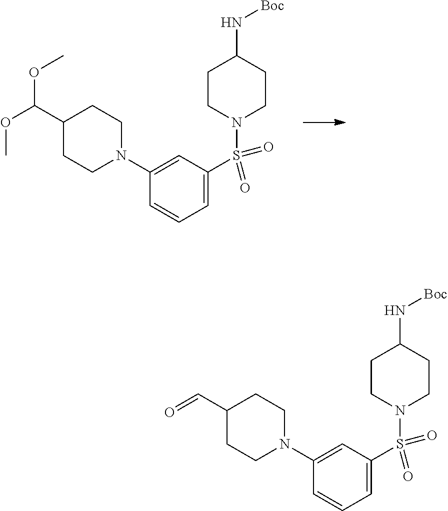 [SMILES: COC(OC)C1CCN(c2cccc(S(=O)(=O)N3CCC(NC(=O)OC(C)(C)C)CC3)c2)CC1 ; CC(C)(C)OC(=O)NC1CCN(S(=O)(=O)c2cccc(N3CCC(C=O)CC3)c2)CC1]

To a mixture of tert-butyl (1-((3-(4-(dimethoxymethyl)piperidin-1-yl)phenyl)sulfonyl) piperidin-4-yl)carbamate (640 mg, 1.29 mmol, 1.00 eq.) in DCM (4.0 mL) was added TFA (4.0 mL) and the mixture was stirred at 45° C. overnight. The reaction mixture was concentrated and dissolved in DCM (5.0 mL) followed by addition of TEA (261 mg, 2.58 mmol, 2.00 eq.) and (Boc)₂O (562 mg, 2.58 mmol, 2.00 eq.). The solution was stirred at RT for 4 h, and concentrated and the residue was purified by silica gel column chromatography eluting with PE/EtOAc (3:1) to give title compound as yellow solid.

### Step 5: tert-Butyl (1-((3-(4-((4-(2-(2,6-dioxopiperidin-3-yl)-1-oxoisoindolin-5-yl)piperazin-1-yl)-methyl)piperidin-1-yl)phenyl)sulfonyl)piperidin-4-yl)carbamate

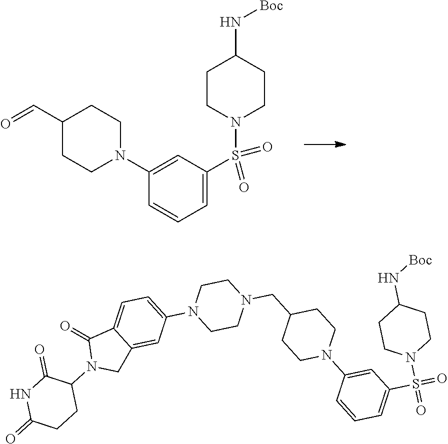 [SMILES: CC(C)(C)OC(=O)NC1CCN(S(=O)(=O)c2cccc(N3CCC(C=O)CC3)c2)CC1 ; CC(C)(C)OC(=O)NC1CCN(S(=O)(=O)c2cccc(N3CCC(CN4CCN(c5ccc6c(c5)CN(C5CCC(=O)NC5=O)C6=O)CC4)CC3)c2)CC1]

The compound was prepared analogously as described in Reference 29, Step 5.

### Reference 31

### Synthesis of 3-(5-(azetidin-3-yl)-1-oxoisoindolin-2-yl)piperidine-2,6-dione

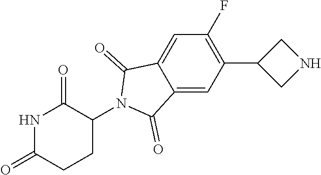 [SMILES: O=C1CCC(N2C(=O)c3cc(F)c(C4CNC4)cc3C2=O)C(=O)N1]

### Step 1: (1-(tert-Butoxycarbonyl)azetidin-3-yl)zinc(II) iodide

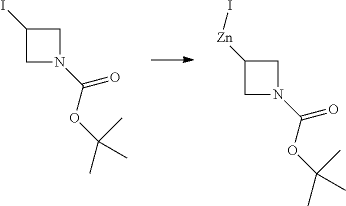

To a mixture of Zn dust (300 mg, 4.59 mmol, 1.30 eq.) in DMA (3.0 mL) was added 1,2-dibromoethene (66 mg, 0.35 mmol, 0.10 eq.) and the mixture was stirred at 65° C. under N₂ for 30 min. The mixture was allowed to cool to RT and TMSCl (38 mg, 0.35 mmol, 0.10 eq.) was added. After stirring the mixture for 30 min., a solution of tert-butyl 3-iodoazetidine-1-carboxylate (1.00 g, 3.53 mmol, 1.00 eq.) in DMA (1.0 mL) was added dropwise. The mixture was stirred at 65° C. under N₂ for 2 h, cooled to RT and used in next step without further purification.

### Step 2: tert-Butyl 3-(2-(2,6-dioxopiperidin-3-yl)-1-oxoisoindolin-5-yl)azetidine-1-carboxylate

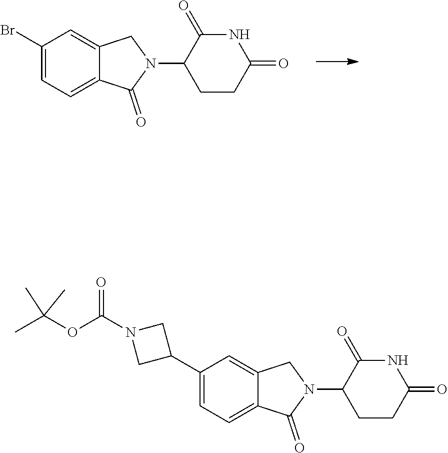 [SMILES: O=C1CCC(N2Cc3cc(Br)ccc3C2=O)C(=O)N1 ; CC(C)(C)OC(=O)N1CC(c2ccc3c(c2)CN(C2CCC(=O)NC2=O)C3=O)C1]

A solution of (1-(tert-butoxycarbonyl)azetidin-3-yl)zinc(II) iodide (600 mg, 1.72 mmol, 3.00 eq.) in DMA was slowly added to a mixture of 3-(5-bromo-1-oxoisoindolin-2-yl)piperidine-2,6-dione (185 mg, 0.57 mmol, 1.00 eq.), CuI (12 mg, 0.06 mmol, 0.10 eq.), Pd(dppf)Cl₂ (44 mg, 0.06 mmol, 0.10 eq.) in DMA (2.0 mL). The mixture was stirred at 90° C. under N₂ overnight. The mixture was concentrated and purified by column chromatography on silica gel (EA) to give the title compound as a brown solid.

### Step 3: 3-(5-(Azetidin-3-yl)-1-oxoisoindolin-2-yl)piperidine-2,6-dione

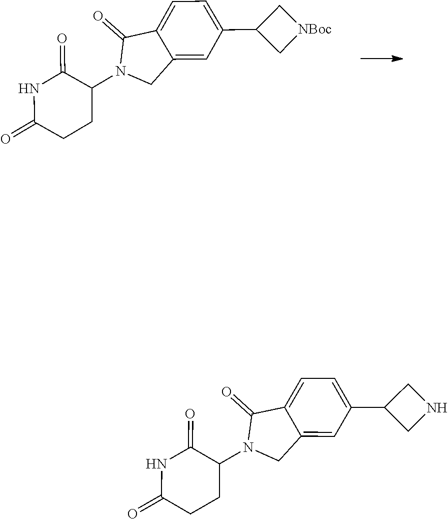 [SMILES: CC(C)(C)OC(=O)N1CC(c2ccc3c(c2)CN(C2CCC(=O)NC2=O)C3=O)C1 ; O=C1CCC(N2Cc3cc(C4CNC4)ccc3C2=O)C(=O)N1]

To a solution of tert-butyl 3-(2-(2,6-dioxopiperidin-3-yl)-1-oxoisoindolin-5-yl)azetidine-1-carboxylate (44 mg, 0.11 mmol, 1.00 eq.) in DCM (1.0 mL) was added TFA (0.2 mL) dropwise and the solution was stirred at RT for 3 h. The resulting mixture was concentrated to give the crude product as a brown oil.

### Reference 32

### Synthesis of tert-butyl (1-((3-(4-((1-(2-(2,6-dioxopiperidin-3-yl)-1-oxoisoindolin-5-yl)piperidin-4-yl)methyl)piperazin-1-yl)phenyl)sulfonyl)piperidin-4-yl)carbamate

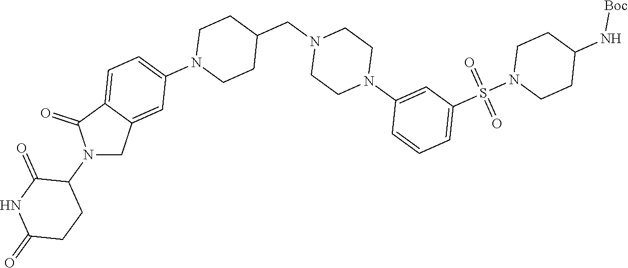 [SMILES: CC(C)(C)OC(=O)NC1CCN(S(=O)(=O)c2cccc(N3CCN(CC4CCN(c5ccc6c(c5)CN(C5CCC(=O)NC5=O)C6=O)CC4)CC3)c2)CC1]

### Step 1: Benzyl 4-(3-((4-((tert-butoxycarbonyl)amino)piperidin-1-yl)sulfonyl)phenyl)piperazine-1-carboxylate

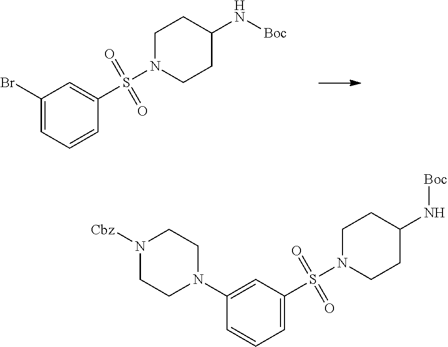 [SMILES: CC(C)(C)OC(=O)NC1CCN(S(=O)(=O)c2cccc(Br)c2)CC1 ; CC(C)(C)OC(=O)NC1CCN(S(=O)(=O)c2cccc(N3CCN(C(=O)OCc4ccccc4)CC3)c2)CC1]

A mixture of tert-butyl (1-((3-bromophenyl)sulfonyl)piperidin-4-yl)carbamate (5.00 g, 11.% mmol, 1.00 eq.), K₂COI (5.78 g, 41.86 mmol, 3.50 eq.), CuI (0.45 g, 2.39 mmol, 0.20 eq.), L-PRO (0.41 g, 3.59 mmol, 0.30 eq.) in DMSO (25.00 mL) and benzyl piperazine-1-carboxylate (3.43 g, 15.55 mmol, 1.30 eq.) was stirred at 100° C. for 12 h. The mixture was quenched with H₂O and extracted with EtOAc. The organic layer was concentrated and the residue was purified by silica gel column chromatography eluting with PE/EtOAc (3:1) to give the title compound as white solid.

### Step 2: tert-B (1-((3-(piperazin-1-yl)phenyl)sulfonyl)piperidin-4-yl)carbamate

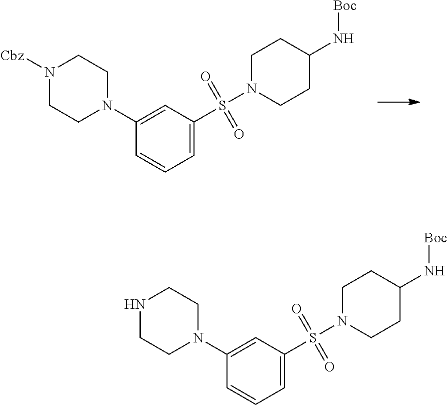 [SMILES: CC(C)(C)OC(=O)NC1CCN(S(=O)(=O)c2cccc(N3CCN(C(=O)OCc4ccccc4)CC3)c2)CC1 ; CC(C)(C)OC(=O)NC1CCN(S(=O)(=O)c2cccc(N3CCNCC3)c2)CC1]

The title compound was prepared analogously as described in Reference 30, Step 2.

### Step 3: Benzyl 4-((4-(3-((4-((tert-butoxycarbonyl)amino)piperidin-1-yl)sulfonyl)phenyl)piperazin-1-yl)methyl)piperidine-1-carboxylate

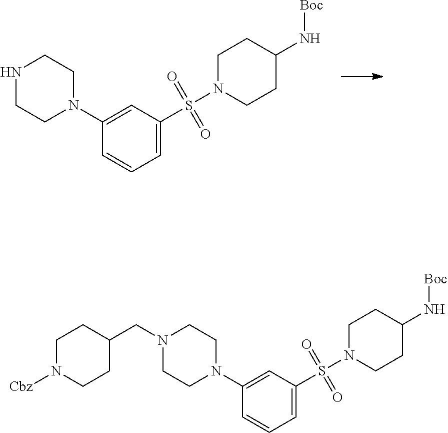 [SMILES: CC(C)(C)OC(=O)NC1CCN(S(=O)(=O)c2cccc(N3CCNCC3)c2)CC1 ; CC(C)(C)OC(=O)NC1CCN(S(=O)(=O)c2cccc(N3CCN(CC4CCN(C(=O)OCc5ccccc5)CC4)CC3)c2)CC1]

A mixture of tert-butyl (1-((3-(piperazin-1-yl)phenyl)sulfonyl)piperidin-4-yl)carbamate (1.07 g, 2.52 mmol, 1.00 eq.), AcOH (3 drops) and benzyl 4-formylpiperidine-1-carboxylate (933 mg, 3.78 mmol, 1.50 eq.) in MeOH (10.0 mL) was stirred at 45° C. for 1 h. The solution cooled to RT and NaBH₃CN (475 mg, 7.56 mmol, 3.00 eq.) was added. The mixture was stirred at RT for 12 h and then diluted with water and extracted with EtOAc. The combined organic layer was washed with brine, dried over anhydrous Na₂SO₄. and concentrated. The residue was purified by silica gel column chromatography eluting with DCM/MeOH (60:1) to give the title compound as white solid.

### Step 4: tert-Butyl (1-((3-(4-((1-(2-(2,6-dioxopiperidin-3-yl)-1-oxoisoindolin-5-yl)piperidin-4-yl)methyl)piperazin-1-yl)phenyl)sulfonyl)piperidin-4-yl)carbamate

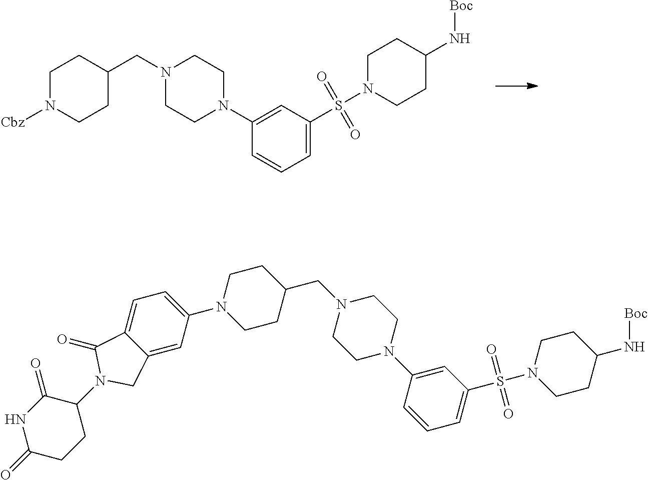 [SMILES: CC(C)(C)OC(=O)NC1CCN(S(=O)(=O)c2cccc(N3CCN(CC4CCN(C(=O)OCc5ccccc5)CC4)CC3)c2)CC1 ; CC(C)(C)OC(=O)NC1CCN(S(=O)(=O)c2cccc(N3CCN(CC4CCN(c5ccc6c(c5)CN(C5CCC(=O)NC5=O)C6=O)CC4)CC3)c2)CC1]

Benzyl 4-((4-(3-((4-((tert-butoxycarbonyl)amino)piperidin-1-yl)sulfonyl)phenyl)piperazin-1-yl)methyl)piperidine-1-carboxylate was converted to the title compound using similar procedure as described in Reference 28, Step 4-7.

### Reference 33

### Synthesis of tert-butyl (1-((3-(4-(1-(2,6-dioxopiperidin-3-yl)-3-methyl-2-oxo-2,3-dihydro-1H-benzo[d]imidazol-4-yl)-[1,4′-bipiperidin]-1′-yl)phenyl)sulfonyl)piperidin-4-yl)carbamate

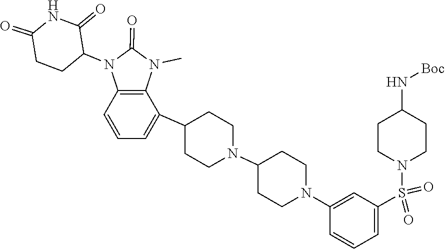 [SMILES: Cn1c(=O)n(C2CCC(=O)NC2=O)c2cccc(C3CCN(C4CCN(c5cccc(S(=O)(=O)N6CCC(NC(=O)OC(C)(C)C)CC6)c5)CC4)CC3)c21]

### Step 1: tert-Butyl (1-((3-(1,4-dioxa-8-azaspiro[4.5]decan-8-yl)phenyl)sulfonyl)piperidin-4-yl)-carbamate

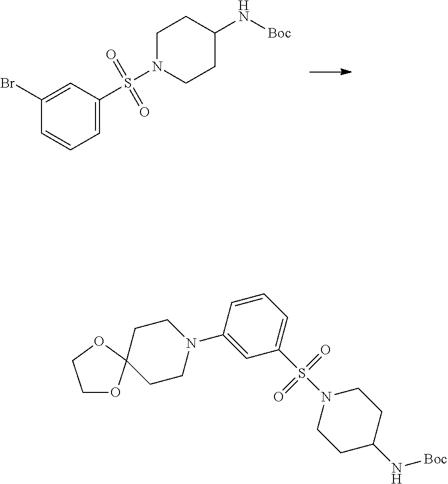 [SMILES: CC(C)(C)OC(=O)NC1CCN(S(=O)(=O)c2cccc(Br)c2)CC1 ; CC(C)(C)OC(=O)NC1CCN(S(=O)(=O)c2cccc(N3CCC4(CC3)OCCO4)c2)CC1]

A mixture of tert-butyl (1-((3-bromophenyl)sulfonyl)piperidin-4-yl)carbamate (1.00 g, 2.40 mmol, 1.00 eq.), K₂CO₃ (1.16 g, 8.40 mmol, 3.50 eq.), CuI (91 mg, 0.480 mmol, 0.20 eq.), L-proline (83 mg, 0.72 mmol, 0.30 eq.) and 1,4-dioxa-8-azaspiro[4.5]decane (412 mg, 2.88 mmol, 1.20 eq.) in DMSO (10.0 mL) was stirred at 90° C. overnight. The reaction mixture was diluted with water and extracted with DCM. The combined organic layer was washed with brine, dried over anhydrous Na₂SO₄, and concentrated. The residue was purified by silica gel column chromatography eluting with PE/EtOAc (1:1) to give the title compound as yellow solid.

### Step 2: tert-Butyl (1-((3-(4-oxopiperidin-1-yl)phenyl)sulfonyl)piperidin-4-yl)carbamate

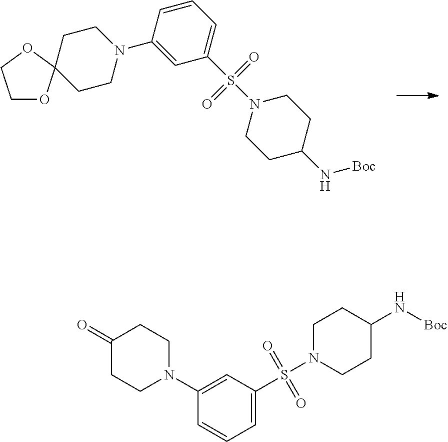 [SMILES: CC(C)(C)OC(=O)NC1CCN(S(=O)(=O)c2cccc(N3CCC4(CC3)OCCO4)c2)CC1 ; CC(C)(C)OC(=O)NC1CCN(S(=O)(=O)c2cccc(N3CCC(=O)CC3)c2)CC1]

A mixture of tert-butyl (1-((3-(1,4-dioxa-8-azaspiro[4.5]decan-8-yl)phenyl)-sulfonyl)piperidin-4-yl)carbamate (624 mg, 1.30 mmol, 1.00 eq.), TsOH·H₂O (49 mg, 0.26 mmol, 0.20 eq.) in acetone (6.0 mL) and H₂O (12.0 mL) was stirred at 60° C. overnight. The mixture was extracted with DCM. The combined organic layer was washed with brine, dried over anhydrous Na₂SO₄. and concentrated. The residue was purified by silica gel column chromatography eluting PE/EtOAc (1:1) to give the title compound as yellow solid.

### Step 3: tert-Butyl (1-((3-(4-(1-(2,6-dioxopiperidin-3-yl)-3-methyl-2-oxo-2,3-dihydro-1H-benzo[d]imidazol-4-yl)-[1,4′-bipiperidin]-1′-yl)phenyl)sulfonyl)piperidin-4-yl)carbamate

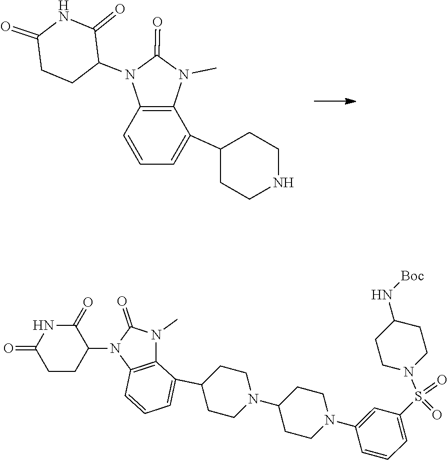 [SMILES: Cn1c(=O)n(C2CCC(=O)NC2=O)c2cccc(C3CCNCC3)c21 ; Cn1c(=O)n(C2CCC(=O)NC2=O)c2cccc(C3CCN(C4CCN(c5cccc(S(=O)(=O)N6CCC(NC(=O)OC(C)(C)C)CC6)c5)CC4)CC3)c21]

tert-Butyl (1-((3-(4-oxopiperidin-1-yl)phenyl)sulfonyl)piperidin-4-yl)carbamate (44 mg, 1.02 mmol, 0.90 eq.) and 1 drop of AcOH was added to a mixture of 3-(3-methyl-2-oxo-4-(piperidin-4-yl)-2,3-dihydro-1H-benzo[d]imidazol-1-yl)piperidine-2,6-dione (387 mg, 1.13 mmol, 1.00 eq.) in THF (5.0 mL). The reaction mixture was stirred at 40° C. for 0.5 h. NaBH₃CN (142 mg, 2.60 mmol, 2.00 eq.) was added at RT and stirred at RT overnight. The reaction mixture was extracted with DCM. The combined organic layer was washed with brine, dried over anhydrous Na₂SO₄, and concentrated. The residue was purified by silica gel column chromatography eluting with DCM/MeOH (10:1) to give the title compound as a yellow solid.

### Reference 34

### Synthesis of 3-(4-(azetidin-3-yl)-3-methyl-2-oxo-2,3-dihydro-1H-benzo[d]imidazol-1-yl)piperidine-2,6-dione

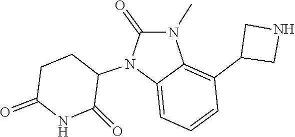 [SMILES: Cn1c(=O)n(C2CCC(=O)NC2=O)c2cccc(C3CNC3)c21]

### Step 1: tert-Butyl 3-(1-(2,6-dioxopiperidin-3-yl)-3-methyl-2-oxo-2,3-dihydro-1H-benzo[d]-imidazol-4-yl)azetidine-1-carboxylate

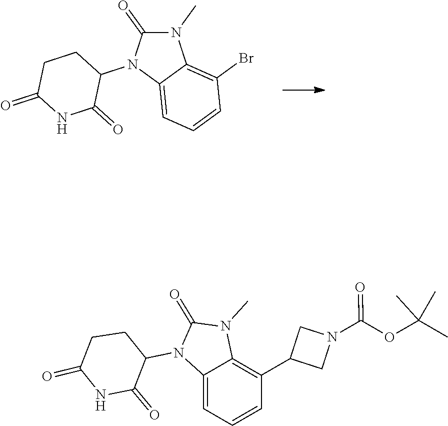 [SMILES: Cn1c(=O)n(C2CCC(=O)NC2=O)c2cccc(Br)c21 ; Cn1c(=O)n(C2CCC(=O)NC2=O)c2cccc(C3CN(C(=O)OC(C)(C)C)C3)c21]

A solution of (1-(tert-butoxycarbonyl)azetidin-3-yl)zinc(II) iodide (600 mg, 1.72 mmol, 3.00 eq.) in DMA was slowly added to a mixture of 3-(4-bromo-3-methyl-2-oxo-2,3-dihydro-1H-benzo[d]imidazol-1-yl)piperidine-2,6-dione (193 mg, 0.57 mmol, 1.00 eq.) in DMA (2.0 mL) CuI (12 mg, 0.06 mmol, 0.10 eq.) and Pd(dppf)C12 (44 mg, 0.06 mmol, 0.10 eq.). The mixture was stirred at 90° C. under N₂ overnight. The mixture was concentrated and purified by column chromatography on silica gel (EA) to the title compound as a yellow solid.

### Step 2: 3-(4-(Azetidin-3-yl)-3-methyl-2-oxo-2,3-dihydro-1H-benzo[d]imidazol-1-yl)piperidine-2,6-dione

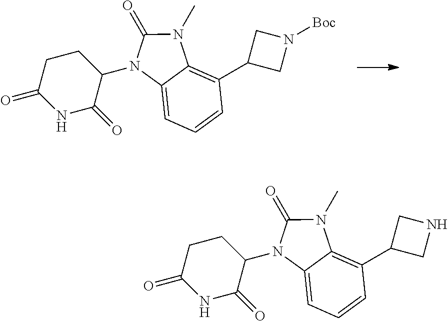 [SMILES: Cn1c(=O)n(C2CCC(=O)NC2=O)c2cccc(C3CN(C(=O)OC(C)(C)C)C3)c21 ; Cn1c(=O)n(C2CCC(=O)NC2=O)c2cccc(C3CNC3)c21]

To a solution of tert-butyl 3-(1-(2,6-dioxopiperidin-3-yl)-3-methyl-2-oxo-2,3-dihydro-1H-benzo[d]imidazol-4-yl)azetidine-1-carboxylate (23 mg, 0.055 mmol, 1.00 eq.) in DCM (1.0 mL) was added TFA (0.2 mL) dropwise and the solution was stirred at RT for 3 h. The resulting mixture was concentrated to give the crude product as a brown oil, which was used in next step without further purification.

### Reference 35

### Synthesis of tert-butyl (1-((3-((4-(2-(2,6-dioxopiperidin-3-yl)-6-fluoro-1-oxoisoindolin-5-yl)piperazin-1-yl)methyl)phenyl)sulfonyl)piperidin-4-yl)carbamate

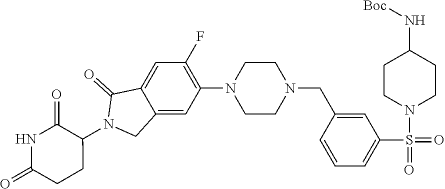 [SMILES: CC(C)(C)OC(=O)NC1CCN(S(=O)(=O)c2cccc(CN3CCN(c4cc5c(cc4F)C(=O)N(C4CCC(=O)NC4=O)C5)CC3)c2)CC1]

### Step 1: tert-Butyl 4-(5-bromo-2-fluoro-4-(methoxycarbonyl)phenyl)piperazine-1-carboxylate

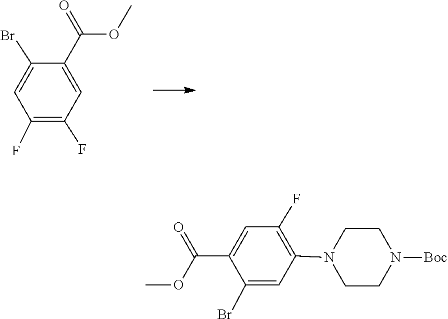 [SMILES: COC(=O)c1cc(F)c(F)cc1Br ; COC(=O)c1cc(F)c(N2CCN(C(=O)OC(C)(C)C)CC2)cc1Br]

A mixture of methyl 2-bromo-4,5-difluorobenzoate (2.00 g, 8.00 mmol, 1.00 eq.) and tert-butyl piperazine-1-carboxylate (2.23 g, 12.00 mmol, 1.50 eq.), K₂CO₃ (1.65 g, 12.00 mmol, 1.50 eq.) in DMA (6.0 mL) was stirred at 80° C. overnight. The mixture was diluted with water and extracted EA. The combined organic layer was washed with brine, dried over Na₂SO₄, concentrated. The residue was purified by flash chromatography (PE:EA=3:1) to give the title compound as a colorless oil.

### Step 2: tert-Butyl 4-(5-cyano-2-fluoro-4-(methoxycarbonyl)phenyl)piperazine-1-carboxylate

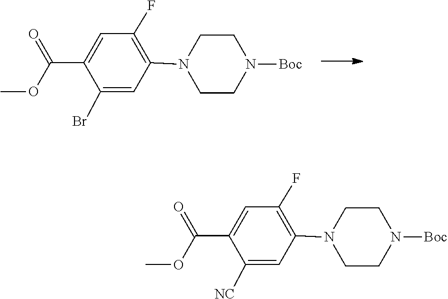 [SMILES: COC(=O)c1cc(F)c(N2CCN(C(=O)OC(C)(C)C)CC2)cc1Br ; COC(=O)c1cc(F)c(N2CCN(C(=O)OC(C)(C)C)CC2)cc1C#N]

A mixture of tert-butyl 4-(5-bromo-2-fluoro-4-(methoxycarbonyl)phenyl)-piperazine-1-carboxylate (1.50 g, 3.60 mmol, 1.00 eq.) and CuCN (484 mg, 5.40 mmol, 1.50 eq.) in DMF (6.0 mL was stirred at 100° C. overnight. The mixture was extracted with EA and washed with NH₃·H₂O. The organic layer was washed with water and brine, dried over Na₂SO₄, concentrated and purified by flash chromatography (PE:EA=3:1) to give the title compound as a white solid.

### Step 3: tert-Butyl 4-(2-(2,6-dioxopiperidin-3-yl)-6-fluoro-1-oxoisoindolin-5-yl)piperazine-1-carboxylate

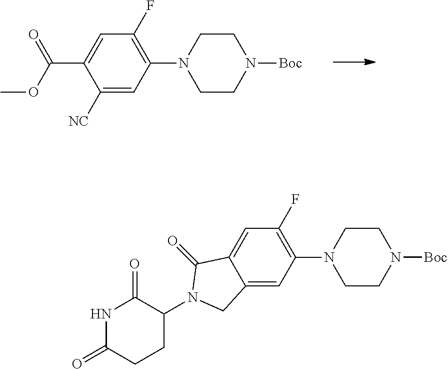 [SMILES: COC(=O)c1cc(F)c(N2CCN(C(=O)OC(C)(C)C)CC2)cc1C#N ; CC(C)(C)OC(=O)N1CCN(c2cc3c(cc2F)C(=O)N(C2CCC(=O)NC2=O)C3)CC1]

tert-Butyl 4-(5-cyano-2-fluoro-4-(methoxycarbonyl)phenyl)piperazine-1-carboxylate was converted to the title compound by proceeding analogously as described in Reference 28, Step 6-7.

### Step 4: 3-(6-Fluoro-1-oxo-5-(piperazin-1-yl)isoindolin-2-yl)piperidine-2,6-dione

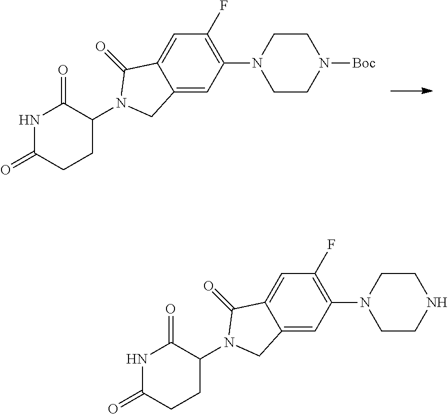 [SMILES: CC(C)(C)OC(=O)N1CCN(c2cc3c(cc2F)C(=O)N(C2CCC(=O)NC2=O)C3)CC1 ; O=C1CCC(N2Cc3cc(N4CCNCC4)c(F)cc3C2=O)C(=O)N1]

To a stirred solution of tert-butyl 4-(2-(2,6-dioxopiperidin-3-yl)-6-fluoro-1-oxoisoindolin-5-yl)piperazine-1-carboxylate (95 mg, 0.21 mmol, 1.00 eq.) in DCM (2.0 mL) was added TFA (0.5 mL) and the mixture was stirred at RT for 2 h. The reaction mixture was concentrated to give the title compound as a yellow oil.

### Step 5: tert-Butyl (1-((3-((4-(2-(2,6-dioxopiperidin-3-yl)-6-fluoro-1-oxoisoindolin-5-yl)piperazin-1-yl)methyl)sulfonyl)piperidin-4-yl)carbamate

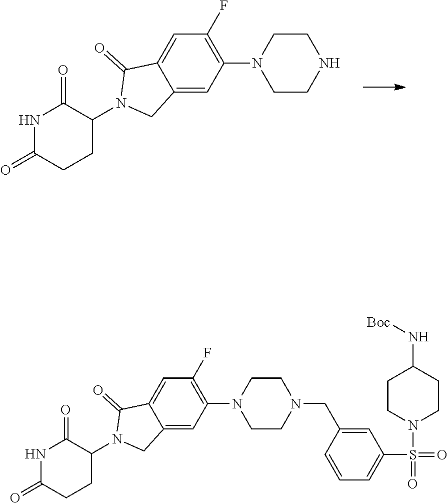 [SMILES: O=C1CCC(N2Cc3cc(N4CCNCC4)c(F)cc3C2=O)C(=O)N1 ; CC(C)(C)OC(=O)NC1CCN(S(=O)(=O)c2cccc(CN3CCN(c4cc5c(cc4F)C(=O)N(C4CCC(=O)NC4=O)C5)CC3)c2)CC1]

A mixture of 3-(6-fluoro-1-oxo-5-(piperazin-1-yl)isoindolin-2-yl)piperidine-2,6-dione (74 mg, 0.33 mmol, 1.00 eq.), tert-butyl (1-((3-(bromomethyl)phenyl)sulfonyl)piperidin-4-yl)carbamate (138 mg, 0.32 mmol, 1.50 eq.), TEA (127 mg, 1.26 mmol, 6.00 eq.) in THF (5.0 mL) was stirred at 55° C. overnight. The mixture was diluted with water and extracted with DCM. The organic layer was washed with brine, dried over Na₂SO₄, concentrated, and the residue was purified by flash chromatography (DCM:MeOH=20:1) to give the tide compound as a yellow solid.

### Reference 36

### Synthesis of tert-butyl (1-((3-((4-(2-(2,6-dioxopiperidin-3-yl)-6-fluoro-1,3-dioxoisoindolin-5-yl)piperazin-1-yl)methyl)phenyl)sulfonyl)piperidin-4-yl)carbamate

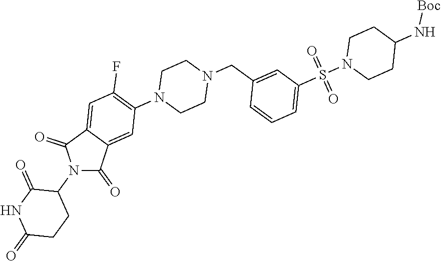 [SMILES: CC(C)(C)OC(=O)NC1CCN(S(=O)(=O)c2cccc(CN3CCN(c4cc5c(cc4F)C(=O)N(C4CCC(=O)NC4=O)C5=O)CC3)c2)CC1]

### Step 1: tert-Butyl 4-(2-(2,6-dioxopiperidin-3-yl)-6-fluoro-1,3-dioxoisoindolin-5-yl)piperazine-1-carboxylate

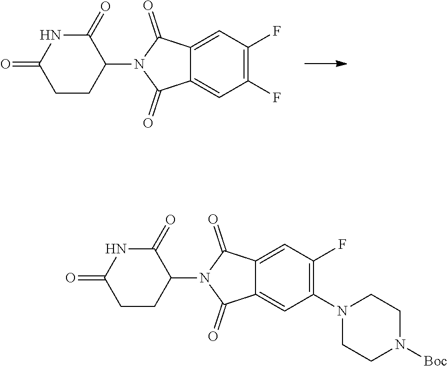 [SMILES: O=C1CCC(N2C(=O)c3cc(F)c(F)cc3C2=O)C(=O)N1 ; CC(C)(C)OC(=O)N1CCN(c2cc3c(cc2F)C(=O)N(C2CCC(=O)NC2=O)C3=O)CC1]

A mixture of tert-butyl piperazine-1-carboxylate (950 mg, 5.10 mmol, 1.00 eq.) and 2-(2,6-dioxopiperidin-3-yl)-5,6-difluoroisoindoline-1,3-dione (1.50 g, 5.10 mmol, 1.00 eq.), DIEA (1.97 g, 15.30 mmol, 3.00 eq.) in NMP (15.0 mL) was stirred at 110° C. overnight. The mixture was diluted with water and extracted EA. The organic layer was washed with brine, dried over Na₂SO₄, concentrated and the residue was purified by flash chromatography (PE:EA=1:2) to give the title compound as a yellow solid.

### Step 2: 2-(2,6-Dioxopiperidin-3-yl)-5-fluoro-6-(piperazin-1-yl)isoindoline-1,3-dione

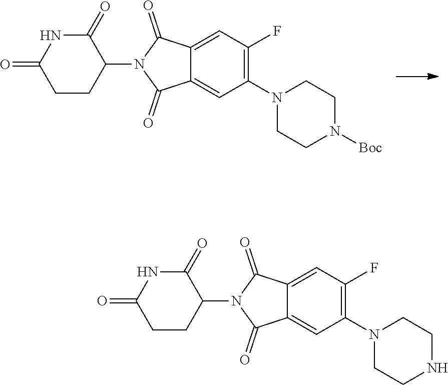 [SMILES: CC(C)(C)OC(=O)N1CCN(c2cc3c(cc2F)C(=O)N(C2CCC(=O)NC2=O)C3=O)CC1 ; O=C1CCC(N2C(=O)c3cc(F)c(N4CCNCC4)cc3C2=O)C(=O)N1]

To a stirred solution of tert-butyl 4-(2-(2,6-dioxopiperidin-3-yl)-6-fluoro-1,3-dioxoisoindolin-5-yl)piperazine-1-carboxylate (800 mg, 1.74 mmol, 1.00 eq.) in DCM (4.0 mL) was added TFA (1.0 mL) and the mixture was stirred at RT for 2 h. The reaction mixture was concentrated to give the title compound as a yellow oil.

### Step 3: 3-(Bromomethyl)benzenesulfonyl chloride

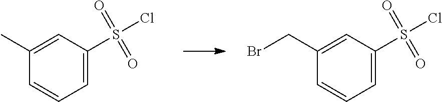

A mixture of 3-methylbenzenesulfonyl chloride (8.00 g, 41.96 mol, 1.00 eq.) NBS (8.22 g, 46.16 mol, 1.10 eq.) and benzoyl peroxide (1.46 g, 4.20 mol, 0.01 eq.) in CCl₄ (80.00 mL) was stirred at 80° C. for 12 h. The solution was filtered and the filtrate was concentrated to give crude product as white oil.

### Step 4: tert-Butyl (1-((3-(bromomethyl)phenyl)sulfonyl)piperidin-4-yl)carbamate

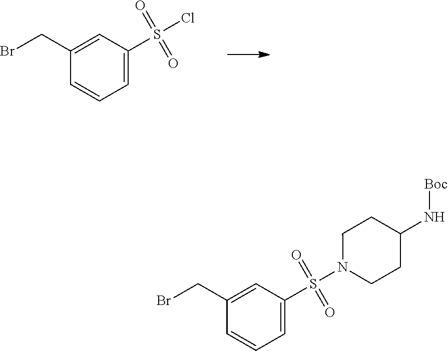 [SMILES: O=S(=O)(Cl)c1cccc(CBr)c1 ; CC(C)(C)OC(=O)NC1CCN(S(=O)(=O)c2cccc(CBr)c2)CC1]

tert-Butyl piperidin-4-ylcarbamate (5.64 g, 21.05 mol, 1.00 eq.) in THF (20.00 mL) was added to a stirred solution of 3-(bromomethyl)benzenesulfonyl chloride (3.79 g, 18.95 mol, 0.90 eq.) in THF (40.00 mL) and TEA (4.25 g, 42.10 mmol, 2.00 eq.) at 0° C. The resulting mixture was stirred at RT for 12 h, quenched with H₂O and then extracted with DCM. The combined organic layer was washed with water, dried over anhydrous Na₂SO₄, filtered, and concentrated. The residue was purified by silica gel column chromatography, eluted with PE/EA (3:1), to afford the title compound as white solid.

### Step 5: tert-Butyl (1-((3-((4-(2-(2,6-dioxopiperidin-3-yl)-6-fluoro-1,3-dioxoisoindolin-5-yl)piperazin-1-yl)methyl)phenyl)sulfonyl)piperidin-4-yl)carbamate

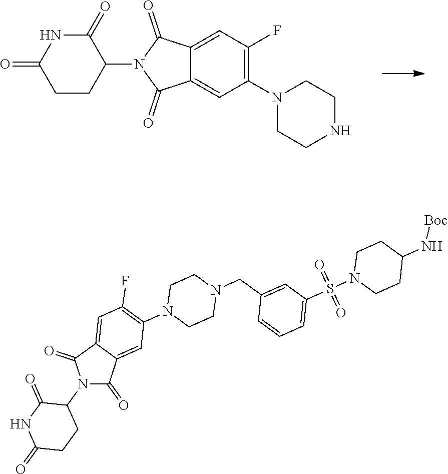 [SMILES: O=C1CCC(N2C(=O)c3cc(F)c(N4CCNCC4)cc3C2=O)C(=O)N1 ; CC(C)(C)OC(=O)NC1CCN(S(=O)(=O)c2cccc(CN3CCN(c4cc5c(cc4F)C(=O)N(C4CCC(=O)NC4=O)C5=O)CC3)c2)CC1]

A mixture of 2-(2,6-dioxopiperidin-3-yl)-5-fluoro-6-(piperazin-1-yl)isoindoline-1,3-dione (509 mg, 1.41 mmol, 1.00 eq.), tert-butyl (1-((3-(bromomethyl)phenyl)sulfonyl)-piperidin-4-yl)carbamate (916 mg, 2.12 mmol, 1.50 eq.) TEA (854 mg, 8.46 mmol, 6.00 eq.) in THF (10.0 mL) was stirred at 55° C. overnight. The mixture was extracted DCM and water. The organic layer was washed with brine, dried over Na₂SO₄, filtered and concentrated. The residue was purified by flash chromatography (DCM:MeOH=20:1) to give the title compound as a yellow solid.

### Reference 37

### Synthesis of tert-butyl (1-((3-((8-(2-(2,6-dioxopiperidin-3-yl)-6-fluoro-1,3-dioxoisoindolin-5-yl)-3,8-diazabicyclo[3.2.1]octan-3-yl)methyl)phenyl)sulfonyl)piperidin-4-yl)carbamate

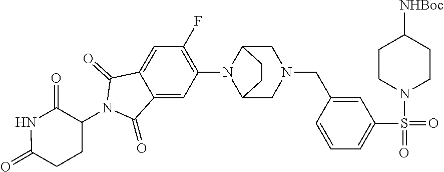 [SMILES: CC(C)(C)OC(=O)NC1CCN(S(=O)(=O)c2cccc(CN3CC4CCC(C3)N4c3cc4c(cc3F)C(=O)N(C3CCC(=O)NC3=O)C4=O)c2)CC1]

### Step 1: tert-Butyl 8-(2-(2,6-dioxopiperidin-3-yl)-6-fluoro-1,3-dioxoisoindolin-5-yl)-3,8-diazabicyclo[3.2.1]octane-3-carboxylate

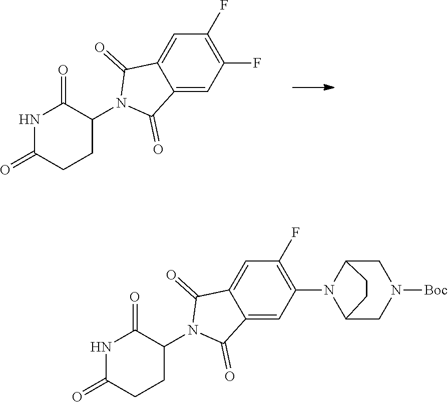 [SMILES: O=C1CCC(N2C(=O)c3cc(F)c(F)cc3C2=O)C(=O)N1 ; CC(C)(C)OC(=O)N1CC2CCC(C1)N2c1cc2c(cc1F)C(=O)N(C1CCC(=O)NC1=O)C2=O]

A mixture of 2-(2,6-dioxopiperidin-3-yl)-5,6-difluoroisoindoline-1,3-dione (200 mg, 6.68 mmol, 1.00 eq.), tert-butyl 3,8-diazabicyclo[3.2.1]octane-3-carboxylate (144 mg, 0.68 mmol, 1.00 eq.) and DIEA (263 mg, 2.04 mmol, 3.00 eq.) in NMP (3.0 mL) was stirred at 110° C. overnight. The reaction mixture was quenched with H₂O and then extracted with DCM. The combined organic layer was washed with water, dried over anhydrous Na₂SO₄, filtered, and concentrated. The residue was purified by silica gel column chromatography eluting with PE/EtOAc (1:1) to give title compound as yellow solid.

### Step 2: tert-Butyl (1-((3-((8-(2-(2,6-dioxopiperidin-3-yl)-6-fluoro-1,3-dioxoisoindolin-5-yl)-3,8-diazabicyclo[3.2.1]octan-3-yl)methyl)phenyl)sulfonyl)piperidin-4-yl)carbamate

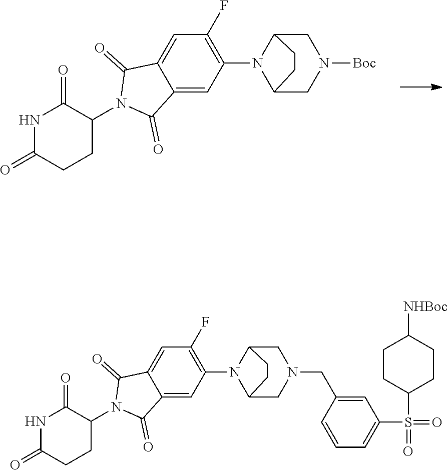 [SMILES: CC(C)(C)OC(=O)N1CC2CCC(C1)N2c1cc2c(cc1F)C(=O)N(C1CCC(=O)NC1=O)C2=O ; CC(C)(C)OC(=O)NC1CCC(S(=O)(=O)c2cccc(CN3CC4CCC(C3)N4c3cc4c(cc3F)C(=O)N(C3CCC(=O)NC3=O)C4=O)c2)CC1]

tert-Butyl 8-(2-(2,6-Dioxopiperidin-3-yl)-6-fluoro-1,3-dioxoisoindolin-5-yl)-3,8-diazabicyclo[3.2.1]octane-3-carboxylate was converted to the title compound proceeding analogously as described in Reference 36, Step 2-5.

### Reference 38

### Synthesis of tert-butyl (1-((3-((4-(2-(2,6-dioxopiperidin-3-yl)-1-oxoisoindolin-5-yl)piperazin-1-yl)-methyl)phenyl)sulfonyl)piperidin-4-yl)carbamate

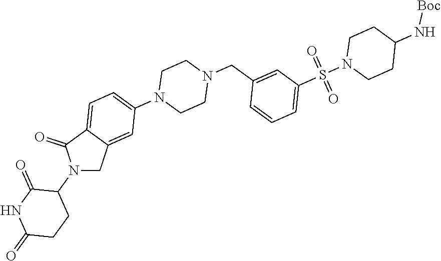 [SMILES: CC(C)(C)OC(=O)NC1CCN(S(=O)(=O)c2cccc(CN3CCN(c4ccc5c(c4)CN(C4CCC(=O)NC4=O)C5=O)CC3)c2)CC1]

### Step 1: tert-Butyl 4-(3-cyano-4-(methoxycarbonyl)phenyl)piperazine-1-carboxylate

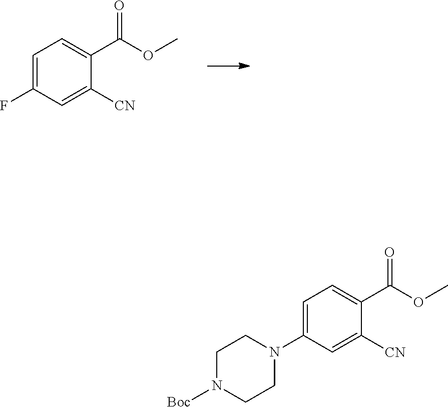 [SMILES: COC(=O)c1ccc(F)cc1C#N ; COC(=O)c1ccc(N2CCN(C(=O)OC(C)(C)C)CC2)cc1C#N]

To a stirred solution of methyl 2-cyano-4-fluorobenzoate (10.00 g, 55.80 mmol, 1.00 eq.) in DMSO (150.0 mL) was added tert-butyl piperazine-1-carboxylate (11.40 g, 61.38 mmol, 1.10 eq.) and DIEA (34.70 g, 268.96 mmol, 4.80 eq.). The resulting mixture was stirred at 110° C. for 12 h. The mixture was extracted with EtOAc washed with brine, concentrated and purified by silica gel column chromatography eluting with PE/EtOAc (3:1) to give the title compound as yellow solid.

### Step 2: tert-Butyl 4-(3-formyl-4-(methoxycarbonyl)phenyl)piperazine-1-carboxylate

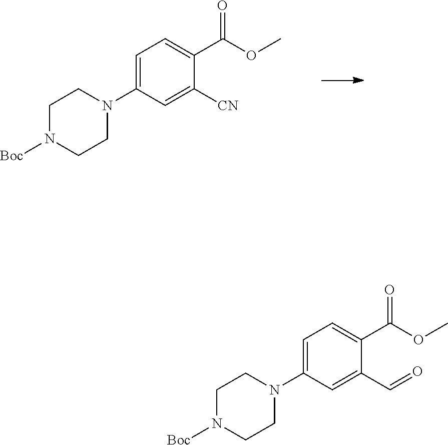 [SMILES: COC(=O)c1ccc(N2CCN(C(=O)OC(C)(C)C)CC2)cc1C#N ; COC(=O)c1ccc(N2CCN(C(=O)OC(C)(C)C)CC2)cc1C=O]

A mixture of tert-butyl 4-(3-cyano-4-(methoxycarbonyl)phenyl)piperazine-1-carboxylate (8.00 g, 23.20 mmol, 1.00 eq.) NaH₂PO₂·H₂O (5.20 g, 48.70 mmol, 2.10 eq.) and Raney-Ni (5.10 g) in pyridine:H₂O:AcOH=2:1:1 (80.0 mL) was stirred at 70° C. for 12 h. The mixture was adjusted pH=7˜8 with aq.NaHCO₃, filtered, and extracted with EtOAc. The organic layer was washed with brine, concentrated and the residue was purified by silica gel column chromatography eluting with PE/EtOAc (3:1) to give the title compound as yellow solid.

### Step 3: tert-Butyl 4-(2-(2,6-dioxopiperidin-3-yl)-1-oxoisoindolin-5-yl)piperazine-1-carboxylate

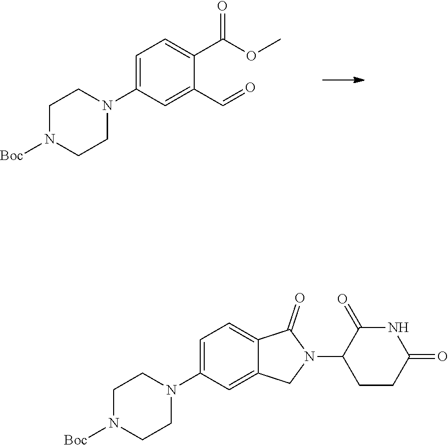 [SMILES: COC(=O)c1ccc(N2CCN(C(=O)OC(C)(C)C)CC2)cc1C=O ; CC(C)(C)OC(=O)N1CCN(c2ccc3c(c2)CN(C2CCC(=O)NC2=O)C3=O)CC1]

A mixture of 3-aminopiperidine-2,6-dione hydrochloride (2.60 g, 15.50 mmol, 1.20 eq.) DIEA (4.03 g, 31.22 mmol, 2.42 eq.), AcOH (10.63 g, 188.76 mmol, 13.78 eq.) and tert-butyl 4-(3-formyl-4-(methoxycarbonyl)phenyl)piperazine-1-carboxylate (4.50 g, 12.90 mmol, 1.00 eq.) in DCM (50.0 mL) was stirred at 35° C. for 4 h and then NaBH(OAc)₃ (8.20 g, 38.70 mmol, 3.00 eq.) was added at RT. The mixture was stirred at 40° C. for 12 h and diluted with water and extracted with EtOAc. The organic layer was washed with brine, concentrated, and the residue was purified by silica gel column chromatography eluting with PE/EtOAc (1:2) to give the title compound as white solid.

### Step 4: 3-(1-Oxo-5-(piperazin-1-yl)isoindolin-2-yl)piperidine-2,6-dione

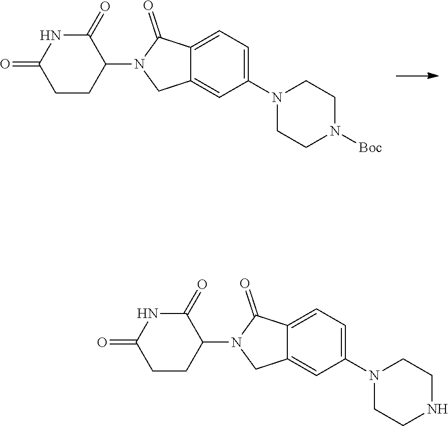 [SMILES: CC(C)(C)OC(=O)N1CCN(c2ccc3c(c2)CN(C2CCC(=O)NC2=O)C3=O)CC1 ; O=C1CCC(N2Cc3cc(N4CCNCC4)ccc3C2=O)C(=O)N1]

To a solution of tert-butyl 4-(2-(2,6-dioxopiperidin-3-yl)-1-oxoisoindolin-5-yl)piperazine-1-carboxylate (72 mg, 0.17 mmol, 1.00 eq.) in DCM (4.0 mL) was added TFA (1.0 mL). The resulting mixture was stirred at RT for 2 h and then concentrated to give the title compound as yellow oil.

### Step 5: tert-Butyl (1-((3-((4-(2-(2,6-dioxopiperidin-3-yl)-1-oxoisoindolin-5-yl)piperazin-1-yl)-methyl)phenyl)sulfonyl)piperidin-4-yl)carbamate

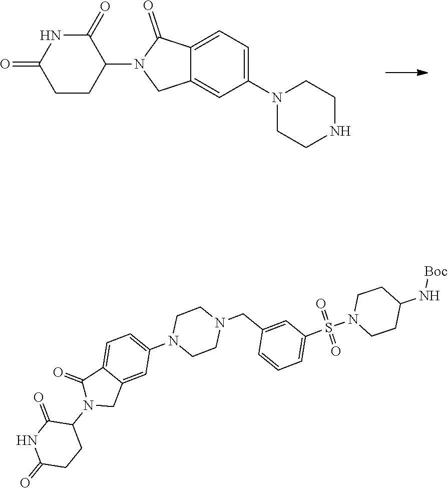 [SMILES: O=C1CCC(N2Cc3cc(N4CCNCC4)ccc3C2=O)C(=O)N1 ; CC(C)(C)OC(=O)NC1CCN(S(=O)(=O)c2cccc(CN3CCN(c4ccc5c(c4)CN(C4CCC(=O)NC4=O)C5=O)CC3)c2)CC1]

To a stirred solution of 3-(1-oxo-5-(piperazin-1-yl)isoindolin-2-yl)piperidine-2,6-dione (55 mg, 0.17 mmol, 1.00 eq.) in THF (2.0 mL) were added TEA (52 mg, 0.51 mmol, 3.00 eq.) and tert-butyl (1-((3-(bromomethyl)phenyl)sulfonyl)piperidin-4-yl)carbamate (95 mg, 0.22 mmol, 1.30 eq.). The reaction mixture was stirred at 55° C. overnight. The reaction mixture was concentrated and purified by silica gel column chromatography eluting with DCM/MeOH (20:1) to give the title compound as a yellow solid.

### Reference 39

### Synthesis of tert-butyl (1-((3-((7-(2-(2,6-dioxopiperidin-3-yl)-1-oxoisoindolin-5-yl)-2,7-diazaspiro-[3.5]nonan-2-yl)methyl)phenyl)sulfonyl)piperidin-4-yl)carbamate

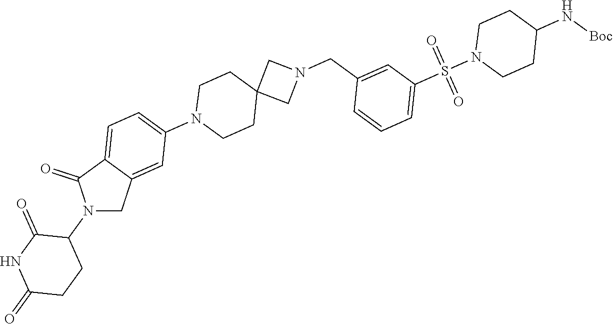 [SMILES: CC(C)(C)OC(=O)NC1CCN(S(=O)(=O)c2cccc(CN3CC4(CCN(c5ccc6c(c5)CN(C5CCC(=O)NC5=O)C6=O)CC4)C3)c2)CC1]

### Step 1: tert-Butyl 7-(3-cyano-4-(methoxycarbonyl)phenyl)-2,7-diazaspiro[3.5]nonane-2-carboxylate

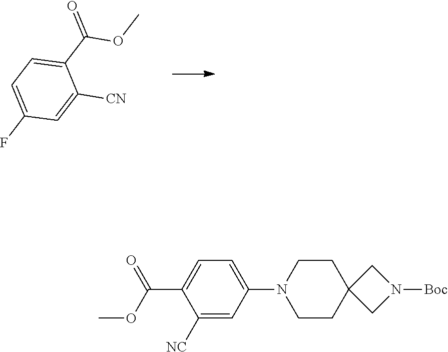 [SMILES: COC(=O)c1ccc(F)cc1C#N ; COC(=O)c1ccc(N2CCC3(CC2)CN(C(=O)OC(C)(C)C)C3)cc1C#N]

A mixture of methyl 2-cyano-4-fluorobenzoate (1.00 g, 5.58 mmol, 1.00 eq.) and tert-butyl 2,7-diazaspiro[3.5]nonane-2-carboxylate (1.39 g, 6.14 mmol, 1.10 eq.) DIEA (719 mg, 16.74 mmol, 3.00 eq.) in DMSO (10.0 mL) was stirred at 110° C. overnight. The mixture was diluted with water and extracted EA The organic layer was washed with brine, dried over Na₂SO₄, and concentrated. The residue was purified by flash chromatography (PE:EA=3:1) to give the title compound as a white solid.

### Step 2: tert-Butyl 7-(2-(2,6-dioxopiperidin-3-yl)-1-oxoisoindolin-5-yl)-2,7-diazaspiro[3.5]nonane-2-carboxyl

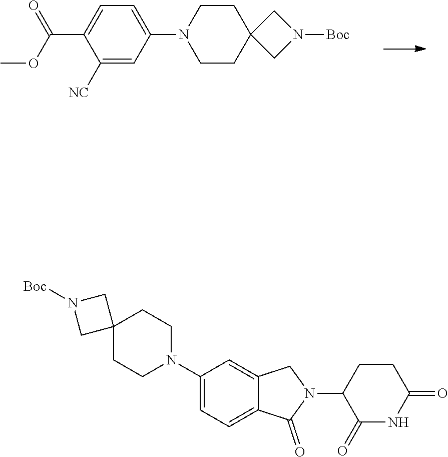 [SMILES: COC(=O)c1ccc(N2CCC3(CC2)CN(C(=O)OC(C)(C)C)C3)cc1C#N ; CC(C)(C)OC(=O)N1CC2(CCN(c3ccc4c(c3)CN(C3CCC(=O)NC3=O)C4=O)CC2)C1]

tert-butyl 7-(3-Cyano-4-(methoxycarbonyl)phenyl)-2,7-diazaspiro[3.5]nonane-2-carboxylate was converted to the title compound by proceeding analogously as described in Reference 28, Step 6-7.

### Step 3: 3-(1-Oxo-5-(2,7-diazaspiro[3.5]nonan-7-yl)isoindolin-2-yl)piperidine-2,6-dione

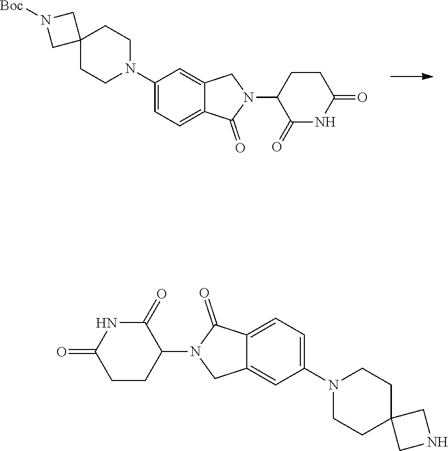 [SMILES: CC(C)(C)OC(=O)N1CC2(CCN(c3ccc4c(c3)CN(C3CCC(=O)NC3=O)C4=O)CC2)C1 ; O=C1CCC(N2Cc3cc(N4CCC5(CC4)CNC5)ccc3C2=O)C(=O)N1]

To a stirred solution of tert-butyl 7-(2-(2,6-dioxopiperidin-3-yl)-1-oxoisoindolin-5-yl)-2,7-diazaspiro[3.5]nonane-2-carboxylate (220 mg, 0.32 mmol, 1.00 eq.) in DCM (2.0 mL) was added TFA (0.5 mL) and the mixture was stirred at RT for 2 It The reaction mixture was concentrated to give title compound as a yellow oil.

### Step 4: tert-Butyl (1-((3-((7-(2-(2,6-dioxopiperidin-3-yl)-1-oxoisoindolin-5-yl)-2,7-diazaspiro-[3.5]nonan-2-yl)methyl)phenyl)sulfonyl)piperidin-4-yl)carbamate

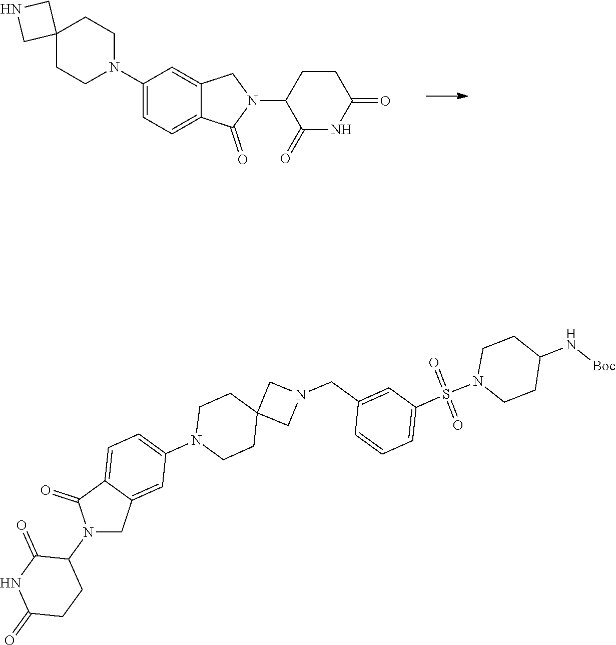 [SMILES: O=C1CCC(N2Cc3cc(N4CCC5(CC4)CNC5)ccc3C2=O)C(=O)N1 ; CC(C)(C)OC(=O)NC1CCN(S(=O)(=O)c2cccc(CN3CC4(CCN(c5ccc6c(c5)CN(C5CCC(=O)NC5=O)C6=O)CC4)C3)c2)CC1]

A mixture of 3-(1-oxo-5-(2,7-diazaspiro[3.5]nonan-7-yl)isoindolin-2-yl)piperidine-2,GP-301,C3 dione (173 mg, 0.47 mmol, 1.00 eq.) and tert-butyl (1-((3-(bromomethyl)phenyl)sulfonyl)-piperidin-4-yl)carbamate (264 mg, 0.61 mmol, 1.30 eq.) TEA (285 mg, 2.82 mmol, 6.00 eq.) in THF (5.0 mL) was stirred at 55° C. overnight. The mixture was diluted with water and extracted DCM. The organic layer was washed with brine, dried over Na₂SO₄, and concentrated. The residue was purified by flash chromatography (DCM:MeOH=20:1) to give the title compound as a yellow solid.

### Reference 40

### Synthesis of rac-tert-butyl ((3R,4S)-1-((3-((4-(2-(2,6-dioxopiperidin-3-yl)-6-fluoro-1,3-dioxoisoindolin-5-yl)piperazin-1-yl)methyl)phenyl)sulfonyl)-3-fluoropiperidin-4-yl)carbamate

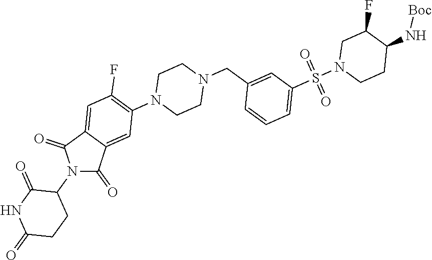 [SMILES: CC(C)(C)OC(=O)N[C@H]1CCN(S(=O)(=O)c2cccc(CN3CCN(c4cc5c(cc4F)C(=O)N(C4CCC(=O)NC4=O)C5=O)CC3)c2)C[C@H]1F]

### Step 1: rac-tert-Butyl ((3R,4S)-1-((3-(bromomethyl)phenyl)sulfonyl)-3-fluoropiperidin-4-yl)carbamate

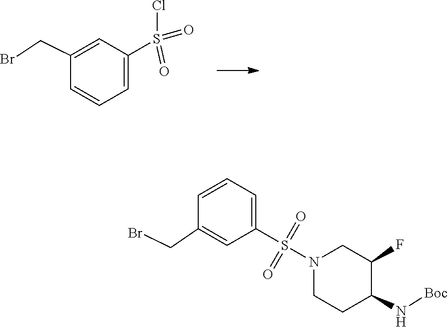 [SMILES: O=S(=O)(Cl)c1cccc(CBr)c1 ; CC(C)(C)OC(=O)N[C@H]1CCN(S(=O)(=O)c2cccc(CBr)c2)C[C@H]1F]

3-(Bromomethyl)-benzene-1-sulfonyl chloride (122 mg, 0.46 mmol, 1.00 eq.) in THF (1.0 ml, was added to a solution of rac-tert-butyl ((3R,4S)-3-fluoropiperidin-4-yl)carbamate (100 mg, 0.46 mmol, 1.00 eq.) and TEA (93 mg, 0.92 mmol, 2.00 eq.) in THF (2.0 mL) slowly at −10° C. for 3 h. The mixture was diluted with water and extracted EA. The organic layer was washed with brine, dried over Na₂SO₄, and concentrated. The residue was purified by flash chromatography (PE:EA=4:1) to give the title compound as a white solid.

### Step 2: rac-tert-Butyl ((3R,4S)-1-((3-((4-(2-(2,6-dioxopiperidin-3-yl)-6-fluoro-1,3-dioxoisoindolin-5-yl)piperazin-1-yl)methyl)phenyl)sulfonyl)-3-fluoropiperidin-4-yl)carbamate

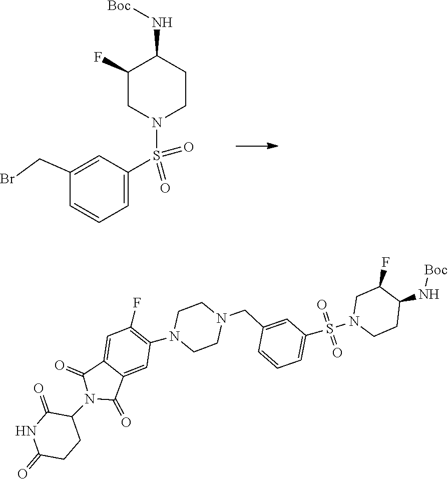 [SMILES: CC(C)(C)OC(=O)N[C@H]1CCN(S(=O)(=O)c2cccc(CBr)c2)C[C@H]1F ; CC(C)(C)OC(=O)N[C@H]1CCN(S(=O)(=O)c2cccc(CN3CCN(c4cc5c(cc4F)C(=O)N(C4CCC(=O)NC4=O)C5=O)CC3)c2)C[C@H]1F]

To a solution of 2-(2,6-dioxopiperidin-3-yl)-5-fluoro-6-(piperazin-I-yl)isoindoline-1,3-dione (94 mg, 0.26 mmol, 1.00 eq.) and rac-tert-butyl ((3R,4S)-1-((3-(bromomethyl)phenyl)-sulfonyl)-3-fluoropiperidin-4-yl)carbamate (141 mg, 0.31 mmol, 1.20 eq.) in THF (4.0 mL) was added TEA (131 mg, 1.30 mmol, 5.00 eq.) and the mixture was stirred at 55° C. overnight. The mixture was diluted with water and extracted DCM. The organic layer was washed with brine, dried over Na₂SO₄, and concentrated. The residue was purified by flash chromatography (DCM:MeOH=20:1) to give the title compound as a yellow solid.

### Reference 41

### Synthesis of tert-butyl (1-((3-((1-((2-(2,6-dioxopiperidin-3-yl)-1-oxoisoindolin-5-yl)-methyl)piperidin-4-yl)oxy)phenyl)sulfonyl)piperidin-4-yl)carbamate

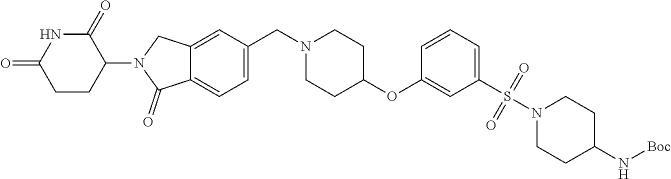 [SMILES: CC(C)(C)OC(=O)NC1CCN(S(=O)(=O)c2cccc(OC3CCN(Cc4ccc5c(c4)CN(C4CCC(=O)NC4=O)C5=O)CC3)c2)CC1]

### Step 1: 2-(2,6-Dioxopiperidin-3-yl)-1-oxoisoindoline-5-carbonitrile

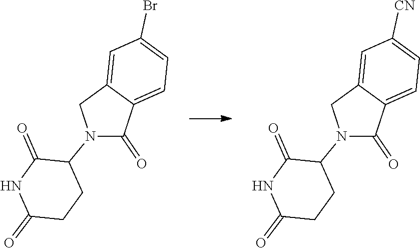 [SMILES: O=C1CCC(N2Cc3cc(Br)ccc3C2=O)C(=O)N1 ; N#Cc1ccc2c(c1)CN(C1CCC(=O)NC1=O)C2=O]

A mixture of 3-(5-bromo-1-oxoisoindolin-2-yl)piperidine-2,6-dione (2.00 g, 6.21 mmol, 1.00 eq.) and Zn(CN)₂ (438 mg, 3.73 mmol, 0.60 eq.) Pd(pph₃)₄ (714 mg) in DMF (30.0 mL) was stirred at 100° C. overnight. The mixture was extracted with DCM and purified by silica gel column chromatography eluting with PE/EtOAc (1:2) to give the title compound as yellow solid.

### Step 2: 2-(2,6-Dioxopiperidin-3-yl)-1-oxoisoindoline-5-carbaldehyde

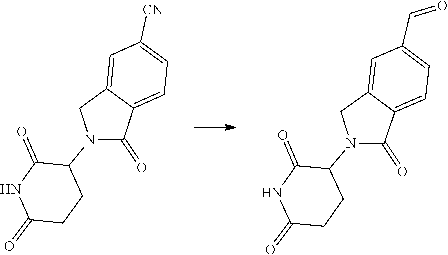 [SMILES: N#Cc1ccc2c(c1)CN(C1CCC(=O)NC1=O)C2=O ; O=Cc1ccc2c(c1)CN(C1CCC(=O)NC1=O)C2=O]

A mixture of 2-(2,6-dioxopiperidin-3-yl)-1-oxoisoindoline-5-carbonitrile (1.20 g, 4.46 mmol, 1.00 eq.), NaH₂PO₂·H₂O (993 mg, 9.37 mmol, 2.10 eq.) and Raney-Ni (500 mg) in pyridine:H₂O:AcOH (40.0 mL, 2:2:1) was stirred at 70° C. overnight. The reaction mixture was filtered and washed with aq. NaHCO₃. The organic layer was concentrated and the residue was purified by silica gel column chromatography eluting PE/EtOAc (1:2) to give the title compound as yellow solid.

### Step 3: Benzyl 4-(3-((4-((tert-butoxycarbonyl)amino)piperidin-1-yl)sulfonyl)phenoxy)piperidine-1-carboxylate

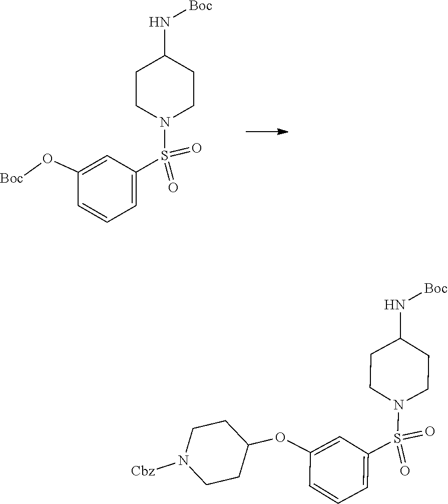 [SMILES: CC(C)(C)OC(=O)NC1CCN(S(=O)(=O)c2cccc(OC(=O)OC(C)(C)C)c2)CC1 ; CC(C)(C)OC(=O)NC1CCN(S(=O)(=O)c2cccc(OC3CCN(C(=O)OCc4ccccc4)CC3)c2)CC1]

A mixture of tert-butyl (1-((3-((tert-butoxycarbonyl)oxy)phenyl)sulfonyl)piperidin-4-yl)carbamate (7.30 g, 16.0 mmol, 1.00 eq.), benzyl 4-((methylsulfonyl)oxy)piperidine-1-carboxylate (7.52 g, 24 mmol, 1.50 eq.) and Cs₂CO₃ (10.4 g, 32 mmol, 2.00 eq.) in DMSO (70.0 mL) was stirred at 90° C. for 4 h and then extracted with EtOAc. The organic layer was concentrated and the residue was purified by silica gel column chromatography eluting PE/EtOAc (3:1) to give the title compound as a yellow solid.

### Step 4: tert-Butyl (1-((3-(piperidin-4-yloxy)phenyl)sulfonyl)piperidin-4-yl)carbamate

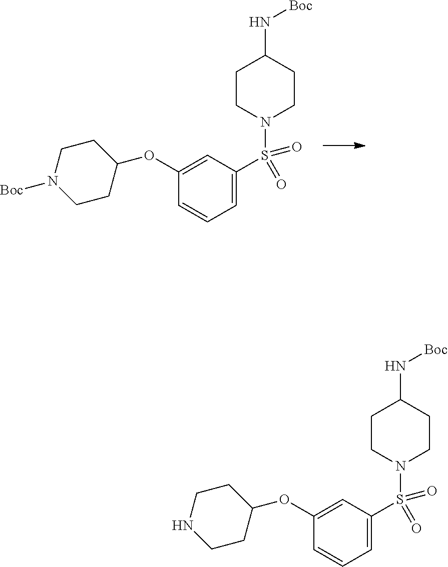 [SMILES: CC(C)(C)OC(=O)NC1CCN(S(=O)(=O)c2cccc(OC3CCN(C(=O)OC(C)(C)C)CC3)c2)CC1 ; CC(C)(C)OC(=O)NC1CCN(S(=O)(=O)c2cccc(OC3CCNCC3)c2)CC1]

A mixture of benzyl 4-(3-((4-((tert-butoxycarbonyl)amino)piperidin-1-yl)sulfonyl)-phenoxy)piperidine-1-carboxylate (6.0 g, 10.47 mmol, 1.00 eq.), HCOONH₄ (3.3 g, 52.35 mmol, 5.00 eq.), and Pd(OH)₂ (1.2 g) in EtOH (60.0 mL) was stirred at 70° C. for 4 h. The mixture was filtered and concentrated to give the title compound as a white solid.

### Step 5: tert-Butyl (1-((3-((1-((2-(2,6-dioxopiperidin-3-yl)-1-oxoisoindolin-5-yl)methyl)piperidin-4-yl)oxy)phenyl)sulfonyl)piperidin-4-yl)carbamate

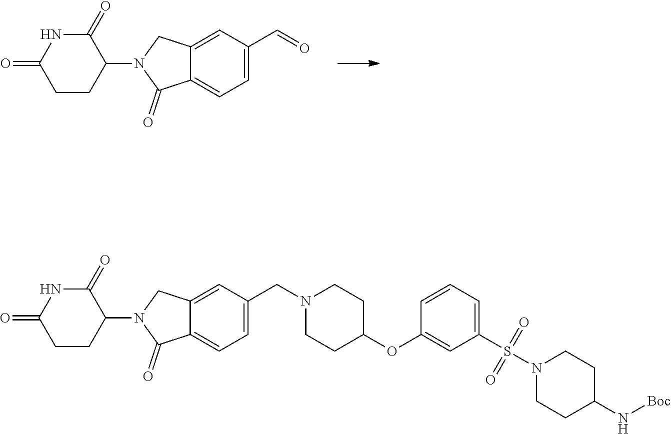 [SMILES: O=Cc1ccc2c(c1)CN(C1CCC(=O)NC1=O)C2=O ; CC(C)(C)OC(=O)NC1CCN(S(=O)(=O)c2cccc(OC3CCN(Cc4ccc5c(c4)CN(C4CCC(=O)NC4=O)C5=O)CC3)c2)CC1]

To a mixture of 2-(2,6-dioxopiperidin-3-yl)-1-oxoisoindoline-5-carbaldehyde (100 mg, 0.37 mmol, 1.00 eq.) in THF (3.0 mL) were added tert-butyl (1-((3-(piperidin-4-yloxy)phenyl)-sulfonyl)piperidin-4-yl)carbamate (169 mg, 0.39 mmol, 1.05 eq.) and 1 drop of AcOH. The mixture was stirred at 40° C. for 0.5 h. NaBH₃CN (47 mg, 0.74 mmol, 2.00 eq.) was added and stirred at RT for 16 h. The reaction mixture was diluted with water and extracted DCM. The organic layer was washed with brine, dried over Na₂SO₄, and concentrated. The residue was purified by silica gel column chromatography eluting with DCM/MeOH (20:1) to give the title compound as a yellow solid.

### Reference 42

### Synthesis of tert-butyl (1-((3-(4-(1-(2-(2,6-dioxopiperidin-3-yl)-1-oxoisoindolin-5-yl)azetidin-3-yl)piperazin-1-yl)phenyl)sulfonyl)piperidin-4-yl)carbamate

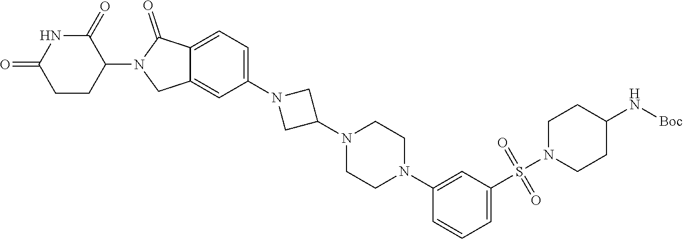 [SMILES: CC(C)(C)OC(=O)NC1CCN(S(=O)(=O)c2cccc(N3CCN(C4CN(c5ccc6c(c5)CN(C5CCC(=O)NC5=O)C6=O)C4)CC3)c2)CC1]

### Step 1: Benzyl 3-(4-(3-((4-((tert-butoxycarbonyl)amino)piperidin-1-yl)sulfonyl)phenyl)piperazin-1-yl)azetidine-1-carboxylate

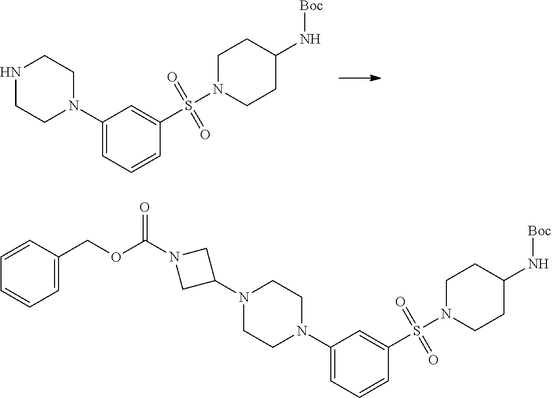

To a mixture of tert-butyl (1-((3-(piperazin-1-yl)phenyl)sulfonyl)piperidin-4-yl)carbamate (740 mg, 1.75 mmol, 1.00 eq.) in THF (10.0 mL) were added AcOH (3 drops) and benzyl 3-oxoazetidine-1-carboxylate (718 mg, 3.50 mmol, 2.00 eq.). The solution was stirred at 45° C. for 0.5 h. The solution was cooled to RT and NaBH₃CN (220 mg, 3.50 mmol, 2.00 eq.) was added. The solution was stirred at RT overnight and then extracted with EtOAc. The mixture was diluted with water and extracted DCM. The organic layer was washed with brine, dried over Na₂SO₄, and concentrated. The residue was purified by silica gel column chromatography eluting DCM/MeOH (20:1) to give the title compound as a white oil.

### Step 2: tert-Butyl (1-((3-(4-(1-(2-(2,6-dioxopiperidin-3-yl)-1-oxoisoindolin-5-yl)azetidin-3-yl)piperazin-1-yl)phenyl)sulfonyl)piperidin-4-yl)carbamate

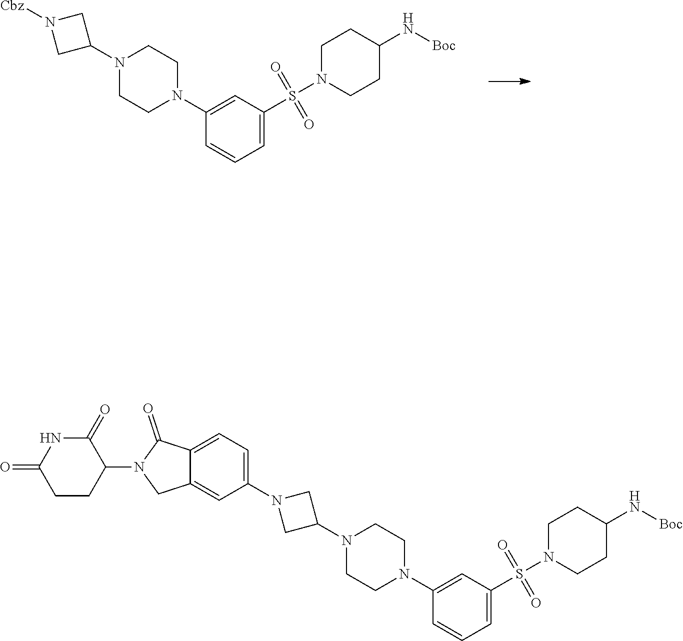 [SMILES: CC(C)(C)OC(=O)NC1CCN(S(=O)(=O)c2cccc(N3CCN(C4CN(C(=O)OCc5ccccc5)C4)CC3)c2)CC1 ; CC(C)(C)OC(=O)NC1CCN(S(=O)(=O)c2cccc(N3CCN(C4CN(c5ccc6c(c5)CN(C5CCC(=O)NC5=O)C6=O)C4)CC3)c2)CC1]

Benzyl 3-(4-(3-((4-((tert-butoxycarbonyl)amino)piperidin-1-yl)sulfonyl)phenyl)-piperazin-1-yl)azetidine-1-carboxylate was converted to the title compound using similar procedure as described in Reference 28, Step 4-7.

### Reference 43

### Synthesis of tert-butyl (1-((3-(3-(4-(2-(2,6-dioxopiperidin-3-yl)-1-oxoisoindolin-5-yl)piperazin-1-yl)-azetidin-1-yl)phenyl)sulfonyl)piperidin-4-yl)carbamate

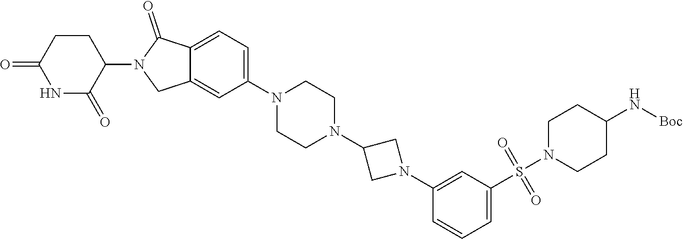 [SMILES: CC(C)(C)OC(=O)NC1CCN(S(=O)(=O)c2cccc(N3CC(N4CCN(c5ccc6c(c5)CN(C5CCC(=O)NC5=O)C6=O)CC4)C3)c2)CC1]

### Step 1: tert-Butyl (1-((3-(3-hydroxyazetidin-1-yl)phenyl)sulfonyl)piperidin-4-yl)carbamate

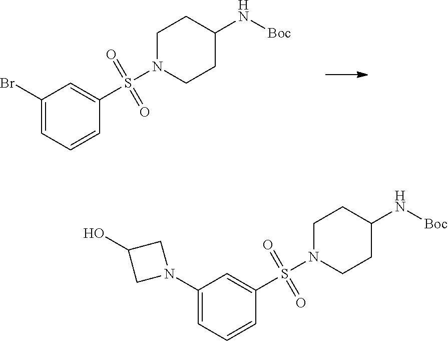 [SMILES: CC(C)(C)OC(=O)NC1CCN(S(=O)(=O)c2cccc(Br)c2)CC1 ; CC(C)(C)OC(=O)NC1CCN(S(=O)(=O)c2cccc(N3CC(O)C3)c2)CC1]

A mixture of tert-butyl (1-((3-bromophenyl)sulfonyl)piperidin-4-yl)carbamate (5.83 g, 13.95 mmol, 1.00 eq.), K₂CO₃ (6.74 g, 48.83 mmol, 3.50 eq.), CuI (0.53 g, 2.79 mmol, 0.20 eq.), L-PRO (481 mg, 4.19 mmol, 0.30 eq.) and 3-hydroxyazetidine (2.28 g, 20.92 mmol, 1.50 eq.) in DMSO (50.00 mL) was stirred at 90° C. for 12 h. The mixture was quenched with H₂O and extracted with EtOAc. The organic layer was concentrated and purified by silica gel column chromatography eluting with PE/EtOAc (2:1) to give the title compound as white solid.

### Step 2: tert-Butyl (1-((3-(3-oxoazetidin-1-yl)phenyl)sulfonyl)piperidin-4-yl)carbamate

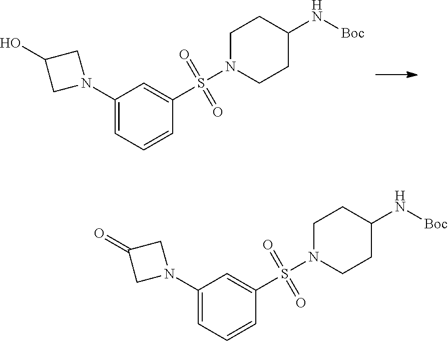 [SMILES: CC(C)(C)OC(=O)NC1CCN(S(=O)(=O)c2cccc(N3CC(O)C3)c2)CC1 ; CC(C)(C)OC(=O)NC1CCN(S(=O)(=O)c2cccc(N3CC(=O)C3)c2)CC1]

To a stirred solution of tert-butyl (1-((3-(3-hydroxyazetidin-1-yl)phenyl)sulfonyl)-piperidin-4-yl)carbamate (0.50 g, 1.22 mmol, 1.00 eq.) in DCM (5.00 mL) was added Dess-Martin (1.03 g, 2.44 mmol, 2.00 eq.) and the mixture was stirred at 0° C. for 3 h. The mixture was diluted with sodium thiosulfate (aq) and extracted with DCM. The organic layer was concentrated and the residue was purified by silica gel column chromatography, eluted with EtOAc/PE (3:1), to give the title compound as a white solid.

### Step 3: tert-Butyl (1-((3-(3-(4-(2-(2,6-dioxopiperidin-3-yl)-1-oxoisoindolin-5-yl)piperazin-1-yl)-azetidin-1-yl)phenyl)sulfonyl)piperidin-4-yl)carbamate

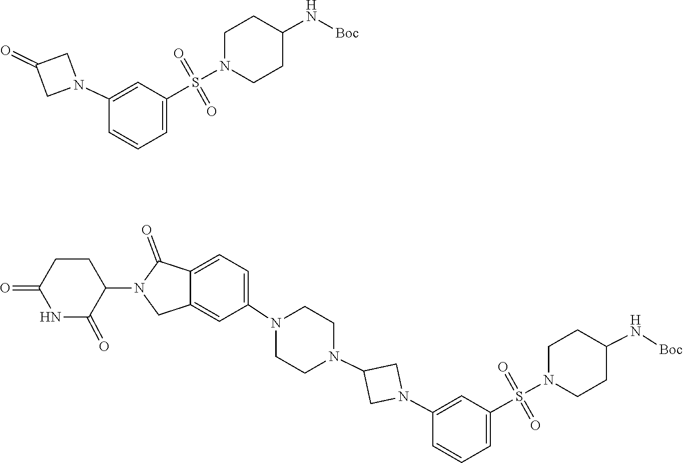 [SMILES: CC(C)(C)OC(=O)NC1CCN(S(=O)(=O)c2cccc(N3CC(=O)C3)c2)CC1 ; CC(C)(C)OC(=O)NC1CCN(S(=O)(=O)c2cccc(N3CC(N4CCN(c5ccc6c(c5)CN(C5CCC(=O)NC5=O)C6=O)CC4)C3)c2)CC1]

To a solution of tert-butyl (1-((3-(3-oxoazetidin-1-yl)phenyl)sulfonyl)piperidin-4-yl)carbamate (40.00 mg, 0.10 mmol, 1.00 eq.) in THF (1.00 mL) and DMF (0.50 mL) were added AcOH (3 drops) and 3-(1-oxo-5-(piperazin-1-yl)isoindolin-2-yl)piperidine-2,6-dione (39.00 mg, 0.12 mmol, 1.20 eq.). The solution was stirred at 45° C. for 45 min. The solution cooled to RT and NaBH₃CN (13.00 mg, 0.20 mmol, 2.00 eq.) was added. The mixture was stirred at RT for 12 h and then diluted with water and extracted with EtOAc. The combined organic layer was washed with brine, dried over anhydrous Na₂SO₄ and concentrated. The residue was purified by TLC, eluted with DCM/MeOH (20:1), to afford the title compound as a white solid.

### Reference 44

### Synthesis of tert-butyl (1-((3-(3-(4-(1-(2,6-dioxopiperidin-3-yl)-3-methyl-2-oxo-2,3-dihydro-1H-benzo[d]imidazol-4-yl)piperidin-1-yl)azetidin-1-yl)phenyl)sulfonyl)piperidin-4-yl)carbamate

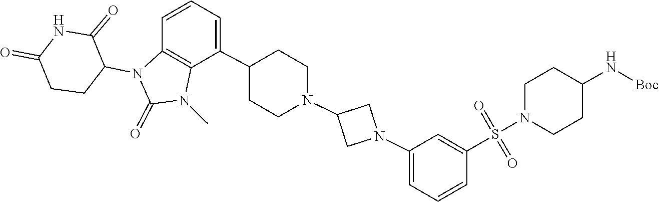 [SMILES: Cn1c(=O)n(C2CCC(=O)NC2=O)c2cccc(C3CCN(C4CN(c5cccc(S(=O)(=O)N6CCC(NC(=O)OC(C)(C)C)CC6)c5)C4)CC3)c21]

To a solution of 3-(3-methyl-2-oxo-4-(piperidin-4-yl)-2,3-dihydro-1H-benzo[d]imidazol-1-yl)piperidine-2,6-dione (41.00 mg, 0.12 mmol, 1.00 eq) in THF (2.00 mL) and DMF (0.50 mL) were added AcOH (3 drops) and tert-butyl (1-((3-(3-oxoazetidin-1-yl)phenyl)sulfonyl)piperidin-4-yl)carbamate (98.00 mg, 0.24 mmol, 2.00 eq.). The solution was stirred at 45° C. for 0.5 h. Then the solution cooled to RT and NaBH₃CN (15.08 mg, 0.24 mmol, 2.00 eq.) was added. The mixture was stirred at RT for 12 h and then diluted with water and extracted with EtOAc. The combined organic layer was washed with brine, dried over anhydrous Na₂SO₄ and concentrated. The residue was purified by TLC, eluted with DCM/MeOH (20:1), to afford the title compound as a white solid.

### Reference 45

### Synthesis of rac-1-(6-(1-(3-(((3R,4S)-4-amino-3-fluoropiperidin-1-yl)sulfonyl)benzyl)piperidin-4-yl)-1-methyl-1H-indazol-3-yl)dihydropyrimidine-2,4(1H,3H)-dione

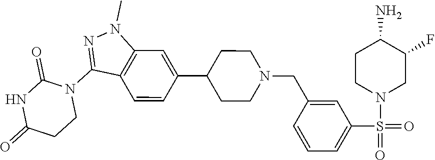 [SMILES: Cn1nc(N2CCC(=O)NC2=O)c2ccc(C3CCN(Cc4cccc(S(=O)(=O)N5CC[C@H](N)[C@H](F)C5)c4)CC3)cc21]

### Step 1: 6-Bromo-1-methyl-1H-indazol-3-amine

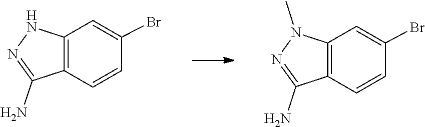

NaH (2.10 g, 52.83 mmol, 2.00 eq.) was added to a stirred solution of 6-bromo-1H-indazol-3-amine (5.60 g, 26.42 mmol, 1.00 eq.) in DMF (20.0 mL) at 0° C. and the mixture was stirred at 0° C. for 1 h. CH₃I (4.10 g, 29.06 mmol, 1.10 eq.) was added and the mixture was stirred at r.t. for 3 h under N₂. The mixture was poured into cold water and filtered. The solid was washed with water, dried to give the title compound as yellow solid.

### Step 2: 3-((6-Bromo-1-methyl-1H-indazol-3-yl)amino)propanoic acid

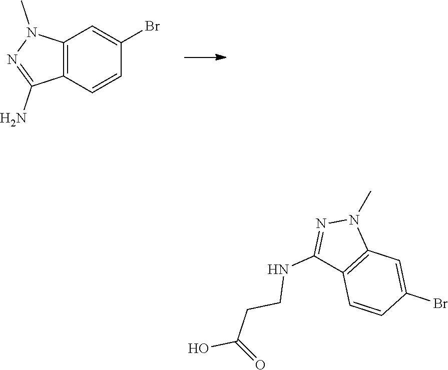 [SMILES: Cn1nc(N)c2ccc(Br)cc21 ; Cn1nc(NCCC(=O)O)c2ccc(Br)cc21]

Acrylic acid (1.60 g, 22.12 mmol, 1.00 eq.) was added to a stirred solution of 6-bromo-1-methyl-1H-indazol-3-amine (5.00 g, 22.12 mmol, 1.00 eq.) in AcOH (3.17 g, 52.43 mmol, 2.37 eq.) and water (5.0 mL) and the mixture was stirred at 105° C. for 20 h under N₂. The mixture was poured into cold water and the mixture was adjusted to pH to 6˜7 by addition of 6N HCl aq. The mixture was filtered and the solid was washed with water, dried to give the title compound as yellow solid.

### Step 3: 1-(6-Bromo-1-methyl-1H-indazol-3-yl)dihydropyrimidine-2,4(1H,3H)-dione

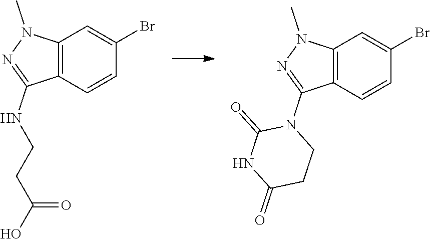 [SMILES: Cn1nc(NCCC(=O)O)c2ccc(Br)cc21 ; Cn1nc(N2CCC(=O)NC2=O)c2ccc(Br)cc21]

A mixture of 3-((6-bromo-1-methyl-1H-indazol-3-yl)amino)propanoic acid (3.11 g, 1.90 mmol, 1.00 eq.) and urea (3.02 g, 50.31 mmol, 5.00 eq.) in AcOH (30.0 mL) was stirred at 120° C. 20 h under N₂. After cooling the mixture to room temperature conc. HCl (6.0 mL) was added and then the mixture was heated again for 30 min. The crude mixture was diluted with water and extracted with EA, and the combined organic layer was washed with brine, dried over Na₂SO₄, and concentrated. The residue was purified by flash column chromatography (EA:PE=0 to 100%) to give the title compound as yellow solid.

### Step 4: tert-Butyl 4-(3-(2,4-dioxotetrahydropyrimidin-1(2H)-yl)-1-methyl-1H-indazol-6-yl)-5,6-dihydropyridine-1(2H)-carboxylate

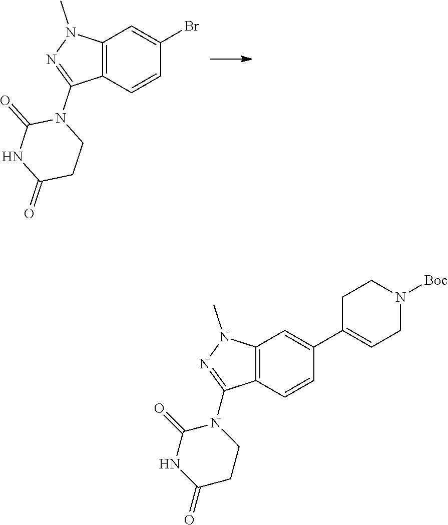 [SMILES: Cn1nc(N2CCC(=O)NC2=O)c2ccc(Br)cc21 ; Cn1nc(N2CCC(=O)NC2=O)c2ccc(C3=CCN(C(=O)OC(C)(C)C)CC3)cc21]

A mixture of 1-(6-bromo-1-methyl-1H-indazol-3-yl)dihydropyrimidine-2,4(1H,3H)-dione (1.10 g, 3.41 mmol, 1.00 eq.), tert-butyl 4-(4,4,5,5-tetramethyl-1,3,2-dioxaborolan-2-yl)-5,6-dihydropyridine-1(2H)-carboxylate (1.60 g, 5.11 mmol, 1.50 eq.), K₃PO₄ (2.20 g, 10.22 mmol, 3.00 eq.) and X-Phos-G3 (289 mg, 0.34 mmol, 0.10 eq.) in 1,4-dioxane/H₂O (10 mL/1 mL) was stirred at 60° C. under N₂ for 3 h. The mixture was diluted with DCM, and the organic layer was washed with water and brine, dried over Na₂SO₄, concentrated, and the residue was purified by column chromatography on silica gel (DCM:MeOH=20:1) to give the title compound as yellow solid.

### Step 5: tert-Butyl 4-(3-(2,4-dioxotetrahydropyrimidin-1(2H)-yl)-1-methyl-1H-indazol-6-yl)piperidine-1-carboxylate

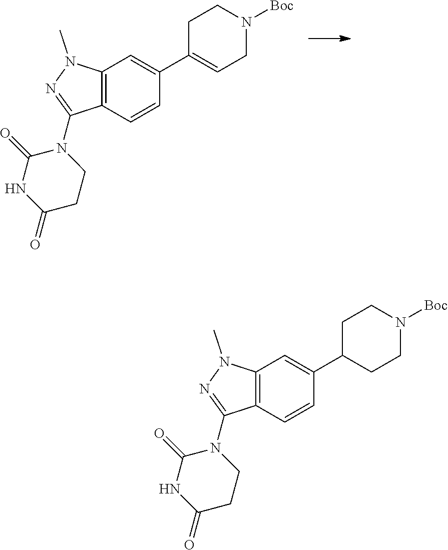 [SMILES: Cn1nc(N2CCC(=O)NC2=O)c2ccc(C3=CCN(C(=O)OC(C)(C)C)CC3)cc21 ; Cn1nc(N2CCC(=O)NC2=O)c2ccc(C3CCN(C(=O)OC(C)(C)C)CC3)cc21]

A mixture of tert-butyl 4-(3-(2,4-dioxotetrahydropyrimidin-1(2H)-yl)-1-methyl-1H-indazol-6-yl)-5,6-dihydropyridine-1(2H)-carboxylate (300 mg, 0.71 mmol, 1.00 eq.), Pd/C (150 mg, 50% wt) and Pd(OH)/C (150 mg, 50% wt) in THF (20 mL) was stirred under 50 psi H₂ at 50° C. overnight. The mixture was filtered, the filtrate was concentrated, and the residue was purified by column chromatography on silica gel (PE:EA=1:1) to give the title compound as yellow solid.

### Step 6: 1-(1-Methyl-6-(piperidin-4-yl)-1H-indazol-3-yl)dihydropyrimidine-2,4(1H,3H)-dione

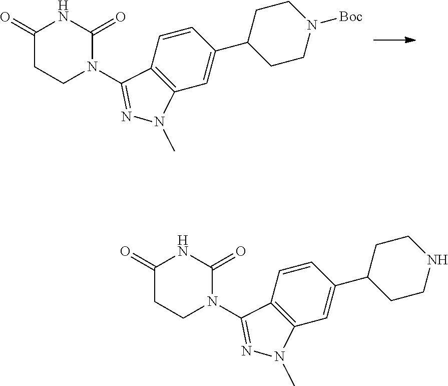 [SMILES: Cn1nc(N2CCC(=O)NC2=O)c2ccc(C3CCN(C(=O)OC(C)(C)C)CC3)cc21 ; Cn1nc(N2CCC(=O)NC2=O)c2ccc(C3CCNCC3)cc21]

TFA (0.5 mL) was added to a solution of tert-butyl 4-(3-(2,4-dioxotetrahydropyrimidin-1(2H)-yl)-1-methyl-1H-indazol-6-yl)piperidine-1-carboxylate (35.0 mg, 0.08 mmol, 1.00 eq.) in DCM (2.5 mL) and the mixture was stirred at r.t for 3 h. The resulting mixture was concentrated to give the crude product a white oil, which was used to next step without further purification.

### Step 7: rac-tert-Butyl ((3R,4S)-1-((3-((4-(3-(2,4-dioxotetrahydropyrimidin-1(2H)-yl)-1-methyl-1H-indazol-6-yl)piperidin-1-yl)methyl)phenyl)sulfonyl)-3-fluoropiperidin-4-yl)carbamate

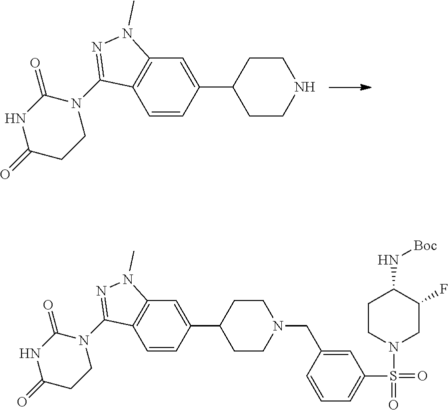 [SMILES: Cn1nc(N2CCC(=O)NC2=O)c2ccc(C3CCNCC3)cc21 ; Cn1nc(N2CCC(=O)NC2=O)c2ccc(C3CCN(Cc4cccc(S(=O)(=O)N5CC[C@H](NC(=O)OC(C)(C)C)[C@H](F)C5)c4)CC3)cc21]

A mixture of 1-(1-methyl-6-(piperidin-4-yl)-1H-indazol-3-yl)dihydropyrimidine-2,4(1H,3H)-dione (82 mg, 0.25 mmol, 1.00 eq.), rac-tert-butyl ((3R,4S)-1-((3-(bromomethyl)-phenyl)sulfonyl)-3-fluoropiperidin-4-yl)carbamate (115 mg, 0.25 mmol, 1.00 eq.) in DCM (4.0 mL), and TEA (76 mg, 0.75 mmol, 3.00 eq.) was stirred at 50° C. for 16 h. The mixture was filtered and the filtrate was concentrated. The residue was purified by column chromatography on silica gel (DCM:MeOH=15:1) to give the title compound as white solid.

### Step 8: rac-1-(6-(1-(3-(((3R,4S)-4-amino-3-fluoropiperidin-1-yl)sulfonyl)benzyl)piperidin-4-yl)-1-methyl-1H-indazol-3-yl)dihydropyrimidine-2,4(1H,3H)-dione

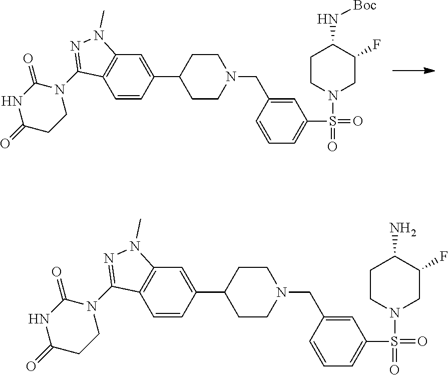 [SMILES: Cn1nc(N2CCC(=O)NC2=O)c2ccc(C3CCN(Cc4cccc(S(=O)(=O)N5CC[C@H](NC(=O)OC(C)(C)C)[C@H](F)C5)c4)CC3)cc21 ; Cn1nc(N2CCC(=O)NC2=O)c2ccc(C3CCN(Cc4cccc(S(=O)(=O)N5CC[C@H](N)[C@H](F)C5)c4)CC3)cc21]

A mixture of tert-butyl ((3R,4S)-1-((3-((4-(3-(2,4-dioxotetrahydropyrimidin-1(2H)-yl)-1-methyl-1H-indazol-6-yl)piperidin-1-yl)methyl)phenyl)sulfonyl)-3-fluoropiperidin-4-yl)carbamate (45 mg, 0.07 mmol, 1.00 eq.) in TFA/DCM (0.5 mL/2.0 mL) was stirred at rt for 2 h. The mixture was concentrated to give the title compound as its TFA salt as brown oil.

### Reference 46

### Synthesis of 3-((4-(Piperidin-4-yl)phenyl)amino)piperidine-2,6-dione

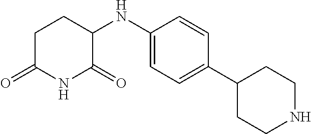 [SMILES: O=C1CCC(Nc2ccc(C3CCNCC3)cc2)C(=O)N1]

### Step 1: tert-Butyl 4-(4-nitrophenyl)-5,6-dihydropyridine-1(2H)-carboxylate

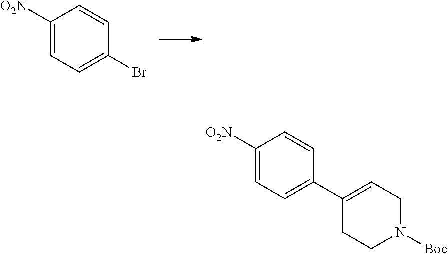 [SMILES: O=[N+]([O-])c1ccc(Br)cc1 ; CC(C)(C)OC(=O)N1CC=C(c2ccc([N+](=O)[O-])cc2)CC1]

A mixture of 1-bromo-4-nitrobenzene (1.0 g, 4.95 mmol, 1.00 eq), tert-butyl 4-(4,4,5,5-tetramethyl-1,3,2-dioxaborolan-2-yl)-5,6-dihydropyridine-1(2H)-carboxylate (2.30 g, 7.43 mmol, 1.50 eq), K₂CO₃ (1.37 g, 9.90 mmol, 2.00 eq), and Pd(dppf)Cl₂ (724 mg, 0.99 mmol, 0.20 eq) in dioxane/H₂O (15 mL, 5/1) was stirred at 100° C. for 4 h. The mixture was filtered and extracted with EA. The combined organic layers were dried over anhydrous Na₂SO₄, filtered, and concentrated. The residue was purified by silica flash column PE/EtOAc (10:1) to give product as yellow solid.

### Step 2: tert-Butyl 4-(4-aminophenyl)piperidine-1-carboxylate

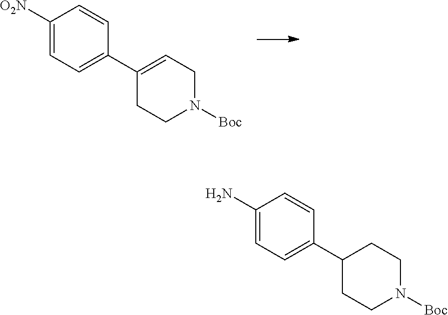 [SMILES: CC(C)(C)OC(=O)N1CC=C(c2ccc([N+](=O)[O-])cc2)CC1 ; CC(C)(C)OC(=O)N1CCC(c2ccc(N)cc2)CC1]

A mixture of tert-butyl 4-(4-nitrophenyl)-5,6-dihydropyridine-1(2H)-carboxylate (1.20 g, 3.95 mmol, 1.00 eq), Pd/C (360 mg) in MeOH/THF (30 mL, 1:1) was stirred at 45° C. under H₂ overnight. The mixture was filtered and concentrated, and the residue was purified by silica flash column PE/EtOAc (3:1) to give product as yellow solid.

### Step 3: tert-Butyl 4-(4-((2,6-dioxopiperidin-3-yl)amino)phenyl)piperidine-1-carboxylate

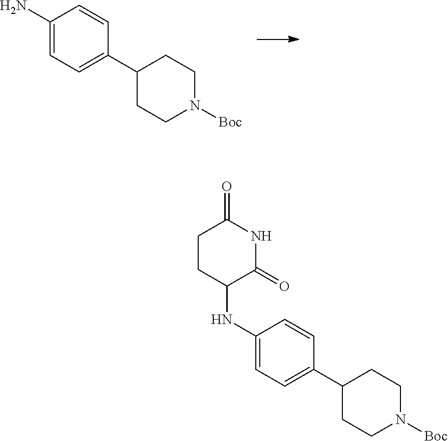 [SMILES: CC(C)(C)OC(=O)N1CCC(c2ccc(N)cc2)CC1 ; CC(C)(C)OC(=O)N1CCC(c2ccc(NC3CCC(=O)NC3=O)cc2)CC1]

A mixture of tert-butyl 4-(4-aminophenyl)piperidine-1-carboxylate (332 mg, 1.20 mmol, 1.00 eq.), 3-bromopiperidine-2,6-dione (242 mg, 1.26 mmol, 1.05 eq.) and NaHCO₃ (302 mg, 3.60 mmol, 3.00 eq.) in DMF (4.0 mL) was stirred at 70° C. for overnight. The mixture was diluted with water and extracted with EA The combined organic layers were dried over anhydrous Na₂SO₄, filtered, and then concentrated. The residue was purified by silica flash column PE/EtOAc (1:1) to give the title compound as yellow solid.

### Step 4: 3-((4-(Piperidin-4-yl)phenyl)amino)piperidine-2,6-dione

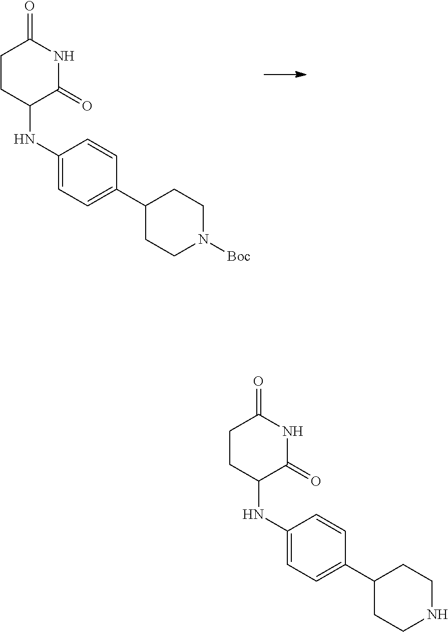 [SMILES: CC(C)(C)OC(=O)N1CCC(c2ccc(NC3CCC(=O)NC3=O)cc2)CC1 ; O=C1CCC(Nc2ccc(C3CCNCC3)cc2)C(=O)N1]

TFA (0.5 mL) was added to a mixture of tert-butyl 4-(4-((2,6-dioxopiperidin-3-yl)amino)phenyl)piperidine-1-carboxylate (100 mg, 0.26 mmol, 1.00 eq.) in DCM (2.0 mL) and the mixture was stirred at rt for 2 h. The solution was concentrated to give the title compound as a yellow solid.

### Reference 47

### Synthesis of 3-(4-(piperazin-1-yl)phenyl)piperidine-2,6-dione

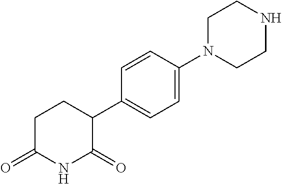 [SMILES: O=C1CCC(c2ccc(N3CCNCC3)cc2)C(=O)N1]

### Step 1: 2,6-Bis(benzyloxy)pyridine

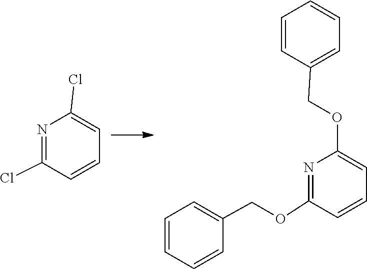 [SMILES: Clc1cccc(Cl)n1 ; c1ccc(COc2cccc(OCc3ccccc3)n2)cc1]

To a stirred solution of phenylmethanol (14.60 g, 135.14 mmol, 2.00 eq.) in THF (250.0 mL) were added t-BuOK (38.00 g, 337.84 mmol, 5.00 eq.) and 2,6-dichloropyridine (10.00 g, 67.57 mmol, 1.00 eq.). The mixture was stirred at 75° C. for 20 h under N₂. The mixture was diluted with water and extracted with EA, and the combined organic layers was washed with brine, dried over Na₂SO₄, concentrated to give the title compound as pale yellow solid.

### Step 2: 2,6-Bis(benzyloxy)-3-bromopyridine

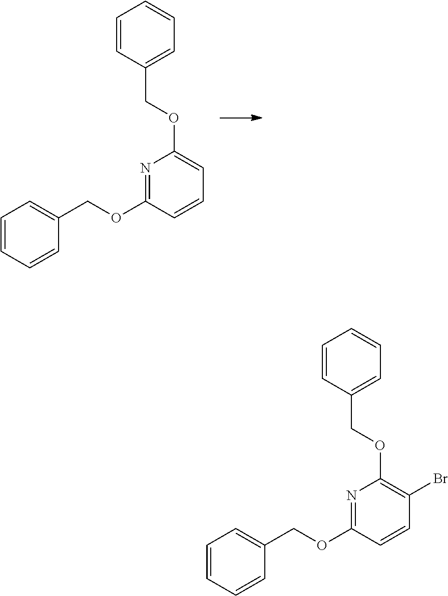 [SMILES: c1ccc(COc2cccc(OCc3ccccc3)n2)cc1 ; Brc1ccc(OCc2ccccc2)nc1OCc1ccccc1]

NBS (8.70 g, 0.05 mol, 0.95 eq.) was added to a stirred solution of 2,6-bis(benzyloxy)-pyridine (15.00 g, 0.05 mol, 1.00 eq.) in MeCN (100.0 mL) and the mixture was stirred at 80° C. for 4 h under N₂. The mixture was diluted with water and extracted with EA. The combined organic layers was washed with brine, dried over Na₂SO₄, concentrated to give the title compound as yellow solid.

### Step 3: 2,6-Bis(benzyloxy)-3-(4,4,5,5-tetramethyl-1,3,2-dioxaborolan-2-yl)pyridine

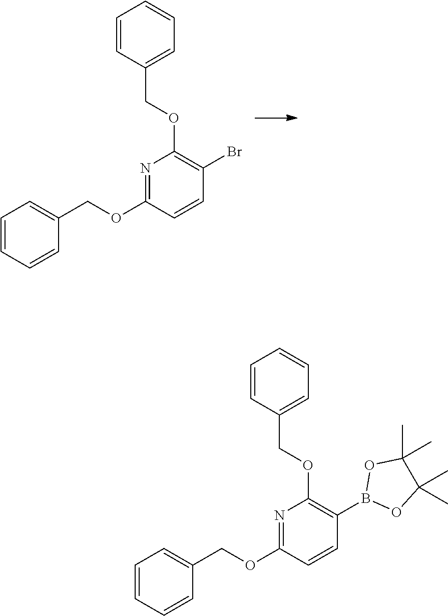 [SMILES: Brc1ccc(OCc2ccccc2)nc1OCc1ccccc1 ; CC1(C)OB(c2ccc(OCc3ccccc3)nc2OCc2ccccc2)OC1(C)C]

A mixture of 2,6-bis(benzyloxy)-3-bromopyridine (19.00 g, 0.05 mol, 1.00 eq.), 4,4,4′,4′,5,5,5′,5′-octamethyl-2,2′-bi(1,3,2-dioxaborolane) (19.60 g, 0.08 mol, 1.50 eq.), KOAc (10.00 g, 0.10 mol, 2.00 eq.), and Pd(dppf)Cl₂ (3.7 g, 5.00 mmol, 0.10 eq.) in 1,4-dioxane (200.0 mL) was stirred at 100° C. for 25 h under N₂. The mixture was diluted with water and extracted with EA, and the combined organic layer was washed with brine, dried over Na₂SO₄, and concentrated. The residue was purified by silica gel column chromatography eluting with EA:PE=0 to 100% to give title compound as yellow solid.

### Step 4: 2,6-Bis(benzyloxy)-3-(4-bromophenyl)pyridine

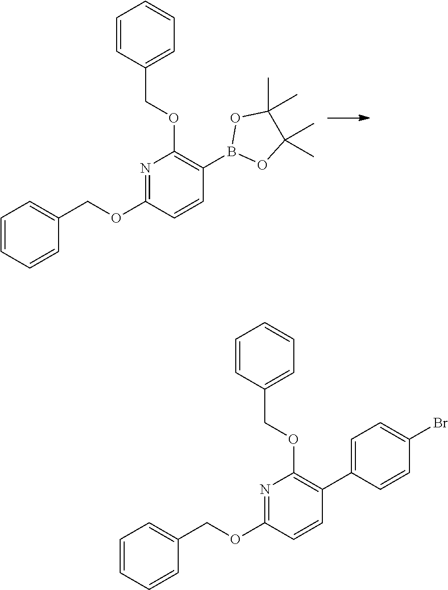 [SMILES: CC1(C)OB(c2ccc(OCc3ccccc3)nc2OCc2ccccc2)OC1(C)C ; Brc1ccc(-c2ccc(OCc3ccccc3)nc2OCc2ccccc2)cc1]

A mixture of 2,6-bis(benzyloxy)-3-(4,4,5,5-tetramethyl-1,3,2-dioxaborolan-2-yl)pyridine (4.42 g, 10.60 mmol, 1.20 eq.), 1-bromo-4-iodobenzene (2.50 g, 8.83 mol, 1.00 eq.), K₃PO₄ (5.63 g, 26.50 mmol, 3.00 eq.), and Pd(PPh₃)₄ (510 mg, 0.44 mmol, 0.05 eq.) in 1,4-dioxane/H₂O=10:1 (40.0 mL) was stirred at 100° C. for 16 h under N₂. The mixture was diluted with water and extracted with EA, and the combined organic layer was washed with brine, dried over Na₂SO₄, and concentrated. The residue was purified by silica gel column chromatography eluting with EA:PE=0 to 100% to give the title compound as yellow solid.

### Step 5: tert-Butyl 4-(4-(2,6-bis(benzyloxy)pyridin-3-yl)phenyl)piperazine-1-carboxylate

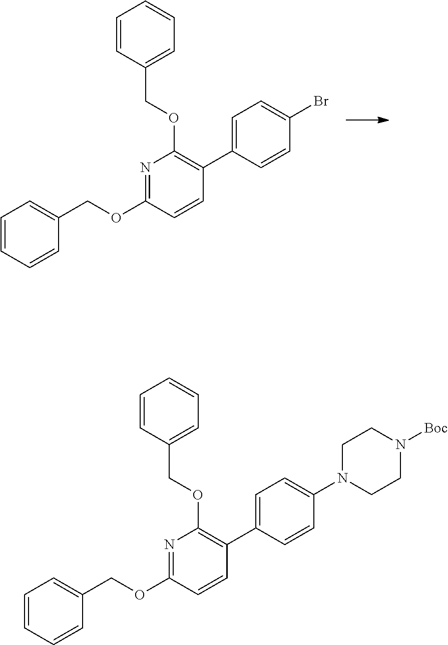 [SMILES: Brc1ccc(-c2ccc(OCc3ccccc3)nc2OCc2ccccc2)cc1 ; CC(C)(C)OC(=O)N1CCN(c2ccc(-c3ccc(OCc4ccccc4)nc3OCc3ccccc3)cc2)CC1]

A mixture of 2,6-bis(benzyloxy)-3-(4-bromophenyl)pyridine (500 mg, 1.12 mmol, 1.00 eq.), tert-butyl piperazine-1-carboxylate (417 mg, 2.24 mmol, 2.00 eq.), Cs₂CO₃ (730 mg, 2.24 mmol, 2.00 eq.), Pd₂(dba)₃ (51 mg, 0.06 mmol, 0.05 eq.), and Ruphos (52 mg, 0.11 mmol, 0.10 eq.) in toluene (15.0 mL) was stirred at 110° C. for 20 h under N₂. The mixture was diluted with water and extracted with EA, and the combined organic layer was washed with brine, dried over Na₂SO₄, concentrated. The residue was purified by silica gel column chromatography eluting with EA:PE=0 to 100% to give the title compound as yellow solid.

### Step 6: tert-Butyl 4-(4-(2,6-dioxopiperidin-3-yl)phenyl)piperazine-1-carboxylate

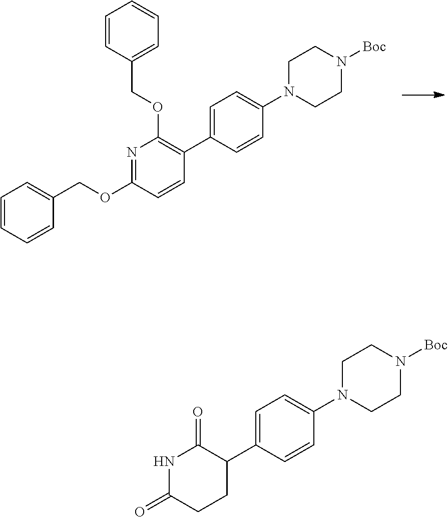 [SMILES: CC(C)(C)OC(=O)N1CCN(c2ccc(-c3ccc(OCc4ccccc4)nc3OCc3ccccc3)cc2)CC1 ; CC(C)(C)OC(=O)N1CCN(c2ccc(C3CCC(=O)NC3=O)cc2)CC1]

A mixture of tert-butyl 4-(4-(2,6-bis(benzyloxy)pyridin-3-yl)phenyl)piperazine-1-carboxylate (260 mg, 0.47 mmol, 1.00 eq.), 10% Pd/C (260 mg) in EA (5.0 mL) and 1,4-dioxane (5.0 mL) was stirred at r.t for 20 h under H₂. The mixture was filtered and the filtrate was concentrated to give the title compound as yellow oil.

### Step 7: 3-(4-(Piperazin-1-yl)phenyl)piperidine-2,6-dione

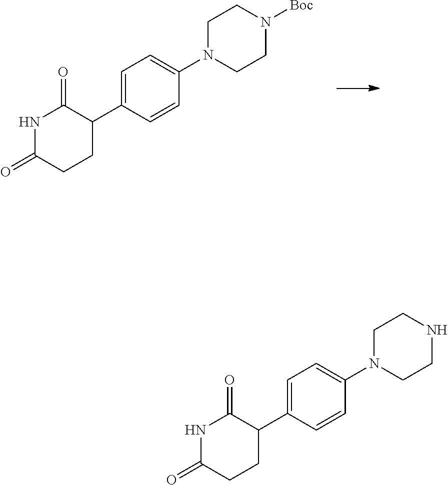 [SMILES: CC(C)(C)OC(=O)N1CCN(c2ccc(C3CCC(=O)NC3=O)cc2)CC1 ; O=C1CCC(c2ccc(N3CCNCC3)cc2)C(=O)N1]

TFA (0.5 mL) was added to a stirred solution of tert-butyl 4-(4-(2,6-dioxopiperidin-3-yl)phenyl)piperazine-1-carboxylate (160 mg, 0.43 mmol, 1.00 eq.) in DCM (2.0 mL) and the mixture was stirred at r.t for 2 h under N₂. The mixture was concentrated to give the title compound as its TFA salt as yellow oil.

### Reference 48

### Synthesis of 8-methyl-2-(methylsulfonyl)pyrido[2,3-d]pyrimidin-7(8H)-one

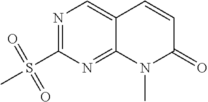 [SMILES: Cn1c(=O)ccc2cnc(S(C)(=O)=O)nc21]

### Step 1: Ethyl 4-(methylamino)-2-(methylthio)pyrimidine-5-carboxylate

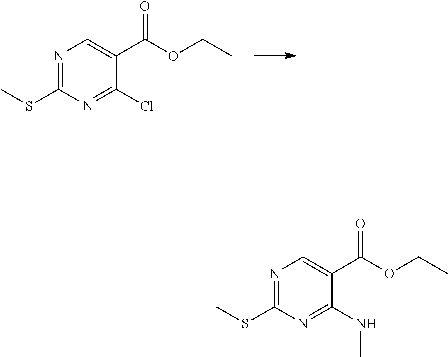

A mixture of ethyl 4-chloro-2-(methylthio)pyrimidine-5-carboxylate (5.00 g, 21.55 mmol, 1.00 eq.), methanamine (8.75 ml) in DCM (120.0 mL) was stirred 12 h at 0° C. The mixture was diluted with water and extracted with DCM. The combined organic layer was washed with brine, dried over anhydrous Na₂SO₄ and concentrated. The residue was purified by silica gel column chromatography eluting with PE/EtOAc (10:1) to afford the title compound as a white solid.

### Step 2: (4-(Methylamino)-2-(methylthio)pyrimidin-5-yl)methanol

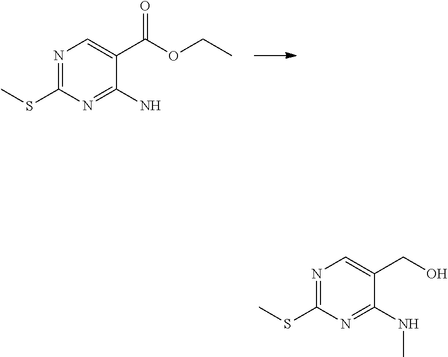

A mixture of ethyl 4-(methylamino)-2-(methylthio)pyrimidine-5-carboxylate (2.90 g, 12.78 mmol, 1.00 eq.) and LiAlH₄ (971 mg, 25.55 mmol, 2.00 eq.) in THF (45 mL) was stirred for 4 h at room temperature, followed by addition of Na₂SO₄·10 H₂O. The mixture was diluted with water and extracted with DCM. The combined organic layer was washed with brine, dried over anhydrous Na₂SO₄ and concentrated to give the title compound as white solid.

### Step 3: 4-(Methylamino)-2-(methylthio)pyrimidine-5-carbaldehyde

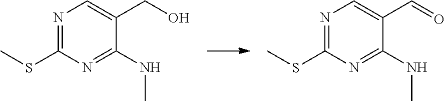

To a stirred solution of (4-(methylamino)-2-(methylthio)pyrimidin-5-yl)methanol (500 mg, 2.70 mmol, 1.00 eq.) in DCM (2.0 mL) was added MnO₂ (2.20 g, 27.00 mmol, 10.00 eq.) at −30° C. and the mixture was stirred 16 h. The mixture was filtered and concentrated to afford the crude title compound as a white solid.

### Step 4: 8-Methyl-2-(methylthio)pyrido[2,3-d]pyrimidin-7(8H)-one

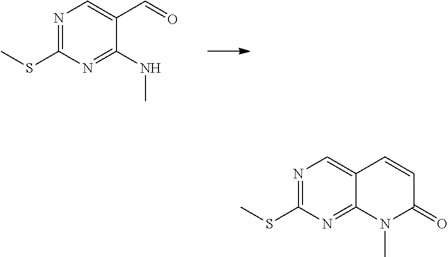

Ethyl acetate (529 mg, 6.00 mmol, 2.20 eq) was added to a stirred solution of LiHMDS (8.2 mL, 8.20 mmol, 3.00 eq.) in THF (8.0 mL) at −70° C. under nitrogen atmosphere. The mixture was stirred for 0.25 h at −70° C., 4-(methylamino)-2-(methylthio)pyrimidine-5-carbaldehyde (500 mg, 2.73 mmol, 1.00 eq.) was added and the mixture was allowed to warm to rt slowly. After 2 h, the mixture was diluted with NH₄Cl aq., and extracted with DCM. The combined organic layer was washed with brine, dried over anhydrous Na₂SO₄, filtered and concentrated. The residue was purified by silica gel column chromatography eluted with PE/EtOAc (1:1) to afford the title compound as a white solid.

### Step 5: 8-Methyl-2-(methylsulfonyl)pyrido[2,3-d]pyrimidin-7(8H)-one

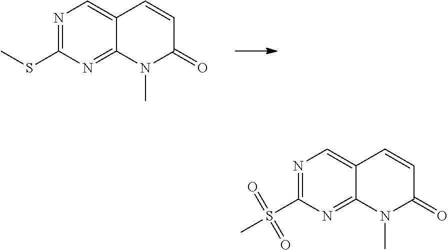 [SMILES: CSc1ncc2ccc(=O)n(C)c2n1 ; Cn1c(=O)ccc2cnc(S(C)(=O)=O)nc21]

Oxone (1.60 g, 2.60 mmol, 2.00 eq.) was added to a mixture of 8-methyl-2-(methylthio)pyrido[2,3-d]pyrimidin-7(8H)-one (270 mg, 1.30 mmol, 1.00 eq.) in THF:MeOH:H₂O=2:2:1 (15.0 mL). The mixture was stirred 2 h at 30° C. under nitrogen atmosphere, then the mixture was diluted with water, and extracted with DCM. The combined organic layer was washed with water, dried over anhydrous Na₂SO₄, filtered, and concentrated to afford the title compound as a yellow solid.

### Reference 49

### Synthesis of 2-(methylsulfonyl)-8-(2,2,2-trifluoroethyl)pyrido[2,3-d]pyrimidin-7(8H)-one

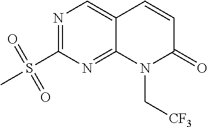 [SMILES: CS(=O)(=O)c1ncc2ccc(=O)n(CC(F)(F)F)c2n1]

### Step 1: Ethyl 4-amino-2-(methylthio)pyrimidine-5-carboxylate

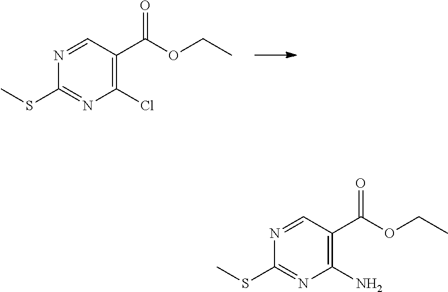

NH₃·H₂O (162 mg, 1.61 mmol, 1.51 eq.) and TEA (3.88 mg, 3.88 mmol, 3.00 eq.) were added to a stirred solution of ethyl 4-chloro-2-(methylthio)pyrimidine-5-carboxylate (300 mg, 1.24 mmol, 1.00 eq.) in THF (5.0 mL), and the mixture was stirred at rt 16 h. The mixture was diluted with water and extracted with EtOAc. The combined organic layer was dried over Na₂SO₄, and concentrated, and the residue was purified by silica gel column chromatography eluting with PE/EtOAc (5:1) to give the title compound as a yellow solid.

### Step 2: (4-Amino-2-(methylthio)pyrimidin-5-yl)methanol

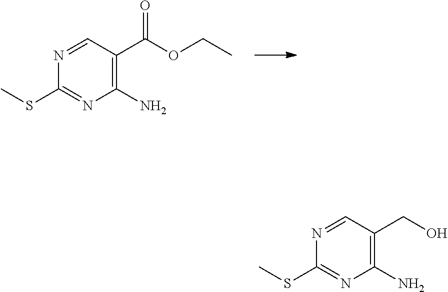

Ethyl 4-amino-2-(methylthio)pyrimidine-5-carboxylate (300 mg, 1.41 mmol, 1.00 eq.) in THF (3.0 mL) was added slowly to a stirred mixture of LiAlH₄ (65 mg, 2.12 mmol, 1.50 eq.) in THF (5.0 mL) at 0° C. under N₂. The mixture was stirred at rt for 4 h, and the mixture was quenched with Na₂SO₄·10H₂O and then filtered. The filtrate was concentrated and the residue was purified by silica gel column chromatography eluting with PE/EtOAc (1:1) to give the title compound as a white solid.

### Step 3: 4-Amino-2-(methylthio)pyrimidine-5-carbaldehyde

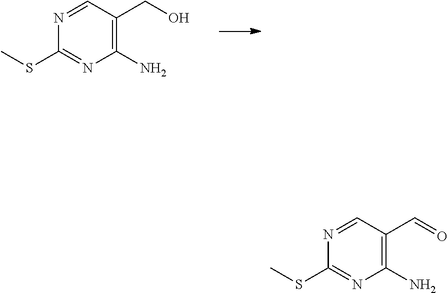

MnO₂ (1759 mg, 20.63 mmol, 8.00 eq.) was added to a stirred solution of (4-amino-2-(methylthio)pyrimidin-5-yl)methanol (442 mg, 2.58 mmol, 1.00 eq.) in DCM, and the mixture was stirred at rt 16 h under N₂. The mixture was filtered, and concentrated. The residue was purified by silica gel column chromatography eluting with PE/EtOAc (3:1) to give the title compound as a yellow solid.

### Step 4: Ethyl (E)-3-(4-amino-2-(methylthio)pyrimidin-5-yl)acrylate

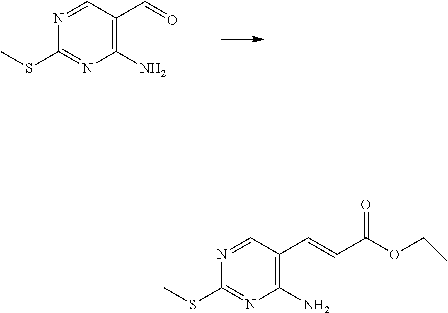 [SMILES: CSc1ncc(C=O)c(N)n1 ; CCOC(=O)/C=C/c1cnc(SC)nc1N]

A mixture of 4-amino-2-(methylthio)pyrimidine-5-carbaldehyde (1.50 g, 8.88 mmol, 1.00 eq.) and ethyl 2-(triphenyl-λ⁵-phosphanylidene)acetate (4.63 g, 13.3 mmol, 1.50 eq.) in THF (40 mL) was refluxed at 75° C. for 3 h under N₂ and then at rt 16 h. The mixture was concentrated and the residue was purified by silica gel column chromatography eluting with PE/EtOAc (5:1) to give the title compound as a white solid.

### Step 5: 2-(Methylthio)pyrido[2,3-d]pyrimidin-7(8H)-one

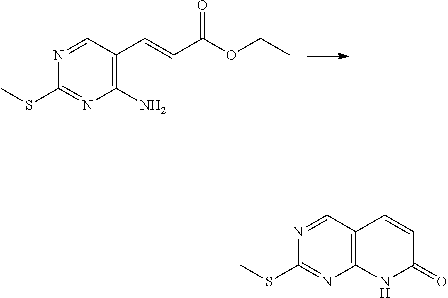

A mixture of ethyl (E)-3-(4-amino-2-(methylthio)pyrimidin-5-yl)acrylate (2.78 g, 11.63 mmol, 1.00 eq.) and NaSCH₃ (1.63 g, 23.26 mmol, 2.00 eq.) in EtOH was stirred at 80° C. for 2 h under N₂. The mixture was concentrated and then diluted with water. 1M HCl aq. was added to the mixture until the pH=5. The resulting white precipitation was filtered and washed with water and EA to give the title compound as a white solid

### Step 6: 2-(Methylthio)-8-(2,2,2-trifluoroethyl)pyrido[2,3-d]pyrimidin-7(8H)-one

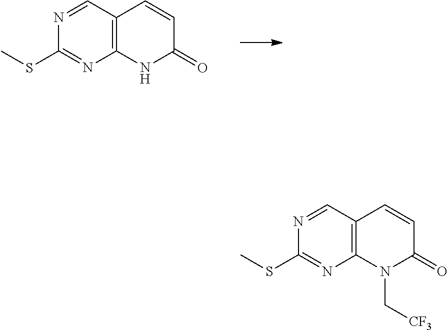 [SMILES: CSc1ncc2ccc(=O)[nH]c2n1 ; CSc1ncc2ccc(=O)n(CC(F)(F)F)c2n1]

NaH (94 mg, 2.33 mmol, 1.50 eq.) was added to a solution of 2-(methylthio)pyrido[2,3-d]-pyrimidin-7(8H)-one (300 mg, 1.55 mmol, 1.00 eq.) in DMF at 0° C. and the mixture was stirred for 15 min. 2,2,2-Trifluoroethyl trifluoromethanesulfonate (1.19 g, 5.13 mmol, 3.30 eq.) in DMF was added to the mixture at 0° C. and the mixture was allowed to warm to rt and stirred for 16 h. The mixture was diluted with water and extracted with EtOAc. The organic layer was dried over Na₂SO₄ and concentrated. The residue was purified by silica gel column chromatography eluting with PE/EtOAc (3:1) to give title compound as a white solid.

### Step 7: 2-(Methylsulfonyl)-8-(2,2,2-trifluoroethyl)pyrido[2,3-d]pyrimidin-7(8H)-one

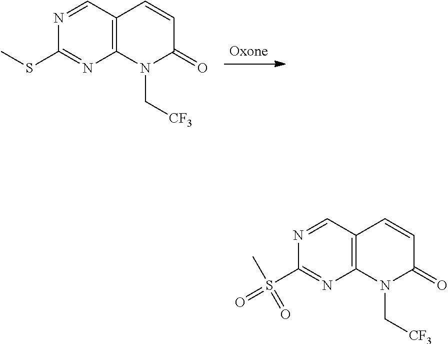 [SMILES: CSc1ncc2ccc(=O)n(CC(F)(F)F)c2n1 ; CS(=O)(=O)c1ncc2ccc(=O)n(CC(F)(F)F)c2n1]

Oxone (447 mg, 0.73 mmol, 2.00 eq.) was added to a solution of 2-(methylthio)-8-(2,2,2-trifluoroethyl)pyrido[2,3-d]pyrimidin-7(8H)-one (100 mg, 0.36 mmol, 1.00 eq.) in THF/H₂O 3:1 (3 ml/1 ml) and the mixture stirred at rt 12 h under N₂. The mixture was diluted with H₂O and extracted with DCM. The organic layer was washed with brine, and dried over Na₂SO₄ and concentrated. The residue was purified by silica gel column chromatography eluting with PE/EtOAc (1:1) to give title compound as a white solid.

### Example 1

### Synthesis of rac-1-(6-(1-(3-(((3R,4S)-3-fluoro-4-((8-methyl-7-oxo-7,8-dihydropyrido[2,3-d]-pyrimidin-2-yl)amino)piperidin-1-yl)sulfonyl)benzyl)piperidin-4-yl)-1-methyl-1H-indazol-3-yl)-dihydropyrimidine-2,4(1H,3H)-dione

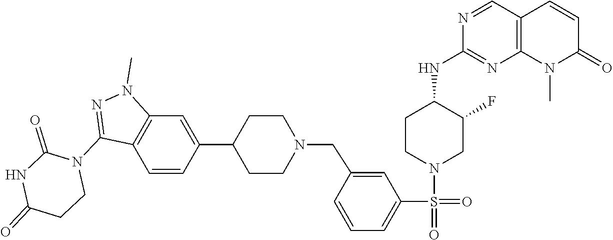 [SMILES: Cn1nc(N2CCC(=O)NC2=O)c2ccc(C3CCN(Cc4cccc(S(=O)(=O)N5CC[C@H](Nc6ncc7ccc(=O)n(C)c7n6)[C@H](F)C5)c4)CC3)cc21]

A mixture of rac-1-(6-(1-(3-(((3R,4S)-4-amino-3-fluoropiperidin-1-yl)sulfonyl)benzyl)-piperidin-4-yl)-1-methyl-1H-indazol-3-yl)dihydropyrimidine-2,4(1H,3H)-dione (38.5 mg, 0.07 mmol, 1.00 eq.), DIEA (26 mg, 0.20 mmol, 3.00 eq.) and 8-methyl-2-(methylsulfonyl)pyrido-[2,3-d]pyrimidin-7(8H)-one (21 mg, 0.09 mmol, 1.30 eq) in DMSO (3.0 mL) was stirred at 65° C. 16 h. The mixture was diluted with water and extracted with DCM. The combined organic layer was washed with water, brine and dried over Na₂SO₄. The organic layer was concentrated and purified by prep-TLC (DCM:MeOH=20:1) to give the title compound as white solid.

### Example 2

### Synthesis of rac-1-(6-(1-(3-(((3R,4S)-3-fluoro-4-((7-oxo-8-(2,2,2-trifluoroethyl)-7,8-dihydropyrido[2,3-d]pyrimidin-2-yl)amino)piperidin-1-yl)sulfonyl)benzyl)piperidin-4-yl)-1-methyl-1H-indazol-3-yl)dihydropyrimidine-2,4(1H,3H)-dione

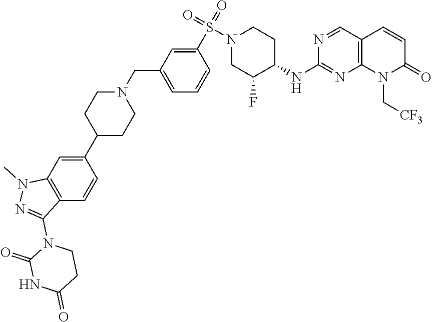 [SMILES: Cn1nc(N2CCC(=O)NC2=O)c2ccc(C3CCN(Cc4cccc(S(=O)(=O)N5CC[C@H](Nc6ncc7ccc(=O)n(CC(F)(F)F)c7n6)[C@H](F)C5)c4)CC3)cc21]

A mixture of rac-1-(6-(1-(3-(((3R,4S)-4-amino-3-fluoropiperidin-1-yl)sulfonyl)benzyl)-piperidin-4-yl)-1-methyl-1H-indazol-3-yl)dihydropyrimidine-2,4(1H,3H)-dione (49 mg, 0.083 mmol, 1.00 eq.), DIPEA (33 mg, 0.25 mmol, 3.00 eq.), and 2-(methylsulfonyl)-8-(2,2,2-trifluoroethyl)pyrido[2,3-d]pyrimidin-7(8H)-one (28 mg, 0.0915 mmol, 1.10 eq.) in DMSO (2.0 mL) was stirred at 65° C. under nitrogen atmosphere overnight. The resulting mixture was diluted with water and extracted with EA. The combined organic layer was washed with brine, dried over anhydrous Na₂SO₄, filtered and then concentrated. The residue was purified by silica gel column chromatography, eluted with DCM/MeOH (20:1) to afford the title compound as a white solid. MS (ES, m/z): [M+1]⁺=825.0

### BIOLOGICAL EXAMPLES

### Example 1

### Inhibition of CDK2: Phospho-Rb Measurement in Cells

Phosphorylation of RB protein at S807/811 were measured using HTRF phospho-RB cellular kits (Cat #64RBS807PEG) from Cisbio.

On Day 1, OVCAR3 (CDK2 dependent cell line) cells were seeded into 96-well tissue-culture treated plates at 20,000 cells/well in 200 μL and incubated overnight at 37° C. in CO₂ atmosphere. On Day 2, the cells were treated with test compounds at concentrations from 0.3 to 10,000 nM using HP D300 digital dispenser. Twenty-four hours after compound treatment, cell culture media was removed by flicking the plate and tapping the plate against clean paper towel. Immediately 30 μL 1× lysis buffer was supplemented from the kit and the plate was incubated at room temperature on shaker for 30 min. After homogenization by pipetting up and down, 8 μL cell lysate from 96-well cell culture plate was transferred to 384-well small volume white detection plate. 2 μL premixed detection solution was added and the plate was covered with sealer. To prepare the detection solution, d2 conjugated-phospho-RB antibody and Eu-cryptate conjugated phosphor-RB antibody were diluted into detection buffer following manufacturer's instruction. Detection plates were incubated for 4 h at room temperature and read on ClarioStar (BMG Labtech) in TR-FRET mode (665 nM and 620 nM). The TR-FRET ratio (665 nM/620 nM) was plotted against the compound concentration and normalized to DMSO controls. Half maximal inhibition concentration (IC₅₀) values are calculated with a four-parameter logistic fit using GraphPad Prism (version 8; La Jolla, CA).

CDK2 IC₅₀ of PROTAC compound 1 and compound 2 in pRB(S807/811) assay are reported in Table 1 below. In the table below, A indicates an IC₅₀ of greater than or equal to 0.1 μM but less than 1.0 μM; and B indicates an IC₅₀ of greater than or equal to 1.0 μM but less than or equal to 10 μM.

TABLE 1

| Cpd No. | pRb |
|---|---|
| (Cpd Table | OVCAR3 |
| I) | (μM) |
| 1 | A |
| 2 | B |

### Example 2

### High-Throughput Measurement of Cellular Endogenous CDK2

Effects of compounds on cellular CDK2 level can be monitored by a high-throughput HTRF assay.

To determine half maximal degradation concentration (DC₅₀) values of compounds, cellular CDK2 level was measured in 96-well format using HTRF total CDK2 cellular kit (Cat #64CDK2TPEG) from Cisbio.

On Day 1, OVCAR3 cells were seeded into 96-well tissue-culture treated plates at 20,000 cells/well in 200 μL and incubated overnight at 37° C. in C02 atmosphere. On Day 2 cells were treated with compounds at concentration ranging from 0.3 to 10,000 nM using HP D300 digital dispenser. 24 hours after compound treatment, cell culture media was removed by flicking the plate and tapping the plate against clean paper towel. Immediately 30 μL 1× lysis buffer was supplemented from the kit and the plate was incubated at room temperature on shaker for 30 min. After homogenization by pipetting up and down, 8 μL cell lysate from 96-well cell culture plate was transferred to 384-well small volume white detection plate. 2 μL premixed detection solution was added and the plate was covered with sealer. To prepare the detection solution, d2 conjugated-CDK2 antibody and Eu-cryptate conjugated CDK2 antibody were diluted into detection buffer following manufacturer's instruction. Detection plates were incubated overnight at room temperature and read on ClarioStar (BMG Labtech) in TR-FRET mode (665 nM and 620 nM). The TR-FRET ratio (665 nM/620 nM) was plotted against the compound concentration and normalized to DMSO controls. Half maximal degradation concentration (DC₅₀) values were calculated with a four-parameter logistic fit using GraphPad Prism (version 8; La Jolla, CA).

TABLE 2

| Cpd | DC₅₀ | |
|---|---|---|
| No. | OVCAR3_6 h | |
| 1 | 41.9 | nM |
| 2 | 1312 | nM |

### FORMULATION EXAMPLES

The following are representative pharmaceutical formulations containing a compound of the present disclosure.

### Tablet Formulation

The following ingredients are mixed intimately and pressed into single scored tablets.

| Ingredient | Quantity per tablet (mg) |
|---|---|
| compound Formula (I) | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| Ingredient | Quantity per capsule (mg) |
|---|---|
| compound Formula (I) | 200 |
| lactose spray dried | 148 |
| magnesium stearate | 2 |

### Injectable Formulation

Compound of the disclosure (e.g., compound 1) in 2% HPMC, 1% Tween 80 in DI water, pH 2.2 with MSA, q.s. to at least 20 mg/mL

### Inhalation Composition

To prepare a pharmaceutical composition for inhalation delivery, 20 mg of a compound disclosed herein is mixed with 50 mg of anhydrous citric acid and 100 mL of 0.9% sodium chloride solution. The mixture is incorporated into an inhalation delivery unit, such as a nebulizer, which is suitable for inhalation administration.

### Topical Gel Composition

To prepare a pharmaceutical topical gel composition, 100 mg of a compound disclosed herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Ophthalmic Solution Composition

To prepare a pharmaceutical ophthalmic solution composition, 100 mg of a compound disclosed herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Nasal Spray Solution

To prepare a pharmaceutical nasal spray solution, 10 g of a compound disclosed herein is mixed with 30 mL of a 0.05M phosphate buffer solution (pH 4.4). The solution is placed in a nasal administrator designed to deliver 100 ul of spray for each application.

What is claimed:

1. A compound of Formula (I): 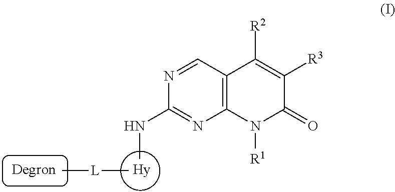
wherein:
Degron is an E3 ligase ligand selected from:
(a) a group of formula (i): 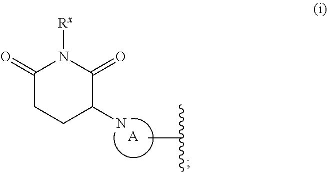
and
(b) a group of formula (ii): 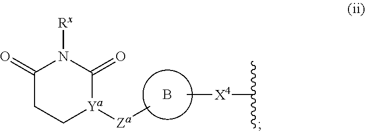
where:
Rₓ is hydrogen;
Y^{a} is CH or N;
Z^{a} is a bond, —CH₂—, —NH—, —O—, or —NHC(O)— where NH of —NHC(O)— is attached to Y^{a};
ring A of the E3 ligase ligand of formula (ii) is a group of formula (a), (b), or (c): 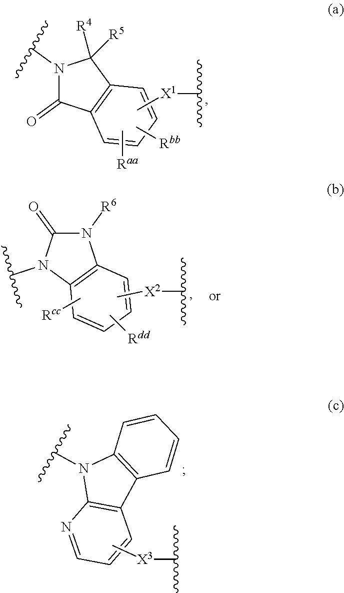
where:
R^{aa}, R^{bb}, R^{cc}, and R^{dd} are independently selected from hydrogen, alkyl, alkoxy, halo, haloalkyl, haloalkoxy, and cyano;
R⁴ and R⁵ are independently hydrogen or alkyl; or R⁴ and R⁵ together with the carbon to which they are attached form >C═O; and
R⁶ is hydrogen or alkyl;
ring B of the E3 ligase ligand of formula (ii) is phenylene, cyclylaminylene, a 5- or 6-membered monocyclic heteroarylene, or a 9- or 10-membered fused bicyclic heteroarylene, wherein each heteroarylene ring contains one to three nitrogen ring atoms and further wherein the phenylene, cyclylaminylene, and each heteroarylene are independently substituted with R^{ee} and R^{ff} independently selected from hydrogen, alkyl, cycloalkyl, alkoxy, halo, haloalkyl, haloalkoxy, and cyano; and
X¹, X², X³, and X⁴ are independently a bond, alkylene, —O—, —(O-alkylene)-, -(alkylene-O)—, —(NR^{gg}-alkylene)-, -(alkylene-NR^{hh})—, 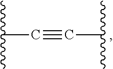 [SMILES: *C#C*]
—NH—, —N(alkyl)-, —C(═O)—, —NR^{jj}(═O)—, or —C(═O)NR^{kk}— where R^{gg}, R^{hh}, R^{jj}, and R^{kk} are independently hydrogen, alkyl, or cycloalkyl and each alkylene is optionally substituted with one or two fluoro;
Hy is cycloalkylene, arylene, heterocyclylene, bicyclic heterocyclylene, spiro heterocyclylene, bridged heterocyclylene, or fused heterocyclylene, where each of the aforementioned rings is substituted with R^{b}, R^{c}, and R^{d} independently selected from hydrogen, deuterium, alkyl, halo, haloalkyl, alkoxy, hydroxy, and cyano;
R¹ is alkyl, haloalkyl, hydroxyalkyl, alkoxyalkyl, aminoalkyl, aryl, heteroaryl, aralkyl, heteroaralkyl, cycloalkylalkyl, or heterocyclylalkyl, wherein aryl, by itself or as part of aralkyl, heteroaryl, by itself or as part of heteroaralkyl, cycloalkyl of cycloalkylalkyl, and heterocyclyl of heterocyclylalkyl are substituted with R^{e}, R^{f}, and R^{g} independently selected from hydrogen, deuterium, alkyl, haloalkyl, alkoxy, haloalkoxy, cyano, halo, and hydroxy;
R² is hydrogen, alkyl, or haloalkyl;
R³ is hydrogen, cyano, halo, NH₂, alkyl, or haloalkyl where alkyl and haloalkyl are optionally substituted with hydroxy, cyano, alkoxy, haloalkoxy, C(O)NH₂, and —C(O)OH; and
L is —Z¹—Z²—Z³—Z⁴—Z⁵—Z⁶— where:
Z¹ is a bond, alkylene, —C(O)NR—, —NR′(CO)—, —S(O)₂NR—, —NR′S(O)₂—, —(O-alkylene)ₐ-, -(alkylene-O)ₐ—, phenylene, monocyclic heteroarylene, or heterocyclylene, where each ring is substituted with R^{h} and R independently selected from hydrogen, deuterium, alkyl, alkoxy, halo, haloalkyl, and haloalkoxy;
Z² is a bond, alkylene, alkynylene, —C(O)—, —C(O)N(R)—, —NR′(CO)—, —(O-alkylene)_{b}-, -(alkylene-O)_{b}—, —O(CH₂)₇—, —O(CH₂)₈—, cycloalkylene, or heterocyclylene, where each ring is substituted with R^{j} and R^{k} independently selected from hydrogen, deuterium, alkyl, alkoxy, halo, haloalkyl, and haloalkoxy;
Z³ is a bond, alkylene, alkynylene, —C(O)NR—, —NR′(CO)—, —O—, —NR″—, —(O-alkylene)_{c}-, -(alkylene-O)_{c}—, cycloalkylene, spiro cyclolalkylene, phenylene, monocyclic heteroarylene, heterocyclylene, bicyclic heterocyclylene, bridged heterocyclylene, fused heterocyclylene, spiro heterocyclylene, or 11 to 13 membered spiro heterocyclylene, where each ring is substituted with R^{m} and Rⁿ independently selected from hydrogen, deuterium, alkyl, alkoxy, halo, haloalkyl, and haloalkoxy;
Z⁴ is a bond, alkylene, alkynylene, -(alkylene-NR″)—, —O—, —C(O)—, —NR″—, —(O-alkylene)_{d}-, -(alkylene-O)_{d}—, cycloalkylene, spiro cyclolalkylene, phenylene, heteroarylene, heterocyclylene, fused heterocyclylene, bridged heterocyclylene, or spiro heterocyclylene, where each ring is substituted with R^{o} and R^{p} independently selected from hydrogen, deuterium, alkyl, alkoxy, halo, haloalkyl, haloalkoxy, cyano, hydroxy, amino, alkylamino, and dialkylamino;
Z⁵ is a bond, alkylene, —NR″—, —O—, —C(O)—, —S(O)₂—, —NR′(CO)—, —C(O)NR—, phenylene, monocyclic heteroarylene, or heterocycylene, where each ring is substituted with R^{q} and R^{r} independently selected from hydrogen, deuterium, alkyl, alkoxy, halo, haloalkyl, and haloalkoxy, and
Z⁶ is a bond, alkylene, —NR″—, —O—, -(alkylene-O)—, —C(O)—, —S(O)₂—, —NR′(CO)—, or —C(O)NR—;
where each R, R′ and R″ is independently hydrogen or alkyl, each a, b, c, and d is independently an integer selected from 1 to 6, and each alkylene of —Z¹—, —Z²—, —Z³—, —Z⁴—, —Z⁵— and —Z⁶— is substituted with R^{s} and R^{t} where R^{s} is hydrogen or deuterium and R^{t} is hydrogen, deuterium, haloalkyl, hydroxy, alkoxy, cyano, cycloalkyl, heterocyclyl, aryl, or monocyclic heteroaryl, wherein cycloalkyl, heterocyclyl, aryl, and monocyclic heteroaryl are substituted with one or two substituents independently selected from hydrogen, alkyl, alkoxy, halo, haloalkyl, haloalkoxy, and cyano; provided that at least one of —Z¹—Z²—Z³—Z⁴—Z⁵—Z⁶— is not a bond; or a pharmaceutically acceptable salt thereof.

2. The compound of claim 1, or a pharmaceutically acceptable salt thereof, wherein
—Z⁵— is 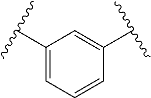 [SMILES: *c1cccc(*)c1]
substituted with R^{q} and R^{r}; and
Z⁶ is —S(O)₂—.

3. The compound of claim 1, or a pharmaceutically acceptable salt thereof, wherein:
X¹, X², X³, X⁴, Z¹, and Z² are each a bond;
Z³ is heterocyclylene, bridged heterocyclylene, or spiro heterocyclylene, where each ring is substituted with R^{m} and Rⁿ independently selected from hydrogen, deuterium, alkyl, alkoxy, halo, haloalkyl, and haloalkoxy;
Z⁴ is alkylene, —O—, cycloalkylene, or heterocyclylene, where each ring is substituted with R^{o} and R^{p} independently selected from hydrogen, deuterium, alkyl, alkoxy, halo, haloalkyl, haloalkoxy, cyano, hydroxy, amino, alkylamino, and dialkylamino;
Z⁵ is phenylene or monocyclic heteroarylene, each ring substituted with R^{q} and R^{r} independently selected from hydrogen, deuterium, alkyl, alkoxy, halo, haloalkyl, and haloalkoxy; and
Z⁶ is —S(O)₂—; and
wherein alkylene is substituted with R^{s} and R^{t}.

4. The compound of claim 1, 2, or 3, or a pharmaceutically acceptable salt thereof, wherein —Z³—Z⁴—Z^{S}—Z⁶— is: 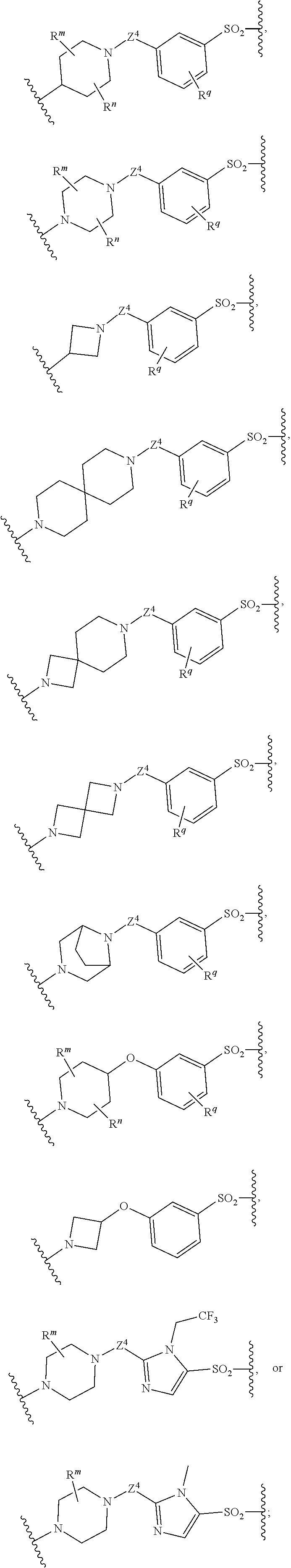
wherein R^{q}, R^{m}, and Rⁿ are independently selected from hydrogen, alkyl, cycloalkyl, halo, haloalkyl, haloalkoxy, alkoxy, and cyano.

5. The compound of claim 4, or a pharmaceutically acceptable salt thereof, wherein —Z³—Z⁴—Z⁵—Z⁶— is: 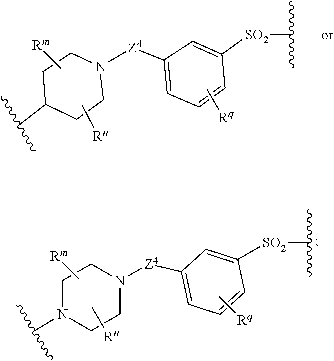
wherein each of R^{q} and R^{m} are independently selected from hydrogen, methyl, fluoro, chloro, cyano, methoxy, difluoromethoxy, cyclopropyl, difluoromethyl, and trifluoromethyl.

6. The compound of any one of claims 1 to 5, or a pharmaceutically acceptable salt thereof, wherein R¹ is alkyl, haloalkyl, hydroxyalkyl, alkoxyalkyl, or aminoalkyl.

7. The compound of any one of claims 1 to 6, or a pharmaceutically acceptable salt thereof, wherein R¹ is alkyl.

8. The compound of any one of claims 1 to 7, or a pharmaceutically acceptable salt thereof, wherein R³ is hydrogen, cyano, halo, haloalkyl, or alkyl optionally substituted with hydroxy.

9. The compound of any one of claims 1 to 8, or a pharmaceutically acceptable salt thereof, wherein R² is hydrogen.

10. The compound of any one of claims 1 to 9, or a pharmaceutically acceptable salt thereof, wherein Hy is heterocyclylene, phenylene, spiro heterocyclylene, or cycloalkylene, wherein each of the aforementioned rings is substituted with R^{b}, R^{c}, and R^{d} where R^{b} and R^{c} are independently selected from hydrogen, deuterium, alkyl, halo, haloalkyl, alkoxy, and hydroxy and R^{d} is hydrogen.

11. The compound of any one of claims 1 to 10, or a pharmaceutically acceptable salt thereof, wherein Hy is 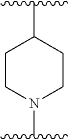 [SMILES: *C1CCN(*)CC1]
where the N atom of the piperidin-1,4-diyl ring is attached to L.

12. The compound of any one of claims 1 to 11, or a pharmaceutically acceptable salt thereof, wherein the Degron is an E3 ligase ligand of formula (i) where ring A:

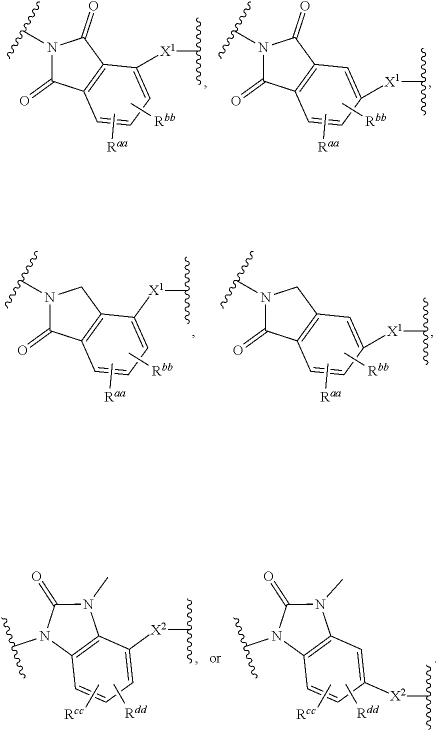

13. The compound of any one of claims 1 to 11, or a pharmaceutically acceptable salt thereof, wherein Degron is the E3 ligase ligand selected from: 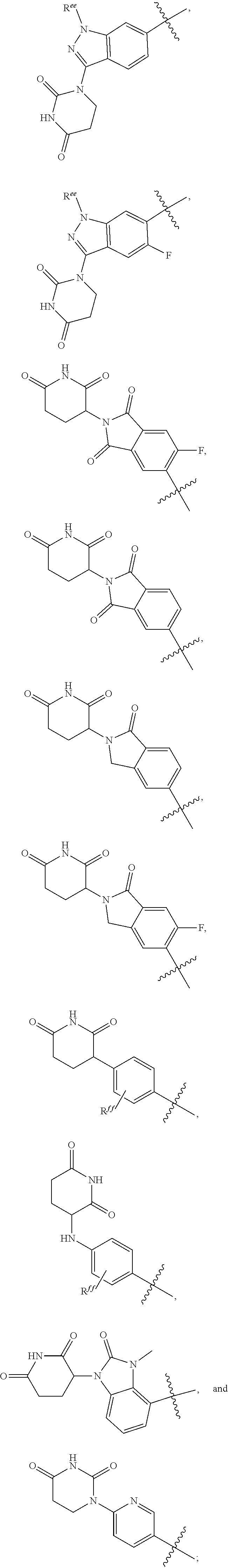
where R^{ee} is hydrogen, methyl, ethyl, cyclopropyl, or 2,2,2-trifluoroethyl and R^{ff} is hydrogen, methyl, cyclopropyl, fluoro, cyano, methoxy, difluoromethoxy, trifluoromethoxy, or trifluoromethyl.

14. A pharmaceutical composition comprising a compound of any one of claims 1 to 13, or a pharmaceutically acceptable salt thereof, and a pharmaceutically acceptable excipient.

15. A method of treating cancer in a patient which method comprises administering to the patient in recognized need thereof, a therapeutically effective amount a compound of any one of claims 1 to 13, or a pharmaceutically acceptable salt thereof, or with a pharmaceutical composition of claim 14.

16. The method of claim 15, wherein a) the compound, or a pharmaceutically acceptable salt thereof, or b) the pharmaceutical composition is administered in combination with at least one other anticancer agent.

17. The method of claim 15 or 16, wherein the cancer is lung cancer, skin cancer, bladder cancer, breast cancer, cervical cancer, colorectal cancer, cancer of the small intestine, colon cancer, rectal cancer, cancer of the anus, endometrial cancer, gastric cancer, head and neck cancer, liver cancer, ovarian cancer, prostate cancer, testicular cancer, uterine cancer, esophageal cancer, gall bladder cancer, pancreatic cancer, stomach cancer, thyroid cancer, or parathyroid cancer.
